(19) United States
(12) Patent Application Publication
(10) Pub. No.: US 2025/0025576 A1
(43) Pub. Date: Jan. 23, 2025

(54) CELL DELIVERY COMPOSITIONS AND METHODS OF USE THEREOF

(71) Applicant: The Regents of the University of California, Oakland, CA (US)

(72) Inventors: Ross C. Wilson, Berkeley, CA (US); Dana V. Foss, Berkeley, CA (US); John Hunsinger, Berkeley, CA (US); Daniel Carr, San Francisco, CA (US); Alexander Marson, San Francisco, CA (US); David-Huy Nguyen, San Francisco, CA (US); Srishti U. Sahu, Berkeley, CA (US)

(21) Appl. No.: 18/713,033

(22) PCT Filed: Nov. 22, 2022
(86) PCT No.: PCT/US2022/080336

§ 371 Date: May 23, 2024

Related U.S. Application Data
(60) Provisional application No. 63/282,622, filed on Nov. 23, 2021.

(51) Int. Cl. A61K 48/00; C07K 16/28; C12N 9/22; C12N 15/11; C12N 15/90

(52) U.S. Cl. CPC A61K 48/0058; A61K 48/0025; C07K 16/28; C12N 9/22; C12N 15/11; C12N 15/907; C07K 2319/01; C12N 2310/20

(57) ABSTRACT

The present disclosure provides a cargo delivery fusion polypeptide comprising an endosomolytic polypeptide and a cell penetrating polypeptide; and compositions comprising the cargo delivery fusion polypeptide. The present disclosure also provides methods of delivering a cargo into a target eukaryotic cell, using a composition comprising an amphiphilic polypeptide.

### CROSS-REFERENCE

This application claims the benefit of U.S. Provisional Patent Application No. 63/282,622, filed Nov. 23, 2021, which application is incorporated herein by reference in its entirety.

### STATEMENT REGARDING FEDERALLY SPONSORED RESEARCH

This invention was made with government support under AI150552 awarded by the National Institutes of Health. The government has certain rights in the invention.

### INCORPORATION-BY-REFERENCE OF MATERIAL ELECTRONICALLY SUBMITTED

A Sequence Listing is provided herewith as a Sequence Listing XML, “BERK-452WO_SEQ_LIST” created on Nov. 21, 2022 and having a size of 230 KB. The contents of the Sequence Listing XML are incorporated by reference herein in their entirety.

### INTRODUCTION

Biological macromolecules offer great potential as therapeutics. For example, DNA vaccines have been researched extensively, as they would provide a rapid and inexpensive approach to vaccination for a wide range of viral pathogens. DNA vaccines involve delivering DNA that encodes a viral protein into the nucleus of cells, where it can be transcribed; the viral protein is then produced and is recognized by the immune system. DNA vaccines have been hampered by poor delivery of the DNA into the target cells, resulting in an immune response that is not potent enough to result in effective vaccination. As a result, DNA vaccines have been delivered via in vivo electroporation, a painful process that requires specialized equipment and may require repeat dosing.

Genome editing holds immense therapeutic promise for correcting the genetic mutations underlying disease, or for preventing or treating non-genetic disease. Delivery of genome editing enzymes, such as Clustered Regularly Interspaced Short Palindromic Repeats (CRISPR)-associated (CRISPR-Cas) effector polypeptides, into the cytosol or nuclei of cells in need of manipulation remains the largest hurdle. Delivering a CRISPR-Cas effector polypeptide in the form of a ribonucleoprotein (RNP) complex with a guide RNA offers many advantages compared to other approaches (e.g. the use of viral vectors carrying DNA or lipid nanoparticles carrying mRNA).

There is a need in the art for compositions and methods for delivery of cargo such as DNA vaccines and genome editing enzymes into eukaryotic cells.

### SUMMARY

The present disclosure provides a cargo delivery fusion polypeptide comprising an endosomolytic polypeptide and a cell penetrating polypeptide; and compositions comprising the cargo delivery fusion polypeptide. The present disclosure also provides methods of delivering a cargo into a target eukaryotic cell, using a composition comprising an amphiphilic polypeptide.

### BRIEF DESCRIPTION OF THE DRAWINGS

FIG. 1 depicts various cargo delivery fusion polypeptides (SEQ ID Nos: 1-37 and 40-60, respectively).

FIG. 2 is a schematic depiction of delivery of CRISPR-Cas9 into target cells using a cargo delivery fusion polypeptide of the present disclosure.

FIG. 3 depicts screening of peptides 1-37 (as shown in FIG. 1) for Cas9-mediated knock-out (KO) at the beta-2 microglobulin (B2M) locus in human primary CD4⁺ T cells, as measured by flow cytometry.

FIG. 4A-4B depict screening of peptides for Cas9-mediated KO at the CD4 locus in human primary CD4+ T cells, as assessed by flow cytometry (FIG. 4A) or by deep sequencing (FIG. 4B).

FIG. 5A-5B depict screening of peptides #40-60 (as shown in FIG. 1) for Cas9-mediated KO at the β2M locus in human primary CD4⁺ T cells, as measured by flow cytometry (FIG. 5A); and live cell counts from each treatment condition as determined by live/dead fixable violet staining and flow cytometry (FIG. 5B).

FIG. 6 depicts the results of a screen of peptides #1-37 (as shown in FIG. 1) allowing Cas9-mediated non-homologous end joining (NHEJ) at the erythrocyte-specific BCL11a enhancer locus in CD34⁺ hematopoietic stem and progenitor cells (HSPCs), as measured by deep sequencing, at either 5 μM or 10 μM concentration of peptide.

FIG. 7A-7C depict screening of peptides #40-58 (as shown in FIG. 1) for promoting Cas9-3×-NLS-mediated NHEJ in HSPCs, as measured by deep sequencing, at either 5 UM or 10 UM final peptide concentration (FIG. 7A); HSPC viability under each condition (FIG. 7B); and a combined score for each peptide calculated by [(% Viability×% NHEJ)×100] (FIG. 7C).

FIG. 8A-8C depict the effect of a non-ionic surfactant additive on peptide-Cas9 formulations.

FIG. 9 depicts screening for Cas9 mediated genome editing in primary murine neural progenitor cells (NPCs) from Ai9 mice, as determined by tdTomato signal detected via flow cytometry.

FIG. 10 depicts screening for Cas9 mediated genome editing in primary murine neural progenitor cells (NPCs) from Ai9 mice, as determined by tdTomato signal by flow cytometry.

FIG. 11 depicts editing at the CD45 locus in B cells, T cells, and NK cells via either peptide coincubation or electroporation.

FIG. 12A-12B depict levels of β2M KO in T cells and B cells, either cultured separately or as a co-culture of T cells and B cells, as measured by flow cytometry (FIG. 12A); and the ratio of editing in T cells over B cells in several treatment conditions (FIG. 12B).

FIG. 13A-13C depict the percentage of primary human B cells displaying KO of β2M under different treatment conditions (FIG. 13A); representative flow scatter plots indicating gating strategy for analyzing β2M KO under four conditions (FIG. 13B); and live cell counts of cells 3 days after the treatments as measured by flow cytometry (FIG. 13C).

FIG. 14A-14C depict Knock-in (KI) via homology-directed repair (HDR) of a FLAG tag into the CD5 locus in primary human CD4⁺ T cells.

FIG. 15A-15C depict Knock in of 19287-chimeric antigen receptor (CAR) at the T-cell receptor (TCR) locus in CD4+ T cells, in which TRAC ribonucleoproteins (RNPs) were delivered through coincubation with A5K peptide (peptide #22) to perform TRAC KO and the CAR locus was delivered with AAV6, at 4 different timings (30 minutes before RNP treatment, at the same time, 30 minutes after, or 2 hours after).

FIG. 16A-16C depict sequential editing of CD4+ primary T cells at three genomic loci (TRAC, CD5, β2M) through coincubation of Cas9 RNPs with A5K peptide (peptide #22) at either 10 μM, 15 μM, or 20 μM.

FIG. 17A-17B depict peptide mediated editing in T cells under various stimulation conditions.

FIG. 18A-18D depict knock in of 1928z-CAR at the TCR locus with subsequent sequential KO in CD3+ Bulk T cells.

FIG. 19A-19D depict knock in of 1928z-CAR at the TCR locus with subsequent sequential KO in CD3+ Bulk T cells, or cells treated only for KO (without AAV KI).

FIG. 20 depicts Adenine to Guanine base editing at the CCR5 locus in primary human T cells delivered via peptide-coincubation with the A5K peptide (peptide #22).

FIG. 21 depicts Adenine to Guanine base editing at the CCR5 locus in primary human T cells delivered via peptide-coincubation with the A5K peptide (peptide #22).

FIG. 22 depicts base editing in primary human HSPCs at the erythrocyte-specific BCL11a enhancer locus.

FIG. 23 is a schematic depiction of DNA delivery via peptides in the context of DNA vaccines.

FIG. 24 depicts peptide-mediated DNA delivery and protein expression in DC2.4 cells.

FIG. 25 depicts antibody titers as measured by enzyme-linked immunosorbent assay (ELISA) in mice injected with plasmid DNA encoding the receptor-binding domain (RBD) of the Spike protein from SARS COV-2, with or without the addition of 200 pmol E5-TAT peptide.

FIG. 26 provides nucleotide sequences of gRNAs used in the Examples (SEQ ID Nos: 125-136, respectively).

FIG. 27A-27C provide amino acid sequences of fusion polypeptides comprising a Cas9 polypeptide and a base editor.

FIG. 28A-28D provide amino acid sequences of a Streptococcus pyogenes Cas9 polypeptide (FIG. 28A) (SEQ ID NO:143), a Staphylococcus aureus Cas9 polypeptide (FIG. 28B) (SEQ ID NO:144), and two Cas12a polypeptides (FIG. 28C (SEQ ID NO: 145) and FIG. 28D (SEQ ID NO:146)).

FIG. 29A-29E provide amino acid sequences of fusion proteins used in some of the Examples (SEQ ID Nos: 147-151, respectively).

FIG. 30 depicts various cargo delivery fusion polypeptides.

FIG. 31 provides the amino acid sequence of a base editor (“ABE8e-SpCas9-NGG dTadA/TadA8e dimer w/C-term BP-SV40/Nuc”) (SEQ ID NO:152).

FIG. 32 provides spacer sequences of guide RNAs used in the Examples (SEQ ID Nos: 153-156, respectively).

FIG. 33A-33B depicts genome editing and cell viability when various cargo delivery fusion polypeptides were used to deliver an RNP to T cells.

FIG. 34A-34B depicts genome editing and cell viability when various cargo delivery fusion polypeptides were used to deliver an RNP to T cells.

FIG. 35 depicts genome editing and cell viability when various cargo delivery fusion polypeptides bearing functional groups were used to deliver an RNP to primary T cells.

FIG. 36 depicts genome editing and cell viability when various cargo delivery fusion polypeptides comprising were used to deliver an RNP to human primary CD34⁺ hematopoietic stem and progenitor cells (HSPCs).

FIG. 37 depicts genome editing and cell viability when various cargo delivery fusion polypeptides comprising were used to deliver an RNP to HSPCs.

FIG. 38 depicts genome editing and cell viability when various cargo delivery fusion polypeptides comprising were used to deliver an RNP to neural progenitor cells (NPCs).

FIG. 39A-39D depict peptide-mediated delivery (i.e., delivery using a cargo delivery fusion polypeptide) of Cas9 RNP to generate CAR-T cells that effect tumor killing in vivo.

FIG. 40A-40D depict peptide-mediated delivery to generate CAR-T cells that effect tumor killing ex vivo.

FIG. 41 depicts a comparison of editing efficiency and viability following peptide-mediated delivery with peptide 22 (A5K (peptide #22); “PERC”) or electroporation (e-por) of various S. pyogenes Cas9 protein constructs.

FIG. 42A-42D depict data showing that peptide-mediated delivery (“PERC”) supports cell viability and maintenance of phenotype in sequential editing.

FIG. 43A-43D depict data showing that PERC supports cell viability in the production of engineered T cells bearing multiple knock-in edits at distinct genomic loci.

FIG. 44A-44F depict data showing that PERC of CRISPR RNP supports T cell engineering while inducing minimal perturbation of T cell phenotype, especially as compared to electroporation of RNP.

FIG. 45 depicts data showing that PERC of CRISPR RNP supports multiplex genome editing with minimal induction of chromosomal translocations, especially as compared to simultaneous electroporation of RNP nucleases targeting multiple genomic loci.

FIG. 46A-46C depict data showing that PERC of CRISPR RNP supports improved cell yields and robust cell expansion over time following T cell engineering, especially as compared to electroporation of RNP.

FIG. 47 depicts data showing that PERC of CRISPR RNP supports precise knock-in of a gene into primary human B cells when AAV6 is used to provide the DNA donor template necessary for enabling homology-directed repair (HDR).

FIG. 48 depicts data showing that PERC of CRISPR RNP supports precise knock-in of a gene into primary human NK cells when AAV6 is used to provide the DNA donor template necessary for enabling HDR.

FIG. 49 depicts data showing that PERC of CRISPR RNP supports high efficiency genome editing of primary human CD34⁺ HSPCs at the BCL11a locus.

FIG. 50 depicts data showing that PERC of CRISPR RNP supports high efficiency genome editing of primary human CD34⁺ HSPCs at the beta-2 microglobulin (B2M; β2M) locus.

FIG. 51 depicts base editing in primary human HSPCs at the erythrocyte-specific BCL11a enhancer locus.

FIG. 52 depicts genome editing in striatal neurons within the murine brain.

FIG. 53A-53B depict quantification of neuronal editing in Ai9 mice.

FIG. 54 depicts data showing that intravenously administered formulations containing Cas9 RNP and peptides can promote genome editing of human primary T cells in vivo.

### DEFINITIONS

The terms “polypeptide,” “peptide,” and “protein,” used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

“Operably linked” refers to a juxtaposition wherein the components so described are in a relationship permitting them to function in their intended manner. For instance, a promoter is operably linked to a coding sequence if the promoter affects its transcription or expression.

A “vector” or “expression vector” is a replicon, such as plasmid, phage, virus, or cosmid, to which another DNA segment, i.e. an “insert”, may be attached so as to bring about the replication of the attached segment in a cell.

“Heterologous,” as used herein, means a nucleotide or polypeptide sequence that is not found in the native (e.g., naturally-occurring) nucleic acid or protein, respectively.

The term “antibody” includes antibodies of any isotype, fragments of antibodies that retain specific binding to antigen, including, but not limited to, Fab, Fv, single-chain Fv (scFv), and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein.

As used herein, the terms “treatment,” “treating,” “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), lagomorphs, etc. In some cases, the individual is a human. In some cases, the individual is a non-human primate. In some cases, the individual is a rodent, e.g., a rat or a mouse. In some cases, the individual is a lagomorph, e.g., a rabbit.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an amphiphilic polypeptide” includes a plurality of such polypeptides and reference to “the cell” includes reference to one or more cells and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides a cargo delivery fusion polypeptide comprising an endosomolytic polypeptide and a cell penetrating polypeptide; and compositions comprising the cargo delivery fusion polypeptide. The present disclosure also provides methods of delivering a cargo into a target eukaryotic cell, using a composition comprising an amphiphilic polypeptide.

### Cargo Delivery Fusion Polypeptides

The present disclosure provides a cargo delivery fusion polypeptide comprising: a) an endosomolytic polypeptide; and b) a cell penetrating polypeptide (CPP). A cargo delivery fusion polypeptide of the present disclosure can be a polypeptide of any one of Formulas I-V, as set out below. A cargo delivery fusion polypeptide of the present disclosure can be a polypeptide of any one of Formulas VI-X, as set out below. A cargo delivery fusion polypeptide of the present disclosure is an amphiphilic polypeptide that provides for: i) delivery of a macromolecular cargo across a eukaryotic cell membrane; and/or ii) escape of the macromolecular cargo from the endosome. Delivery of a cargo (e.g., a ribonucleoprotein (RNP) or other cargo) to a cell (in vitro or in vivo) using a cargo delivery fusion polypeptide of the present disclosure is also referred to herein as “peptide-mediated delivery” or “PERC.”

A cargo delivery fusion polypeptide of the present disclosure provides for delivery of a cargo into a cell (e.g., a eukaryotic cell), e.g., for delivery of a cargo to the cytoplasm of a eukaryotic cell. A cargo delivery fusion polypeptide of the present disclosure provides for delivery of a cargo into a eukaryotic cell, where use of the cargo delivery fusion polypeptide to delivery a cargo into a eukaryotic cell is less toxic to the cell than, e.g., electroporation. For example, at least 50%, at least 60%, at least 70%, at least 75%, at least 80%, at least 85%, at least 90%, or more than 90%, of a population of cells contacted with a cargo delivery fusion polypeptide of the present disclosure remains viable for at least 48 hours following the contacting.

In some cases, the CPP portion of a cargo delivery fusion polypeptide of the present disclosure is an arginine-rich peptide having a length of from about 8 amino acids to about 12 amino acids (e.g., 8, 9, 10, 11, or 12 amino acids). Examples of arginine-rich CPPs include, e.g., YGRKKRRQRR (SEQ ID NO:160), GRKKRRQRRR (SEQ ID NO:161), GRKKRRQRR (SEQ ID NO: 162), and the like.

In some cases, an endosomolytic polypeptide present in a cargo delivery fusion polypeptide of the present disclosure is derived from an influenza hemagglutinin polypeptide. For example, in some cases, an endosomolytic polypeptide present in a cargo delivery fusion polypeptide of the present disclosure is derived from an E5 polypeptide (e.g., a peptide having the sequence: GLFEAIAEFIENGWEGLIEGWYG (SEQ ID NO:163)). As another example, in some cases, an endosomolytic polypeptide present in a cargo delivery fusion polypeptide of the present disclosure is derived from an INF7 polypeptide (e.g., a peptide having the sequence: GLFEAIEGFIENGWEGMIDGWYG (SEQ ID NO:164)). An endosomolytic polypeptide present in a cargo delivery fusion polypeptide of the present disclosure can have a length of from 21 amino acids to 25 amino acids (e.g., 21, 22, 23, 24, or 25 amino acids).

The total length of a cargo delivery fusion polypeptide of the present disclosure is from about 29 amino acids to about 37 amino acids (e.g., 29, 30, 31, 32, 33, 34, 35, 36, or 37 amino acids). In some cases, the total length of a cargo delivery fusion polypeptide of the present disclosure is from about 32 amino acids to about 35 amino acids.

In some cases, a cargo delivery fusion polypeptide of the present disclosure comprises: a) an endosomolytic polypeptide; and b) a cell penetrating polypeptide, wherein the fusion polypeptide has a length of from about 32 amino acids to about 35 amino acids, and wherein any two adjacent amino acids are independently linked by an amide bond or a non-amide bond, wherein the cargo delivery fusion polypeptide comprises one or more of: i) a positively charged amino acid at the N-terminus; ii) a positively charged amino acid within 5 amino acids of the N-terminus; and iii) a positively charged amino acid at position 22. In some cases, the fusion polypeptide comprises an amino acid sequence of any one of Formulas I-VIII.

In some cases, a cargo delivery fusion polypeptide of the present disclosure comprises a positively-charged amino acid as the N-terminal amino acid; for example, in some cases, a cargo delivery fusion polypeptide of the present disclosure comprises a His or a Lys as the N-terminal amino acid. In some cases, a cargo delivery fusion polypeptide comprises: i) a positively-charged amino acid as the N-terminal amino acid (e.g., comprises a His or a Lys as the N-terminal amino acid); and ii) a positively-charged amino acid within 5 amino acids of the N-terminus. For example, in a cargo delivery fusion polypeptide comprises: i) a positively-charged amino acid as the N-terminal amino acid (e.g., comprises a His or a Lys as the N-terminal amino acid); and ii) an Arg or a Lys at amino acid 5. In some cases, the N-terminal amino acid is His and amino acid 5 is Arg. In some cases, the N-terminal amino acid is His and amino acid 5 is Lys. In some cases, a cargo delivery fusion polypeptide of the present disclosure comprises a positively-charged amino acid at amino acid 22; for example, in some cases, a cargo delivery fusion polypeptide comprises a Lys or an Arg at position 22. In some cases, a cargo delivery fusion polypeptide of the present disclosure comprises: i) a positively-charged amino acid as the N-terminal amino acid (e.g., a His or a Lys as the N-terminal amino acid); ii) a positively-charged amino acid within 5 amino acids of the N-terminus (e.g., an Arg or a Lys at amino acid 5); and iii) a positively-charged amino acid at amino acid 22 (e.g., a Lys or an Arg at amino acid 22).

In some cases, a cargo delivery fusion polypeptide of the present disclosure is a polypeptide of Formula I: KLFEX₁IEGFIENGWEX₂MIDX₃WX₄GX₅GRKKRRQRR (SEQ ID NO:165), where: X₁ is A, R, or K; X₂ is A or G; X₃ is L or G; X₄ is N or Y; and X₅, if present, is Y.

Polypeptides of Formula I include:

(peptide 19; SEQ ID NO: 19)
KLFEAIEGFIENGWEGMIDGWYGYGRKKRRQRR;
(peptide 40; SEQ ID NO: 40)
KLFEAIEGFIENGWEGMIDGWYGGRKKRRQRR;
(peptide 44; SEQ ID NO: 44)
KLFERIEGFIENGWEGMIDGWYGYGRKKRRQRR;
(peptide 45; SEQ ID NO: 45)
KLFEKIEGFIENGWEGMIDGWYGYGRKKRRQRR;
(peptide 46; SEQ ID NO: 46)
KLFEAIEGFIENGWEAMIDGWYGYGRKKRRQRR;
(peptide 47; SEQ ID NO: 47)
KLFEAIEGFIENGWEGMIDLWYGYGRKKRRQRR;
(peptide 48; SEQ ID NO: 48)
KLFEAIEGFIENGWEGMIDGWNGYGRKKRRQRR;
(peptide 53; SEQ ID NO: 53)
KLFEAIEGFIENGWEAMIDLWYGYGRKKRRQRR;
(peptide 54; SEQ ID NO: 54)
KLFEAIEGFIENGWEAMIDGWNGYGRKKRRQRR;
(peptide 55; SEQ ID NO: 55)
KLFEAIEGFIENGWEGMIDLWNGYGRKKRRQRR;
(peptide 56; SEQ ID NO: 56)
KLFEAIEGFIENGWEAMIDLWNGYGRKKRRQRR;
and
(peptide 57; SEQ ID NO: 57)
KLFEKIEGFIENGWEAMIDLWNGYGRKKRRQRR.

In some cases, a cargo delivery fusion polypeptide of the present disclosure is a polypeptide of Formula II: X₁LFEX₂IEGFIENGWEGMIDGWYGYGRKKRRQRR (SEQ ID NO:166), where: X₁ is R or G; and X₂ is R or K.

Polypeptides of Formula II include:

(peptide 42; SEQ ID NO: 42)
RLFERIEGFIENGWEGMIDGWYGYGRKKRRQRR;
(peptide 43; SEQ ID NO: 43)
RLFEKIEGFIENGWEGMIDGWYGYGRKKRRQRR;
and
(peptide 41; SEQ ID NO: 41)
GLFERIEGFIENGWEGMIDGWYGYGRKKRRQRR.

In some cases, a cargo delivery fusion polypeptide of the present disclosure is a polypeptide of Formula III: GLFEAIEGFIENGWEX₁MIDX₂WNGYGRKKRRQRR (SEQ ID NO:167), where: X₁ is A or G; and X₂ is G or L.

Polypeptides of Formula III include:

(peptide 50; SEQ ID NO: 50)
GLFEAIEGFIENGWEAMIDGWNGYGRKKRRQRR;
(peptide 51; SEQ ID NO: 51)
GLFEAIEGFIENGWEGMIDLWNGYGRKKRRQRR;
and
(peptide 52; SEQ ID NO: 52)
GLFEAIEGFIENGWEAMIDLWNGYGRKKRRQRR.

In some cases, a cargo delivery fusion polypeptide of the present disclosure is a polypeptide of Formula IV: GLFEAIEGFIENGWEX₁X₂IX₃LWYGYGRKKRRQRR (SEQ ID NO:168), where: X₁ is A or G; X₂ is L or M; and X₃ is D or E.

Polypeptides of Formula V include:

(peptide 49; SEQ ID NO: 49)
GLFEAIEGFIENGWEAMIDLWYGYGRKKRRQRR;
and
(peptide 58; SEQ ID NO: 58)
GLFEAIEGFIENGWEGLIELWYGYGRKKRRQRR.

In some cases, a cargo delivery fusion polypeptide of the present disclosure is a polypeptide of Formula V: GLFX₁AIAX₂FIX₃NGWX₄GLIX₅GWYGGRKKRRQRRR (SEQ ID NO: 169), wherein each of X₁, X₂, X₃, X₄, and X₅ is independently a non-coded amino acid. In some cases, the non-coded amino acid is α-aminoadipic acid. In some cases, a polypeptide of Formula V has the amino acid sequence: GLFαAIAαFIαNGWαGLIαGWYGGRKKRRQRRR (peptide 59; SEQ ID NO:59); or GLFαAIAαFIENGWEGLIDGWYGGRKKRRQRRR (peptide 60; SEQ ID NO:60), where “α” is α-aminoadipic acid.

In some cases, a cargo delivery fusion polypeptide of the present disclosure is a polypeptide of Formula VI: KLFEX₁IX₂X₃FIENGWEGMIX₄X₅WX₆GYGRKKRRQRX₇ (SEQ ID NO: 170), wherein: X₁ is A or H; X₂ is E or A; X₃ is G or E; X₄ is D or E; X₅ is G or L; X₆ is E, H, K, R, or N; and X₇, if present, is R.

Polypeptides of Formula VI include:

(peptide 62; SEQ ID NO: 62)
KLFEAIEGFIENGWEGMIDLWEGYGRKKRRQRR;
(peptide 63; SEQ ID NO: 63)
KLFEAIEGFIENGWEGMIDLWHGYGRKKRRQRR;
(peptide 64; SEQ ID NO: 64)
KLFEAIEGFIENGWEGMIDLWKGYGRKKRRQRR;
(peptide 65; SEQ ID NO: 65)
KLFEAIEGFIENGWEGMIDLWRGYGRKKRRQRR;
(peptide 69; SEQ ID NO: 69)
KLFEAIEGFIENGWEGMIDLWNGYGRKKRRQR;
(peptide 71; SEQ ID NO: 71)
KLFEAIEGFIENGWEGMIELWNGYGRKKRRQRR;
(peptide 72; SEQ ID NO: 72)
KLFEAIAEFIENGWEGMIDLWNGYGRKKRRQRR;
(peptide 105; SEQ ID NO: 105)
KLFEHIEGFIENGWEGMIDGWYGYGRKKRRQRR;
(peptide 107; SEQ ID NO: 107)
KLFEHIEGFIENGWEGMIDLWYGYGRKKRRQRR;
and
(peptide 109; SEQ ID NO: 109)
KLFEHIEGFIENGWEGMIDLWKGYGRKKRRQRR.

In some cases, a cargo delivery fusion polypeptide of the present disclosure is a polypeptide of Formula VII: GLFEX₂IX₂X₃FIENGWEGMIDX₄WX₅GYGRKKRRQRR (SEQ ID NO: 171), wherein: X₁ is R, H, A, or K; X₂ is E or A; X₃ is G or E; X₄ is L or G; and X₅ is N, Y, K, or E.

Polypeptides of Formula VII include:

(peptide 66; SEQ ID NO: 66)
GLFERIEGFIENGWEGMIDLWNGYGRKKRRQRR;
(peptide 68; SEQ ID NO: 68)
GLFEHIEGFIENGWEGMIDLWNGYGRKKRRQRR;
(peptide 70; SEQ ID NO: 70)
GLFEAIAEFIENGWEGMIDGWYGYGRKKRRQRR;
(peptide 73; SEQ ID NO: 73)
GLFEKIEGFIENGWEAMIDGWYGYGRKKRRQRR;
(peptide 74; SEQ ID NO: 74)
GLFEKIEGFIENGWEGMIDLWYGYGRKKRRQRR;
(peptide 75; SEQ ID NO: 31)
GLFEAIEGFIENGWEGMIDGWNGYGRKKRRQRR;
(peptide 76; SEQ ID NO: 24)
GLFEAIEEFIENGWEGMIDGWYGYGRKKRRQRR;
(peptide 98; SEQ ID NO: 98)
GLFEKIEGFIENGWEGMIDGWNGYGRKKRRQRR;
(peptide 99; SEQ ID NO: 99)
GLFEKIEGFIENGWEGMIDGWKGYGRKKRRQRR;
(peptide 100; SEQ ID NO: 100)
GLFEKIEGFIENGWEGMIDGWEGYGRKKRRQRR;
(peptide 101; SEQ ID NO: 101)
GLFEKIEGFIENGWEGMIDLWNGYGRKKRRQRR;
(peptide 102; SEQ ID NO: 102)
GLFEKIEGFIENGWEGMIDLWKGYGRKKRRQRR;
and
(peptide 103; SEQ ID NO: 103)
GLFEKIEGFIENGWEGMIDLWEGYGRKKRRQRR.

In some cases, a cargo delivery fusion polypeptide of the present disclosure is a polypeptide of Formula VIII: HLFEX₁IEGFIENGWEGMIDX₂WX₃GYGRKKRRQRR (SEQ ID NO: 172), wherein: X₁ is A or K; X₂ is G or L; and X₃ is N, K, E, or Y.

Polypeptides of Formula VIII include:

(peptide 92; SEQ ID NO: 92)
HLFEAIEGFIENGWEGMIDGWNGYGRKKRRQRR;
(peptide 93; SEQ ID NO: 93)
HLFEAIEGFIENGWEGMIDGWKGYGRKKRRQRR;
(peptide 94; SEQ ID NO: 94)
HLFEAIEGFIENGWEGMIDGWEGYGRKKRRQRR;
(peptide 95; SEQ ID NO: 67)
HLFEAIEGFIENGWEGMIDLWNGYGRKKRRQRR;
(peptide 96; SEQ ID NO: 96)
HLFEAIEGFIENGWEGMIDLWKGYGRKKRRQRR;
(peptide 97; SEQ ID NO: 97)
HLFEAIEGFIENGWEGMIDLWEGYGRKKRRQRR;
(peptide 104; SEQ ID NO: 104)
HLFEKIEGFIENGWEGMIDGWYGYGRKKRRQRR;
(peptide 106; SEQ ID NO: 106)
HLFEKIEGFIENGWEGMIDLWYGYGRKKRRQRR;
and
(peptide 108; SEQ ID NO: 108)
HLFEKIEGFIENGWEGMIDLWKGYGRKKRRQRR.

In some cases, a fusion polypeptide of the present disclosure comprises an amino acid sequence of Formula IX: KLFEAIEGFIENGWEGMIDLWNX₁X₂YGRKKRRQRR (SEQ ID NO:173), wherein: X₁, if present, is Gly; and X₂ is Cys(methyltetrazine) or Cys(3-nitro-2-pyridinesulfenyl). In some cases, the fusion polypeptide comprises an amino acid sequence selected from: KLFEAIEGFIENGWEGMIDLWNC*YGRKKRRQRR (peptide 87; SEQ ID NO:87), wherein “C*” is Cys(methyltetrazine); KLFEAIEGFIENGWEGMIDLWNGC*YGRKKRRQRR (peptide 88; SEQ ID NO: 88), wherein “C*” is Cys(methyltetrazine); KLFEAIEGFIENGWEGMIDLWNC*YGRKKRRQRR (peptide 89; SEQ ID NO:89), wherein “C*” is Cys(3-nitro-2-pyridinesulfenyl); and KLFEAIEGFIENGWEGMIDLWNGC*YGRKKRRQRR (peptide 90; SEQ ID NO:90), wherein “C*” is Cys(3-nitro-2-pyridinesulfenyl).

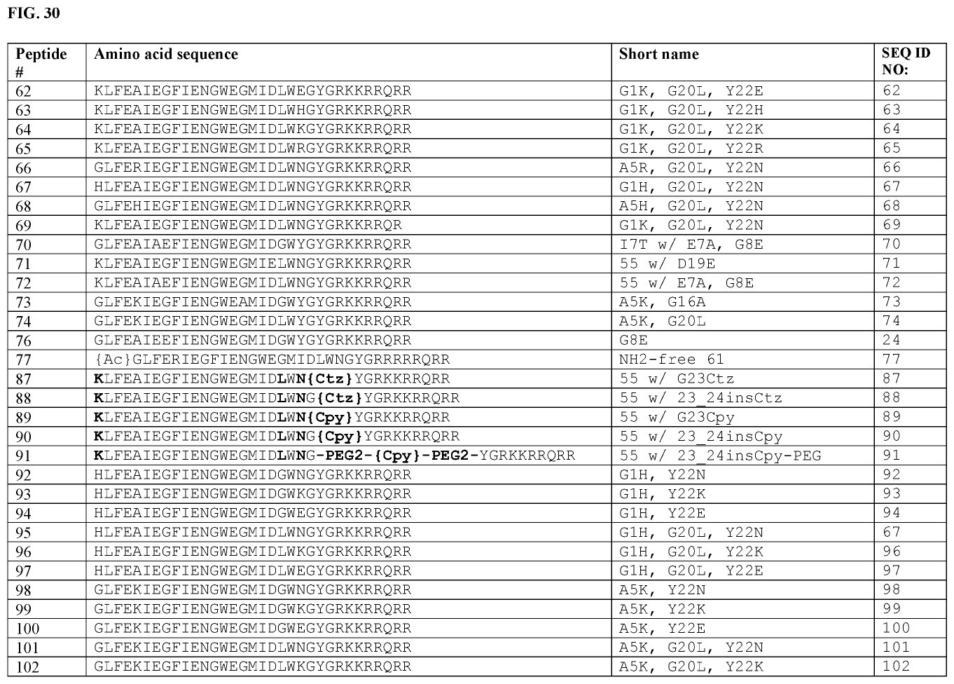

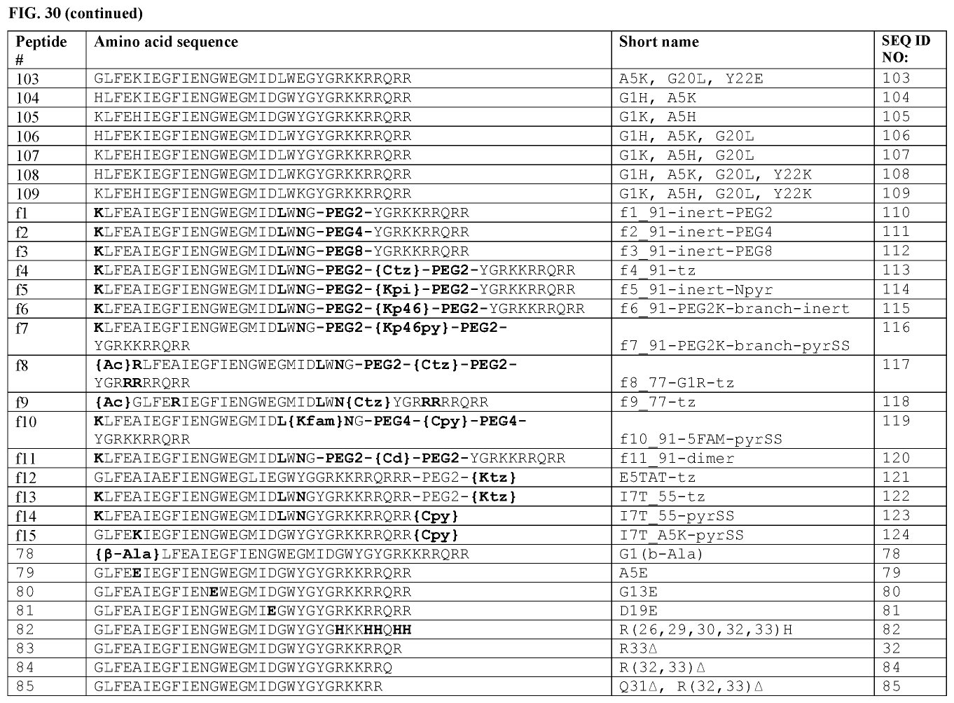

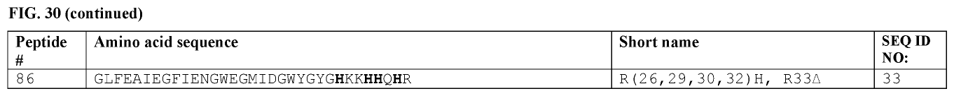

In some cases, a fusion polypeptide of the present disclosure comprises an amino acid sequence of Formula X: KLFEAIEGFIENGWEGMIDLWNGX₁YGRKKRRQRRX₂ (SEQ ID NO:174), wherein: X₁ is Cys(methyltetrazine-PEG4-maleimide), Cys(maleimide), Lys(PEG₂₃)₂, Lys(3-nitro-pyridine-2-carboxylic acid), Lys(PEG₂₃)₂, Lys(PEG₂₃)₂-(3-nitro-pyridine-2-carboxylic acid), or Cys (1,4-bis(bromomethyl)-benzene); and X₂ is Cys(3-nitro-2-pyridine-sulfenyl) or Lys(methyltetrazine-PEG4). In some cases, a fusion polypeptide comprises an amino acid sequence selected from the amino acid sequence of peptide f1, peptide f2, peptide f3, peptide f4, peptide f4, peptide f6, peptide f7, peptide f11, peptide f13, and peptide f14 depicted in FIG. 30.

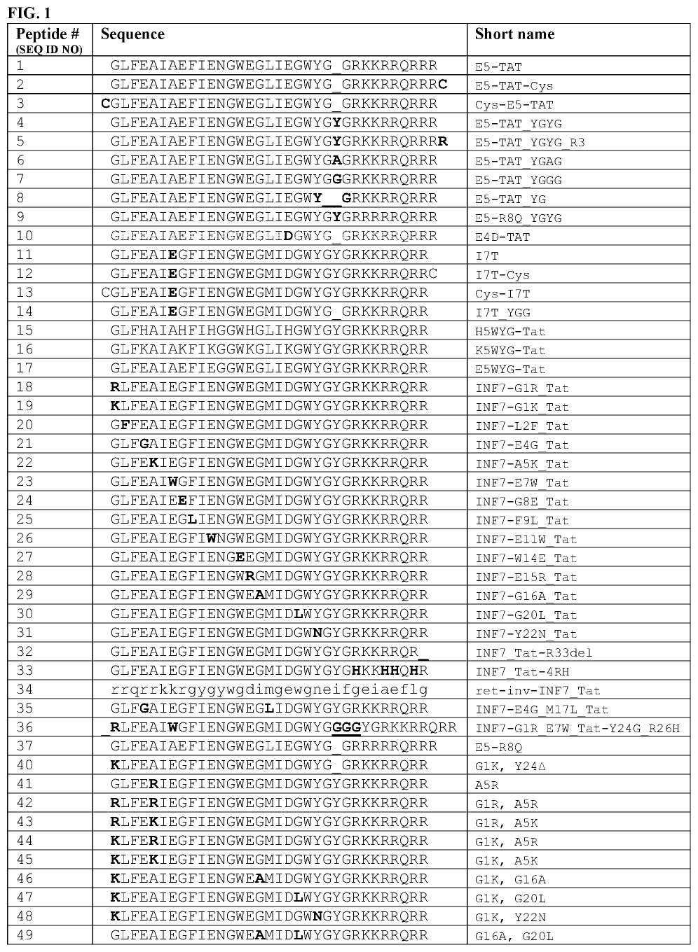

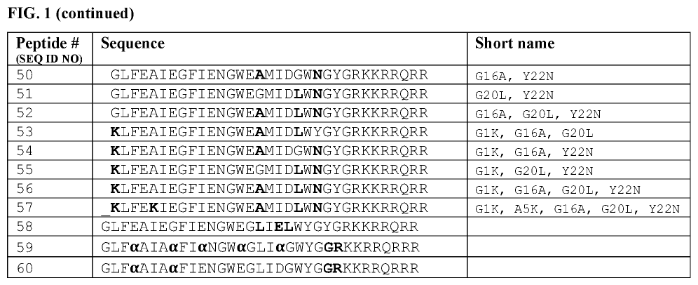

Non-limiting examples of suitable cargo delivery fusion polypeptides of the present disclosure include peptide #19 and #40-60, as depicted in FIG. 1.

Non-limiting examples of suitable cargo delivery fusion polypeptides of the present disclosure include the polypeptides designated peptide #62-74, 76, 77, 87-109, and f1-f15 in FIG. 30.

In FIG. 30, the following descriptions of modified amino acids and/or non-amide bond linkages apply:
i) {Ac} is an acetyl group;
ii) {β-Ala} is beta alanine;
iii) {Ctz} refers to a cysteine that has been chemically modified using methyltetrazine-PEG4-maleimide;
iv) {Cpy} refers to Cys(3-nitro-2-pyridinesulfenyl) incorporated into the peptide backbone;
v) -PEG2- refers to incorporation of 2 copies (monomeric units) of polyethylene glycol into the peptide backbone;
vi) -PEG4- refers to incorporation of 4 copies (monomeric units) of polyethylene glycol into the peptide backbone;
vii) -PEG8- refers to incorporation of 8 copies (monomeric units) of polyethylene glycol into the peptide backbone;
viii) {Kpi} refers to Lys(3-Nitro-pyridine-2-carboxylic acid);
ix) {Kp46} refers to Lys(PEG₂₃)₂, a branched PEG moiety extending off of the peptide backbone;
x) {Kp46py} refers to Lys(PEG₂₃)₂)-(3-Nitro-pyridine-2-carboxylic acid) extending off of the peptide backbone;
xi) {Kfam} refers to a Lys that is chemically modified via conjugation to a succinimidyl ester form of 5-FAM (5-carboxyfluorescein);
xii) {Cd} refers to Cys that is chemically modified post-synthesis with 1,4-bis(bromomethyl)-benzene, creating a peptide dimer; and
xiii) {Ktz} refers to Lys that is chemically modified via conjugation to methyltetrazine-PEG4-NHS ester.

In some cases, a cargo delivery fusion polypeptide of the present disclosure does not comprise the amino acid sequence of one or more of the polypeptides designated peptide 1-18 and 20-37 in FIG. 1. In some cases, a fusion polypeptide comprising the amino acid sequence of peptide 1 depicted in FIG. 1 is specifically excluded. In some cases, a fusion polypeptide comprising the amino acid sequence of peptide 2 depicted in FIG. 1 is specifically excluded. In some cases, a fusion polypeptide comprising the amino acid sequence of peptide 3 depicted in FIG. 1 is specifically excluded. In some cases, a fusion polypeptide comprising the amino acid sequence of peptide 4 depicted in FIG. 1 is specifically excluded. In some cases, a fusion polypeptide comprising the amino acid sequence of peptide 5 depicted in FIG. 1 is specifically excluded. In some cases, a fusion polypeptide comprising the amino acid sequence of peptide 6 depicted in FIG. 1 is specifically excluded. In some cases, a fusion polypeptide comprising the amino acid sequence of peptide 7 depicted in FIG. 1 is specifically excluded. In some cases, a fusion polypeptide comprising the amino acid sequence of peptide 8 depicted in FIG. 1 is specifically excluded. In some cases, a fusion polypeptide comprising the amino acid sequence of peptide 9 depicted in FIG. 1 is specifically excluded. In some cases, a fusion polypeptide comprising the amino acid sequence of peptide 10 depicted in FIG. 1 is specifically excluded. In some cases, a fusion polypeptide comprising the amino acid sequence of peptide 11 depicted in FIG. 1 is specifically excluded. In some cases, a fusion polypeptide comprising the amino acid sequence of peptide 12 depicted in FIG. 1 is specifically excluded. In some cases, a fusion polypeptide comprising the amino acid sequence of peptide 13 depicted in FIG. 1 is specifically excluded. In some cases, a fusion polypeptide comprising the amino acid sequence of peptide 14 depicted in FIG. 1 is specifically excluded. In some cases, a fusion polypeptide comprising the amino acid sequence of peptide 15 depicted in FIG. 1 is specifically excluded. In some cases, a fusion polypeptide comprising the amino acid sequence of peptide 16 depicted in FIG. 1 is specifically excluded. In some cases, a fusion polypeptide comprising the amino acid sequence of peptide 17 depicted in FIG. 1 is specifically excluded. In some cases, a fusion polypeptide comprising the amino acid sequence of peptide 18 depicted in FIG. 1 is specifically excluded. In some cases, a fusion polypeptide comprising the amino acid sequence of peptide 20 depicted in FIG. 1 is specifically excluded. In some cases, a fusion polypeptide comprising the amino acid sequence of peptide 21 depicted in FIG. 1 is specifically excluded. In some cases, a fusion polypeptide comprising the amino acid sequence of peptide 22 depicted in FIG. 1 is specifically excluded. In some cases, a fusion polypeptide comprising the amino acid sequence of peptide 23 depicted in FIG. 1 is specifically excluded. In some cases, a fusion polypeptide comprising the amino acid sequence of peptide 24 depicted in FIG. 1 is specifically excluded. In some cases, a fusion polypeptide comprising the amino acid sequence of peptide 25 depicted in FIG. 1 is specifically excluded. In some cases, a fusion polypeptide comprising the amino acid sequence of peptide 26 depicted in FIG. 1 is specifically excluded. In some cases, a fusion polypeptide comprising the amino acid sequence of peptide 27 depicted in FIG. 1 is specifically excluded. In some cases, a fusion polypeptide comprising the amino acid sequence of peptide 28 depicted in FIG. 1 is specifically excluded. In some cases, a fusion polypeptide comprising the amino acid sequence of peptide 29 depicted in FIG. 1 is specifically excluded. In some cases, a fusion polypeptide comprising the amino acid sequence of peptide 30 depicted in FIG. 1 is specifically excluded. In some cases, a fusion polypeptide comprising the amino acid sequence of peptide 31 depicted in FIG. 1 is specifically excluded. In some cases, a fusion polypeptide comprising the amino acid sequence of peptide 32 depicted in FIG. 1 is specifically excluded. In some cases, a fusion polypeptide comprising the amino acid sequence of peptide 33 depicted in FIG. 1 is specifically excluded. In some cases, a fusion polypeptide comprising the amino acid sequence of peptide 34 depicted in FIG. 1 is specifically excluded. In some cases, a fusion polypeptide comprising the amino acid sequence of peptide 35 depicted in FIG. 1 is specifically excluded. In some cases, a fusion polypeptide comprising the amino acid sequence of peptide 36 depicted in FIG. 1 is specifically excluded. In some cases, a fusion polypeptide comprising the amino acid sequence of peptide 37 depicted in FIG. 1 is specifically excluded.

In some cases, one or more of the peptides designated peptides 78-86 in FIG. 30 is specifically excluded. In some cases, a polypeptide comprising the amino acid sequence LFEAIEGFIENGWEGMIDGWYGYGRKKRRQRR (SEQ ID NO:175) is specifically excluded. In some cases, a polypeptide comprising the amino acid sequence {β-Ala} LFEAIEGFIENGWEGMIDGWYGYGRKKRRQRR (SEQ ID NO:78) is specifically excluded; where {β-Ala} is an acetyl group at the N-terminus of the polypeptide. In some cases, a polypeptide comprising the amino acid sequence GLFEEIEGFIENGWEGMIDGWYGYGRKKRRQRR (SEQ ID NO: 79) is specifically excluded. In some cases, a polypeptide comprising the amino acid sequence GLFEAIEGFIENEWEGMIDGWYGYGRKKRRQRR (SEQ ID NO:80) is specifically excluded. In some cases, a polypeptide comprising the amino acid sequence GLFEAIEGFIENGWEGMIEGWYGYGRKKRRQRR (SEQ ID NO:81) is specifically excluded. In some cases, a polypeptide comprising the amino acid sequence GLFEAIEGFIENGWEGMIDGWYGYGHKKHHQHH (SEQ ID NO:82) is specifically excluded. In some cases, a polypeptide comprising the amino acid sequence GLFEAIEGFIENGWEGMIDGWYGYGRKKRRQR (SEQ ID NO:32) is specifically excluded. In some cases, a polypeptide comprising the amino acid sequence GLFEAIEGFIENGWEGMIDGWYGYGRKKRRQ (SEQ ID NO:84) is specifically excluded. In some cases, a polypeptide comprising the amino acid sequence GLFEAIEGFIENGWEGMIDGWYGYGRKKRR (SEQ ID NO:85) is specifically excluded. In some cases, a polypeptide comprising the amino acid sequence GLFEAIEGFIENGWEGMIDGWYGYGHKKHHQHR (SEQ ID NO:33) is specifically excluded.

### Modified Amino Acids

A cargo delivery fusion polypeptide of the present disclosure can include one or more modified amino acids. In some cases, an amino acid present in a cargo delivery fusion polypeptide comprises a modification (a “functional moiety”) that provides for linkage to a second polypeptide or other moiety. Suitable modifications include thiol-reactive moieties; amine-reactive moieties; haloacetyl groups (e.g., iodoacetamide; chloroacetamide; and the like); and members of click chemistry pairs. For example, thiol-reactive groups include, e.g., haloacetyls, maleimides, aziridines, acryloyls, arylating agents, vinylsulfones, and pyridyl disulfides. Click chemistry pairs include, e.g., i) azide and dibenzocyclooctyne; and ii) tetrazine and trans-cyclooctene.

Functional moieties that provide for conjugation include, but are not limited to, an azido group, an alkynyl group, a phosphine group, a cysteine residue, a C-terminal thioester, aryl azides, maleimides, carbodiimides, N-hydroxysuccinimide (NHS)-esters, hydrazides, PFP-esters, hydroxymethyl phosphines, psoralens, imidoesters, pyridyl disulfides, isocyanates, aminooxy-, aldehyde, keto, chloroacetyl, bromoacetyl, and vinyl sulfone. Suitable functional moieties include, e.g., isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, and fluorophenyl esters.

Examples of cargo delivery fusion polypeptide of the present disclosure include, e.g., peptides designated peptide #77, peptide #87, peptide #88, peptide #89, peptide #90, peptide #91, peptide #f4, peptide #f5, peptide #f6, peptide #f7, peptide #f8, peptide #f9, peptide #f10, peptide #f11, peptide #f12, peptide #f13, peptide #f14, and peptide #f15, as depicted in FIG. 30.

### Non-Amide Bond Linkages

In some cases, the amino acids of a cargo delivery fusion polypeptide of the present disclosure are all linked by amide bonds. In some cases, a cargo delivery fusion polypeptide of the present disclosure comprises one or more linkages other than an amide bond. For example, in some cases, a cargo delivery fusion polypeptide comprises one or more PEG moieties in place of an amide bond between two adjacent amino acids. In some cases, a cargo delivery fusion polypeptide comprises a single PEG moeity (e.g., a PEG2 moiety, a PEG4 moiety, a PEG8 moiety, or the like) in place of an amide bond between two adjacent amino acids. Suitable PEG moieties include PEG moieties having from 2 to 8 ethylene glycol units. For example, in some cases, a cargo delivery fusion polypeptide comprises a PEG2 linkage. As another example, in some cases, a cargo delivery fusion polypeptide comprises a PEG4 linkage. As another example, in some cases, a cargo delivery fusion polypeptide comprises a PEG8 linkage. Non-limiting examples are depicted in FIG. 30.

### Compositions

The present disclosure provides a composition comprising a cargo delivery fusion polypeptide of the present disclosure. A composition of the present disclosure can comprise, in addition to a cargo delivery fusion polypeptide of the present disclosure, one or more of: a salt, e.g., NaCl, MgCl₂, KCl, MgSO₄, etc.; a buffering agent, e.g., a Tris buffer, N-(2-Hydroxyethyl) piperazine-N′-(2-ethanesulfonic acid) (HEPES), 2-(N-Morpholino) ethanesulfonic acid (MES), 2-(N-Morpholino) ethanesulfonic acid sodium salt (MES), 3-(N-Morpholino) propanesulfonic acid (MOPS), N-tris [Hydroxymethyl]methyl-3-aminopropanesulfonic acid (TAPS), etc.; a solubilizing agent; a protease inhibitor; a detergent, e.g., a non-ionic detergent such as Tween-20, Tween-80, etc.; a nuclease inhibitor; glycerol; a reducing agent (e.g., dithiothreitol (DTT)); a solubilizing agent (e.g., dimethylsulfoxide (DMSO)); a non-ionic surfactant; a synthetic polymer; and the like.

In some cases, a composition of the present disclosure comprises: a) a cargo delivery fusion polypeptide of the present disclosure; and b) DMSO. In some cases, a cargo delivery fusion polypeptide of the present disclosure is maintained in a solution comprising DMSO in a concentration of from about 9% DMSO to about 15% DMSO; e.g., about 10% DMSO) for a period of time before being contacted with the cargo. When a cargo delivery fusion polypeptide is prepared in a solution of less than 10% DMSO, the peptides may bind to each other and in some cases may not productively associate with the cargo. Thus, in some cases, cargo delivery fusion polypeptide is kept in a solution of about 10% DMSO for a period of time; after which the peptide is contacted with the cargo that is present in a solution (e.g., a buffered aqueous solution) without DMSO. The cargo delivery fusion polypeptide/cargo solution may thus contain from 1% DMSO to 5% DMSO. In some cases, a cargo delivery fusion polypeptide/cargo solution contains less than 1% DMSO; for example, in some cases, a cargo delivery fusion polypeptide/cargo solution includes from 0.01% to 1% DMSO.

In some cases, a composition of the present disclosure comprises: a) a cargo delivery fusion polypeptide of the present disclosure; and b) an organic solvent other than DMSO. Suitable organic solvents include, e.g., ethanol, methanol, dimethylformamide, gamma-butyrolactone, N-methyl-2-pyrrolidone, and dimethylacetamide.

In some cases, a composition of the present disclosure comprises: a) a cargo delivery fusion polypeptide of the present disclosure; and b) poly(ethyleneglycol) (PEG). In some cases, a composition of the present disclosure comprises: a) a cargo delivery fusion polypeptide of the present disclosure; and b) a non-ionic surfactant. Suitable non-ionic surfactants include, e.g., polysorbate 80; polyoxyethylene (23) lauryl ether (brij-L23); a poloxamer (i.e., a nonionic triblock copolymer composed of a central hydrophobic chain of polyoxypropylene (poly(propylene oxide)) flanked by two hydrophilic chains of polyoxyethylene (poly(ethylene oxide)); and the like.

In some cases, a composition of the present disclosure comprises: a) a cargo delivery fusion polypeptide of the present disclosure; and b) saline (e.g., 0.9% NaCl). In some cases, the composition is sterile. In some cases, the composition is suitable for administration to a human subject, e.g., where the composition is sterile and is free of detectable pyrogens and/or other toxins. Thus, the present disclosure provides a composition comprising: a) a cargo delivery fusion polypeptide of the present disclosure; and b) saline (e.g., 0.9% NaCl), where the composition is sterile and is free of detectable pyrogens and/or other toxins. In some cases, the composition further comprises a cargo to be delivered.

In some cases, a composition of the present disclosure comprises: a) a cargo delivery fusion polypeptide of the present disclosure; and b) a cargo to be delivered into a eukaryotic cell. Any of a variety of cargos can be included in a composition of the present disclosure. In some cases, the cargo is a nucleic acid. In some cases, the cargo is a nucleic acid comprising a nucleotide sequence encoding a gene product of interest. In some cases, the cargo is a polypeptide. In some cases, the cargo is a CRISPR-Cas effector polypeptide, or a nucleic acid comprising a nucleotide sequence encoding a CRISPR-Cas effector polypeptide. In some cases, the cargo is a ribonucleoprotein (RNP) comprising: i) a CRISPR-Cas effector polypeptide; and ii) a CRISPR-Cas guide nucleic acid. In some cases, the cargo is an RNP comprising: i) a CRISPR-Cas effector polypeptide; and ii) a guide nucleic acid that comprises an activator segment comprising a nucleotide sequence that binds to the CRISPR-Cas effector polypeptide and a targeting segment comprising a nucleotide sequence that hybridizes to a target nucleic acid. In some cases, the cargo is a DNA molecule comprising a nucleotide sequence encoding both a CRISPR-Cas effector protein and a CRISPR-Cas guide RNA. In some cases, the cargo comprises: i) an mRNA encoding a CRISPR-Cas effector polypeptide; and ii) a CRISPR-Cas guide nucleic acid (e.g., a CRISPR-Cas guide RNA, such as a single-molecule guide RNA (sgRNA)).

In some cases, the cargo is not covalently linked to the cargo delivery fusion polypeptide. In some cases, the cargo is covalently linked, directly or indirectly (e.g., indirectly, via a linker), to the cargo delivery fusion polypeptide.

In some cases, the cargo delivery fusion polypeptide is present in the composition in a concentration of from about 2 μM to about 50 μM. For example, in some cases, the cargo delivery fusion polypeptide is present in the composition in a concentration of from about 2 μM to about 5 μM, from about 5 μM to about 10 μM, from about 10 μM to about 15 μM, from about 15 μM to about 20 μM, from about 20 μM to about 25 μM, from about 25 μM to about 30 μM, from about 30 μM to about 40 μM, or from about 40 μM to about 50 μM.

### Nucleic Acid Cargo

As noted above, in some cases, the cargo is a nucleic acid. Thus, in some cases, a composition of the present disclosure comprises: a) a cargo delivery fusion polypeptide of the present disclosure; and b) a nucleic acid cargo to be delivered into a eukaryotic cell. In some cases, the cargo is a nucleic acid comprising a nucleotide sequence encoding a gene product of interest (a “cargo gene product”). Gene products of interest include nucleic acids and polypeptides. For example, in some cases, the cargo is a DNA molecule comprising a nucleotide sequence (a “cargo nucleotide sequence”) encoding a gene product of interest. In some cases, the cargo comprises two or more DNA molecules, each comprising a nucleotide sequence encoding a different gene product of interest. In some cases, the cargo is a DNA molecule comprising nucleotide sequences encoding two or more gene products of interest.

In some cases, the cargo nucleotide sequence encoding the gene product of interest is operably linked to one or more transcriptional control elements, such as a promoter. In some cases, the cargo nucleotide sequence encoding the gene product of interest is operably linked to a promoter that is operable in a cell type of choice (e.g., a eukaryotic cell, a plant cell, an animal cell, a mammalian cell, a primate cell, a rodent cell, a human cell, a neuron, a dendritic cell, an epithelial cell, a T cell, a natural killer (NK) cell, a hematopoietic stem cell, etc.).

In some cases, the promoter is a constitutively active promoter. In some cases, the promoter is a regulatable promoter. In some cases, the promoter is an inducible promoter. In some cases, the promoter is a tissue-specific promoter. In some cases, the promoter is a cell type-specific promoter. In some cases, the transcriptional control element (e.g., the promoter) is functional in a targeted cell type or targeted cell population.

In some cases, the cargo nucleotide sequence encodes an RNA gene product. RNA gene products of interest include, e.g., inhibitory RNAs, CRISPR-Cas guide RNAs, and the like. RNA gene products of interest include an RNA that inhibits or reduces production of a deleterious or otherwise undesired protein.

In some cases, the cargo nucleotide sequence encodes a cargo polypeptide. Cargo polypeptide gene products of interest include, e.g., therapeutic polypeptides, immunogenic polypeptides, growth factors, cytokines, enzymes, anti-angiogenic polypeptides, soluble receptors, antibodies, synthetic polypeptides (e.g., chimeric antigen receptors), blood clotting factors, peptide hormones, and the like.

In some cases, the cargo nucleotide sequence encoding the cargo polypeptide of interest is operably linked to a nucleotide sequence encoding a secretion signal peptide (for example, 5′-ATGAAGATCATCCTGTGGCTGTGTGTGTTCGGCCTGTTCCTGGCCACCATGTTCCCCATCAG CTGGCAGATGCCCGTGGAGTCGGCCTGTCCTCCGAGGACTCCGCCAGCTCCGAGAGCTTCG CC-3′ (SEQ ID NO:176); Jeong et al (2012) J. Control Release 159 (3): 368-75). In some cases, a nucleotide sequence encoding a furin cleavage site (RGRR) is inserted between the nucleotide sequence encoding the secretion signal peptide and the nucleotide sequence encoding the cargo polypeptide of interest. The secretion signal peptide of the expressed protein is deleted in the Golgi apparatus, allowing secretion of the desired polypeptide.

In some cases, the cargo nucleic acid comprises a nucleotide sequence encoding an antibody. In some cases, the encoded antibody is a therapeutic antibody. In some instances, the nucleotide sequence encoding the antibody comprises a nucleotide sequence encoding a secretion signal peptide as described above such that upon expression of the cargo in the targeted cell, the therapeutic polypeptide will be secreted out of the cell.

### Targeting Antibodies

In some cases, the cargo comprises a targeting antibody. The targeting antibody may be linked to the cargo (e.g. an RNP comprising a CRISPR Cas effector polypeptide or other gene editing machinery), where linkage (e.g., covalent linkage) can be direct or via a linker. In some cases, the linker is a proteolytically cleavable linker. The targeting antibody can be specific for a cell surface protein on the surface of a cell (the “target cells”) to which the cargo is delivered. In some cases, the cell surface protein targeted by the targeting antibody is a cancer-associated antigen (e.g. EpCAM, E-cadherin, EMA, HER2/neu, alpha-fetoprotein, beta-hCG, bladder tumor antigen, BCR-ABL, CEA, CD19, CD22, CD30, MCAM (Muc18), metadherin, glypigan 2, PSMA, human transferrin receptor, EGRF complex, AXL, PTK7 and the like); specific cell type receptors (e.g. CD4, CD8, TCR in T cells); a stem cell marker (e.g. TRA-1-60, TRA-1-81, SSEA); or a lineage marker (e.g. CD14 (Monocytes), CD16 (NK cells, granulocytes), CD19 (B lymphocytes), CD20 (B lymphocytes), and CD56 (NK cells) in humans). In some cases, targeting of the cargo is achieved using an alternate targeting moiety such as an aptamer, small molecules, peptides, toxins, carbohydrates, vitamins or transferrin.

### Polypeptide Cargo

As noted above, in some cases, the cargo is polypeptide. Thus, in some cases, a composition of the present disclosure comprises: a) a cargo delivery fusion polypeptide of the present disclosure; and b) a polypeptide cargo to be delivered into a eukaryotic cell. Cargo polypeptides of interest include, e.g., therapeutic polypeptides, immunogenic polypeptides, growth factors, cytokines, enzymes, anti-angiogenic polypeptides, soluble receptors, antibodies, synthetic polypeptides (e.g., chimeric antigen receptors), blood clotting factors, peptide hormones, and the like.

In some cases, the cargo polypeptide comprises a targeting moiety to aid in delivery of the cargo to a desired cell or tissue type. In some cases, the targeting moiety is an antibody or fragment thereof, a darpin, an aptamer or the like.

### Antibodies

In some cases, the cargo polypeptide is an antibody. Thus, in some cases, a composition of the present disclosure comprises: a) a cargo delivery fusion polypeptide of the present disclosure; and b) an antibody to be delivered into a eukaryotic cell. Suitable antibodies are described elsewhere herein. The antibody can be any antigen-binding antibody-based polypeptide, a wide variety of which are known in the art. In some instances, the antibody is a single chain Fv (scFv). Other antibody-based recognition domains that are suitable for use include cAb VHH (camelid antibody variable domains) and humanized versions; IgNAR VH (shark antibody variable domains) and humanized versions; sdAb VH (single domain antibody variable domains); and “camelized” antibody variable domains. In some instances, the cargo polypeptide comprises T-cell receptor (TCR)-based recognition domains such as single chain TCR (scTv, single chain two-domain TCR containing VaVB). In some cases, the cargo antibody is a therapeutic antibody.

An antibody can be specific for an antigen such as CD49f, CD34, CD90, CD117, CXCR4, CD79, CD22, RP105, CD71, CD28, CD94 (KLDR1), CD56, XCR1, CD205, CD370, CD209, CD54, CD335, NCR1, CD94, NKG2D, NKp30, CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, and the like. In some cases, the antibody is specific for a cytokine. In some cases, the antibody is specific for a cytokine receptor. In some cases, the antibody is specific for a growth factor. In some cases, the antibody is specific for a growth factor receptor. In some cases, the antibody is specific for a cell-surface receptor. In some cases, the antibody is an anti-CD3 antibody.

### Immunogenic Polypeptides

In some cases, the cargo is an immunogenic polypeptide. An immunogenic protein is suitable for stimulating an immune response to the antigenic protein in a mammalian host (e.g., a human, a non-human primate, a bovine (e.g., a cow), an ovine (e.g., a sheep), an equine (e.g., a horse), a porcine (e.g. a pig), and the like). The immunogenic polypeptide can be derived from an autoantigen, an allergen, a tumor-associated antigen, a pathogenic virus, a pathogenic bacterium, a pathogenic protozoan, a pathogenic helminth, or any other pathogenic organism that infects a mammalian host.

A viral antigen can be an antigen of any of a variety of viral pathogens.

In some cases, the viral pathogen is a virus of the adenoviridae, arenaviridae, astroviridae, bunyaviridae, caliciviridae, coronaviridae, filoviridae, flaviviridae, hepadnaviridae, hepeviridae, orthomyxoviridae, papillomaviridae, Paramyxoviridae, Parvoviridae, picornaviridae, polyomaviridae, Poxviridae, reoviridae, retroviridae, rhabdoviridae, or togaviridae family. In some cases, the virus is an adenovirus, coronavirus, coxsackievirus, Epstein-Barr virus, hepatitis A virus, hepatitis B virus, hepatitis C virus, herpes simplex virus type 2, cytomegalovirus, human herpes virus type 8, human immunodeficiency virus, influenza virus, measles virus, mumps virus, human papillomavirus, parainfluenza virus, poliovirus, rabies virus, respiratory syncytial virus, rubella virus, or varicella-zoster virus. The virus can be selected from Adenoviridae (e.g., adenovirus), Arenaviridae (e.g., Machupo virus), Bunyaviridae (e.g., Hantavirus or Rift Valley fever virus), Coronaviridae, Orthomyxoviridae (e.g., influenza viruses), Filoviridae (e.g., Ebola virus and Marburg virus), Flaviviridae (e.g., Japanese encephalitis virus and Yellow fever virus), Hepadnaviridae (e.g., hepatitis B virus), Herpesviridae (e.g., herpes simplex viruses), Papovaviridae (e.g., papilloma viruses), Paramyxoviridae (e.g., respiratory syncytial virus, measles virus, mumps virus, or parainfluenza virus), Parvoviridae, Picornaviridae (e.g., polioviruses), Poxviridae (e.g., variola viruses), Reoviridae (e.g., rotaviruses), Retroviridae (e.g., human T cell lymphotropic viruses (HTLV) and human immunodeficiency viruses (HIV)), Rhabdoviridae (e.g., rabies virus), and Togaviridae (e.g., encephalitis viruses, yellow fever virus, and rubella virus)).

In some cases, the viral antigen is an antigen of a viral pathogen selected from among Adenoviruses, Alphaviruses (Togaviruses), Eastern equine encephalitis virus, Eastern equine encephalomyelitis virus, Venezuelan equine encephalomyelitis vaccine strain TC-83, Western equine encephalomyelitis virus, Arenaviruses, Lymphocytic choriomeningitis virus (non-neurotropic strains), Tacaribe virus complex, Bunyaviruses, Bunyamwera virus, Rift Valley fever virus vaccine strain MP-12, Calciviruses, Coronaviruses. Flaviviruses (Togaviruses)-Group B Arboviruses, Dengue virus serotypes 1, 2, 3, and 4, Yellow fever virus vaccine strain 17D, Hepatitis A, B, C, D, and E viruses, the Cytomegalovirus, Epstein Barr virus, Herpes simplex types 1 and 2, Herpes zoster, Human herpesvirus types 6 and 7, Influenza viruses types A, B, and C, Papovaviruses, Papilloma viruses, Newcastle disease virus, Measles virus, Mumps virus, Parainfluenza viruses types 1, 2, 3, and 4, polyomaviruses (JC virus, BK virus), Respiratory syncytial virus, Human parvovirus (B 19), Coxsackie viruses types A and B, Echoviruses, Polioviruses, Rhinoviruses, Alastrim (Variola minor virus), Smallpox (Variola major virus), Whitepox Reoviruses, Coltivirus, human Rotavirus, and Orbivirus (Colorado tick fever virus), Rabies virus, Vesicular stomatitis virus, Rubivirus (rubella), Semliki Forest virus, St. Louis encephalitis virus, Venezuelan equine encephalitis virus, Venezuelan equine encephalomyelitis virus, Arenaviruses (a.k.a. South American Hemorrhagic Fever virus), Flexal, Lymphocytic choriomeningitis virus (LCM) (neurotropic strains), Hantaviruses including Hantaan virus, Rift Valley fever virus, Japanese encephalitis virus, Yellow fever virus, Monkeypox virus, Human immunodeficiency virus (HIV) types 1 and 2, Human T cell lymphotropic virus (HTLV) types 1 and 2. Simian immunodeficiency virus (SIV), Vesicular stomatitis virus, Guanarito virus, Lassa fever virus, Junin virus, Machupo virus, Sabia, Crimean-Congo hemorrhagic fever virus, Ebola viruses, Marburg virus, Tick-borne encephalitis virus complex (flavi) including Central European tick-borne encephalitis, Far Eastern tick-borne encephalitis, Hanzalova, Hypr, Kumlinge, Kyasanur Forest disease, Omsk hemorrhagic fever, and Russian Spring Summer encephalitis viruses, Herpesvirus simiae (Herpes B or Monkey B virus), Cercopithecine herpesvirus 1 (Herpes B virus), Equine morbillivirus (Hendra and Hendra-like viruses), Nipah virus, Variola major virus (Smallpox virus), Variola minor virus (Alastrim), African swine fever virus, African horse sickness virus, Akabane virus, Avian influenza virus (highly pathogenic), Blue tongue virus, Camel pox virus, Classical swine fever virus, Cowdria ruminantium (heartwater), Foot and mouth disease virus, Goat pox virus, Japanese encephalitis virus, Lumpy skin disease virus, Malignant catarrhal fever virus, Menangle virus, Newcastle disease virus (VVND), Peste Des Petits Ruminants virus, Rinderpest virus, Sheep pox virus, Swine vesicular disease virus, and Vesicular stomatitis virus.

In some cases, the bacterial antigen is an antigen of a bacterium, such as Bacillus (e.g., B. anthracis), Enterobacteriaceae (e.g., Salmonella, Escherichia coli, Yersinia pestis, Klebsiella, and Shigella), Yersinia (e.g., Y. pestis or Y. enterocolitica), Staphylococcus (e.g., S. aureus), Streptococcus, Gonorrheae, Enterococcus (e.g., E. faecalis), Listeria (e.g., L. monocytogenes), Brucella (e.g., B. abortus, B. melitensis, or B. suis), Vibrio (e.g., V. cholerae), Corynebacterium diphtheria, Pseudomonas (e.g., P. pseudomallei or P. aeruginosa), Burkholderia (e.g., B. mallei or B. pseudomallei), Shigella (e.g., S. dysenteriae), Rickettsia (e.g., R. rickettsii, R. prowazekii, or R. typhi), Francisella tularensis, Chlamydia psittaci, Coxiella burnetii, Mycoplasma (e.g., M. mycoides), etc.

In some cases, the bacterial antigen is an antigen of a bacterial pathogen selected from Acinetobacter baumannii (formerly Acinetobacter calcoaceticus); Actinobacillus; Actinomyces pyogenes (formerly Corynebacterium pyogenes); Actinomyces israelii; Nocardia asteroides; N. brasiliensis; Aeromonas hydrophila; Amycolata autotrophica; Archanobacterium haemolyticum (formerly Corynebacterium haemolyticum); Arizona hinshawii—all serotypes; Bacillus anthracis; Bacteroides fragilis; Bartonella henselae; B. quintana; B. vinsonii; Bordetella including B. pertussis; Borrelia recurrentis; B. burgdorferi; Burkholderia (formerly Pseudomonas species), Campylobacter coli, C. fetus, C. jejuni, Chlamydia psittaci, C. trachomatis, C. pneumonia, Clostridium botulinum (neurotoxin producing species), Cl. chauvoei, Cl. haemolyticum, Cl. histolyticum, Cl. novyi, Cl. septicum, Cl. tetani, Cl. perfirngens, Corynebacterium diphtheriae, C. pseudotuberculosis, C. renale, Dermatophilus congolensis, Edwardsiella tarda, Erysipelothrix rhusiopathiae, Escherichia coli—all enteropathogenic, enterotoxigenic, enteroinvasive and strains bearing K1 antigen, including E. coli O157:H7; Haemophilus ducreyi, H. influenzae; Helicobacter pylori, Klebsiella—all species; Legionella including L. pneumophila; Leptospira interrogans—all serotypes; Listeria, Moraxella, Mycobacterium, including M. avium complex, M. asiaticum, M. bovis BCG vaccine strain, M. chelonei, M. fortuitum, M. kansasii, M. leprae, M. malmoense, M. marinum, M. paratuberculosis, M. scrofulaceum, M. simiae, M. szulgai, M. ulcerans, M. xenopi; Mycoplasma; Neisseria gonorrhoeae, N. meningitides, Nocardia asteroides, N. brasiliensis, N. otitidiscaviarum, N. transvalensis; Proteus mirabilis; P. vulgaris; Rhodococcus equi; Salmonella including S. arizonae, S. cholerasuis, S. enteritidis, S. gallinarum-pullorum, S. meleagridis, S. paratyphi, A, B, C. S. typhi; S. typhimurium; Shigella including S. boydii, S. dysenteriae, type 1, S. flexneri, S. sonnei; Sphenophorus necrophorus; Staphylococcus aureus; Streptobacillus moniliformis; Streptococcus including S. pneumoniae, S. pyogenes; Treponema pallidum, T. carateum; Vibrio cholerae. V. parahemolyticus, V. vulnificus; Yersinia enterocolitica; Bartonella; Brucella including B. abortus, B. canis, B. suis, B. melitensis; Burkholderia (Pseudomonas) mallei; B. pseudomallei; Coxiella burnetiid; Francisella tularensis; Mycobacterium bovis, M. tuberculosis; Mycobacteria; Pasteurella multocida type B-“buffalo” and other virulent strains; Rickettsia akari, R. australis, R. canada, R. conorii, R. prowazekii, R. rickettsii, R, siberica, R. tsutsugamushi, R. typhi (R. mooseri); and Yersinia pestis.

In some cases, the antigen is a protozoan antigen, e.g., an antigen from a protozoan such as Cryptosporidium parvum, Encephalitozoa, Plasmodium (e.g., Plasmodium falciparum), Toxoplasma gondii, Acanthamoeba, Entamoeba histolytica, Giardia lamblia, Trichomonas vaginalis, Leishmania, or Trypanosoma (e.g., T. brucei; T. cruzi); etc.

In some cases, the immunogenic polypeptide is a cancer-associated antigen. Cancer-associated antigens include, but are not limited to, CD19, CD22, a MUC1 polypeptide, a human papillomavirus (HPV) E6 polypeptide, an LMP2 polypeptide, an HPV E7 polypeptide, an epidermal growth factor receptor (EGFR) vIII polypeptide, a HER-2/neu polypeptide, a melanoma antigen family A. 3 (MAGE A3) polypeptide, a p53 polypeptide, a mutant p53 polypeptide, an NY-ESO-1 polypeptide, a folate hydrolase (prostate-specific membrane antigen; PSMA) polypeptide, a carcinoembryonic antigen (CEA) polypeptide, a melanoma antigen recognized by T-cells (melanA/MART1) polypeptide, a Ras polypeptide, a gp100 polypeptide, a proteinase3 (PR1) polypeptide, a ber-abl polypeptide, a tyrosinase polypeptide, a survivin polypeptide, a prostate specific antigen (PSA) polypeptide, an hTERT polypeptide, a sarcoma translocation breakpoints polypeptide, a synovial sarcoma X (SSX) breakpoint polypeptide, an EphA2 polypeptide, an acid phosphatase, prostate (PAP) polypeptide, a melanoma inhibitor of apoptosis (ML-IAP) polypeptide, an alpha-fetoprotein (AFP) polypeptide, an epithelial cell adhesion molecule (EpCAM) polypeptide, an ERG (TMPRSS2 ETS fusion) polypeptide, a NA17 polypeptide, a paired-box-3 (PAX3) polypeptide, an anaplastic lymphoma kinase (ALK) polypeptide, an androgen receptor polypeptide, a cyclin B1 polypeptide, an N-myc proto-oncogene (MYCN) polypeptide, a Ras homolog gene family member C (RhoC) polypeptide, a tyrosinase-related protein-2 (TRP-2) polypeptide, a mesothelin polypeptide, a prostate stem cell antigen (PSCA) polypeptide, a melanoma associated antigen-1 (MAGE A1) polypeptide, a cytochrome P450 1B1 (CYP1B1) polypeptide, a placenta-specific protein 1 (PLAC1) polypeptide, a BORIS polypeptide (also known as CCCTC-binding factor or CTCF), an ETV6-AML polypeptide, a breast cancer antigen NY-BR-1 polypeptide (also referred to as ankyrin repeat domain-containing protein 30A), a regulator of G-protein signaling (RGS5) polypeptide, a squamous cell carcinoma antigen recognized by T-cells (SART3) polypeptide, a carbonic anhydrase IX polypeptide, a paired box-5 (PAX5) polypeptide, an OY-TES1 (testis antigen; also known as acrosin binding protein) polypeptide, a sperm protein 17 polypeptide, a lymphocyte cell-specific protein-tyrosine kinase (LCK) polypeptide, a high molecular weight melanoma associated antigen (HMW-MAA), an A-kinase anchoring protein-4 (AKAP-4), a synovial sarcoma X breakpoint 2 (SSX2) polypeptide, an X antigen family member 1 (XAGE1) polypeptide, a B7 homolog 3 (B7H3; also known as CD276) polypeptide, a legumain polypeptide (LGMN1; also known as asparaginyl endopeptidase), a tyrosine kinase with Ig and EGF homology domains-2 (Tie-2; also known as angiopoietin-1 receptor) polypeptide, a P antigen family member 4 (PAGE4) polypeptide, a vascular endothelial growth factor receptor 2 (VEGF2) polypeptide, a MAD-CT-1 polypeptide, a fibroblast activation protein (FAP) polypeptide, a platelet derived growth factor receptor beta (PDGFB) polypeptide, a MAD-CT-2 polypeptide, a Fos-related antigen-1 (FOSL) polypeptide, or a Wilms tumor-1 (WT-1) polypeptide.

### CRISPR-Cas Effector Polypeptides

In some cases, the cargo is a CRISPR-Cas effector polypeptide. A CRISPR-Cas effector polypeptide suitable for inclusion in a composition of the present disclosure is a class 2 CRISPR effector polypeptide, also referred to herein as a class 2 CRISPR-Cas effector polypeptide. For example, in some cases, the CRISPR-Cas effector polypeptide is a type II CRISPR-Cas effector polypeptide. In some cases, the type II CRISPR-Cas effector polypeptide is a Cas9 polypeptide. In some cases, the CRISPR-Cas effector polypeptide is a type V CRISPR-Cas effector polypeptide, e.g., a Cas12a, a Cas12b, a Cas12c, a Cas12d, or a Cas12e polypeptide. In some cases, the CRISPR-Cas effector polypeptide is a type VI CRISPR-Cas effector polypeptide, e.g., a Cas13a polypeptide, a Cas13b polypeptide, a Cas13c polypeptide, or a Cas13d polypeptide. In some cases, the CRISPR-Cas effector polypeptide is a Cas14 polypeptide. In some cases, the CRISPR-Cas effector polypeptide is a Cas14a polypeptide, a Cas14b polypeptide, or a Cas14c polypeptide. A CRISPR-Cas effector polypeptide suitable for inclusion in a composition of the present disclosure includes a CRISPRi polypeptide. See, e.g., Qi et al. (2013) Cell 152:1173; and Jensen et al. (2021) Genome Research doi: 10.1101/gr.275607.121. A CRISPR-Cas effector polypeptide suitable for inclusion in a composition of the present disclosure includes a CRISPRa polypeptide. See, e.g., Jensen et al. (2021) Genome Research doi: 10.1101/gr.275607.121; and Breinig et al. (2019) Nature Methods 16:51. A CRISPR-Cas effector polypeptide suitable for inclusion in a composition of the present disclosure includes a CRISPRoff polypeptide. See, e.g., Nuñez et al. (2021) Cell 184:2503. A CRISPR-Cas effector polypeptide suitable for inclusion in a composition of the present disclosure includes a nickase. A CRISPR-Cas effector polypeptide suitable for inclusion in a composition of the present disclosure includes a catalytically inactive CRISPR-Cas effector polypeptide that retains binding (when complexed with a guide RNA) to a target nucleic acid. A CRISPR-Cas effector polypeptide suitable for inclusion in a composition of the present disclosure includes a fusion polypeptide comprising: i) a CRISPR-Cas effector polypeptide; and ii) one or more heterologous fusion partners (also referred to as “heterologous polypeptides”).

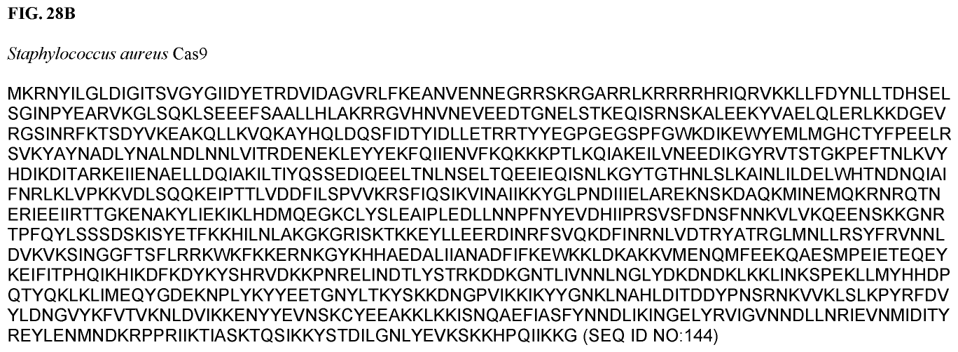

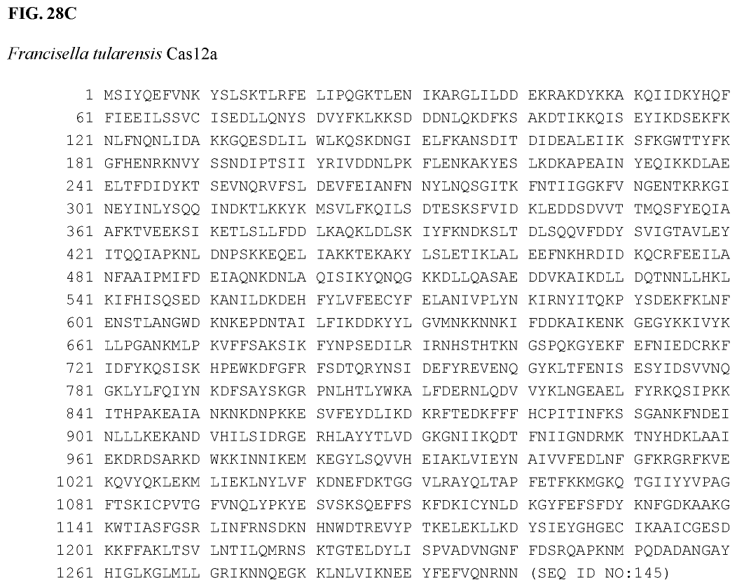

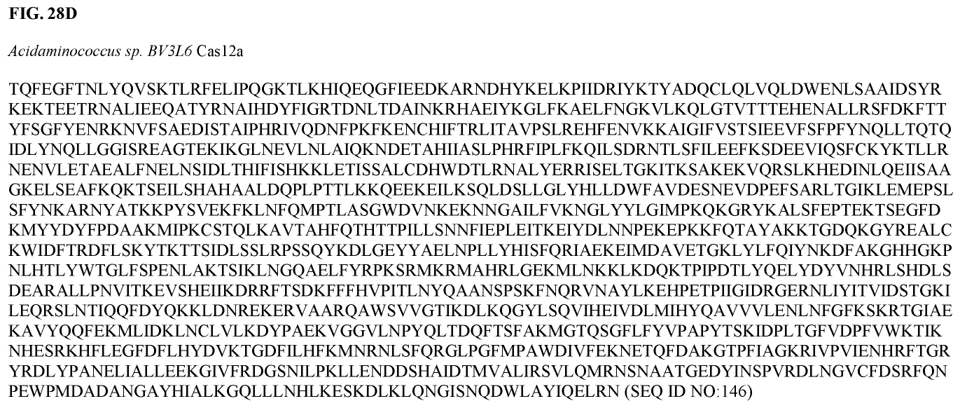

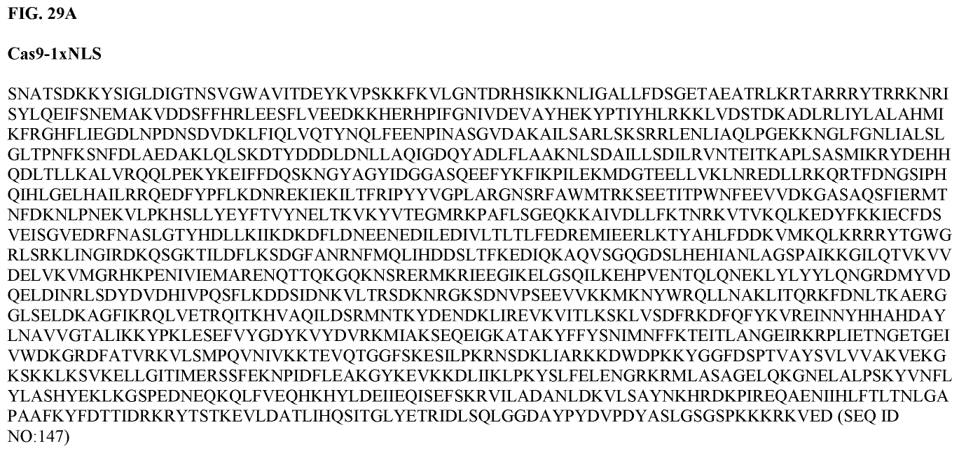

In some cases, a CRISPR-Cas effector polypeptide suitable for inclusion in a composition of the present disclosure is a Cas9 polypeptide. In some cases, a Cas9 polypeptide comprises an amino acid sequence having at least 50%, at least 60%, at least 70%, at least 80%, at least 90%, at least 95%, at least 98%, at least 99%, or more than 99%, amino acid sequence identity to the Streptococcus pyogenes Cas9 depicted in FIG. 28A.

In some cases, the Cas9 polypeptide is a Staphylococcus aureus Cas9 (saCas9) polypeptide. In some cases, the saCas9 polypeptide comprises an amino acid sequence having at least 85%, at least 90%, at least 95%, at least 98%, at least 99%, or 100%, amino acid sequence identity to any known saCas9 amino acid sequence, e.g., a saCas9 amino acid sequence depicted in FIG. 28B.

In some cases, a suitable Cas9 polypeptide is a high-fidelity (HF) Cas9 polypeptide. Kleinstiver et al. (2016) Nature 529:490. For example, amino acids N497, R661, Q695, and Q926 of the amino acid sequence depicted in FIG. 28A are substituted, e.g., with alanine. For example, an HF Cas9 polypeptide can comprise an amino acid sequence having at least 90%, at least 95%, at least 98%, or at least 99%, amino acid sequence identity to the amino acid sequence depicted in FIG. 28A, where amino acids N497, R661, Q695, and Q926 are substituted, e.g., with alanine. In some cases, a suitable Cas9 polypeptide exhibits altered PAM specificity. See, e.g., Kleinstiver et al. (2015) Nature 523:481.

In some cases, a suitable Cas9 polypeptide comprises an R691A substitution. For example, in some cases, a suitable Cas9 polypeptide comprise an amino acid sequence having at least 90%, at least 95%, at least 98%, or at least 99%, amino acid sequence identity to the amino acid sequence depicted in FIG. 28A, where amino acid 691 is Ala.

In some cases, a suitable Cas9 polypeptide comprises D1135V. R1335Q, and T1337R substitutions. For example, in some cases, a suitable Cas9 polypeptide comprise an amino acid sequence having at least 90%, at least 95%, at least 98%, or at least 99%, amino acid sequence identity to the amino acid sequence depicted in FIG. 28A, where amino acid 1135 is Val, amino acid 1335 is Gln, and amino acid 1337 is Arg, and where the Cas9 polypeptide exhibits relaxed PAM requirements.

In some cases, a suitable Cas9 polypeptide is a SpRY variant. See, e.g., Zhang and Zhang (2020) Trends Genetics 36:546; and Walton et al. (2020) Science 368:290; and U.S. Patent Publication No. 2021/0284978. SpRY is a variant of S. pyogenes Cas9; this variant has reduced PAM requirements. For example, in some cases, a suitable Cas9 polypeptide comprises D1135L, S1136W, G1218K, E1219Q, R1335Q, and T1337R substitutions. For example, in some cases, a suitable Cas9 polypeptide comprise an amino acid sequence having at least 90%, at least 95%, at least 98%, or at least 99%, amino acid sequence identity to the amino acid sequence depicted in FIG. 28A, where amino acid 1135 is Leu, amino acid 1136 is Trp, amino acid 1218 is Lys, amino acid 1219 is Gln, amino acid 1335 is Gln, and amino acid 1337 is Arg. In some cases, a suitable Cas9 polypeptide comprise an amino acid sequence having at least 90%, at least 95%, at least 98%, or at least 99%, amino acid sequence identity to the amino acid sequence depicted in FIG. 28A; and comprises amino acid substitution(s) at one, two, three, four, five, or all six of the following positions: at E1219 (e.g., an E1219Q, an E1219H, an E1219S, or an E1219V substitution); S1136 (e.g., an S1136W, an S1136F, an S1136A, or an S1136V substitution); D1135 (e.g., a D1135L, a D1135A, a D1135W, or a D1135F substitution); G1218 (e.g., a G1218R, a G1218K, or a G1218S substitution); R1335 (e.g., an R1335Q substitution); and T1337 (e.g., a T1337R or a T1337K substitution).

In some cases, a suitable Cas9 polypeptide is an xCas9 polypeptide or a Cas9-NG polypeptide. See, e.g., Zhong et al. (2019) Molec. Plant 12:1027; Hu et al. (2018) Nature 556:57; and Nishimasu et al. (2018) Science 361:1259.

In some cases, a suitable CRISPR-Cas effector polypeptide is a type V CRISPR-Cas effector polypeptide. In some cases, a type V CRISPR-Cas effector polypeptide is a Cas12a protein. In some cases, a Cas12a protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to any known Cas12a protein, e.g., a Cas12a amino acid sequence depicted in FIG. 28C or FIG. 28D.

In some cases, the CRISPR-Cas effector polypeptide is a CRISPR-Cas effector fusion polypeptide comprising: a) a CRISPR-Cas effector polypeptide; and b) one or more heterologous polypeptides (also referred to as fusion partners). In some cases, the one or more heterologous polypeptides comprises a single stranded nuclease, a double strand nuclease, a helicase, a methylase, a demethylase, an acetylase, a deacetylase, a deaminase, an integrase, a recombinase, a base editor, or a prime editor. In some cases, the one or more heterologous polypeptides comprises a nuclear localization signal. In some cases, the fusion partner (heterologous polypeptide) is a reverse transcriptase. In some cases, the fusion partner is a base editor. In some cases, the fusion partner (heterologous polypeptide) is a deaminase.

In some cases, the heterologous polypeptide is a reverse transcriptase polypeptide. Thus, in some cases, the CRISPR-Cas effector polypeptide is a CRISPR-Cas effector fusion polypeptide comprising: a) a CRISPR-Cas effector polypeptide; and b) a reverse transcriptase. Such a fusion polypeptide is useful for prime editing. See, e.g., Anzalone et al. (2019) Nature 576:149; and Scholefield and Harrison (2021) Gene Therapy 28:396. In some cases, the CRISPR-Cas effector polypeptide portion of the fusion polypeptide is catalytically inactive. Suitable reverse transcriptases include, e.g., a murine leukemia virus reverse transcriptase; a Rous sarcoma virus reverse transcriptase; a human immunodeficiency virus type I reverse transcriptase; a Moloney murine leukemia virus reverse transcriptase; and the like. In some cases, a fusion polypeptide comprising a CRISPR-Cas effector polypeptide and a reverse transcriptase uses a modified gRNA. For example, in some cases, the gRNA is modified to include sequence information that is incorporated into the genome near the site of spacer-directed CRISPR domain binding

In some cases, the heterologous polypeptide is a nuclease. Suitable nucleases include, but are not limited to, a homing nuclease polypeptide; a FokI polypeptide; a transcription activator-like effector nuclease (TALEN) polypeptide; a MegaTAL polypeptide; a meganuclease polypeptide; a zinc finger nuclease (ZFN); an ARCUS nuclease; and the like. The meganuclease can be engineered from an LADLIDADG homing endonuclease (LHE). A megaTAL polypeptide can comprise a TALE DNA binding domain and an engineered meganuclease. See, e.g., WO 2004/067736 (homing endonuclease); Urnov et al. (2005) Nature 435:646 (ZFN); Mussolino et al. (2011) Nucle. Acids Res. 39:9283 (TALE nuclease); Boissel et al. (2013) Nucl. Acids Res. 42:2591 (MegaTAL).

In some cases, the heterologous polypeptide is a base editor. Suitable base editors include, e.g., an adenosine deaminase; a cytidine deaminase (e.g., an activation-induced cytidine deaminase (AID)); APOBEC3G; and the like); and the like.

A suitable adenosine deaminase is any enzyme that is capable of deaminating adenosine in DNA. In some cases, the deaminase is a TadA deaminase.

In some cases, a suitable adenosine deaminase comprises an amino acid sequence having at least 80%, at least 85%, at least 90%, at least 95%, at least 98%, at least 99%, or 100%, amino acid sequence identity to the following amino acid sequence:

(SEQ ID NO: 177)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTD

In some cases, a suitable adenosine deaminase comprises an amino acid sequence having at least 80%, at least 85%, at least 90%, at least 95%, at least 98%, at least 99%, or 100%, amino acid sequence identity to the following amino acid sequence:

(SEQ ID NO: 178)
MRRAFITGVFFLSEVEFSHEYWMRHALTLAKRAWDEREVPVGAVL
VHNNRVIGEGWNRPIGRHDPTAHAEIMALRQGGLVMQNYRLIDAT
LYVTLEPCVMCAGAMIHSRIGRVVFGARDAKTGAAGSLMDVLHHP
GMNHRVEITEGILADECAALLSDFFRMRRQEIKAQKKAQSSTD.

In some cases, a suitable adenosine deaminase comprises an amino acid sequence having at least 80%, at least 85%, at least 90%, at least 95%, at least 98%, at least 99%, or 100%, amino acid sequence identity to the following Staphylococcus aureus TadA amino acid sequence:

(SEQ ID NO: 179)
MGSHMTNDIYFMTLAIEEAKKAAQLGEVPIGAIITKDDEVIARAH
NLRETLQQPTAHAEHIAIERAAKVLGSWRLEGCTLYVTLEPCVMC
AGTIVMSRIPRVVYGADDPKGGCSGSLMNLLQQSNFNHRAIVDKG
VLKEACSTLLTTFFKNLRANKKSTN:

In some cases, a suitable adenosine deaminase comprises an amino acid sequence having at least 80%, at least 85%, at least 90%, at least 95%, at least 98%, at least 99%, or 100%, amino acid sequence identity to the following Bacillus subtilis TadA amino acid sequence:

(SEQ ID NO: 180)
MTQDELYMKEAIKEAKKAEEKGEVPIGAVLVINGEIIARAHNLRE
TEQRSIAHAEMLVIDEACKALGTWRLEGATLYVTLEPCPMCAGAV
VLSRVEKVVFGAFDPKGGCSGTLMNLLQEERFNHQAEVVSGVLEE
ECGGMLSAFFRELRKKKKAARKNLSE.

In some cases, a suitable adenosine deaminase comprises an amino acid sequence having at least 80%, at least 85%, at least 90%, at least 95%, at least 98%, at least 99%, or 100%, amino acid sequence identity to the following Salmonella typhimurium TadA:

(SEQ ID NO: 181)
MPPAFITGVTSLSDVELDHEYWMRHALTLAKRAWDEREVPVGAVL
VHNHRVIGEGWNRPIGRHDPTAHAEIMALRQGGLVLQNYRLLDTT
LYVTLEPCVMCAGAMVHSRIGRVVFGARDAKTGAAGSLIDVLHHP
GMNHRVEIIEGVLRDECATLLSDFFRMRRQEIKALKKADRAEGAG
PAV.

In some cases, a suitable adenosine deaminase comprises an amino acid sequence having at least 80%, at least 85%, at least 90%, at least 95%, at least 98%, at least 99%, or 100%, amino acid sequence identity to the following Shewanella putrefaciens TadA amino acid sequence:

(SEQ ID NO: 182)
MDEYWMQVAMQMAEKAEAAGEVPVGAVLVKDGQQIATGYNLSISQ
HDPTAHAEILCLRSAGKKLENYRLLDATLYITLEPCAMCAGAMVH
SRIARVVYGARDEKTGAAGTVVNLLQHPAFNHQVEVTSGVLAEAC
SAQLSRFFKRRRDEKKALKLAQRAQQGIE.

In some cases, a suitable adenosine deaminase comprises an amino acid sequence having at least 80%, at least 85%, at least 90%, at least 95%, at least 98%, at least 99%, or 100%, amino acid sequence identity to the following Haemophilus influenzae F3031 TadA amino acid sequence:

(SEQ ID NO: 187)
MDAAKVRSEFDEKMMRYALELADKAEALGEIPVGAVLVDDARNII
GEGWNLSIVQSDPTAHAEIIALRNGAKNIQNYRLLNSTLYVTLEP
CTMCAGAILHSRIKRLVFGASDYKTGAIGSRFHFFDDYKMNHTLE
ITSGVLAEECSQKLSTFFQKRREEKKIEKALLKSLSDK

In some cases, a suitable adenosine deaminase comprises an amino acid sequence having at least 80%, at least 85%, at least 90%, at least 95%, at least 98%, at least 99%, or 100%, amino acid sequence identity to the following Caulobacter crescentus TadA amino acid sequence:

(SEQ ID NO: 183)
MRTDESEDQDHRMMRLALDAARAAAEAGETPVGAVILDPSTGEVI
ATAGNGPIAAHDPTAHAEIAAMRAAAAKLGNYRLTDLTLVVTLEP
CAMCAGAISHARIGRVVFGADDPKGGAVVHGPKFFAQPTCHWRPE
VTGGVLADESADLLRGFFRARRKAKI

In some cases, a suitable adenosine deaminase comprises an amino acid sequence having at least 80%, at least 85%, at least 90%, at least 95%, at least 98%, at least 99%, or 100%, amino acid sequence identity to the following Geobacter sulfurreducens TadA amino acid sequence:

(SEQ ID NO: 184)
MSSLKKTPIRDDAYWMGKAIREAAKAAARDEVPIGAVIVRDGAVI
GRGHNLREGSNDPSAHAEMIAIRQAARRSANWRLTGATLYVTLEP
CLMCMGAIILARLERVVFGCYDPKGGAAGSLYDLSADPRLNHQVR
LSPGVCQEECGTMLSDFFRDLRRRKKAKATPALFIDERKVPPEP.

Cytidine deaminases suitable for inclusion in a CRISPR/Cas effector polypeptide fusion polypeptide include any enzyme that is capable of deaminating cytidine in DNA.

In some cases, the cytidine deaminase is a deaminase from the apolipoprotein B mRNA-editing complex (APOBEC) family of deaminases. In some cases, the APOBEC family deaminase is selected from the group consisting of APOBEC1 deaminase, APOBEC2 deaminase, APOBEC3A deaminase, APOBEC3B deaminase, APOBEC3C deaminase, APOBEC3D deaminase, APOBEC3F deaminase, APOBEC3G deaminase, and APOBEC3H deaminase. In some cases, the cytidine deaminase is an activation induced deaminase (AID).

In some cases, a suitable cytidine deaminase comprises an amino acid sequence having at least 80%, at least 85%, at least 90%, at least 95%, at least 98%, at least 99%, or 100%, amino acid sequence identity to the following amino acid sequence:

(SEQ ID NO: 185)
MDSLLMNRRKFLYQFKNVRWAKGRRETYLCYVVKRRDSATSFSLD
FGYLRNKNGCHVELLFLRYISDWDLDPGRCYRVTWFTSWSPCYDC
ARHVADFLRGNPNLSLRIFTARLYFCEDRKAEPEGLRRLHRAGVQ
IAIMTFKDYFYCWNTFVENHERTFKAWEGLHENSVRLSRQLRRIL
LPLYEVDDLRDAFRTLGL.

In some cases, a suitable cytidine deaminase is an AID and comprises an amino acid sequence having at least 80%, at least 85%, at least 90%, at least 95%, at least 98%, at least 99%, or 100%, amino acid sequence identity to the following amino acid sequence: MDSLLMNRRK

(SEQ ID NO: 186)
FLYQFKNVRW AKGRRETYLC YVVKRRDSAT
SFSLDFGYLR NKNGCHVELL FLRYISDWDL
DPGRCYRVTW FTSWSPCYDC ARHVADFLRG
NPNLSLRIFT ARLYFCEDRK AEPEGLRRLH
RAGVQIAIMT FKENHERTFK AWEGLHENSV
RLSRQLRRIL LPLYEVDDLR DAFRTLGL.

In some cases, a suitable cytidine deaminase is an AID and comprises an amino acid sequence having at least 80%, at least 85%, at least 90%, at least 95%, at least 98%, at least 99%, or 100%, amino acid sequence identity to the following amino acid sequence: MDSLLMNRRK

(SEQ ID NO: 185)
FLYQFKNVRW AKGRRETYLC YVVKRRDSAT
SFSLDFGYLR NKNGCHVELL FLRYISDWDL
DPGRCYRVTW FTSWSPCYDC ARHVADFLRG
NPNLSLRIFT ARLYFCEDRK AEPEGLRRLH
RAGVQIAIMT FKDYFYCWNT FVENHERTFK
AWEGLHENSV RLSRQLRRIL LPLYEVDDLR
DAFRTLGL.

In some cases, a CRISPR-Cas fusion polypeptide comprises one or more nuclear localization signals (NLSs). In some cases, a CRISPR-Cas fusion polypeptide comprises both one or more NLSs, and a heterologous effector polypeptide. In some cases, a CRISPR-Cas fusion polypeptide includes one or more NLSs (e.g., 2 or more, 3 or more, 4 or more, or 5 or more NLSs). In some cases, one or more NLSs (2 or more, 3 or more, 4 or more, or 5 or more NLSs) are positioned at or near (e.g., within 50 amino acids of) the N-terminus and/or the C-terminus. In some cases, one or more NLSs (2 or more, 3 or more, 4 or more, or 5 or more NLSs) are positioned at or near (e.g., within 50 amino acids of) the N-terminus. In some cases, one or more NLSs (2 or more, 3 or more, 4 or more, or 5 or more NLSs) are positioned at or near (e.g., within 50 amino acids of) the C-terminus. In some cases, one or more NLSs (3 or more, 4 or more, or 5 or more NLSs) are positioned at or near (e.g., within 50 amino acids of) both the N-terminus and the C-terminus. In some cases, an NLS is positioned at the N-terminus and an NLS is positioned at the C-terminus.

Non-limiting examples of NLSs include an NLS sequence derived from: the NLS of the SV40 virus large T-antigen, having the amino acid sequence PKKKRKV (SEQ ID NO: 188); the NLS from nucleoplasmin (e.g., the nucleoplasmin bipartite NLS with the sequence KRPAATKKAGQAKKKK (SEQ ID NO:189)); the c-myc NLS having the amino acid sequence PAAKRVKLD (SEQ ID NO:190) or RQRRNELKRSP (SEQ ID NO:191); the hRNPA1 M9 NLS having the sequence NQSSNFGPMKGGNFGGRSSGPYGGGGQYFAKPRNQGGY (SEQ ID NO:192); the sequence RMRIZFKNKGKDTAELRRRRVEVSVELRKAKKDEQILKRRNV (SEQ ID NO:193) of the IBB domain from importin-alpha; the sequences VSRKRPRP (SEQ ID NO: 194) and PPKKARED (SEQ ID NO:195) of the myoma T protein; the sequence PQPKKKPL (SEQ ID NO: 196) of human p53; the sequence SALIKKKKKMAP (SEQ ID NO: 197) of mouse c-abl IV; the sequences DRLRR (SEQ ID NO: 198) and PKQKKRK (SEQ ID NO: 199) of the influenza virus NS1; the sequence RKLKKKIKKL (SEQ ID NO:200) of the Hepatitis virus delta antigen; the sequence REKKKFLKRR (SEQ ID NO:201) of the mouse Mx1 protein; the sequence KRKGDEVDGVDEVAKKKSKK (SEQ ID NO:202) of the human poly(ADP-ribose) polymerase; the sequence RKCLQAGMNLEARKTKK (SEQ ID NO:203) of the steroid hormone receptors (human) glucocorticoid; and a bipartite SV40 NLS of the following amino acid sequence: KRTADGSEFESPKKKRKV (SEQ ID NO:138).

In some cases, an NLS comprises the amino acid sequence K-K/R-X-K/R, where X is any amino acid; and where the NLS has a length of from 7 to 17 amino acids, from 5 to 15 amino acids, or from 15 to 20 amino acids. In some cases, an NLS comprises the amino acid sequence RPAATKKAGQAKKKKLD (SEQ ID NO:204) and has a length of 17 amino acids. In some cases, an NLS comprises the amino acid sequence PKKKRKV (SEQ ID NO: 188) and has a length of 7 amino acids. In some cases, an NLS comprises the amino acid sequence PKKKRKVED (SEQ ID NO:205); and has a length of 9 amino acids. In some cases, an NLS comprises the amino acid sequence PKKKRKVDT (SEQ ID NO:206); and has a length of 9 amino acids.

In some cases, the CRISPR-Cas effector polypeptide, or the CRISPR-Cas fusion polypeptide, comprises a covalently linked antibody or non-antibody-based recognition scaffold. Suitable non-antibody-based recognition scaffolds include an avimer, a DARPin, an adnectin, an avimer, an affibody, an anticalin, or an affilin. The covalently linked antibody or non-antibody-based recognition scaffold can be linked to the CRISPR-Cas effector polypeptide via a proteolytically cleavable linker. The covalently linked antibody or non-antibody-based recognition scaffold can target the CRISPR-Cas effector polypeptide or CRISPR-Cas fusion polypeptide to a target eukaryotic cell.

### CRISPR-Cas Guide Nucleic Acids

In some cases, the cargo is a CRISPR-Cas guide nucleic acid. As noted above, in some cases, a composition of the present disclosure comprises: a) a cargo delivery fusion polypeptide of the present disclosure; and b) a nucleic acid. In some cases, the nucleic acid is a CRISPR-Cas guide nucleic acid, or a nucleic acid comprising a nucleotide sequence encoding a CRISPR-Cas guide nucleic acid. Also as noted above, in some cases, a composition of the present disclosure comprises an RNP complex comprising: i) a class 2 CRISPR-Cas effector polypeptide, or a nucleic acid comprising a nucleotide sequence encoding the class 2 CRISPR-Cas effector polypeptide; ii) a guide nucleic acid, or a nucleic acid comprising a nucleotide sequence encoding the guide nucleic acid.

A nucleic acid that binds to a class 2 CRISPR-Cas endonuclease (e.g., a type II, a type V, or a type VI CRISPR-Cas protein) and targets the complex to a specific location within a target nucleic acid is referred to herein as a guide nucleic acid (e.g., a “guide RNA” or “CRISPR-Cas guide nucleic acid” or “CRISPR-Cas guide RNA”). A guide nucleic acid provides target specificity to the complex (the RNP complex) by including a targeting segment, which includes a guide sequence (also referred to herein as a targeting sequence), which is a nucleotide sequence that is complementary to a sequence of a target nucleic acid.

A guide nucleic acid (can be said to include two segments, a first segment (referred to herein as a “targeting segment”); and a second segment (referred to herein as a “protein-binding segment”). By “segment” it is meant a segment/section/region of a molecule, e.g., a contiguous stretch of nucleotides in a nucleic acid molecule. A segment can also mean a region/section of a complex such that a segment may comprise regions of more than one molecule. The “targeting segment” is also referred to herein as a “variable region” of a guide RNA. The “protein-binding segment” is also referred to herein as a “constant region” of a guide RNA. In some cases, the guide RNA is a Cas9 guide RNA.

A targeting segment of a guide nucleic acid comprises a guide sequence. The “guide sequence” (also referred to as the “targeting sequence”) can be modified so that the guide RNA can target a CRISPR-Cas effector polypeptide to any desired sequence of any desired target nucleic acid, with the exception that the protospacer adjacent motif (PAM) sequence can be taken into account. A guide nucleic acid suitable for inclusion in a composition of the present disclosure comprises a targeting sequence complementary to a nucleotide sequence in an HBB gene, where the nucleotide sequence in the HBB gene comprises one or more β-thalassemia-associated mutations.

In some cases, the guide RNA is a single-molecule (or “single guide”) guide RNA (an “sgRNA”). In some cases, the guide RNA is a dual-molecule (or “dual-guide”) guide RNA (“dgRNA”).

In some cases, a guide nucleic acid (e.g., a sgRNA) has a total length of from 35 nucleotides (nt) to 150 nt. In some cases, a guide nucleic acid (e.g., a sgRNA) has a total length of from 35 nt to 40 nt, from 40 nt to 45 nt, from 45 nt to 50 nt, from 50 nt to 60 nt, from 60 nt to 70 nt, from 70 nt to 80 nt, from 80 nt to 90 nt, from 90 nt to 100 nt, from 100 nt to 125 nt, or from 125 nt to 150 nt.

The targeting segment of a guide nucleic acid (e.g., a sgRNA) can have a length of 7 or more nucleotides (nt) (e.g., 8 or more, 9 or more, 10 or more, 12 or more, 15 or more, 20 or more, 25 or more, 30 or more, or 40 or more nucleotides). In some cases, the targeting segment can have a length of from 7 to 100 nucleotides (nt) (e.g., from 7 to 80 nt, from 7 to 60 nt, from 7 to 40 nt, from 7 to 30 nt, from 7 to 25 nt, from 7 to 22 nt, from 7 to 20 nt, from 7 to 18 nt, from 8 to 80 nt, from 8 to 60 nt, from 8 to 40 nt, from 8 to 30 nt, from 8 to 25 nt, from 8 to 22 nt, from 8 to 20 nt, from 8 to 18 nt, from 10 to 100 nt, from 10 to 80 nt, from 10 to 60 nt, from 10 to 40 nt, from 10 to 30 nt, from 10 to 25 nt, from 10 to 22 nt, from 10 to 20 nt, from 10 to 18 nt, from 12 to 100 nt, from 12 to 80 nt, from 12 to 60 nt, from 12 to 40 nt, from 12 to 30 nt, from 12 to 25 nt, from 12 to 22 nt, from 12 to 20 nt, from 12 to 18 nt, from 14 to 100 nt, from 14 to 80 nt, from 14 to 60 nt, from 14 to 40 nt, from 14 to 30 nt, from 14 to 25 nt, from 14 to 22 nt, from 14 to 20 nt, from 14 to 18 nt, from 16 to 100 nt, from 16 to 80 nt, from 16 to 60 nt, from 16 to 40 nt, from 16 to 30 nt, from 16 to 25 nt, from 16 to 22 nt, from 16 to 20 nt, from 16 to 18 nt, from 18 to 100 nt, from 18 to 80 nt, from 18 to 60 nt, from 18 to 40 nt, from 18 to 30 nt, from 18 to 25 nt, from 18 to 22 nt, or from 18 to 20 nt).

In some cases, a guide nucleic acid suitable for inclusion in a composition of the present disclosure comprises a nucleotide sequence that hybridizes with a contiguous stretch of from about 7 nucleotides (nt) to about 50 nt (e.g. 7 nt, 8, nt, 9 nt, 10 nt, from 10 nt to 15 nt, from 15 nt to 20 nt, from 20 nt to 25 nt, from 25 nt to 30 nt, from 30 nt to 35 nt, from 35 nt to 40 nt, form 40 nt to 45 nt, or from 45 nt to 50 nt) of a target nucleic acid.

### RNPs

In some cases, the cargo is an RNP. In some cases, a composition of the present disclosure comprises: a) a cargo delivery fusion polypeptide of the present disclosure; and b) an RNP comprising: i) a CRISPR-Cas effector polypeptide; and ii) a CRISPR-Cas guide nucleic acid. In some case, the molar ratio of the cargo delivery fusion polypeptide to the RNP is at least 3:1. In some case, the molar ratio of the cargo delivery fusion polypeptide to the RNP is from about 3:1 to about 50:1. In some case, the molar ratio of the cargo delivery fusion polypeptide to the RNP is from about 3:1 to about 5:1, from about 5:1 to about 10:1, from about 10:1 to about 20:1, from about 20:1 to about 30:1, from about 30:1 to about 40:1, or from about 40:1 to about 50:1.

### Donor Nucleic Acids

In some cases, a composition of the present disclosure includes a donor nucleic acid. In some cases, a donor template nucleic acid suitable for inclusion in a composition of the present disclosure is a donor DNA template comprising a nucleotide sequence that provides for correction a deleterious mutation in a target nucleic acid. In some cases, a donor template nucleic acid suitable for inclusion in a composition of the present disclosure is a donor DNA template comprising a nucleotide sequence that encodes a heterologous polypeptide, e.g., a therapeutic polypeptide (e.g., a CAR). In some cases, the donor template is single stranded (e.g., single-stranded DNA; ssDNA). In some cases, the donor template is double stranded (e.g., double-stranded DNA; dsDNA). In some cases, the donor template comprises both ssDNA and dsDNA. In some cases, the donor temple is present in a recombinant viral vector, e.g., a recombinant adenoassociated virus (AAV) vector.

By a “donor nucleic acid” or “donor sequence” or “donor polynucleotide” or “donor template” or “template” or “repair template” or “homology-directed repair template” (“HDRT”) is meant a nucleic acid sequence to be inserted at the site cleaved by a CRISPR-Cas effector protein (e.g., after dsDNA cleavage, after nicking a target DNA, after dual nicking a target DNA, and the like). The donor polynucleotide can contain sufficient homology to a genomic sequence at the target site, e.g. 70%, 80%. 85%, 90%, 95%, or 100% homology with the nucleotide sequences flanking the target site, e.g. within about 50 bases or less of the target site, e.g. within about 30 bases, within about 15 bases, within about 10 bases, within about 5 bases, or immediately flanking the target site, to support homology-directed repair between it and the genomic sequence to which it bears homology. Approximately 25, 50, 100, or 200 nucleotides, or more than 200 nucleotides, of sequence homology between a donor and a genomic sequence (or any integral value between 10 and 200 nucleotides, or more) can support homology-directed repair. Donor polynucleotides can be of any length, e.g. 10 nucleotides or more, 50 nucleotides or more, 100 nucleotides or more, 250 nucleotides or more, 500 nucleotides or more, 1000 nucleotides or more, 5000 nucleotides or more, etc.

The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair (e.g., for gene correction, e.g., to convert a disease-causing base pair or a non disease-causing base pair).

In some cases, the donor template DNA oligonucleotide has a length of from 50 nucleotides to 100 nucleotides. In some cases, the donor template DNA oligonucleotide has a length of from 50 nucleotides (nt) to 60 nt, from 60 nt to 70 nt, from 70 nt to 80 nt, form 80 nt to 90 nt, or from 90 nt to 100 nt.

Methods of Delivering a Cargo into a Eukaryotic Cell Using a Cargo Delivery Fusion Polypeptide of the Disclosure

The present disclosure provides a method of delivering a cargo into a eukaryotic cell, the method comprising contacting the eukaryotic cell with a composition of the present disclosure, thereby generating a modified eukaryotic cell comprising the cargo. The present disclosure provides a method of delivering a cargo into a target population of eukaryotic cells, the method comprising contacting a target population of eukaryotic cells with a composition of the present disclosure, thereby generating a modified target population of eukaryotic cells comprising the cargo. In some cases, at least 50%, at least 60%, at least 70%, at least 75%, at least 80%, at least 85%, at least 90%, or more than 90%, of the target population of cells is modified to contain the cargo. In some cases, the eukaryotic cell, or target population of eukaryotic cells, is in vitro. In some cases, the eukaryotic cell, or target population of eukaryotic cells, is in vivo.

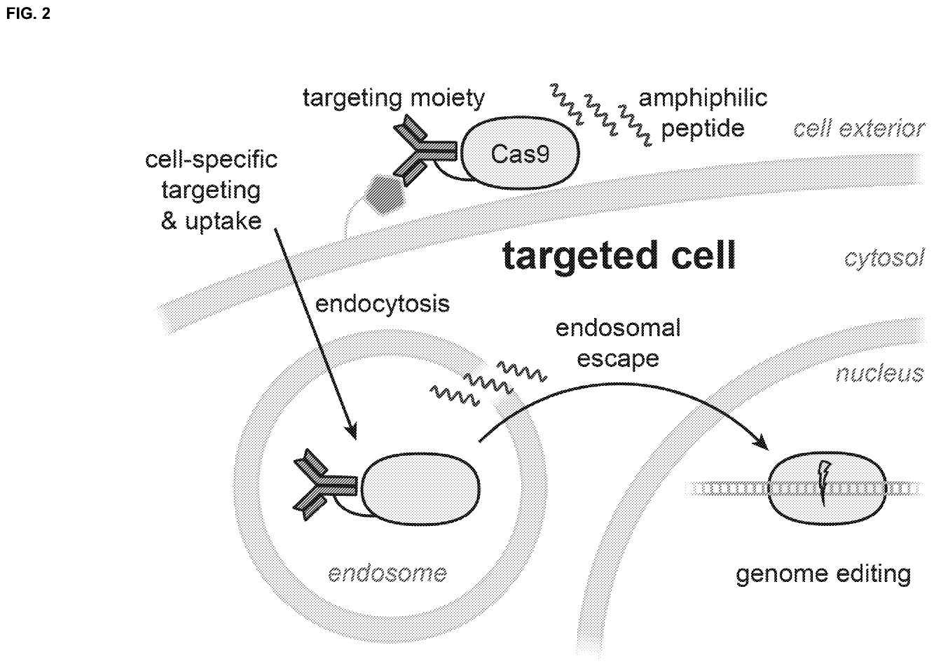

FIG. 2 provides a schematic depiction of delivery of a cargo (in this illustration, the cargo is CRISPR-Cas9) into target cells using a cargo delivery fusion polypeptide. An antibody linked to Cas9 triggers endocytosis by engaging a cell surface receptor, allowing internalization. Amphiphilic peptides allow endosome escape of the internalized cargo. CRISPR-Cas9 is then able to traffic to the nucleus via nuclear localization signals to perform genome editing. FIG. 2 shows Cas9 linked to an antibody; however, in some cases, the cargo (e.g., Cas9 or other cargo) is not linked to an antibody. The amphiphilic peptide provides cell-penetrating activity, allowing translocation of macromolecular cargo across the cell membrane without engaging specific receptors.

In some cases, a method of the present disclosure is less toxic to cells than electroporation. For example, following contacting a target population of eukaryotic cells with a composition of the present disclosure, at least 50%, at least 60%, at least 70%, at least 75%, at least 80%, at least 85%, at least 90%, or more than 90%, of the target population of cells remains viable for a period of time of at least 24 hours, at least 48 hours, at least 72 hours, or at least 5 days following contact of the target population of cells with a composition of the present disclosure. In some cases, a method of the present disclosure provides for modification of a target nucleic acid in at least 30%, at least 40%, at least 50%, at least 60%, at least 70%, at least 80%, or at least 90% of a target population of eukaryotic cells, while maintaining at least viability of at least 50%, at least 60%, at least 70%, at least 75%, at least 80%, at least 85%, at least 90%, or more than 90%, of the target population of cells for a period of time of at least 24 hours, at least 48 hours, at least 72 hours, or at least 5 days following contact of the target population of cells with a composition of the present disclosure.

Eukaryotic cells that can be modified to contain a cargo, using a method of the present disclosure, include, e.g., mammalian cells, e.g., human cells, non-human primate cells, murine cells, etc. Mammalian cells that can be modified to contain a cargo, using a method of the present disclosure, include, e.g., immune cells (e.g., a T cell (e.g., a regulatory T cell, a CD4⁺ T cell, a CD8⁺ T cell), a natural killer (NK) cell, and the like); stem cells; renal cells; neural cells; and the like.

Suitable cells include a stem cell (e.g. an embryonic stem (ES) cell, an induced pluripotent stem (iPS) cell); a germ cell (e.g., an oocyte, a sperm, an oogonia, a spermatogonia, etc.); a somatic cell, e.g. a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogeneic cells, allogenic cells, and post-natal stem cells.

In some cases, the mammalian cell is an immune cell, a neuron, an epithelial cell, and endothelial cell, or a stem cell. In some cases, the immune cell is a T cell, a B cell, a monocyte, a natural killer cell, a dendritic cell, or a macrophage. In some cases, the immune cell is a cytotoxic T cell. In some cases, the immune cell is a helper T cell. In some cases, the immune cell is a regulatory T cell (Treg).

In some cases, the mammalian cell is a stem cell. Stem cells include adult stem cells. Adult stem cells are also referred to as somatic stem cells.

Adult stem cells are resident in differentiated tissue, but retain the properties of self-renewal and ability to give rise to multiple cell types, usually cell types typical of the tissue in which the stem cells are found. Numerous examples of somatic stem cells are known to those of skill in the art, including muscle stem cells; hematopoietic stem cells; epithelial stem cells; neural stem cells; mesenchymal stem cells; mammary stem cells; intestinal stem cells; mesodermal stem cells; endothelial stem cells; olfactory stem cells; neural crest stem cells; and the like.

Stem cells of interest include mammalian stem cells, where the term “mammalian” refers to any animal classified as a mammal, including humans: non-human primates: domestic and farm animals; and zoo, laboratory; sports, or pet animals, such as dogs, horses, cats, cows, mice, rats, rabbits, etc. In some cases, the stem cell is a human stem cell. In some cases, the stem cell is a rodent (e.g., a mouse: a rat) stem cell. In some cases, the stem cell is a non-human primate stem cell.

Stem cells can express one or more stem cell markers, e.g., SOX9, KRT19, KRT7, LGR5, CA9, FXYD2, CDH6, CLDN18, TSPAN8, BPIFB1, OLFM4, CDH17, and PPARGCIA.

In some cases, the stem cell is a hematopoietic stem cell (HSC). HSCs are mesoderm-derived cells that can be isolated from bone marrow, blood, cord blood, fetal liver and yolk sac. HSCs are characterized as CD34 positive and CD38 negative (CD34+ and CD38). HSCs can repopulate the erythroid, neutrophil-macrophage, megakaryocyte and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

In other cases, the stem cell is a neural stem cell (NSC). Neural stem cells (NSCs) are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells respectively. Methods of obtaining NSCs are known in the art.

In other cases, the stem cell is a mesenchymal stem cell (MSC). MSCs originally derived from the embryonal mesoderm and isolated from adult bone marrow, can differentiate to form muscle, bone, cartilage, fat, marrow stroma, and tendon.

In some cases, the target eukaryotic cell is a T cell (or a population of T cells; or a mixed population of cells comprising T cells). In some cases, the target eukaryotic cell is a B cell (or a population of B cells; or a mixed population of cells comprising B cells). In some cases, the target eukaryotic cell is an NK cell (or a population of NK cells; or a mixed population of cells comprising NK cells). In some cases, the target eukaryotic cell is a CD4⁺ T cell (or a population of CD4⁺ T cells; or a mixed population of cells comprising CD4⁺ T cells). In some cases, the target eukaryotic cell is a CD8⁺ T cell (or a population of CD8⁺ T cells; or a mixed population of cells comprising CD8⁺ T cells). In some cases, the target eukaryotic cell is a regulatory T cell (Treg) (or a population of Tregs; or a mixed population of cells comprising Tregs).

In some cases, the target eukaryotic cell is an antigen-presenting cell (APC) (or a population of APCs; or a mixed population of cells comprising APCs). In some cases, the target eukaryotic cell is a dendritic cell (DC) (or a population of DCs; or a mixed population of cells comprising DCs).

In some cases, the target eukaryotic cell is a muscle cell (or a population of muscle cells; or a mixed population of cells comprising muscle cells). In some cases, the muscle cells are skeletal muscle cells.

In some cases, a method of the present disclosure comprises: a) contacting eukaryotic cell, or a target population of eukaryotic cells, with a composition of the present disclosure, thereby generating a modified eukaryotic cell or a modified target population of eukaryotic cells; and b) introducing into the modified eukaryotic cell, or the modified target population of eukaryotic cells, a second cargo. In some cases, step (b) is carried out using electroporation. In some cases, step (b) is carried out using transfection (e.g., contacting the modified eukaryotic cell, or the modified target population of eukaryotic cells, with a recombinant expression vector (e.g., a recombinant viral vector) comprising a nucleotide sequence encoding a cargo). In some cases, step (b) is carried out using a second composition of the present disclosure comprising a second cargo that is different from the cargo delivered in step (a).

In some cases, a method of the present disclosure comprises contacting a target eukaryotic cell, or a target population of eukaryotic cells, with: a) a composition of the present disclosure comprising: i) a cargo delivery fusion polypeptide of the present disclosure; and ii) an RNP comprising a CRISPR-Cas effector polypeptide (or a nucleic acid comprising a nucleotide sequence encoding the CRISPR-Cas effector polypeptide) and a guide RNA (or a nucleic acid comprising a nucleotide sequence encoding the guide RNA); and b) a donor template. In some cases, the donor template is provided in a recombinant vector, such as a recombinant AAV vector. In some cases, the donor template comprises a nucleotide sequence encoding a polypeptide that is heterologous (not naturally present in or produced by) a target eukaryotic cell. In some cases, the donor template comprises a nucleotide sequence encoding a therapeutic polypeptide. As one non-limiting example of this embodiment, T cells obtained from a patient (e.g., a patient having a cancer) are contacted ex vivo with: a) a composition comprising: i) a cargo delivery fusion polypeptide of the present disclosure; and ii) an RNP comprising a CRISPR-Cas effector polypeptide (or a nucleic acid comprising a nucleotide sequence encoding the CRISPR-Cas effector polypeptide) and a guide RNA (or a nucleic acid comprising a nucleotide sequence encoding the guide RNA); and b) a recombinant AAV comprising a donor template encoding a chimeric antigen receptor (CAR), where the CAR comprises a scFv specific for a cancer-associated antigen, where the contacting step results in genetic modification of the T cells such that the T cells produce the CAR and express the CAR on the cell surface. The genetically modified T cells can then be introduced into the patient.

Methods of Delivering a Cargo into a Eukaryotic Cell

The present disclosure provides methods of delivering a cargo into a eukaryotic cell, or a target population of eukaryotic cells. The methods comprising contacting the eukaryotic cell with a composition comprising an amphiphilic polypeptide. Cargos of interest include: i) a DNA molecule comprising a nucleotide sequence encoding an immunogenic polypeptide; and ii) an RNP, where the RNP comprises a CRISPR-Cas effector polypeptide and a guide nucleic acid. In some cases, the cargo comprises a targeting moiety.

FIG. 2 provides a schematic depiction of delivery of a cargo (in this illustration, the cargo is CRISPR-Cas9) into target cells. An antibody linked to Cas9 triggers endocytosis by engaging a cell surface receptor, allowing internalization. Amphiphilic peptides allow endosome escape of the internalized cargo. CRISPR-Cas9 is then able to traffic to the nucleus via nuclear localization signals to perform genome editing. FIG. 2 shows Cas9 linked to an antibody; however, in some cases, the cargo (e.g., Cas9 or other cargo) is not linked to an antibody. The amphiphilic peptide provides cell-penetrating activity, allowing translocation of macromolecular cargo across the cell membrane without engaging specific receptors.

In some cases, the eukaryotic cell, or target population of eukaryotic cells, is in vitro. In some cases, the eukaryotic cell, or target population of eukaryotic cells, is in vivo. In some cases, the eukaryotic cell, or target population of eukaryotic cells, is ex vivo.

Eukaryotic cells that can be modified to contain a cargo, using a method of the present disclosure, include, e.g., mammalian cells, e.g., human cells, non-human primate cells, murine cells, etc. Mammalian cells that can be modified to contain a cargo, using a method of the present disclosure, include, e.g., immune cells (e.g., a T cell, an NK cell, and the like); stem cells; renal cells; neural cells; and the like.

Suitable cells include a stem cell (e.g. an ES cell, an iPS cell); a germ cell (e.g., an oocyte, a sperm, an oogonia, a spermatogonia, etc.); a somatic cell, e.g. a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogeneic cells, allogenic cells, and post-natal stem cells.

In some cases, the mammalian cell is an immune cell, a neuron, an epithelial cell, and endothelial cell, or a stem cell. In some cases, the immune cell is a T cell, a B cell, a monocyte, a natural killer cell, a dendritic cell, or a macrophage. In some cases, the immune cell is a cytotoxic T cell. In some cases, the immune cell is a helper T cell. In some cases, the immune cell is a regulatory T cell (Treg).

In some cases, the mammalian cell is a stem cell. Stem cells include adult stem cells. Adult stem cells are also referred to as somatic stem cells.

Adult stem cells are resident in differentiated tissue, but retain the properties of self-renewal and ability to give rise to multiple cell types, usually cell types typical of the tissue in which the stem cells are found. Numerous examples of somatic stem cells are known to those of skill in the art, including muscle stem cells; hematopoietic stem cells; epithelial stem cells; neural stem cells; mesenchymal stem cells; mammary stem cells; intestinal stem cells; mesodermal stem cells; endothelial stem cells; olfactory stem cells; neural crest stem cells; and the like.

Stem cells of interest include mammalian stem cells, where the term “mammalian” refers to any animal classified as a mammal, including humans: non-human primates: domestic and farm animals; and zoo, laboratory; sports, or pet animals, such as dogs, horses, cats, cows, mice, rats, rabbits, etc. In some cases, the stem cell is a human stem cell. In some cases, the stem cell is a rodent (e.g., a mouse: a rat) stem cell. In some cases, the stem cell is a non-human primate stem cell.

Stem cells can express one or more stem cell markers, e.g., SOX9, KRT19, KRT7, LGR5, CA9, FXYD2, CDH6, CLDN18, TSPAN8, BPIFB1, OLFM4, CDH17, and PPARGCIA.

In some cases, the stem cell is an HSC. In other cases, the stem cell is an NSC. In other cases, the stem cell is an MSC.

In some cases, the target eukaryotic cell is a T cell (or a population of T cells; or a mixed population of cells comprising T cells). In some cases, the target eukaryotic cell is a B cell (or a population of B cells; or a mixed population of cells comprising B cells). In some cases, the target eukaryotic cell is an NK cell (or a population of NK cells; or a mixed population of cells comprising NK cells). In some cases, the target eukaryotic cell is a CD4⁺ T cell (or a population of CD4⁺ T cells; or a mixed population of cells comprising CD4⁺ T cells). In some cases, the target eukaryotic cell is a CD8⁺ T cell (or a population of CD8⁺ T cells; or a mixed population of cells comprising CD8⁺ T cells). In some cases, the target eukaryotic cell is a regulatory T cell (Treg) (or a population of Tregs; or a mixed population of cells comprising Tregs).

In some cases, the target eukaryotic cell is an antigen-presenting cell (APC) (or a population of APCs; or a mixed population of cells comprising APCs). In some cases, the target eukaryotic cell is a dendritic cell (DC) (or a population of DCs; or a mixed population of cells comprising DCs).

In some cases, the RNP being delivered to a cell comprises a CRISPR-Cas effector polypeptide, or a CRISPR-Cas fusion polypeptide, that comprises a covalently linked antibody or non-antibody-based recognition scaffold. Suitable non-antibody-based recognition scaffolds include an avimer, a DARPin, an adnectin, an avimer, an affibody, an anticalin, or an affilin. The covalently linked antibody or non-antibody-based recognition scaffold can be linked to the CRISPR-Cas effector polypeptide via a proteolytically cleavable linker. The covalently linked antibody or non-antibody-based recognition scaffold can target the CRISPR-Cas effector polypeptide or CRISPR-Cas fusion polypeptide to a target eukaryotic cell.

### Amphiphilic Polypeptide and Compositions

An amphiphilic polypeptide suitable for use in a method of the present disclosure comprises: i) an endosomolytic polypeptide; and ii) a cell penetrating polypeptide.

Suitable endosomolytic polypeptides include, e.g., a polypeptide comprising the amino acid sequence: GLFEAIAEFIENGWEGLIEGWYG (SEQ ID NO:163), or a polypeptide having from 1 to 5 amino acid substitutions relative to GLFEAIAEFIENGWEGLIEGWYG (SEQ ID NO:163); where the endosomolytic polypeptide has a length of from about 20 amino acids to about 25 amino acids.

Suitable endosomolytic polypeptides include, e.g., a polypeptide comprising the amino acid sequence: GLFEAIEGFIENGWEGMIDGWYG (SEQ ID NO:164), or a polypeptide having from 1 to 5 amino acid substitutions relative to GLFEAIEGFIENGWEGMIDGWYG (SEQ ID NO:164); where the endosomolytic polypeptide has a length of from about 20 amino acids to about 25 amino acids.

Suitable cell penetrating polypeptides include, e.g., YGRKKRRQRRR (SEQ ID NO: 207), YGRKKRRQRR (SEQ ID NO:160), or GRKKRRQRRR (SEQ ID NO:161), where the cell penetrating polypeptide has a length of from 10 amino acids to 15 amino acids.

In some cases, the total length of an amphiphilic polypeptide is from 35 amino acids to 50 amino acids. In some cases, the total length of an amphiphilic polypeptide is from 35 amino acids to 40 amino acids. In some cases, the total length of an amphiphilic polypeptide is from 35 amino acids to 45 amino acids. In some cases, the total length of an amphiphilic polypeptide is from 40 amino acids to 45 amino acids. In some cases, the total length of an amphiphilic polypeptide is from 40 amino acids to 50 amino acids.

In some cases, a suitable amphiphilic polypeptide comprises the amino acid sequence of any one of the peptides identified in FIG. 1 as Peptide #1-#60. In some cases, a suitable amphiphilic polypeptide comprises an amino acid sequence having from 1 to 5 amino acid substitutions compared to any one of the peptides identified in FIG. 1 as Peptide #1-#60. In some cases, the total length of an amphiphilic polypeptide is from 35 amino acids to 50 amino acids. In some cases, the total length of an amphiphilic polypeptide is from 35 amino acids to 40 amino acids. In some cases, the total length of an amphiphilic polypeptide is from 35 amino acids to 45 amino acids. In some cases, the total length of an amphiphilic polypeptide is from 40 amino acids to 45 amino acids. In some cases, the total length of an amphiphilic polypeptide is from 40 amino acids to 50 amino acids.

In some cases, a suitable amphiphilic polypeptide comprises the amino acid sequence of any one of the peptides identified in FIG. 1 as Peptide #1-#18, or Peptide #20-37. In some cases, a suitable amphiphilic polypeptide comprises an amino acid sequence having from 1 to 5 amino acid substitutions compared to any one of the peptides identified in FIG. 1 as Peptide #1-#18, or Peptide #20-37. In some cases, the total length of an amphiphilic polypeptide is from 35 amino acids to 50 amino acids. In some cases, the total length of an amphiphilic polypeptide is from 35 amino acids to 40 amino acids. In some cases, the total length of an amphiphilic polypeptide is from 35 amino acids to 45 amino acids. In some cases, the total length of an amphiphilic polypeptide is from 40 amino acids to 45 amino acids. In some cases, the total length of an amphiphilic polypeptide is from 40 amino acids to 50 amino acids.

In some cases, a suitable amphiphilic polypeptide comprises the amino acid sequence of any one of the peptides identified in FIG. 1 as Peptide #19 or Peptide #40-60. In some cases, a suitable amphiphilic polypeptide comprises an amino acid sequence having from 1 to 5 amino acid substitutions compared to any one of the peptides identified in FIG. 1 as Peptide #19 or Peptide #40-60. In some cases, the total length of an amphiphilic polypeptide is from 35 amino acids to 50 amino acids. In some cases, the total length of an amphiphilic polypeptide is from 35 amino acids to 40 amino acids. In some cases, the total length of an amphiphilic polypeptide is from 35 amino acids to 45 amino acids. In some cases, the total length of an amphiphilic polypeptide is from 40 amino acids to 45 amino acids. In some cases, the total length of an amphiphilic polypeptide is from 40 amino acids to 50 amino acids.

In some cases, an amphiphilic polypeptide composition of the present disclosure comprises: a) an amphiphilic polypeptide; and b) DMSO. In some cases, an amphiphilic polypeptide is maintained in a solution comprising DMSO in a concentration of from about 9% DMSO to about 15% DMSO; e.g., about 10% DMSO) for a period of time before being contacted with the cargo. When an amphiphilic polypeptide is prepared in a solution of less than 10% DMSO, the peptides may bind to each other and in some cases may not productively associate with the cargo. Thus, in some cases, an amphiphilic polypeptide is kept in a solution of about 10% DMSO for a period of time; after which the peptide is contacted with the cargo that is present in a solution (e.g., a buffered aqueous solution) without DMSO. The amphiphilic polypeptide/cargo solution may thus contain from 1% DMSO to 5% DMSO.

In some cases, a composition of the present disclosure comprises: a) an amphiphilic polypeptide; and b) saline (e.g., 0.9% NaCl). In some cases, the composition is sterile. In some cases, the composition is suitable for administration to a human subject, e.g., where the composition is sterile and is free of detectable pyrogens and/or other toxins. Thus, the present disclosure provides a composition comprising: a) an amphiphilic polypeptide; and b) saline (e.g., 0.9% NaCl), where the composition is sterile and is free of detectable pyrogens and/or other toxins. In some cases, the composition further comprises a cargo to be delivered.

### Cargo

As noted above, cargos of interest include: i) a DNA molecule comprising a nucleotide sequence encoding an immunogenic polypeptide; and ii) an RNP, where the RNP comprises a CRISPR-Cas effector polypeptide and a guide nucleic acid.

### DNA Encoding Immunogenic Polypeptide

In some cases, the cargo is a DNA molecule comprising a nucleotide sequence encoding an immunogenic polypeptide. An immunogenic protein is suitable for stimulating an immune response to the antigenic protein in a mammalian host (e.g., a human, a non-human primate, a bovine (e.g., a cow), an ovine (e.g., a sheep), an equine (e.g., a horse), a porcine (e.g, a pig), and the like). The immunogenic polypeptide can be derived from an autoantigen, an allergen, a tumor-associated antigen, a pathogenic virus, a pathogenic bacterium, a pathogenic protozoan, a pathogenic helminth, or any other pathogenic organism that infects a mammalian host. Suitable immunogenic polypeptides can be derived from any of a variety of pathogens, as described above.

### RNPs

In some cases, the cargo is an RNP comprising a CRISPR-Cas effector polypeptide and a guide nucleic acid. Suitable CRISPR-Cas effector polypeptides and guide nucleic acids are described above. In some case, the molar ratio of the cargo delivery fusion polypeptide to the RNP is at least 3:1. In some case, the molar ratio of the cargo delivery fusion polypeptide to the RNP is from about 3:1 to about 50:1. In some case, the molar ratio of the cargo delivery fusion polypeptide to the RNP is from about 3:1 to about 5:1, from about 5:1 to about 10:1, from about 10:1 to about 20:1, from about 20:1 to about 30:1, from about 30:1 to about 40:1, or from about 40:1 to about 50:1.

In some cases, a method of the present disclosure comprises contacting a target eukaryotic cell, or a target population of eukaryotic cells, with: a) a composition of the present disclosure comprising: i) an amphiphilic polypeptide (as described above); and ii) an RNP comprising a CRISPR-Cas effector polypeptide (or a nucleic acid comprising a nucleotide sequence encoding the CRISPR-Cas effector polypeptide) and a guide RNA (or a nucleic acid comprising a nucleotide sequence encoding the guide RNA); and b) a donor template. In some cases, the donor template is provided in a recombinant vector, such as a recombinant AAV vector. As one non-limiting example of this embodiment, T cells obtained from a patient (e.g., a patient having a cancer) are contacted ex vivo with: a) a composition comprising: i) an amphiphilic polypeptide (as described above); and ii) an RNP comprising a CRISPR-Cas effector polypeptide (or a nucleic acid comprising a nucleotide sequence encoding the CRISPR-Cas effector polypeptide) and a guide RNA (or a nucleic acid comprising a nucleotide sequence encoding the guide RNA); and b) a recombinant AAV comprising a donor template encoding a chimeric antigen receptor (CAR), where the CAR comprises a scFv specific for a cancer-associated antigen, where the contacting step results in genetic modification of the T cells such that the T cells produce the CAR and express the CAR on the cell surface. The genetically modified T cells can then be introduced into the patient.

In some cases, the RNP being delivered to a cell comprises a CRISPR-Cas effector polypeptide, or a CRISPR-Cas fusion polypeptide, that comprises a covalently linked antibody or non-antibody-based recognition scaffold. Suitable non-antibody-based recognition scaffolds include an avimer, a DARPin, an adnectin, an avimer, an affibody, an anticalin, or an affilin. The covalently linked antibody or non-antibody-based recognition scaffold can be linked to the CRISPR-Cas effector polypeptide via a proteolytically cleavable linker. The covalently linked antibody or non-antibody-based recognition scaffold can target the CRISPR-Cas effector polypeptide or CRISPR-Cas fusion polypeptide to a target eukaryotic cell.

### Compositions

The cargo to be delivered using a method of the present disclosure can be present in a composition. For example, the composition can comprise, in addition to an amphiphilic polypeptide and a cargo, one or more of: a salt, e.g., NaCl, MgCl₂, KCl, MgSO₄, etc.; a buffering agent, e.g., a Tris buffer, N-(2-Hydroxyethyl) piperazine-N′-(2-ethanesulfonic acid) (HEPES), 2-(N-Morpholino) ethanesulfonic acid (MES), 2-(N-Morpholino) ethanesulfonic acid sodium salt (MES), 3-(N-Morpholino) propanesulfonic acid (MOPS), N-tris [Hydroxymethyl]methyl-3-aminopropanesulfonic acid (TAPS), etc.; a solubilizing agent; a detergent, e.g., a non-ionic detergent such as Tween-20, etc.; a nuclease inhibitor; glycerol; and the like.

Such a composition can be administered to an individual in need thereof, using any of a variety of routes of administration, including local and systemic routes of administration. Suitable routes of administration include intravenous, intramuscular, subcutaneous, peritumoral, and the like. In some cases, the composition can be administered to an individual in need thereof by administering the composition into or near a target organ.

### Examples of Non-Limiting Aspects of the Disclosure

### Aspects Set A

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A cargo delivery fusion polypeptide comprising:
a) an endosomolytic polypeptide; and
b) a cell penetrating polypeptide,
wherein the fusion polypeptide comprises an amino acid sequence of a formula selected from:

i)
(SEQ ID NO: 165)
KLFEX₁IEGFIENGWEX₂MIDX₃WX₄GX₅GRKKRRQRR,

wherein
X₁ is A, R, or K;
X₂ is A or G;
X₃ is L or G;
X₄ is N or Y; and
X₅, if present, is Y;
ii) X₁LFEX₂IEGFIENGWEGMIDGWYGYGRKKRRQRR (SEQ ID NO:166), wherein
X₁ is R or G; and
X₂ is R or K;
iii) GLFEAIEGFIENGWEX₁MIDX₂WNGYGRKKRRQRR (SEQ ID NO:167), wherein
X₁ is A or G; and
X₂ is G or L;
iv) GLFEAIEGFIENGWEX₁X₂IX₃LWYGYGRKKRRQRR (SEQ ID NO:168), wherein
X₁ is A or G;
X₂ is L or M; and
X₃ is D or E; and
v) GLFX₁AIAX₂FIX₃NGWX₄GLIX₅GWYGGRKKRRQRRR (SEQ ID NO:208), wherein each of X₁, X₂, X₃, X₄, and X₅ is independently a non-coded amino acid, and
wherein the fusion polypeptide has a length of from about 32 amino acids to about 35 amino acids.

Aspect 2. The fusion polypeptide of aspect 1, wherein the fusion polypeptide comprises an amino acid sequence of the formula: KLFEX₁IEGFIENGWEX₂MIDX₃WX₄GX₅GRKKRRQRR (SEQ ID NO: 165), wherein
X₁ is A, R, or K;
X₂ is A or G;
X₃ is L or G;
X₄ is N or Y; and
X₅, if present, is Y.

Aspect 3. The fusion polypeptide of aspect 2, wherein the fusion polypeptide comprises an amino acid sequence selected from the group consisting of:

(peptide 19; SEQ ID NO: 19)
KLFEAIEGFIENGWEGMIDGWYGYGRKKRRQRR;
(peptide 40; SEQ ID NO: 40)
KLFEAIEGFIENGWEGMIDGWYGGRKKRRQRR;
(peptide 44; SEQ ID NO: 44)
KLFERIEGFIENGWEGMIDGWYGYGRKKRRQRR;
(peptide 45; SEQ ID NO: 45)
KLFEKIEGFIENGWEGMIDGWYGYGRKKRRQRR;
(peptide 46; SEQ ID NO: 46)
KLFEAIEGFIENGWEAMIDGWYGYGRKKRRQRR;
(peptide 47; SEQ ID NO: 47)
KLFEAIEGFIENGWEGMIDLWYGYGRKKRRQRR;
(peptide 48; SEQ ID NO: 48)
KLFEAIEGFIENGWEGMIDGWNGYGRKKRRQRR;
(peptide 53; SEQ ID NO: 53)
KLFEAIEGFIENGWEAMIDLWYGYGRKKRRQRR;
(peptide 54; SEQ ID NO: 54)
KLFEAIEGFIENGWEAMIDGWNGYGRKKRRQRR;
(peptide 55; SEQ ID NO: 55)
KLFEAIEGFIENGWEGMIDLWNGYGRKKRRQRR;
(peptide 56; SEQ ID NO: 56)
KLFEAIEGFIENGWEAMIDLWNGYGRKKRRQRR;
and
(peptide 57; SEQ ID NO: 57)
KLFEKIEGFIENGWEAMIDLWNGYGRKKRRQRR.

Aspect 4. The fusion polypeptide of aspect 1, wherein the fusion polypeptide comprises an amino acid sequence of the formula: X₁LFEX₂IEGFIENGWEGMIDGWYGYGRKKRRQRR (SEQ ID NO: 166), wherein X₁ is R or G; and X₂ is R or K.

Aspect 5. The fusion polypeptide of aspect 4, wherein the fusion polypeptide comprises an amino acid sequence selected from the group consisting of:

(peptide 42; SEQ ID NO: 42)
RLFERIEGFIENGWEGMIDGWYGYGRKKRRQRR;
(peptide 43; SEQ ID NO: 43)
RLFEKIEGFIENGWEGMIDGWYGYGRKKRRQRR;
and
(peptide 41; SEQ ID NO: 41)
GLFERIEGFIENGWEGMIDGWYGYGRKKRRQRR.

Aspect 6. The fusion polypeptide of aspect 1, wherein the fusion polypeptide comprises an amino acid sequence of the formula: GLFEAIEGFIENGWEX₁MIDX₂WNGYGRKKRRQRR (SEQ ID NO: 167), wherein X₁ is A or G; and X₂ is G or L.

Aspect 7. The fusion polypeptide of aspect 6, wherein the fusion polypeptide comprises an amino acid sequence selected from the group consisting of:

(peptide 50; SEQ ID NO: 50)
GLFEAIEGFIENGWEAMIDGWNGYGRKKRRQRR;
(peptide 51; SEQ ID NO: 51)
GLFEAIEGFIENGWEGMIDLWNGYGRKKRRQRR;
and
(peptide 51; SEQ ID NO: 52)
GLFEAIEGFIENGWEAMIDLWNGYGRKKRRQRR.

Aspect 8. The fusion polypeptide of aspect 1, wherein the fusion polypeptide comprises an amino acid sequence of the formula: GLFEAIEGFIENGWEX₁X₂IX₃LWYGYGRKKRRQRR (SEQ ID NO: 168), wherein:
X₁ is A or G;
X₂ is L or M; and
X₃ is D or E.

Aspect 9. The fusion polypeptide of aspect 8, wherein the fusion polypeptide comprises the amino acid sequence:

(peptide 49; SEQ ID NO: 49)
GLFEAIEGFIENGWEAMIDLWYGYGRKKRRQRR;
or
(peptide 58; SEQ ID NO: 58)
GLFEAIEGFIENGWEGLIELWYGYGRKKRRQRR.

Aspect 10. The fusion polypeptide of aspect 1, wherein the fusion polypeptide comprises an amino acid sequence of the formula: GLFX₁AIAX₂FIX₃NGWX₄GLIX₅GWYGGRKKRRQRRR (SEQ ID NO:208), wherein each of X₁, X₂, X₃, X₄, and X₅ is independently a non-coded amino acid.

Aspect 11. The fusion polypeptide of aspect 10, wherein the fusion polypeptide comprises the amino acid sequence:

(peptide 59; SEQ ID NO: 59)
GLFαAIAαFIαNGWαGLIαGWYGGRKKRRQRRR;
or
(peptide 60; SEQ ID NO: 60)
GLFαAIAαFIENGWEGLIDGWYGGRKKRRQRRR.

Aspect 12. The fusion polypeptide of aspect 10 or aspect 11, wherein each of X₁, X₂, X₃, X₄, and X₅ is α-aminoadipic acid.

Aspect 13. A composition comprising the cargo delivery fusion polypeptide of any one of aspects 1-12.

Aspect 14. The composition of aspect 13, further comprising a cargo, wherein the cargo comprises one or more of a nucleic acid, a polypeptide, and a ribonucleoprotein complex.

Aspect 15. The composition of aspect 14, wherein the cargo comprises a targeting moiety.

Aspect 16. The composition of aspect 13, comprising a nucleic acid comprising a nucleotide sequence encoding a gene product of interest.

Aspect 17. The composition of aspect 16, wherein the gene product of interest is an antigen.

Aspect 18. The composition of aspect 16 or aspect 7, wherein the nucleic acid is a recombinant expression vector.

Aspect 19. The composition of aspect 18, wherein the recombinant expression vector is a recombinant viral vector.

Aspect 20. The composition of aspect 13, comprising:
a) a CRISPR-Cas effector polypeptide; or
b) a fusion polypeptide comprising:
   i) a CRISPR-Cas effector polypeptide; and
   ii) one or more heterologous polypeptides.

Aspect 21. The composition of aspect 20, comprising a CRISPR-Cas guide nucleic acid.

Aspect 22. The composition of aspect 21, comprising a donor DNA template.

Aspect 23. The composition of any one of aspects 20-22, wherein the CRISPR-Cas effector polypeptide is a type II CRISPR-Cas effector polypeptide, a type V CRISPR-Cas effector polypeptide, or a type VI CRISPR-Cas effector polypeptide.

Aspect 24. The composition of aspect 21, wherein the CRISPR-Cas guide nucleic acid is RNA.

Aspect 25. The composition of aspect 24, wherein the CRISPR-Cas guide nucleic acid is a single-molecule guide RNA or a dual-molecule guide RNA.

Aspect 26. The composition of any one of aspects 20-25, wherein the composition comprises a CRISPR-Cas effector fusion polypeptide comprising: i) a CRISPR-Cas effector fusion polypeptide; and ii) one or more nuclear localization signals.

Aspect 27. The composition of any one of aspects 20-25, wherein the composition comprises a CRISPR-Cas effector fusion polypeptide comprising: i) a CRISPR-Cas effector polypeptide; and ii) one or more heterologous effector polypeptides.

Aspect 28. The composition of aspect 27, wherein at least one of the one or more heterologous effector polypeptides is a single stranded nuclease, a double strand nuclease, a helicase, a methylase, a demethylase, an acetylase, a deacetylase, a deaminase, an integrase, a recombinase, a base editor, or a prime editor.

Aspect 29. The composition of any one of aspects 20-28, wherein the CRISPR-Cas effector polypeptide, or the CRISPR-Cas effector fusion polypeptide, comprises a covalently linked targeting moiety.

Aspect 30. The composition of aspect 29, wherein the targeting moiety is protein A, protein G, an aptamer, a darpin, or an antibody.

Aspect 31. The composition of aspect 30, comprising an antibody non-covalently bound to the affinity moiety.

Aspect 32. The composition of aspect 30 or aspect 31, where the antibody specifically binds an epitope on the surface of a eukaryotic cell, thereby targeting the composition to the cell.

Aspect 33. The composition of any one of aspects 20-32, wherein the CRISPR-Cas effector polypeptide, or the CRISPR-Cas effector fusion polypeptide, comprises a non-polypeptide polymer.

Aspect 34. The composition of aspect 33, wherein the non-polypeptide polymer is poly(ethylene glycol).

Aspect 35. The composition of any one of aspects 21-34, wherein the CRISPR-Cas effector polypeptide and the guide nucleic acid are in a ribonucleoprotein (RNP) complex.

Aspect 36. The composition of aspect 35, wherein the molar ratio of cargo delivery fusion polypeptide to RNP is at least 3:1.

Aspect 37. The composition of aspect 36, wherein the molar ratio of cargo delivery fusion polypeptide to RNP is from 10:1 to 50:1.

Aspect 38. The composition of any one of aspects 13-37, wherein the cargo delivery fusion polypeptide is present in the composition in a concentration of from about 2 μM to about 50 μM.

Aspect 39. The composition of any one of aspects 13-38, comprising one or more of a solubilizing agent, a surfactant, a buffer, a salt, and a protease inhibitor.

Aspect 40. The composition of any one of aspects 13-38, comprising poly(ethylene glycol), a non-ionic surfactant, or both.

Aspect 41. A method of delivering a cargo into a target population of eukaryotic cells, the method comprising contacting the cell with the composition of any one of aspects 14-38, thereby generating a modified target population of eukaryotic cells comprising the cargo.

Aspect 42. The method of aspect 41, wherein the target population of eukaryotic cells comprises a T cell, a stem cell, a natural killer cell, a renal cell or a neural cell.

Aspect 43. The method of aspect 41, wherein the target eukaryotic cell is a hematopoietic stem cell or a hematopoietic progenitor cell.

Aspect 44. The method of any one of aspects 41-43, wherein the cell is in vitro.

Aspect 45. The method of aspect 44, wherein at least 50% of the target population of eukaryotic cells retain viability after said contacting.

Aspect 46. The method of aspect 44 or aspect 45, wherein the method comprises introducing into the modified target population of eukaryotic cells a second composition comprising a second cargo.

Aspect 47. The method of aspect 46, wherein said introducing is via electroporation or transfection.

Aspect 48. The method of aspect 47, wherein said transfection comprises contacting the modified target population of eukaryotic cells with a recombinant viral vector.

Aspect 49. The method of any one of aspects 41-43, wherein the cell is in vivo.

Aspect 50. A method of delivering a DNA molecule into a eukaryotic cell, the method comprising contacting the cell with a composition comprising:
a) an amphiphilic cargo delivery fusion polypeptide comprising:
   i) an endosomolytic polypeptide; and
   ii) a cell penetrating polypeptide; and
b) a DNA molecule comprising a nucleotide sequence encoding an immunogenic polypeptide.

Aspect 51. The method of aspect 50, wherein the immunogenic polypeptide is a viral polypeptide.

Aspect 52. The method of aspect 50 or aspect 51, wherein the endosomolytic polypeptide:
a) comprises the amino acid sequence GLFEAIAEFIENGWEGLIEGWYG (SEQ ID NO: 163);
b) comprises from 1 to 5 amino acid substitutions relative to GLFEAIAEFIENGWEGLIEGWYG (SEQ ID NO:163);
c) comprises the amino acid sequence GLFEAIEGFIENGWEGMIDGWYG (SEQ ID NO: 164); or
d) comprises from 1 to 5 amino acid substitutions relative to GLFEAIEGFIENGWEGMIDGWYG (SEQ ID NO:164),
wherein the endosomolytic polypeptide has a length of from about 20 amino acids to about 30 amino acids.

Aspect 53. The method of any one of aspects 50-52, wherein the cell penetrating polypeptide comprises the amino acid sequence YGRKKRRQRRR (SEQ ID NO:207), YGRKKRRQRR (SEQ ID NO:160), or GRKKRRQRRR (SEQ ID NO:161), and has a length of from 10 amino acids to 15 amino acids.

Aspect 54. A method of delivering a ribonucleoprotein (RNP) into a eukaryotic cell, the method comprising contacting the cell with a composition comprising:
a) an amphiphilic cargo delivery fusion polypeptide comprising:
   i) an endosomolytic polypeptide; and
   ii) a cell penetrating polypeptide; and
b) an RNP comprising:
   i) a CRISPR-Cas effector polypeptide; and
   ii) a guide nucleic acid.

Aspect 55. The method of aspect 54, wherein the endosomolytic polypeptide:
a) comprises the amino acid sequence GLFEAIAEFIENGWEGLIEGWYG (SEQ ID NO: 163);
b) comprises from 1 to 5 amino acid substitutions relative to GLFEAIAEFIENGWEGLIEGWYG (SEQ ID NO:163);
c) comprises the amino acid sequence GLFEAIEGFIENGWEGMIDGWYG (SEQ ID NO: 164); or
d) comprises from 1 to 5 amino acid substitutions relative to GLFEAIEGFIENGWEGMIDGWYG (SEQ ID NO:164),
wherein the endosomolytic polypeptide has a length of from about 20 amino acids to about 30 amino acids.

Aspect 56. The method of aspect 54 or aspect 55, wherein the cell penetrating polypeptide comprises the amino acid sequence YGRKKRRQRRR (SEQ ID NO:207), YGRKKRRQRR (SEQ ID NO:160), or GRKKRRQRRR (SEQ ID NO:161), and has a length of from 10 amino acids to 15 amino acids.

Aspect 57. The method of any one of aspects 54-56, comprising introducing into the cell a DNA donor template.

Aspect 58. The method of aspect 57, wherein the donor template is present in a recombinant viral vector.

Aspect 59. The method of aspect 58, wherein the recombinant viral vector is a recombinant adenoassociated viral vector.

Aspect 60. The method of any one of aspects 57-59, wherein the donor template comprises a nucleotide sequence encoding a polypeptide.

Aspect 61. The method of aspect 60, wherein the polypeptide is a chimeric antigen receptor comprising a single-chain Fv or a nanobody specific for a cancer-associated antigen.

Aspect 62. The method of any one of aspects 54-61, wherein the eukaryotic cell is an immune cell.

Aspect 63. The method of aspect 62, wherein the immune cell is a T cell.

Aspect 64. The method of any one of aspects 54-63, wherein the eukaryotic cell is in vivo.

Aspect 65. The method of any one of aspects 54-63, wherein the eukaryotic cell is in vitro.

### Aspects Set B

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A cargo delivery fusion polypeptide comprising: a) an endosomolytic polypeptide; and b) a cell penetrating polypeptide, wherein the fusion polypeptide comprises an amino acid sequence of any one of Formulas I-VIII, wherein the fusion polypeptide has a length of from about 32 amino acids to about 35 amino acids, and wherein any two adjacent amino acids are independently linked by an amide bond or a non-amide bond.

Aspect 2. The fusion polypeptide of aspect 1, wherein the fusion polypeptide comprises an amino acid sequence of Formula I: KLFEX₁IEGFIENGWEX₂MIDX₃WX₄GX₅GRKKRRQRR (SEQ ID NO: 165), wherein
X₁ is A, R, or K;
X₂ is A or G;
X₃ is L or G;
X₄ is N or Y; and
X₅, if present, is Y.

Aspect 3. The fusion polypeptide of aspect 2, wherein the fusion polypeptide comprises an amino acid sequence selected from the group consisting of:

(peptide 19; SEQ ID NO: 19)
KLFEAIEGFIENGWEGMIDGWYGYGRKKRRQRR;
(peptide 40; SEQ ID NO: 40)
KLFEAIEGFIENGWEGMIDGWYGGRKKRRQRR;
(peptide 44; SEQ ID NO: 44)
KLFERIEGFIENGWEGMIDGWYGYGRKKRRQRR;
(peptide 45; SEQ ID NO: 45)
KLFEKIEGFIENGWEGMIDGWYGYGRKKRRQRR;
(peptide 46; SEQ ID NO: 46)
KLFEAIEGFIENGWEAMIDGWYGYGRKKRRQRR;
(peptide 47; SEQ ID NO: 47)
KLFEAIEGFIENGWEGMIDLWYGYGRKKRRQRR;
(peptide 48; SEQ ID NO: 48)
KLFEAIEGFIENGWEGMIDGWNGYGRKKRRQRR;
(peptide 53; SEQ ID NO: 53)
KLFEAIEGFIENGWEAMIDLWYGYGRKKRRQRR;
(peptide 54; SEQ ID NO: 54)
KLFEAIEGFIENGWEAMIDGWNGYGRKKRRQRR;
(peptide 55; SEQ ID NO: 55)
KLFEAIEGFIENGWEGMIDLWNGYGRKKRRQRR;
(peptide 56; SEQ ID NO: 56)
KLFEAIEGFIENGWEAMIDLWNGYGRKKRRQRR;
and
(peptide 57; SEQ ID NO: 57)
KLFEKIEGFIENGWEAMIDLWNGYGRKKRRQRR.

Aspect 4. The fusion polypeptide of aspect 1, wherein the fusion polypeptide comprises an amino acid sequence of Formula II: X₁LFEX₂IEGFIENGWEGMIDGWYGYGRKKRRQRR (SEQ ID NO: 166), wherein X₁ is R or G; and X₂ is R or K.

Aspect 5. The fusion polypeptide of aspect 4, wherein the fusion polypeptide comprises an amino acid sequence selected from the group consisting of:

(peptide 42; SEQ ID NO: 42)
RLFERIEGFIENGWEGMIDGWYGYGRKKRRQRR;
(peptide 43; SEQ ID NO: 43)
RLFEKIEGFIENGWEGMIDGWYGYGRKKRRQRR;
and
(peptide 41; SEQ ID NO: 41)
GLFERIEGFIENGWEGMIDGWYGYGRKKRRQRR.

Aspect 6. The fusion polypeptide of aspect 1, wherein the fusion polypeptide comprises an amino acid sequence of Formula III: GLFEAIEGFIENGWEX₁MIDX₂WNGYGRKKRRQRR (SEQ ID NO: 167), wherein X₁ is A or G; and X₂ is G or L.

Aspect 7. The fusion polypeptide of aspect 6, wherein the fusion polypeptide comprises an amino acid sequence selected from the group consisting of:

(peptide 50; SEQ ID NO: 50)
GLFEAIEGFIENGWEAMIDGWNGYGRKKRRQRR;
(peptide 51; SEQ ID NO: 51)
GLFEAIEGFIENGWEGMIDLWNGYGRKKRRQRR;
and
(peptide 51; SEQ ID NO: 51)
GLFEAIEGFIENGWEAMIDLWNGYGRKKRRQRR.

Aspect 8. The fusion polypeptide of aspect 1, wherein the fusion polypeptide comprises an amino acid sequence of Formula IV: GLFEAIEGFIENGWEX₁X₂IX₃LWYGYGRKKRRQRR (SEQ ID NO: 168), wherein:
X₁ is A or G;
X₂ is L or M; and
X₃ is D or E.

Aspect 9. The fusion polypeptide of aspect 8, wherein the fusion polypeptide comprises the amino acid sequence:

(peptide 49; SEQ ID NO: 49)
GLFEAIEGFIENGWEAMIDLWYGYGRKKRRQRR;
or
(peptide 58; SEQ ID NO: 58)
GLFEAIEGFIENGWEGLIELWYGYGRKKRRQRR.

Aspect 10. The fusion polypeptide of aspect 1, wherein the fusion polypeptide comprises an amino acid sequence of Formula V: GLFX₁AIAX₂FIX₃NGWX₄GLIX₅GWYGGRKKRRQRRR (SEQ ID NO:208), wherein each of X₁, X₂, X₃, X₄, and X₅ is independently a non-coded amino acid.

Aspect 11. The fusion polypeptide of aspect 10, wherein the fusion polypeptide comprises the amino acid sequence:

(peptide 59; SEQ ID NO: 59)
GLFαAIAαFIαNGWαGLIαGWYGGRKKRRQRRR;
or
(peptide 60; SEQ ID NO: 60)
GLFαAIAαFIENGWEGLIDGWYGGRKKRRQRRR.

Aspect 12. The fusion polypeptide of aspect 10 or aspect 11, wherein each of X₁, X₂, X₃, X₄, and X₅ is α-aminoadipic acid.

Aspect 13. The fusion polypeptide of aspect 1, wherein the fusion polypeptide comprises an amino acid sequence of Formula VI: KLFEX₁IX₂X₃FIENGWEGMIX₄X₅WX₆GYGRKKRRQRX₇ (SEQ ID NO:170), wherein: X₁ is A or H; X₂ is E or A; X₃ is G or E; X₄ is D or E; X₅ is G or L; X₆ is E, H, K, R, or N; and X₇, if present, is R.

Aspect 14. The fusion polypeptide of aspect 13, wherein the fusion polypeptide comprises an amino acid sequence selected from:

(peptide 62; SEQ ID NO: 62)
KLFEAIEGFIENGWEGMIDLWEGYGRKKRRQRR;
(peptide 63; SEQ ID NO: 63)
KLFEAIEGFIENGWEGMIDLWHGYGRKKRRQRR;
(peptide 64; SEQ ID NO: 64)
KLFEAIEGFIENGWEGMIDLWKGYGRKKRRQRR;
(peptide 65; SEQ ID NO: 65)
KLFEAIEGFIENGWEGMIDLWRGYGRKKRRQRR;
(peptide 69; SEQ ID NO: 69)
KLFEAIEGFIENGWEGMIDLWNGYGRKKRRQR;
(peptide 71; SEQ ID NO: 71)
KLFEAIEGFIENGWEGMIELWNGYGRKKRRQRR;
(peptide 72; SEQ ID NO: 72)
KLFEAIAEFIENGWEGMIDLWNGYGRKKRRQRR;
(peptide 105; SEQ ID NO: 105)
KLFEHIEGFIENGWEGMIDGWYGYGRKKRRQRR;
(peptide 107; SEQ ID NO: 107)
KLFEHIEGFIENGWEGMIDLWYGYGRKKRRQRR;
and
(peptide 109; SEQ ID NO: 109)
KLFEHIEGFIENGWEGMIDLWKGYGRKKRRQRR.

Aspect 15. The fusion polypeptide of aspect 1, wherein the fusion polypeptide comprises an amino acid sequence of Formula VII: GLFEX₁IX₂X₃FIENGWEGMIDX₄WX₅GYGRKKRRQRR (SEQ ID NO:171), wherein: X₁ is R, H, A, or K; X₂ is E or A; X₃ is G or E; X₄ is L or G; and X₅ is N, Y, K, or E.

Aspect 16. The fusion polypeptide of aspect 15, wherein the fusion polypeptide comprises an amino acid sequence selected from:

(peptide 66; SEQ ID NO: 66)
GLFERIEGFIENGWEGMIDLWNGYGRKKRRQRR;
(peptide 68; SEQ ID NO: 68)
GLFEHIEGFIENGWEGMIDLWNGYGRKKRRQRR;
(peptide 70; SEQ ID NO: 70)
GLFEAIAEFIENGWEGMIDGWYGYGRKKRRQRR;
(peptide 73; SEQ ID NO: 73)
GLFEKIEGFIENGWEAMIDGWYGYGRKKRRQRR;
(peptide 74; SEQ ID NO: 74)
GLFEKIEGFIENGWEGMIDLWYGYGRKKRRQRR;
(peptide 75; SEQ ID NO: 31)
GLFEAIEGFIENGWEGMIDGWNGYGRKKRRQRR;
(peptide 76; SEQ ID NO: 24)
GLFEAIEEFIENGWEGMIDGWYGYGRKKRRQRR;
(peptide 98; SEQ ID NO: 98)
GLFEKIEGFIENGWEGMIDGWNGYGRKKRRQRR;
(peptide 99; SEQ ID NO: 99)
GLFEKIEGFIENGWEGMIDGWKGYGRKKRRQRR;
(peptide 100; SEQ ID NO: 100)
GLFEKIEGFIENGWEGMIDGWEGYGRKKRRQRR;
(peptide 101; SEQ ID NO: 101)
GLFEKIEGFIENGWEGMIDLWNGYGRKKRRQRR;
(peptide 102; SEQ ID NO: 102)
GLFEKIEGFIENGWEGMIDLWKGYGRKKRRQRR;
and
(peptide 103; SEQ ID NO: 103)
GLFEKIEGFIENGWEGMIDLWEGYGRKKRRQRR.

Aspect 17. The fusion polypeptide of aspect 1, wherein the fusion polypeptide comprises an amino acid sequence of Formula VIII: HLFEX₁IEGFIENGWEGMIDX₂WX₃GYGRKKRRQRR (SEQ ID NO:172), wherein: X₁ is A or K; X₂ is G or L; and X₃ is N, K, E, or Y.

Aspect 18. The fusion polypeptide of aspect 17, wherein the fusion polypeptide comprises an amino acid sequence selected from:

(peptide 92; SEQ ID NO: 92)
HLFEAIEGFIENGWEGMIDGWNGYGRKKRRQRR;
(peptide 93; SEQ ID NO: 93)
HLFEAIEGFIENGWEGMIDGWKGYGRKKRRQRR;
(peptide 94; SEQ ID NO: 94)
HLFEAIEGFIENGWEGMIDGWEGYGRKKRRQRR;
(peptide 95; SEQ ID NO: 67)
HLFEAIEGFIENGWEGMIDLWNGYGRKKRRQRR;
(peptide 96; SEQ ID NO: 96)
HLFEAIEGFIENGWEGMIDLWKGYGRKKRRQRR;
(peptide 97; SEQ ID NO: 97)
HLFEAIEGFIENGWEGMIDLWEGYGRKKRRQRR;
(peptide 104; SEQ ID NO: 104)
HLFEKIEGFIENGWEGMIDGWYGYGRKKRRQRR;
(peptide 106; SEQ ID NO: 106)
HLFEKIEGFIENGWEGMIDLWYGYGRKKRRQRR;
and
(peptide 108; SEQ ID NO: 108)
HLFEKIEGFIENGWEGMIDLWKGYGRKKRRQRR.

Aspect 19. The fusion polypeptide of any one of aspects 1-18, wherein all of the amino acids in the fusion polypeptide are linked by an amide bond.

Aspect 20. The fusion polypeptide of any one of aspects 1-18, wherein at least two adjacent amino acids are linked by a non-amide bond.

Aspect 21. The fusion polypeptide of any one of aspects 1-20, wherein one or more of the amino acids in the polypeptide comprises a modification.

Aspect 22. The fusion polypeptide of aspect 21, wherein the modification comprises a maleimide group, a methyltetrazine group, a 3-nitro-pyridine-2-carboxylic acid group, a 1,4-bis(bromomethyl)-benzene group, a poly(ethylene glycol) group, a 5-carboxyfluorescein group, a nitropyridine group, a pyridyl disulfide, and a pyridine.

Aspect 23. The fusion polypeptide of aspect 21 or 22, wherein the fusion polypeptide comprises an amino acid sequence of Formula IX: KLFEAIEGFIENGWEGMIDLWNX₁X₂YGRKKRRQRR (SEQ ID NO:173), wherein: X₁, if present, is Gly; and X₂ is Cys(methyltetrazine) or Cys(3-nitro-2-pyridinesulfenyl).

Aspect 24. The fusion polypeptide of aspect 23, wherein the fusion polypeptide comprises an amino acid sequence selected from:
KLFEAIEGFIENGWEGMIDLWNC*YGRKKRRQRR (peptide 87; SEQ ID NO:87), wherein “C*” is Cys(methyltetrazine);
KLFEAIEGFIENGWEGMIDLWNGC*YGRKKRRQRR (peptide 88; SEQ ID NO:88), wherein “C*” is Cys(methyltetrazine);
KLFEAIEGFIENGWEGMIDLWNC*YGRKKRRQRR (peptide 89; SEQ ID NO:89), wherein “C*” is Cys(3-nitro-2-pyridinesulfenyl); and
KLFEAIEGFIENGWEGMIDLWNGC*YGRKKRRQRR (peptide 90; SEQ ID NO:90), wherein “C*” is Cys(3-nitro-2-pyridinesulfenyl).

Aspect 25. The fusion polypeptide of aspect 21 or 22, wherein the fusion polypeptide comprising an amino acid sequence of Formula X: KLFEAIEGFIENGWEGMIDLWNGX₁YGRKKRRQRRX₂ (SEQ ID NO:174), wherein: X₁ is Cys(methyltetrazine-PEG4-maleimide), Cys(maleimide), Lys(PEG₂₃)₂, Lys(3-nitro-pyridine-2-carboxylic acid), Lys(PEG₂₃)₂, Lys(PEG₂₃)₂-(3-nitro-pyridine-2-carboxylic acid), or Cys (1,4-bis(bromomethyl)-benzene); and X₂ is Cys(3-nitro-2-pyridine-sulfenyl) or Lys(methyltetrazine-PEG4).

Aspect 26. The fusion polypeptide of aspect 25, wherein the fusion polypeptide comprises an amino acid sequence selected from the amino acid sequence of peptide f1, peptide f2, peptide f3, peptide f4, peptide f4, peptide f6, peptide f7, peptide f11, peptide f13, and peptide f14 depicted in FIG. 30.

Aspect 27. The fusion polypeptide of any one of aspects 1-26, wherein at least two adjacent amino acids are linked by a linker comprising one or more ethylene glycol monomers.

Aspect 28. The fusion polypeptide of aspect 27, wherein the linker is a polymer comprising 2, 4, 6, or 8 ethylene glycol monomers.

Aspect 29. The fusion polypeptide of aspect 1, wherein the fusion polypeptide comprises the amino acid sequence of any one of the peptides 19 and 40-60 depicted in FIG. 1.

Aspect 30. The fusion polypeptide of aspect 1, wherein the fusion polypeptide comprises the amino acid sequence of any one of the peptides depicted in FIG. 30.

Aspect 31. The fusion polypeptide of aspect 1, wherein the fusion polypeptide does not comprise the amino acid sequence of any one of the peptides designated 1-18 or 21-27 and depicted in FIG. 1.

Aspect 32. A composition comprising the cargo delivery fusion polypeptide of any one of aspects 1-31.

Aspect 33. The composition of aspect 32, further comprising a cargo, wherein the cargo comprises one or more of a nucleic acid, a polypeptide, and a ribonucleoprotein complex.

Aspect 34. The composition of aspect 33, wherein the cargo comprises a targeting moiety.

Aspect 35. The composition of aspect 32, comprising a nucleic acid comprising a nucleotide sequence encoding a gene product of interest.

Aspect 36. The composition of aspect 35, wherein the gene product of interest is an antigen.

Aspect 37. The composition of aspect 35 or aspect 36, wherein the nucleic acid is a recombinant expression vector.

Aspect 38. The composition of aspect 37, wherein the recombinant expression vector is a recombinant viral vector.

Aspect 39. The composition of aspect 32, comprising:
a) a CRISPR-Cas effector polypeptide; or
b) a fusion polypeptide comprising:
   i) a CRISPR-Cas effector polypeptide; and
   ii) one or more heterologous polypeptides.

Aspect 40. The composition of aspect 39, comprising a CRISPR-Cas guide nucleic acid.

Aspect 41. The composition of aspect 39 or aspect 40, comprising a donor DNA template.

Aspect 42. The composition of any one of aspects 39-41, wherein the CRISPR-Cas effector polypeptide is a type II CRISPR-Cas effector polypeptide, a type V CRISPR-Cas effector polypeptide, or a type VI CRISPR-Cas effector polypeptide.

Aspect 43. The composition of aspect 40, wherein the CRISPR-Cas guide nucleic acid is RNA.

Aspect 44. The composition of aspect 43, wherein the CRISPR-Cas guide nucleic acid is a single-molecule guide RNA or a dual-molecule guide RNA.

Aspect 45. The composition of any one of aspects 39-44, wherein the composition comprises a CRISPR-Cas effector fusion polypeptide comprising: i) a CRISPR-Cas effector fusion polypeptide; and ii) one or more nuclear localization signals.

Aspect 46. The composition of any one of aspects 39-44, wherein the composition comprises a CRISPR-Cas effector fusion polypeptide comprising: i) a CRISPR-Cas effector polypeptide; and ii) one or more heterologous effector polypeptides.

Aspect 47. The composition of aspect 46, wherein at least one of the one or more heterologous effector polypeptides is a single stranded nuclease, a double strand nuclease, a helicase, a methylase, a demethylase, an acetylase, a deacetylase, a deaminase, an integrase, a recombinase, a base editor, or a prime editor.

Aspect 48. The composition of any one of aspects 39-47, wherein the CRISPR-Cas effector polypeptide, or the CRISPR-Cas effector fusion polypeptide, comprises a covalently linked targeting moiety.

Aspect 49. The composition of aspect 48, wherein the targeting moiety is protein A, protein G, an aptamer, a darpin, or an antibody.

Aspect 50. The composition of aspect 49, comprising an antibody non-covalently bound to the affinity moiety.

Aspect 51. The composition of aspect 49 or aspect 50, where the antibody specifically binds an epitope on the surface of a eukaryotic cell, thereby targeting the composition to the cell.

Aspect 52. The composition of any one of aspects 39-51, wherein the CRISPR-Cas effector polypeptide, or the CRISPR-Cas effector fusion polypeptide, comprises a non-polypeptide polymer.

Aspect 53. The composition of aspect 52, wherein the non-polypeptide polymer is poly(ethylene glycol).

Aspect 54. The composition of any one of aspects 40-53, wherein the CRISPR-Cas effector polypeptide and the guide nucleic acid are in a ribonucleoprotein (RNP) complex.

Aspect 55. The composition of aspect 54, wherein the molar ratio of cargo delivery fusion polypeptide to RNP is at least 3:1.

Aspect 56. The composition of aspect 55, wherein the molar ratio of cargo delivery fusion polypeptide to RNP is from 10:1 to 50:1.

Aspect 57. The composition of any one of aspects 32-56, wherein the cargo delivery fusion polypeptide is present in the composition in a concentration of from about 2 μM to about 50 μM.

Aspect 58. The composition of any one of aspects 32-57, comprising one or more of a solubilizing agent, a surfactant, a buffer, a salt, and a protease inhibitor.

Aspect 59. The composition of any one of aspects 32-58, comprising poly(ethylene glycol), a non-ionic surfactant, or both.

Aspect 60. A method of delivering a cargo into a target population of eukaryotic cells, the method comprising contacting the cell with the composition of any one of aspects 32-59, thereby generating a modified target population of eukaryotic cells comprising the cargo.

Aspect 61. The method of aspect 60, wherein the target population of eukaryotic cells comprises a T cell, a stem cell, a natural killer cell, a renal cell or a neural cell.

Aspect 62. The method of aspect 60, wherein the target eukaryotic cell is a hematopoietic stem cell or a hematopoietic progenitor cell.

Aspect 63. The method of any one of aspects 60-4362 wherein the cell is in vitro.

Aspect 64. The method of aspect 63, wherein at least 50% of the target population of eukaryotic cells retain viability after said contacting.

Aspect 65. The method of aspect 63 or aspect 64, wherein the method comprises introducing into the modified target population of eukaryotic cells a second composition comprising a second cargo.

Aspect 66. The method of aspect 65, wherein said introducing is via electroporation or transfection.

Aspect 67. The method of aspect 66, wherein said transfection comprises contacting the modified target population of eukaryotic cells with a recombinant viral vector.

Aspect 68. The method of any one of aspects 60-62, wherein the cell is in vivo.

Aspect 69. A method of delivering a DNA molecule into a eukaryotic cell, the method comprising contacting the cell with a composition comprising:
a) an amphiphilic cargo delivery fusion polypeptide comprising:
   i) an endosomolytic polypeptide; and
   ii) a cell penetrating polypeptide; and
b) a DNA molecule comprising a nucleotide sequence encoding an immunogenic polypeptide.

Aspect 70. The method of aspect 69, wherein the immunogenic polypeptide is a viral polypeptide.

Aspect 71. The method of aspect 69 or aspect 70, wherein the endosomolytic polypeptide:
a) comprises the amino acid sequence GLFEAIAEFIENGWEGLIEGWYG (SEQ ID NO: 163);
b) comprises from 1 to 5 amino acid substitutions relative to GLFEAIAEFIENGWEGLIEGWYG (SEQ ID NO:163);
c) comprises the amino acid sequence GLFEAIEGFIENGWEGMIDGWYG (SEQ ID NO: 164); or
d) comprises from 1 to 5 amino acid substitutions relative to GLFEAIEGFIENGWEGMIDGWYG (SEQ ID NO:164),
wherein the endosomolytic polypeptide has a length of from about 20 amino acids to about 30 amino acids.

Aspect 72. The method of any one of aspects 69-71, wherein the cell penetrating polypeptide comprises the amino acid sequence YGRKKRRQRRR (SEQ ID NO:207), YGRKKRRQRR (SEQ ID NO:160), or GRKKRRQRRR (SEQ ID NO:161), and has a length of from 10 amino acids to 15 amino acids.

Aspect 73. A method of delivering a ribonucleoprotein (RNP) into a eukaryotic cell, the method comprising contacting the cell with a composition comprising:
a) an amphiphilic cargo delivery fusion polypeptide comprising:
   i) an endosomolytic polypeptide; and
   ii) a cell penetrating polypeptide; and
b) an RNP comprising:
   i) a CRISPR-Cas effector polypeptide; and
   ii) a guide nucleic acid.

Aspect 74. The method of aspect 73, wherein the endosomolytic polypeptide:
a) comprises the amino acid sequence GLFEAIAEFIENGWEGLIEGWYG (SEQ ID NO: 163);
b) comprises from 1 to 5 amino acid substitutions relative to GLFEAIAEFIENGWEGLIEGWYG (SEQ ID NO:163);
c) comprises the amino acid sequence GLFEAIEGFIENGWEGMIDGWYG (SEQ ID NO: 164); or
d) comprises from 1 to 5 amino acid substitutions relative to GLFEAIEGFIENGWEGMIDGWYG (SEQ ID NO:164),
wherein the endosomolytic polypeptide has a length of from about 20 amino acids to about 30 amino acids.

Aspect 75. The method of aspect 73 or aspect 74, wherein the cell penetrating polypeptide comprises the amino acid sequence YGRKKRRQRRR (SEQ ID NO:207), YGRKKRRQRR (SEQ ID NO:160), or GRKKRRQRRR (SEQ ID NO:161), and has a length of from 10 amino acids to 15 amino acids.

Aspect 76. The method of any one of aspects 73-75, comprising introducing into the cell a DNA donor template.

Aspect 77. The method of aspect 76, wherein the donor template is present in a recombinant viral vector.

Aspect 78. The method of aspect 77, wherein the recombinant viral vector is a recombinant adenoassociated viral vector.

Aspect 79. The method of any one of aspects 73-78, wherein the donor template comprises a nucleotide sequence encoding a polypeptide.

Aspect 80. The method of aspect 79, wherein the polypeptide is a chimeric antigen receptor comprising a single-chain Fv or a nanobody specific for a cancer-associated antigen.

Aspect 81. The method of any one of aspects 73-80, wherein the eukaryotic cell is an immune cell.

Aspect 82. The method of aspect 81, wherein the immune cell is a T cell, a B cell, or an NK cell.

Aspect 83. The method of any one of aspects 73-82, wherein the eukaryotic cell is in vivo.

Aspect 84. The method of any one of aspects 73-82, wherein the eukaryotic cell is in vitro.

### Aspects Set C

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A cargo delivery fusion polypeptide comprising:
a) an endosomolytic polypeptide; and
b) a cell penetrating polypeptide,
wherein the fusion polypeptide has a length of from about 32 amino acids to about 35 amino acids, and
wherein any two adjacent amino acids are independently linked by an amide bond or a non-amide bond,
wherein the cargo delivery fusion polypeptide comprises one or more of:
i) a positively charged amino acid at the N-terminus;
ii) a positively charged amino acid within 5 amino acids of the N-terminus; and
iii) a positively charged amino acid at position 22,
optionally wherein the positively charged amino acid is Lys, His, or Arg.

Aspect 2. The fusion polypeptide of aspect 1, wherein the fusion polypeptide comprises an amino acid sequence of any one of Formulas I-VIII.

Aspect 3. The fusion polypeptide of aspect 1, wherein the fusion polypeptide comprises an amino acid sequence of Formula I: KLFEX₁IEGFIENGWEX₂MIDX₃WX₄GX₅GRKKRRQRR (SEQ ID NO: 165), wherein
X₁ is A, R, or K;
X₂ is A or G;
X₃ is L or G;
X₄ is N or Y; and
X₅, if present, is Y.

Aspect 4. The fusion polypeptide of aspect 3, wherein the fusion polypeptide comprises an amino acid sequence selected from the group consisting of:

(peptide 19; SEQ ID NO: 19)
KLFEAIEGFIENGWEGMIDGWYGYGRKKRRQRR;
(peptide 40; SEQ ID NO: 40)
KLFEAIEGFIENGWEGMIDGWYGGRKKRRQRR;
(peptide 44; SEQ ID NO: 44)
KLFERIEGFIENGWEGMIDGWYGYGRKKRRQRR;
(peptide 45; SEQ ID NO: 45)
KLFEKIEGFIENGWEGMIDGWYGYGRKKRRQRR;
(peptide 46; SEQ ID NO: 46)
KLFEAIEGFIENGWEAMIDGWYGYGRKKRRQRR;
(peptide 47; SEQ ID NO: 47)
KLFEAIEGFIENGWEGMIDLWYGYGRKKRRQRR;
(peptide 48; SEQ ID NO: 48)
KLFEAIEGFIENGWEGMIDGWNGYGRKKRRQRR;
(peptide 53; SEQ ID NO: 53)
KLFEAIEGFIENGWEAMIDLWYGYGRKKRRQRR;
(peptide 54; SEQ ID NO: 54)
KLFEAIEGFIENGWEAMIDGWNGYGRKKRRQRR;
(peptide 55; SEQ ID NO: 55)
KLFEAIEGFIENGWEGMIDLWNGYGRKKRRQRR;
(peptide 56; SEQ ID NO: 56)
KLFEAIEGFIENGWEAMIDLWNGYGRKKRRQRR;
and
(peptide 57; SEQ ID NO: 57)
KLFEKIEGFIENGWEAMIDLWNGYGRKKRRQRR.

Aspect 5. The fusion polypeptide of aspect 1, wherein the fusion polypeptide comprises an amino acid sequence of Formula II: X₁LFEX₂IEGFIENGWEGMIDGWYGYGRKKRRQRR (SEQ ID NO: 166), wherein X₁ is R or G; and X₂ is R or K.

Aspect 6. The fusion polypeptide of aspect 5, wherein the fusion polypeptide comprises an amino acid sequence selected from the group consisting of:

(peptide 42; SEQ ID NO: 42)
RLFERIEGFIENGWEGMIDGWYGYGRKKRRQRR;
(peptide 43; SEQ ID NO: 43)
RLFEKIEGFIENGWEGMIDGWYGYGRKKRRQRR;
and
(peptide 41; SEQ ID NO: 41)
GLFERIEGFIENGWEGMIDGWYGYGRKKRRQRR.

Aspect 7. The fusion polypeptide of aspect 1, wherein the fusion polypeptide comprises an amino acid sequence of Formula III: GLFEAIEGFIENGWEX₁MIDX₂WNGYGRKKRRQRR (SEQ ID NO: 167), wherein X₁ is A or G; and X₂ is G or L.

Aspect 8. The fusion polypeptide of aspect 7, wherein the fusion polypeptide comprises an amino acid sequence selected from the group consisting of:

(peptide 50; SEQ ID NO: 50)
GLFEAIEGFIENGWEAMIDGWNGYGRKKRRQRR;
(peptide 51; SEQ ID NO: 51)
GLFEAIEGFIENGWEGMIDLWNGYGRKKRRQRR;
and
(peptide 51; SEQ ID NO: 51)
GLFEAIEGFIENGWEAMIDLWNGYGRKKRRQRR.

Aspect 9. The fusion polypeptide of aspect 1, wherein the fusion polypeptide comprises an amino acid sequence of Formula IV: GLFEAIEGFIENGWEX₁X₂IX₃LWYGYGRKKRRQRR (SEQ ID NO: 168), wherein:
X₁ is A or G;
X₂ is L or M; and
X₃ is D or E.

Aspect 10. The fusion polypeptide of aspect 9, wherein the fusion polypeptide comprises the amino acid sequence:

(peptide 49; SEQ ID NO: 49)
GLFEAIEGFIENGWEAMIDLWYGYGRKKRRQRR;
or
(peptide 58; SEQ ID NO: 58)
GLFEAIEGFIENGWEGLIELWYGYGRKKRRQRR.

Aspect 11. The fusion polypeptide of aspect 1, wherein the fusion polypeptide comprises an amino acid sequence of Formula V: GLFX₁AIAX₂FIX₃NGWX₄GLIX₅GWYGGRKKRRQRRR (SEQ ID NO:208), wherein each of X₁, X₂, X₃, X₄, and X₅ is independently a non-coded amino acid.

Aspect 12. The fusion polypeptide of aspect 11, wherein the fusion polypeptide comprises the amino acid sequence:

(peptide 59; SEQ ID NO: 59)
GLFaAIAaFIaNGWaGLIaGWYGGRKKRRQRRR;
or
(peptide 60; SEQ ID NO: 60)
GLFaAIAaFIENGWEGLIDGWYGGRKKRRQRRR.

Aspect 13. The fusion polypeptide of aspect 11 or aspect 12, wherein each of X₁, X₂, X₃, X₄, and X₅ is α-aminoadipic acid.

Aspect 14. The fusion polypeptide of aspect 1, wherein the fusion polypeptide comprises an amino acid sequence of Formula VI: KLFEX₁IX₂X₃FIENGWEGMIX₄X₅WX₆GYGRKKRRQRX₇ (SEQ ID NO:170), wherein: X₁ is A or H; X₂ is E or A; X₃ is G or E; X₄ is D or E; X₅ is G or L; X₆ is E, H, K, R, or N; and X₇, if present, is R.

Aspect 15. The fusion polypeptide of aspect 14, wherein the fusion polypeptide comprises an amino acid sequence selected from:

(peptide 62; SEQ ID NO: 62)
KLFEAIEGFIENGWEGMIDLWEGYGRKKRRQRR;
(peptide 63; SEQ ID NO: 63)
KLFEAIEGFIENGWEGMIDLWHGYGRKKRRQRR;
(peptide 64; SEQ ID NO: 64)
KLFEAIEGFIENGWEGMIDLWKGYGRKKRRQRR;
(peptide 65; SEQ ID NO: 65)
KLFEAIEGFIENGWEGMIDLWRGYGRKKRRQRR;
(peptide 69; SEQ ID NO: 69)
KLFEAIEGFIENGWEGMIDLWNGYGRKKRRQR;
(peptide 71; SEQ ID NO: 71)
KLFEAIEGFIENGWEGMIELWNGYGRKKRRQRR;
(peptide 72; SEQ ID NO: 72)
KLFEAIAEFIENGWEGMIDLWNGYGRKKRRQRR;
(peptide 105; SEQ ID NO: 105)
KLFEHIEGFIENGWEGMIDGWYGYGRKKRRQRR;
(peptide 107; SEQ ID NO: 107)
KLFEHIEGFIENGWEGMIDLWYGYGRKKRRQRR;
and
(peptide 109; SEQ ID NO: 109)
KLFEHIEGFIENGWEGMIDLWKGYGRKKRRQRR.

Aspect 16. The fusion polypeptide of aspect 1, wherein the fusion polypeptide comprises an amino acid sequence of Formula VII: GLFEX₁IX₂X₃FIENGWEGMIDX₄WX₅GYGRKKRRQRR (SEQ ID NO:171), wherein: X₁ is R, H, A, or K; X₂ is E or A; X₃ is G or E; X₄ is L or G; and X₅ is N, Y, K, or E.

Aspect 17. The fusion polypeptide of aspect 16, wherein the fusion polypeptide comprises an amino acid sequence selected from:

(peptide 66; SEQ ID NO: 66)
GLFERIEGFIENGWEGMIDLWNGYGRKKRRQRR;
(peptide 68; SEQ ID NO: 68)
GLFEHIEGFIENGWEGMIDLWNGYGRKKRRQRR;
(peptide 70; SEQ ID NO: 70)
GLFEAIAEFIENGWEGMIDGWYGYGRKKRRQRR;
(peptide 73; SEQ ID NO: 73)
GLFEKIEGFIENGWEAMIDGWYGYGRKKRRQRR;
(peptide 74; SEQ ID NO: 74)
GLFEKIEGFIENGWEGMIDLWYGYGRKKRRQRR;
(peptide 75; SEQ ID NO: 31)
GLFEAIEGFIENGWEGMIDGWNGYGRKKRRQRR;
(peptide 76; SEQ ID NO: 24)
GLFEAIEEFIENGWEGMIDGWYGYGRKKRRQRR;
(peptide 98; SEQ ID NO: 98)
GLFEKIEGFIENGWEGMIDGWNGYGRKKRRQRR;
(peptide 99; SEQ ID NO: 99)
GLFEKIEGFIENGWEGMIDGWKGYGRKKRRQRR;
(peptide 100; SEQ ID NO: 100)
GLFEKIEGFIENGWEGMIDGWEGYGRKKRRQRR;
(peptide 101; SEQ ID NO: 101)
GLFEKIEGFIENGWEGMIDLWNGYGRKKRRQRR;
(peptide 102; SEQ ID NO: 102)
GLFEKIEGFIENGWEGMIDLWKGYGRKKRRQRR;
and
(peptide 103; SEQ ID NO: 103)
GLFEKIEGFIENGWEGMIDLWEGYGRKKRRQRR.

Aspect 18. The fusion polypeptide of aspect 1, wherein the fusion polypeptide comprises an amino acid sequence of Formula VIII: HLFEX₁IEGFIENGWEGMIDX₂WX₃GYGRKKRRQRR (SEQ ID NO:172), wherein: X₁ is A or K; X₂ is G or L; and X₃ is N, K, E, or Y.

Aspect 19. The fusion polypeptide of aspect 18, wherein the fusion polypeptide comprises an amino acid sequence selected from:

(peptide 92; SEQ ID NO: 92)
HLFEAIEGFIENGWEGMIDGWNGYGRKKRRQRR;
(peptide 93; SEQ ID NO: 93)
HLFEAIEGFIENGWEGMIDGWKGYGRKKRRQRR;
(peptide 94; SEQ ID NO: 94)
HLFEAIEGFIENGWEGMIDGWEGYGRKKRRQRR;
(peptide 95; SEQ ID NO: 67)
HLFEAIEGFIENGWEGMIDLWNGYGRKKRRQRR;
(peptide 96; SEQ ID NO: 96)
HLFEAIEGFIENGWEGMIDLWKGYGRKKRRQRR;
(peptide 97; SEQ ID NO: 97)
HLFEAIEGFIENGWEGMIDLWEGYGRKKRRQRR;
(peptide 104; SEQ ID NO: 104)
HLFEKIEGFIENGWEGMIDGWYGYGRKKRRQRR;
(peptide 106; SEQ ID NO: 106)
HLFEKIEGFIENGWEGMIDLWYGYGRKKRRQRR;
and
(peptide 108; SEQ ID NO: 108)
HLFEKIEGFIENGWEGMIDLWKGYGRKKRRQRR.

Aspect 20. The fusion polypeptide of any one of aspects 1-19, wherein all of the amino acids in the fusion polypeptide are linked by an amide bond.

Aspect 21. The fusion polypeptide of any one of aspects 1-19, wherein at least two adjacent amino acids are linked by a non-amide bond.

Aspect 22. The fusion polypeptide of any one of aspects 1-21, wherein one or more of the amino acids in the polypeptide comprises a modification.

Aspect 23. The fusion polypeptide of aspect 22, wherein the modification comprises a maleimide group, a methyltetrazine group, a 3-nitro-pyridine-2-carboxylic acid group, a 1,4-bis(bromomethyl)-benzene group, a poly(ethylene glycol) group, a 5-carboxyfluorescein group, a nitropyridine group, a pyridyl disulfide, and a pyridine.

Aspect 24. The fusion polypeptide of aspect 22 or 23, wherein the fusion polypeptide comprises an amino acid sequence of Formula IX: KLFEAIEGFIENGWEGMIDLWNX₁X₂YGRKKRRQRR (SEQ ID NO:173), wherein: X₁, if present, is Gly; and X₂ is Cys(methyltetrazine) or Cys(3-nitro-2-pyridinesulfenyl).

Aspect 25. The fusion polypeptide of aspect 24, wherein the fusion polypeptide comprises an amino acid sequence selected from:
KLFEAIEGFIENGWEGMIDLWNC*YGRKKRRQRR (peptide 87; SEQ ID NO:87), wherein “C*” is Cys(methyltetrazine);
KLFEAIEGFIENGWEGMIDLWNGC*YGRKKRRQRR (peptide 88; SEQ ID NO:88), wherein “C*” is Cys(methyltetrazine);
KLFEAIEGFIENGWEGMIDLWNC*YGRKKRRQRR (peptide 89; SEQ ID NO:89), wherein “C*” is Cys(3-nitro-2-pyridinesulfenyl); and
KLFEAIEGFIENGWEGMIDLWNGC*YGRKKRRQRR (peptide 90; SEQ ID NO:90), wherein “C*” is Cys(3-nitro-2-pyridinesulfenyl).

Aspect 26. The fusion polypeptide of aspect 22 or 23, wherein the fusion polypeptide comprising an amino acid sequence of Formula X: KLFEAIEGFIENGWEGMIDLWNGX₁YGRKKRRQRRX₂ (SEQ ID NO:174), wherein: X₁ is Cys(methyltetrazine-PEG4-maleimide), Cys(maleimide), Lys(PEG₂₃)₂, Lys(3-nitro-pyridine-2-carboxylic acid), Lys(PEG₂₃)₂, Lys(PEG₂₃)₂-(3-nitro-pyridine-2-carboxylic acid), or Cys (1,4-bis(bromomethyl)-benzene); and X₂ is Cys(3-nitro-2-pyridine-sulfenyl) or Lys(methyltetrazine-PEG4).

Aspect 27. The fusion polypeptide of aspect 26, wherein the fusion polypeptide comprises an amino acid sequence selected from the amino acid sequence of peptide f1, peptide f2, peptide f3, peptide f4, peptide f4, peptide f6, peptide f7, peptide f11, peptide f13, and peptide f14 depicted in FIG. 30.

Aspect 28. The fusion polypeptide of any one of aspects 1-27, wherein at least two adjacent amino acids are linked by a linker comprising one or more ethylene glycol monomers Aspect 29. The fusion polypeptide of aspect 28, wherein the linker is a polymer comprising 2, 4, 6, or 8 ethylene glycol monomers.

Aspect 30. The fusion polypeptide of aspect 1, wherein the fusion polypeptide comprises the amino acid sequence of any one of the peptides 19 and 40-60 depicted in FIG. 1.

Aspect 31. The fusion polypeptide of aspect 1, wherein the fusion polypeptide comprises the amino acid sequence of any one of the peptides depicted in FIG. 30.

Aspect 32. The fusion polypeptide of aspect 1, wherein the fusion polypeptide does not comprise the amino acid sequence of any one of the peptides designated 1-18 or 21-27 and depicted in FIG. 1.

Aspect 33. A composition comprising the cargo delivery fusion polypeptide of any one of aspects 1-32.

Aspect 34. A method of delivering a cargo into a target population of eukaryotic cells, the method comprising contacting the cell with the composition of aspect 33, thereby generating a modified target population of eukaryotic cells comprising the cargo.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular (ly); i.p., intraperitoneal (ly); s.c., subcutaneous (ly); and the like.

### Materials and Methods

The following Materials and Methods were used in the Examples provided below.

### T Cells

Primary human CD4+ T cells were isolated from leukopacks (purchased from Stemcell Tech or Allcells Inc) PBMCs using negative isolation (Stem Cell Technologies). Cells were either used fresh or stored frozen at −80° C. Cells were suspended in growth media (Xvivo-15+5% FBS+55 μM β-mercaptoethanol (βME)+10 μM N-acetyl cysteine) at 1.0×10⁶ cells/mL. Cells were stimulated for 48 hours with CD3/CD28 Dynabeads (Gibco CAT #11131D) at 1 bead/cell ratio, 200 U/mL human IL-2 (Proleukin), 5 ng/ml IL-7 (Peptrotech CAT #200-07), 5 ng/ml IL-15 (Peptrotech CAT #200-15). The day of applying Cas9 delivery complexes, cells were removed from beads, washed with media, resuspended in fresh growth media at 1×10⁶ cells/mL supplemented with IL-2 at 300 U/mL. When Cas9 RNP delivery complexes were ready, cells were centrifuged at 300×g, resuspended in OptiMEM and placed in 96-well round-bottom culture plates, with 200×10³ cells/well.

### NK Cells

Primary human NK cells were isolated from healthy blood donor leukopaks (Allcells) using a NK Cell Isolation kit (STEMCELL, as per the manufacturer's instructions). Freshly isolated NK cells were cultured in X-VIVO 15 medium (Lonza) with 5% fetal bovine serum, 50 μM 2-mercaptoethanol, and 10 mM N-acetyl L-cysteine, together with IL-2 (at 1,000 U/ml) and MACSiBead Particles pre-loaded with anti-human CD335 (NKp46) and anti-human CD2 antibodies (Miltenyi Biotec). Cells were cultured for 5 days, beads were removed by magnetic separation, and then either coincubated with peptide-delivered RNPs or electroporated with the same protocol as T cells (200 k cells plus RNP in P3 buffer, pulse code EH-115). After treatment, cells were rescued by adding their normal growth media as described above with 1000 U/mL IL2. Media was replaced every 3 days.

### B Cells

Primary adult blood cells from anonymous healthy human donors were purchased as leukapheresis packs from StemCell Technologies, Inc. Primary human B cells were isolated by negative selection using EasySep Magnetic B cell isolation kits (StemCell, Cat #17954) based on manufacturer guidelines. Following isolation, B cells were activated and cultured at 1×10⁶ cells mL 1 for 2 days in IMDM medium (ThermoFisher) with 10% fetal bovine serum, 50 μM 2-mercaptoethanol, 100 ng mL 1 MEGACD40L (Enzo), 200 ng mL 1 anti-human RP105 (Biolegend), 500 U mL 1 IL-2 (UCSF Pharmacy), 50 ng mL 1 IL-10 (ThermoFisher), and 10 ng mL-1 IL-15 (R&D Systems). Prior to editing on Day 2, B cells were collected and treated with either electroporation or peptide co-incubation as indicated. B cells were then rescued with prewarmed growth media and transferred to fresh plates at 0.5-1.0×10⁶ cells mL 1 in growth medium. Fresh media and B cell activation cocktail were added every 2-3 days.

### HSPCs

G-CSF and Plerixafor mobilized human primary CD34+ HSPCs (StemCell Technologies) were thawed and cultured in StemSpan SFEM II media (StemCell Technologies) with StemSpan CC110 cytokine cocktail for 48 hours. Cell density was maintained between 200,000-500,000 cells per mL. Cells were plated into 96 well round bottom plates, 20×10³ cells per well, in 80 μL of SFEMII with 1.25× CC110. RNP was diluted to 20 μL in SFEM II and incubated for 10 min at room temperature. Peptide was combined with the RNP-SFEM II mix and incubated for 10 min at room temperature. The peptide-RNP-SFEM II mix was added to 20,000 HSPCs to a final volume of 100 μL per well in a 96-well round bottom plate. Cell density was also increased to 100,000 HSPCS per 100 μL for some experiments. After 48 hours the cytokines were refreshed: 100 μL StemSpan SFEM II supplemented with StemSpan CC110 cytokine cocktail (STEMCELL Technologies) was added to each well two days after co-incubation.

### Cell Viability Assays

Cell viability after treatments and compared to untreated cells was measured by CellTiter-Glo assay (Promega G7570) according to manufacturer's instructions, with luminescence measured via a Spark plate reader.

### Peptides

Peptides made by solid phase synthesis (CPC Scientific Peptide Company) and stored lyophilized or as 10 mM dimethyl sulfoxide (DMSO) stocks at −20° C. in a desiccator. Peptide Sequences are listed in FIG. 1 and FIG. 30.

### Cas9 Fusion Proteins

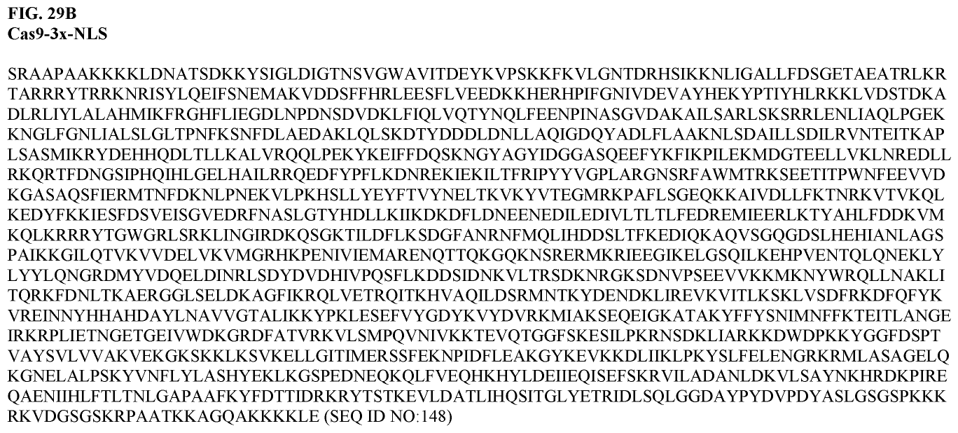

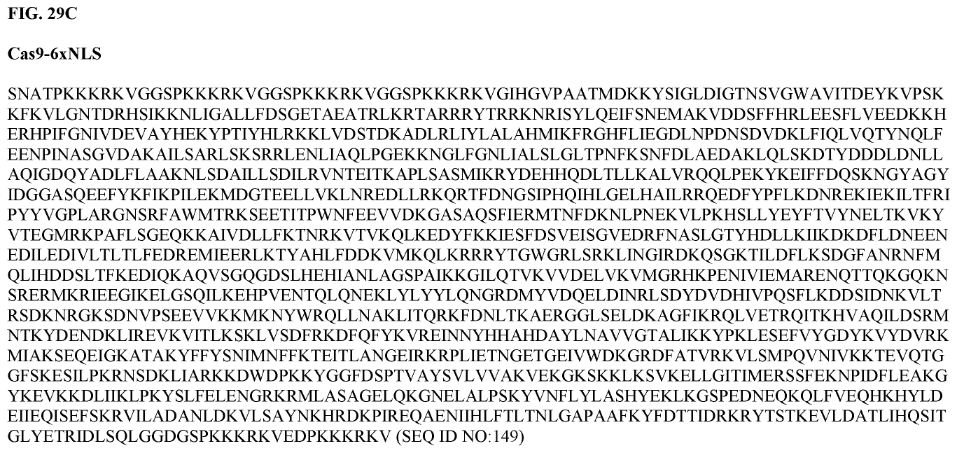

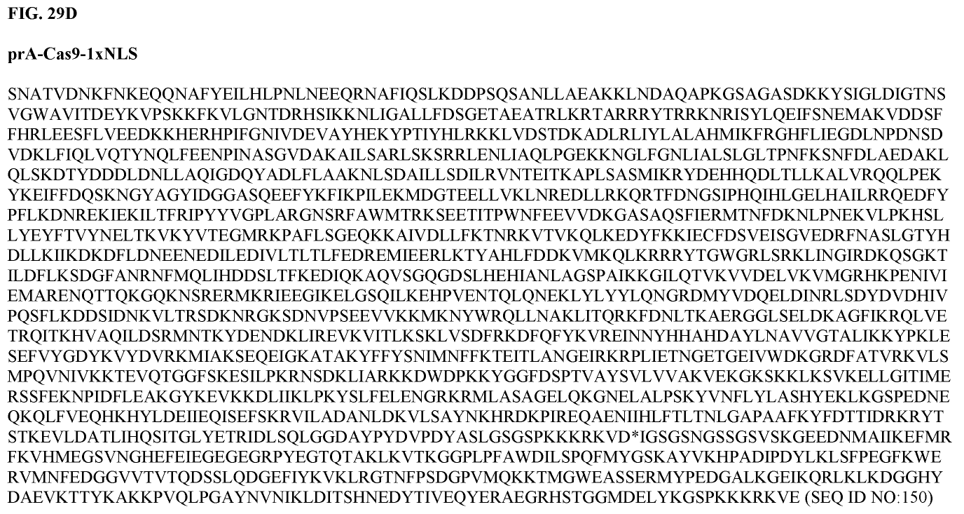

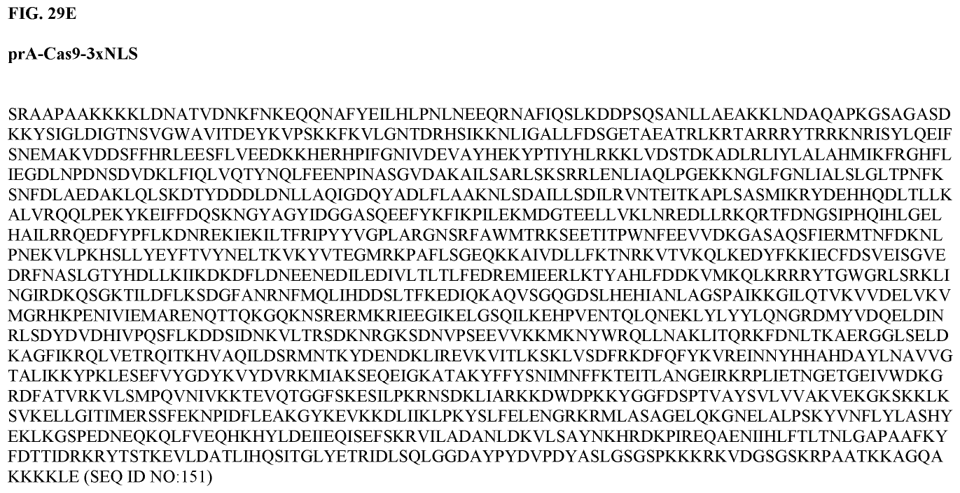

Cas9 proteins and fusion proteins were expressed in E. coli and purified via nickel affinity chromatography, ion exchange chromatography, and size exclusion chromatography as previously described. Rouet et al. (2018) J. Am. Chem. Soc. 140:6596. Purified proteins concentrated to ˜50 μM in 20 mM HEPES-KOH PH 7.5, 150 mM NaCl, 10% (v/v) glycerol and stored at −80° C. The “Cas9-1×NLS” contains a C-terminal SV40 nuclear localization signal (NLS), “Cas9-3×NLS” construct (also referred to as “triNLS” in some text and/or figures) contains an N-terminal cMyc NLS, and a C-terminal SV40 and NP sequence. Wu et al. (2019) Nature Med. 25:776. “Cas9-6×NLS” (also referred to as 4+2×NLS in some text and/or figures) contains 4× N-terminal SV40 NLS and 2× C-terminal SV40 NLS sequences (Staahl et al. (2017) Nat. Biotechnol. 35:431) and was stored in 25 mM sodium phosphate pH 7.25, 300 mM NaCl, 200 mM trehalose. “prA-Cas9-3×NLS” contains an N-terminal protein A domain, which allows binding with the Fc domains of IgGs as well as the same NLS configurations as “Cas9-3×NLS”. Sequences of fusion proteins are provided in FIG. 29A-28E.

sgRNAs

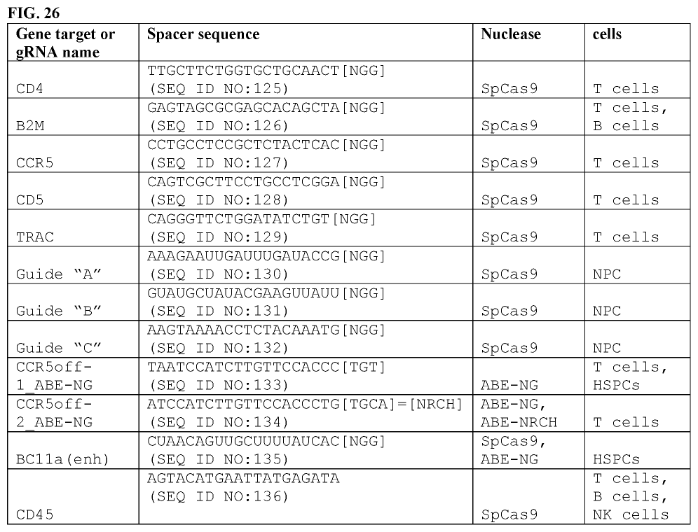

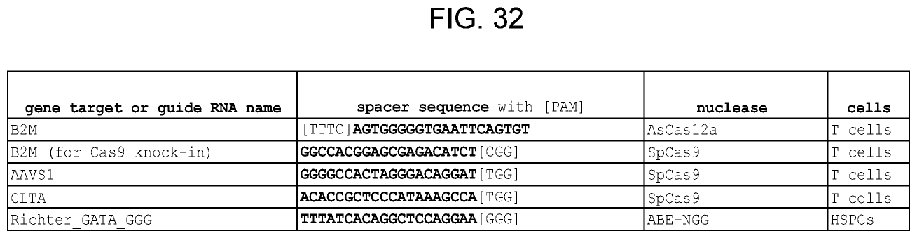

Single-molecule guide RNAs (sgRNAs) were purchased from Synthego or IDT and lyophilized stocks were dissolved in water. Before use, the sgRNAs were suspended in 20 mM HEPES pH 7.5, 150 mM NaCl and refolded via warming to 95° C. for 5 minutes and slow cooling to room temperature over 25 minutes. Spacer sequences provided in FIG. 26 and FIG. 32.

### RNP Formation

Cas9 proteins were diluted to 10 μM in “RNP buffer” (20 mM HEPES pH 7.5, 150 mM NaCl, 10% Glycerol, 2 mM MgCl₂). sgRNA was diluted to 15 μM in 20 mM HEPES PH 7.5, 150 mM NaCl. Cas9 protein was mixed with guide RNA in equal volumes to give 5 μM RNP complexes at 1:1.5 molar ratio of Cas9: guide RNA.

### Antibodies

Anti-CD3 antibodies (OKT3, Invitrogen, 16-0037-85) were concentrated to 20 μM (Amicon centrifugal filter unit, UFC500396) and re-quantified via nanodrop. Control antibodies (“IgG”) are γ-globins from human blood (Sigma G4386) which were resuspended in phosphate-buffered saline (PBS) pH 7.4 at 20 μM. Anti-CD79b (CB3-1, BD Biosciences #555678), anti-CD22 (S-HCL-1, Biolegend #363502), anti-CD22 (HIB22 (Biolegend #302502), (anti-RP105 MHR73-11, Biolegend #312913), anti-CD71 (EPR4012, Abcam #108985) were concentrated to 6.7 μM (Amicon centrifugal filter unit, UFC500396) and re-quantified via nanodrop.

### Delivery Complexes for Co-Incubation

Cas9 RNPs were mixed with antibodies at 1:1 molar ratio and allowed to bind for a minimum of 15 minutes at room temperature. Peptides in DMSO were diluted in H₂O to 1 mM and added to RNP: Ab mixture. Complexes were added to a 96 well round-bottom plate, and cells in OptiMEM were added directly on top of the RNP/Ab/peptide complexes (200×10³ cells/well in 100 μL of optimem). Final concentration of RNP: Ab complexes on cells is 500 nM and peptides was 10 UM unless otherwise stated. After 1 hour of treatment, cells with their treatments were split into two plates with 50 μL in each plate, and 150 μL of recovery media (Xvivo15+7.5% fetal bovine serum (FBS)+450 U/mL IL2) was added per well, thus diluting but not removing the treatment.

### Cas9 RNP Electroporation

Cas9 RNPs were electroporated (nucleofected) into T cells using P3 Primary Cell 4D nucleofector with the P3 buffer+supplement (Lonza #V4XP-3032) and using the EH-115 pulse code.

### Sequential Editing

For each sequential editing treatment, stimulated CD4⁺ T cells were treated with 50 pmol of prA-Cas9-3×NLS complexed with OKT3 antibody and combined with A5K (peptide #22) at 10 μM. The first treatment targeted β2M, then 2 days later the edited cells were pooled, mixed, and split into two groups, with one group going on for further editing of the CD4 locus and the other group remaining a singly edited control group. 2 days later, the β2M and CD4 edited cell group was then split into two groups where CD5 was edited in one group and the second group remained a doubly (β2M and CD4) edited control set. Cells were analyzed by flow cytometry 2 days later to give levels of KO at the β2M, CD4, and CD5 on all the treatment groups.

### Neural Progenitor Cell Editing

Neural progenitor cells (NPCs) were harvested from Ai9 mice (Jackson Laboratories, Stock No: 007909) which contain a loxP-flanked STOP cassette preventing transcription of a CAG promoter-driven red fluorescent protein variant (tdTomato). SpCas9 guide RNAs which have been previously described were applied to cut at the STOP cassette, thereby allowing expression of TdTomato. Guides “A” and “B” are applied as paired guides to cut out the cassette (Tabebordbar et al. (2016) Science 351:407) or guide “C”, aka sg298 (Staahl et al. (2017) supra) was applied as a solo guide. Spacer sequences provided in FIG. 26.

NPCs were cultured in DMEM/F12 with Glutamax supplemented with 10 mM HEPES pH 7.5, 1×NEAA (Fisher Sci 11-140-050), Pen-Strep (100 U/mL) (Fisher Sci 15140-122), 1× B27 (Fisher Scientific 17504-044) and 1× N2 supplement (Thermo 17502048). Cells were plated at a density of either 15000 or 50000 cells per well in a 96 well plate. Delivery complexes were applied to cells and allowed to incubate for 5 days, until being analyzed via flow cytometry for TdTomato signal.

### Non-Viral Knock-In (KI)

DNA oligonucleotide (“oligo”) design and HDR strategy: A HDR template to create a N terminal fusion of FLAG with the extracellular portion of the CD5 molecule was synthesized as a single strand 160 mer oligonucleotide with 40 base left and 40 base right homology arms, plus truncated Cas9 binding sites. Nguyen et al. (2020) Nat. Biotechnol. 38:44; and Shy et al. (2021) BioRxiv doi: 10.1101/2021.09.02.458799. At day 5 post knockin, cells were stained for CD5 expression and FLAG-tag expression and analyzed on an Attune NXT flow cytometer.

### Targeted Knockin Using an AAV Vector

Knockins were performed in T cells using recombinant AAV6 containing homology arms targeting the beginning of the first exon of the TRAC locus. Eyquem et al. (2017) Nature 543:113. The cargo was a 19287. CAR flanked by P2A sequences, such that after knockin the CAR was transcribed cocistronically with the mRNA encoding TCRα but translated as a separate protein, and the TCR remained not surface-expressed. The CAR contained a CD8A signal sequence. SJ25C1 scFv. CD28 hinge, transmembrane, and costimulatory domain, and CD35 signaling domain.

Knockins Performed with RNP Electroporation to Knock Out TCRα

After two days of activation. T cells were detached from Dynabeads, pelleted by centrifugation, and resuspended in Lonza P3 primary cell solution. TRAC sgRNA (Synthego) and Cas9 protein (QB3 MacroLab. “6×NLS”) were incubated at a 2:1 ratio (37° C. 15 min), and 60 pmol RNP was combined with 2×10⁶ cells per well of an Amaxa nucleofector plate. Electroporation was carried out using a Lonza nucleofection instrument and EH115 cell type protocol, and cells were rescued with serum-free medium to 2×10⁶ cells/ml and returned to the tissue culture incubator for 1 h. Then. 0.2×10⁶ cells in 100 μl (or in the multiplexed editing experiment 4×10⁶ cells at the same concentration) were treated with AAV at a multiplicity of infection of 5×104. After overnight incubation, cells were split into two wells and pelleted by centrifugation, and the AAV-containing medium was exchanged for fresh serum-containing medium.

Knockins Performed with RNP and A5K Coincubation to Knock Out TCRα

After two days of activation. T cells were detached from Dynabeads, pelleted by centrifugation, and resuspended in Opti-MEM as 0.2×10⁶ cells per well. 50 pmol TRAC-targeting RNP (sgRNA-to-Cas9 ratio of 1.2:1) and 1000 pmol A5K (or 1500 pmol for the sequential editing experiment) were combined, and cells were incubated with this mix, bringing the volume in the well to 100 μl (tissue culture incubator, 1 h). Cells were treated with AAV at various time points with respect to treatment with the RNP-peptide mix: −1 h, −30 min, +1 min, +30 min, and +1 h, and otherwise kept in the tissue culture incubator. Cells were then split into two wells, and, depending on the condition either 50 μl or 150 μl serum-free medium was added to arrive at 1×10⁶ cells/ml or 0.5×10⁶ cells/ml, respectively. In the “wash” condition, cells were instead pelleted by centrifugation without splitting, and the supernatant was exchanged for 100 μl serum-free medium to arrive at 2×10⁶ cells/ml. Cells were treated with AAV and passaged as done for the electroporation conditions.

### Flow Cytometry

Flow cytometry was performed on an Attune NXT flow cytometer with a 96-well autosampler (ThermoFisher Scientific). Cells were resuspended in FACS buffer and stained with the surface marker-targeting antibodies as well as live-dead stain according to manufacturer's instructions. Analysis was performed using FlowJo.

### Amplicon Sequencing

Genome editing was quantified via next generation sequencing (NGS) of amplicons around the Cas9 target site. Cells were pelleted by centrifugation at 500×g for 5 minutes, washed twice with PBS, and then resuspended in 50 μL of Quick Extract (Lucigen) per well and incubated at room temperature for 20 min. Samples were then heated at 65° C. for 10 min then 95° C. for 5 min. Genomic DNA was stored at −20° C. Polymerase chain reaction (PCR) amplification was performed with GXL polymerase according to manufacturer's instructions. The amplicons were cleaned up via SPRI beads (UC Berkeley sequencing core) and quantified via nanodrop.

Samples were deep sequenced on an Illumina MiSeq at 300 bp paired-end reads to a depth of at least 10,000 reads per sample. Cortado (https:(//)github.com/staciawyam/cortado) was used to analyze editing outcomes. Briefly, reads were adapter trimmed and then merged into single reads. These joined reads were then aligned to the target reference sequence to identify editing events at the cut site. NHEJ rates were calculated by counting any reads with an insertion or deletion overlapping the cut site, or occurring within a 3 base pair window on either side of the cut site. SNPs occurring within the window around the cutsite were not counted towards NHEJ. Total NHEJ reads were then divided by the total number of aligned reads to get % NHEJ. For base editing experiments, the joined reads were aligned to the target reference sequence to identify base editing events at the targeted loci. The rate of base editing was calculated as the number of reads with an edit over the total aligned reads, calculated individually for each site.

### DNA Vaccine

### Plasmids

For cell reporter assays, plasmid DNA encoded the nanoluciferase gene on a CMV promoter. For in vivo mouse studies, plasmid DNA encoded the RBD subunit of the SARS-COV-2 spike protein as a vaccine antigen, also on a cytomegalovirus (CMV) promoter.

### Cas9 and Guide RNAs

crRNAs (IDT) were designed to target several sites around the plasmids. RNPs were formulated with each crRNA and a tracrRNA (IDT) with dead Cas9 (Alt-R® S.p. dCas9 Protein V3,IDT) so that the RNPs will bind to (but not cut) the plasmid DNA. Dual-guide RNAs formed by mixing crRNA and tracrRNA together in equal mols, diluting to 12 μM in 20 mM HEPES pH 7.5, 150 mM NaCl and heating at 37° C. for 30 minutes. dCas9 was diluted to 10 UM in 20 mM HEPES-KOH PH 7.5, 150 mM NaCl, 10% (v/v) glycerol and mixed in equal volumes with the dgRNA, incubated at 37° C. for 15 minutes, giving a final RNP at 5 UM with 1:1.2 ratio of Cas9 to dgRNA.

### Delivery Complex Formation

The plasmid DNA was mixed with Cas9 RNPs (1:1 molar ratio of each RNP, 0-8 RNP binding sites per plasmid) to allow binding, then mixed with peptides at specified concentrations and applied to cells.

### DC2.4 Cell Luciferase Assays

DC2.4 cells were cultured in DMEM with glutamax, 10% FBS, 1% NEAA, and 1% sodium pyruvate. Delivery complexes were applied to DC2.4 cells. After 24 hrs or 48 hrs, cell lysates were harvested with M-Per lysis reagent (ThermoFisher 78501) and luciferase expression was quantified using the Nano-Glo Luciferase Assay kit (Promega, N1120) and measured via a Spark plate reader. Protein content was measured in each sample via Bradford protein assay, with bovine serum albumin (BSA) standards as a reference. Luciferase expression was normalized to protein levels in each sample.

### Mouse Studies

Delivery complexes were injected into the Balb/c mice via intramuscular hind leg injection on day 0 and again at 2-3 weeks. An additional purified recombinant Spike protein antigen was administered intramuscularly (IM) at week 3-5 post vaccination. At indicated time points, blood was collected to assess serum levels of anti-Spike RBD IgG antibody by ELISA, which in clinical trials has correlated well with clinical protection against disease.

### Cas12a Protein

Cas12a protein was purchased from IDT: Alt-R A.s. Cas12a Ultra (10001273), which is an engineered Acidaminococcus sp. construct (Zhang et al. (2021) Nat. Comm. volume 12, Article number: 3908).

### Adenine Base Editor Proteins

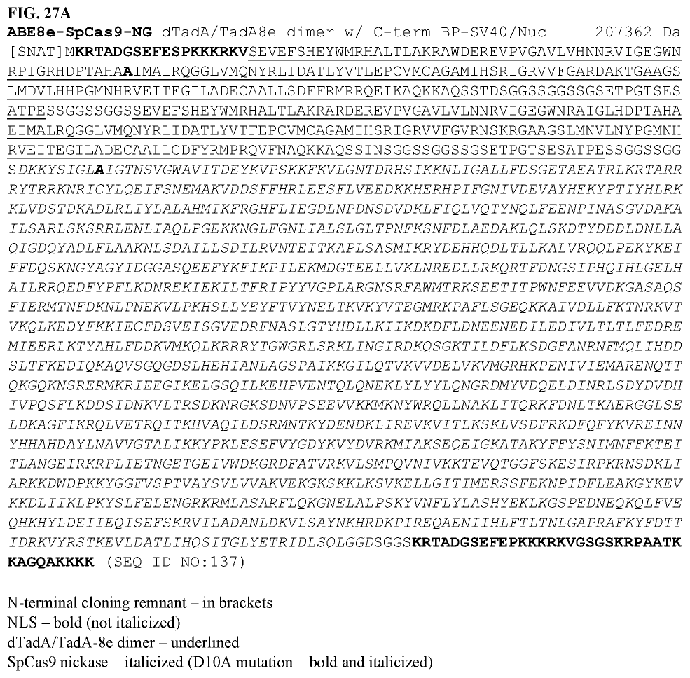

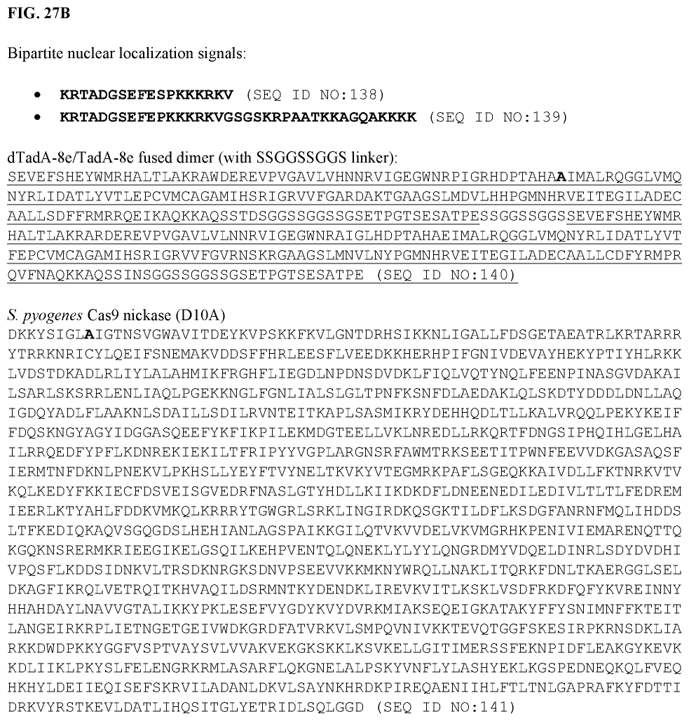

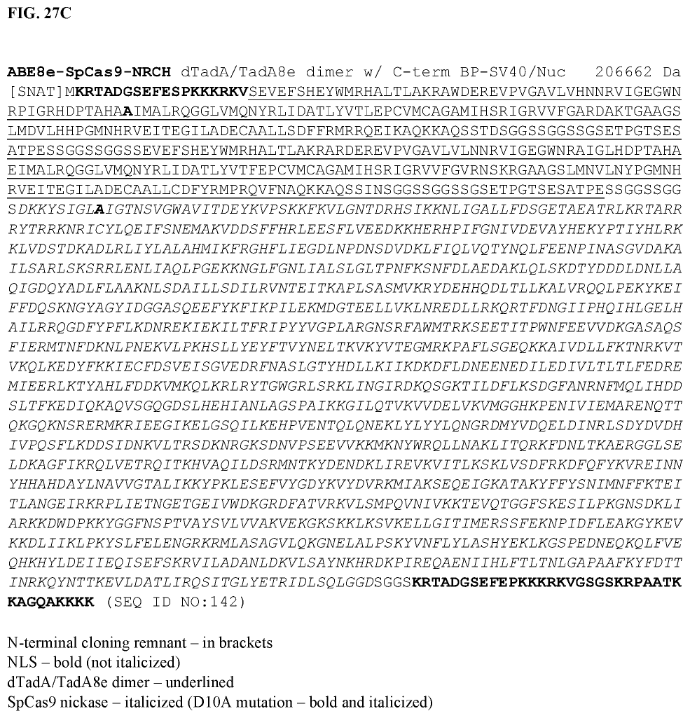

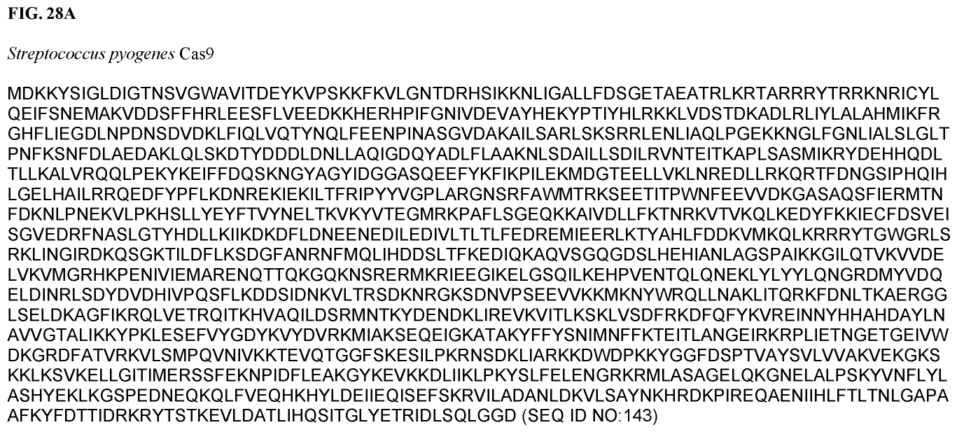

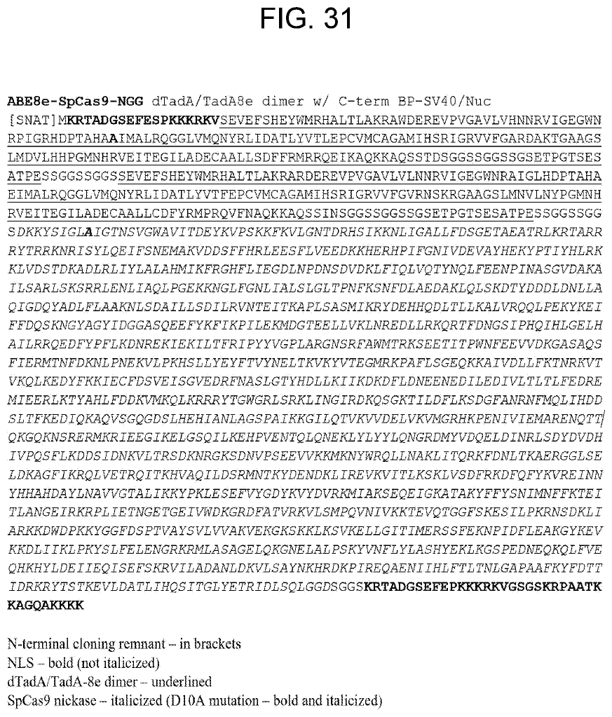

Adenine base editor (ABE) proteins contain a “nickase” version of S. pyogenes CRISPR Cas9 (D10A mutation to disable one catalytic center), with an N-terminally fused pair of fused, evolved “8e” (Richter et al. Nat. Biotechnol. (2020) 38:7) TadA deaminase domains (the N-terminal-most of which is catalytically disabled) and NLS sequences appended to the N- and C-termini of the ABE fusion protein. Three versions of the nickase Cas9 were employed, each with distinct protospacer-adjacent motif (PAM) recognition properties: the wild-type NGG version, the engineered NG version (Nishimasu et al. (2018) Science 361:6408), or the engineered NRCH version (Miller et al. 2020 Nat Biotechnol. (2020) 38:4). ABE proteins were expressed in E. coli and purified via nickel affinity chromatography, ion exchange chromatography, and size exclusion chromatography as previously described (Rouet et al. (2018) J. Am. Chem. Soc. 140:6596). Amino acid sequences are provided in FIG. 27 and FIG. 31.

### Targeted Knockin Using an AAV Vector

In primary human B cells and NK cells, knockins were performed using recombinant AAV6 containing homology arms targeting the beginning of the first exon of the CLTA locus to create an N-terminal fusion of sfGFP and Clathrin (adapted from Roth et al. 2018 Nature 549:405).

Knock-Ins Performed with RNP and ASK Coincubation to Knockin sfGFP-Clathrin Fusion

For B cell sfGFP-CLTA knockin. B cells were cultured for 2 days as above, pelleted by centrifugation, and resuspended in Opti-MEM as 0.2×10⁶ cells per well. For NK cell sfGFP-CLTA knockin, NK cells were cultured for 5 days as above, removed from beads by magnetic separation, pelleted by centrifugation, and then resuspended in Opti-MEM as 0.2×10⁶ cells per well. For both B and NK editing. 50 pmol CLTA-targeting RNP (sgRNA-to-Cas9 ratio of 1.2:1) and 1000 pmol A5K were combined, and cells were incubated with this mix, bringing the volume in the well to 100 μl (tissue culture incubator, 1 h). Cells were then treated with AAV at a multiplicity of infection of 5×10\hat{ }4. After overnight incubation, cells were split into two wells and pelleted by centrifugation, and the AAV-containing medium was exchanged for fresh serum-containing medium.

### In Vivo Editing of Human T Cells in Humanized (PBMC NSG) Mice

NOD SCID gamma (NSG) mice (Shultz et al. (2007) Nat. Rev. Immunol. 7:118) infused with peripheral blood mononuclear cells (PBMCs) from a single human donor were administered with prepared formulations via tail vein injections at 6-8 weeks of age. CRISPR-Cas9 RNP was prepared with sgRNA targeting the β2M locus paired with prA-Cas9-triNLS. 500 pmol RNP was mixed with either 100 pmol of Fc-silenced CD3 (OKT3) antibody, or no antibody, and 5 nmol of either peptide 1 (E5-TAT) or peptide #22 (A5K) with 0.05% tween-80 in all formulations. This resulted in four samples with RNP: RNP with peptide 1 ±OKT3; RNP with peptide 22 ±OKT3. RNP Formulations were filtered using a 0.22 um sterile filter prior to administration. Animals were sacrificed 7 days after injection, splenocytes were isolated, and editing efficiency assessed by amplicon-based deep sequencing. PCR primers used for amplicon generation were specific to the human genome and did not produce an amplicon when mouse genomic DNA was included as template. Some splenocyte samples were sorted by fluorescent activated cell sorting (FACS) to separate out CD5⁺ T-cells and CD5⁻ non-T-cells (predominantly B-cells), and indels were detected via amplicon-based deep sequencing.

### In Vivo Mouse Brain Editing

7 μL of RNP formulations was stereotactically injected into the striatum (coordinates: AP: +0.5 mm rel. bregma; MR and ML relative to midline: 2.0 mm; DV 2.5 mm) of Ai9 mouse brain using convection-enhanced delivery (CED), with an infusion rate of 1 μL/min. Left striatum was injected with an RNP-tween formulation; the right striatum was injected with an RNP-peptide-tween formulation (the peptide was #55). RNP samples contained triNLS SpCas9 protein-which was the basis of reported RNP concentrations-complexed with a 0.75× molar equivalent of an Ai9 “A” gRNA and an Ai9 “B” gRNA (cumulatively representing a 1.5× molar excess of gRNA over Cas9 protein). These two gRNA sequences are capable of making two concerted DNA breaks, excising a repressor cassette that activates downstream tdTomato expression. In the 7 μL formulation injected, the RNP concertation was 54.4 μM in the sample with peptide 55, and 52.6 μM in the sample without peptide 55; peptide 55 concentration was 2 mM; tween-80 concentration was 0.05%. Mouse age at delivery was 10 weeks; three male mice and two female mice were injected. Brains were harvested 21 days after administration of formulations. Histological sections were stained for NeuN (neurons; GeneTex chicken anti-NeuN), DAPI (nuclei), and tdTomato (Invitrogen polyclonal rabbit anti-RFP). A Dmi8 fluorescent microscope was used; for highly edited regions, at least 3 20× confocal regions of interest were captured per animal for quantification. The percentage of tdTomato⁺ neurons was quantified using QuPath software, using merged 20× confocal images.

### Transcriptome Analysis

CD4⁺ T cells were edited with a Cas9-RNP targeting AAVS1 as described above. RNA was extracted from treated cells (n.t., DMSO, ASK, electroporation) from four donors at three time points (6 hours, 1 day, and 7 days after editing) using the RNeasy Micro kit (Qiagen #74004), quantified using a NanoDrop, and stored at −80° C. RNA samples were submitted to the UCSF-HDFCCC Laboratory for Cell Analysis shared resource facility for nCounter analysis using the NanoString CAR-T characterization panel. Gene expression fold changes and Benjamini-Yekutieli-adjusted p-values for Volcano plots were determined using nSolver software. For each treatment comparison of interest, nSolver analysis was run for each time point separately and for all time points incorporated. The latter approach made use of more data as input, it was more sensitive at identifying significantly affected genes; and it was less biased to time point-specific outcomes.

### Translocation Analysis

Cells were pelleted by centrifugation at 300×g for 5 minutes, resuspended in 40 μL of QuickExtract solution (Lucigen) per well, and incubated at 65° C. for 10 min, then 95° C. for 5 min. Genomic DNA was stored at −20° C. Primers and 5′ 6-FAM-labeled probes were synthesized by IDT (ZEN-3′ Iowa Black FQ quencher). Droplet digital PCR reactions were prepared using ddPCR Supermix for probes (no dUTP) (Bio-Rad #1863023), and droplets were generated and read using a QX200 droplet generator and reader. Data were analyzed using the Bio-Rad QuantaSoft Analysis Pro software.

### Example 1: Peptide Screening

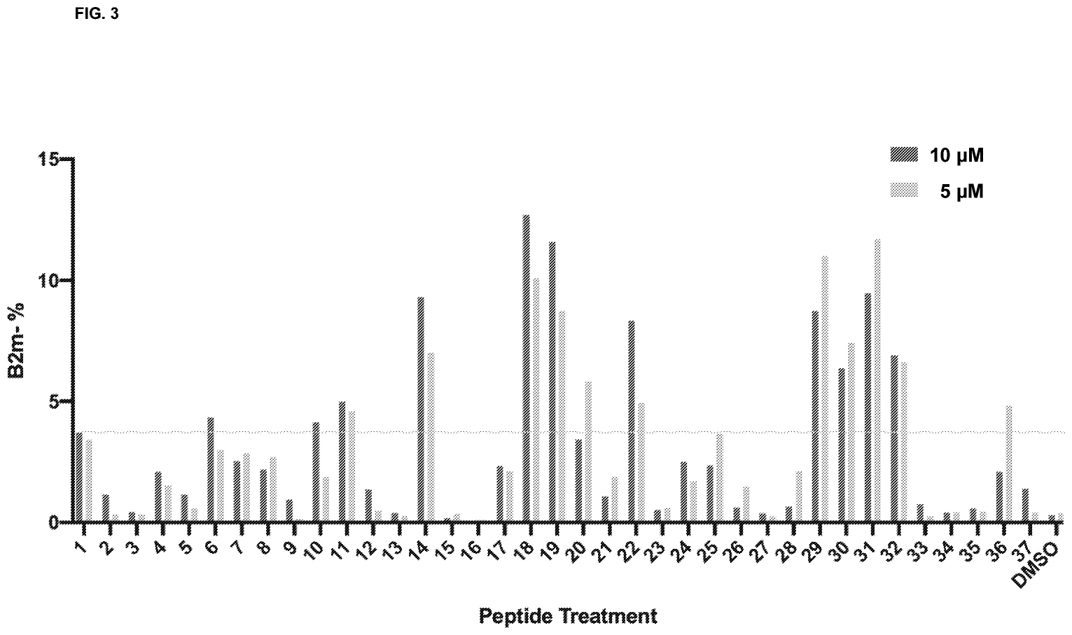

Peptides 1-60 (FIG. 1) were screened for Cas9-mediated KO at the β2M locus in human primary CD4⁺ T cells. Peptides 1-58 (FIG. 1) were screened for Cas9-mediated NHEJ at the erythrocyte-specific BCL11a enhancer locus in CD34⁺ HSPCs. The effect of a non-ionic surfactant on peptide-mediated genome editing was assessed. The results are shown in FIG. 3-8.

FIG. 3. Screening of peptides 1-37 for Cas9-mediated KO at the β2M locus in human primary CD4+ T cells, as measured by flow cytometry. prA-Cas9-1×NLS with OKT3 was combined with peptides at either 5 μM or 10 μM concentration. The DMSO control features RNP mixed with the concentration of DMSO that is introduced by the peptide stock solution (but no peptide). This screen demonstrates that specific single amino acid changes from the original E5-TAT (peptide no. 1) or INF7-TAT (peptide no. 11) can result in substantially higher efficiency of delivery as determined by the resulting editing rates.

As shown in FIG. 3, primary human CD4+ T cells can be edited to varying degrees when distinct peptides are co-incubated with a S. pyogenes Cas9 ribonucleoprotein (RNP) complex targeting the β2M locus. Peptide-mediated delivery of Cas9 RNP cargo was effective at final peptide concentrations of either 5 μM or 10 μM. In particular, peptide 19 (the G1K variant of INF7-TAT) edits T cells much more efficiently than INF7-TAT. This experiment was performed in the absence of any targeting antibody, and employed a “one NLS” Cas9 construct that is less potent than Cas9 constructs featuring more fused NLS sequences.

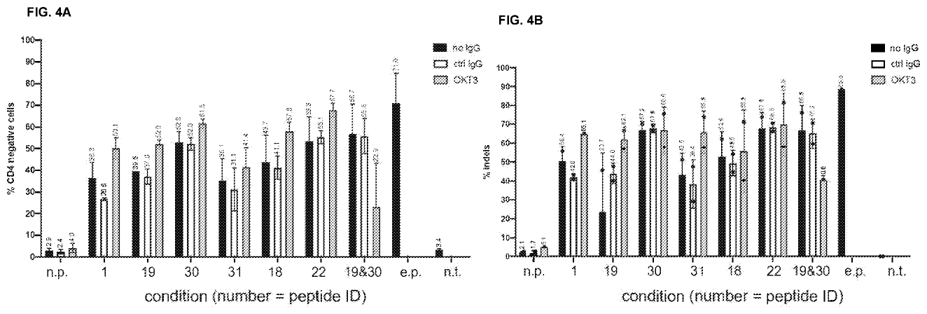

FIG. 4A and FIG. 4B. Screening of peptides for Cas9-mediated KO at the CD4 locus in human primary CD4+ T cells, as assessed by flow cytometry (panel A) or by deep sequencing (panel B). prA-Cas9-3×NLS was combined with no IgG, or with equivalent amounts of either a control non-targeting heterogeneous IgG mixture (ctrl IgG) or an anti-CD3 targeting antibody (OKT3), and applied to cells with peptides at 10 UM final concentration. The “19&30” condition features an equimolar mixture of peptides 19 & 30. OKT3-mediated enhancement of editing varies depending on the peptide used. Replicates represent results in two distinct human cell donors.

As shown in FIG. 4A and FIG. 4B, primary human CD4+ T cells can be edited to varying degrees when distinct peptides are co-incubated with a S. pyogenes Cas9 ribonucleoprotein (RNP) complex targeting the CD4 locus. Peptide-mediated delivery of Cas9 RNP cargo (sometimes paired with an antibody) was effective and could be enhanced by the inclusion of cell targeting antibody. In this example, the Cas9 construct features fusion to the IgG-binding protein A domain as well as three NLS sequences. This Cas9 construct is capable of binding to the anti-CD3 mAb OKT3, which is known to induce endocytosis into T cells through its interactions with the T cell receptor. The presence of OKT3 can enhance peptide-mediated editing efficiency, as evidenced by the bars labeled in gray, often showing editing rates that are substantially higher than the “no IgG” (black) or “ctrl IgG” (white) bars.

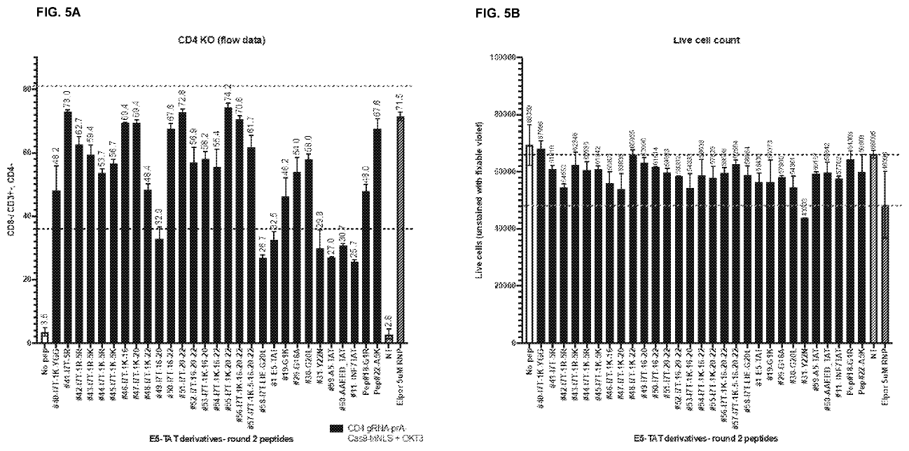

FIG. 5A and FIG. 5B. (A) Screening of peptides #40-60 (as well as select comparator peptides from the screen of “parent peptides” #1-37) for Cas9-mediated KO at the ß2M locus in human primary CD4+ T cells, as measured by flow cytometry. prA-Cas9-1×NLS was combined with anti-CD3 antibody OKT3 and applied to cells with peptides at 10 UM concentration. (B) Live cell counts from each treatment condition as determined by live/dead fixable violet staining (Thermofisher) and flow cytometry. Replicates are from two distinct human cell donors. In both (A) and (B), “No pep”=Cas9 without peptide; “NT”=cells that were not treated with Cas9 or peptide.

As shown in FIG. 5A and FIG. 5B, screening of additional peptides revealed that some—but not all—peptide sequences featuring sequence elements drawn from “parent peptides” #1-37 promoted genome editing in T cells that is more efficient than the editing mediated by any of the parent peptides, and is more efficient than electroporation-mediated editing. Importantly, after treatment with Cas9 and peptide, the T cell viability is close to that of the “no peptide” and “non-treated” control conditions, and viability is superior as compared to the cells that were electroporated (with peptide #31 being the exception).

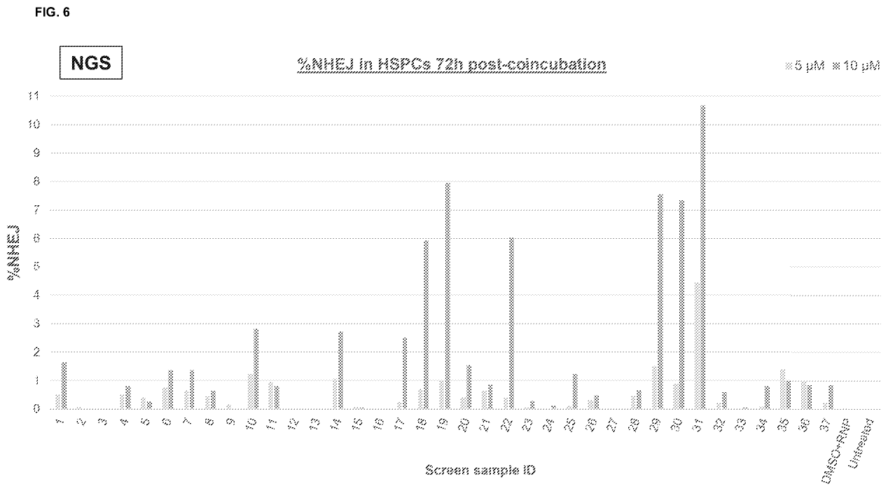

FIG. 6. Screen of peptides #1-37 allowing Cas9-mediated NHEJ at the erythrocyte-specific BCL11a enhancer locus in CD34+ HSPCs, as measured by deep sequencing, at either 5 μM or 10 μM concentration. The results of this first screen demonstrate that specific amino acid changes from the original E5-TAT (peptide no. 1) or INF7-TAT (peptide no. 11) can result in higher efficiency delivery as measured by editing levels.

As shown in FIG. 6. primary human CD34+ HSPCs can be edited to varying degrees when distinct peptides are co-incubated with a S. pyogenes Cas9 ribonucleoprotein (RNP) complex targeting the erythroid-specific Bcl11a enhancer locus. Peptide-mediated delivery of Cas9 RNP cargo was effective at final peptide concentrations of either 5 μM or 10 μM. In particular, peptide 19 (the G1K variant of INF7-TAT) edits CD34+ HSPCs much more efficiently than INF7-TAT. This experiment was performed in the absence of any targeting antibody, and employed a Cas9 construct fused to three NLS sequences.

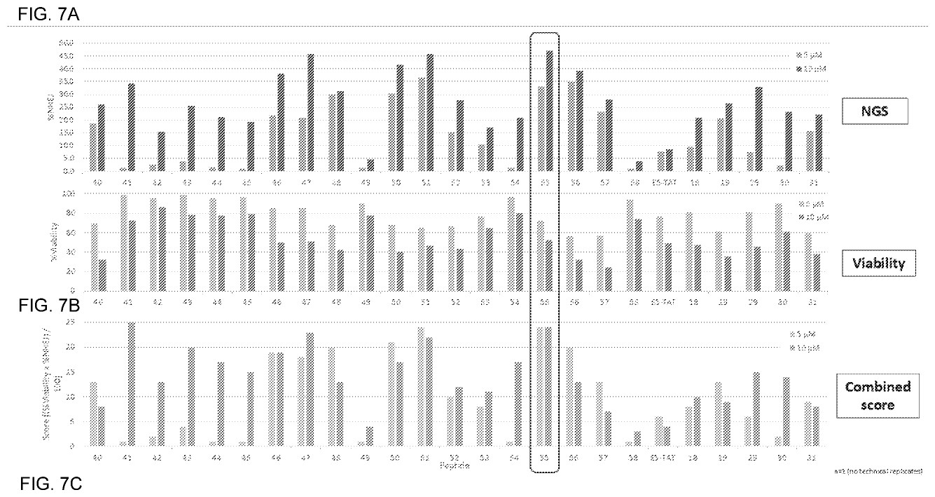

FIG. 7A-7C. (A) Screening of peptides #40-58 (and comparing to a selection of peptides from the first round of screening) for promoting Cas9-3×NLS mediated NHEJ in HSPCs, as measured by deep sequencing, at either 5 μM or 10 μM final peptide concentration. NHEJ at the erythrocyte-specific BCL11a enhancer locus in CD34+ HSPCs was detected via amplicon-based NGS. (B) HSPC viability under each condition as determined by CellTiter-Glo assay (Promega). (C) A combined score for each peptide calculated by [(% Viability×% NHEJ)×100]. Circled=best performing peptide overall for balancing activity with toxicity. Note that E5-TAT is peptide #1 (FIG. 1).

As shown in FIG. 7A-7C, screening of additional peptides revealed that some—but not all—peptides featuring sequence elements drawn from “parent peptides” #1-37 promoted genome editing in HSPCs that is more efficient than the editing mediated by any of the parent peptides. Peptide #55 is one example of a peptide that facilitated efficient genome editing and preserved cell viability.

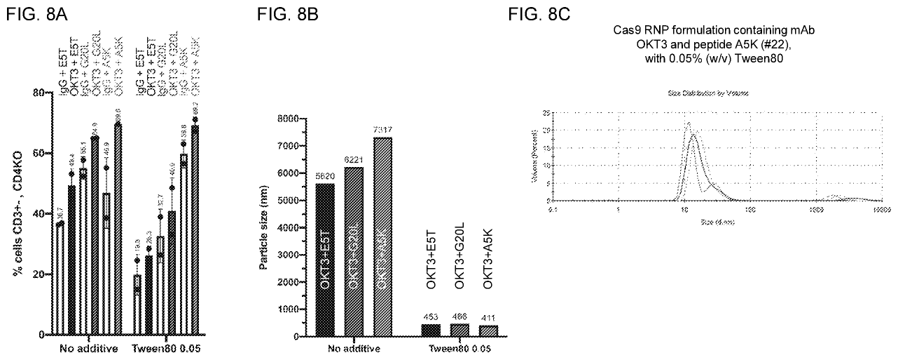

FIG. 8A-8C. The impact of non-ionic surfactant additive on peptide-Cas9 formulations. (A) Peptide-mediated genome editing of human primary T cells in the absence or presence of 0.05% (w/v) Tween-80 (concentration reported is in the RNP formulation before it is applied to cells & media). Cas9 RNP targeting CD4 for knock-out was pre-complexed with either negative control, non-targeting IgG or with anti-CD3, which can promote endocytosis into T cells. Delivery (and genome editing) was promoted by the inclusion of 5 μM (final concentration) E5-TAT (peptide #1), G20L (peptide #30) or A5K (peptide #22). Knock-out of CD4 was detected by flow cytometry. (B) Dynamic light scattering (DLS) was used to measure the average particle size in various formulation samples from panel (A). (C) Representative data from the OKT3+A5K, 0.05% Tween-80 sample shown in in panels (A) and (B).

As shown in FIG. 8A-8C, peptides can mediate delivery of Cas9 RNP for efficient genome editing of human primary T cells, even in the presence of a non-ionic surfactant such as Tween-80™. This is important because Tween-80 can promote smaller particle sizes, which can be important for tissue distribution for in vivo applications. A5K (peptide #22) was able to retain full potency as part of a formulation containing 0.05% Tween-80, and such a sample exhibited a predominant particle size of 10-20 nm, consistent with the 3D structure of the Cas9 RNP.

### Example 2: Gene Editing

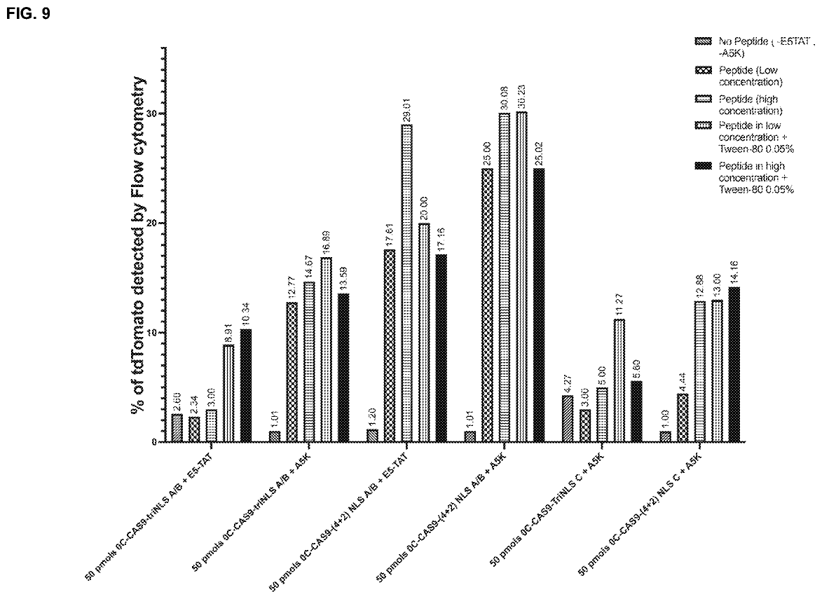

The effect of various peptides (FIG. 1) on gene editing in various types of cells was tested. The results are shown in FIG. 9-19.

FIG. 9. Screening for Cas9 mediated genome editing in primary murine neural progenitor cells (NPCs) from Ai9 mice, as determined by tdTomato signal detected via flow cytometry. Peptides E5-TAT (Peptide 1) and A5K (Peptide 22) were examined at either 10 μM (“high concentration”) or 5 μM (“low concentration”) in combination with either Cas9 3×NLS or Cas9 (4+2×NLS), with either equivalent loading of A/B guides targeting two loci or the C guide targeting a single locus. In some conditions, the additive Tween-80 was also included in the RNP and peptide formulation at 0.05% concentration (w/v). Cells were plated at 50,000 cells per well. Flow cytometry was performed 5 days after treatments were applied to cells.

As shown in FIG. 9, co-incubation of peptides promoted genome editing in primary murine neural progenitor cells. Peptide #22 (the A5K derivative of INF7-TAT) promoted editing more efficiently than did peptide #1 (E5-TAT). Cells were derived from the Ai9 reporter mouse, and they express fluorescent protein tdTomato if genome editing creates a small excision that enables gene expression. Peptides can still mediate delivery of Cas9 RNP (and genome editing) in the presence of non-ionic surfactant tween-80 (aka polysorbate 80), which can prevent peptide-mediated aggregation and promote monodisperse solutions of Cas9 RNP.

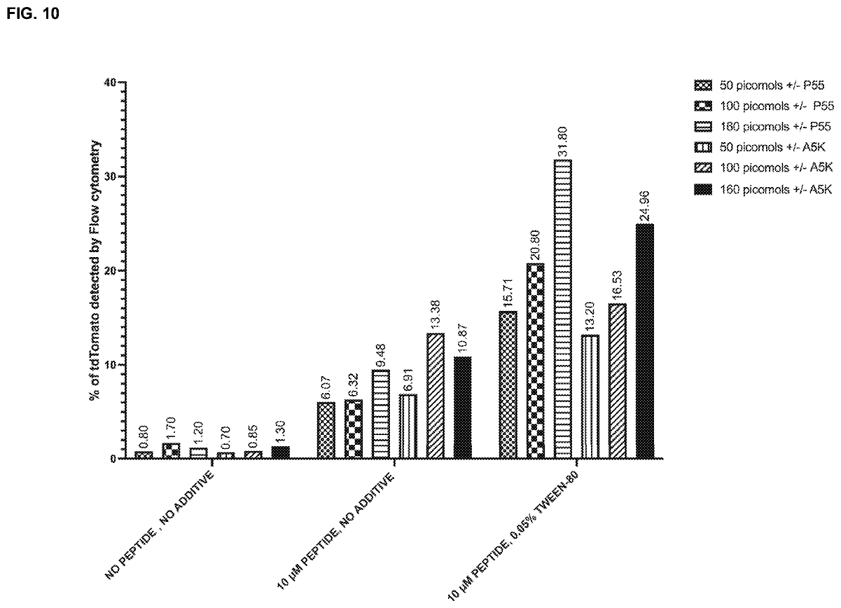

FIG. 10. Screening for Cas9 mediated genome editing in primary murine neural progenitor cells (NPCs) from Ai9 mice, as determined by tdTomato signal by flow cytometry. Peptides A5K (Peptide #22) and P55 (Peptide #55) were used at 10 μM final concentration in combination with Cas9-(4+2) formed as an RNP with the A and B guides (equimolar mixture) which allow de-repression of the tdTomato locus, allowing expression when editing occurs. In some conditions, the additive Tween-80 was also included in the RNP and peptide formulation at 0.05% concentration (w/v). Cells were plated at 15,000 cells per well. Flow cytometry was performed 5 days after treatments were applied to cells.

As shown in FIG. 10, co-incubation of peptides promoted genome editing in primary murine neural progenitor cells. Peptide #55 (the INF7-TAT derivative featuring three amino acid substitutions: G1K, G20L, Y22N) promoted editing more efficiently than did peptide #22 (the A5K derivative of INF7-TAT) when comparable amounts of Cas9 RNP were used. Cells were derived from the Ai9 reporter mouse, and they express fluorescent protein tdTomato if genome editing creates a small excision that enables gene expression. Peptides mediated delivery of Cas9 RNP (and genome editing) in the presence of non-ionic surfactant tween-80 (aka polysorbate 80), which can prevent peptide-mediated aggregation and promote monodisperse solutions of Cas9 RNP. In this experiment, Tween-80 enhanced editing efficiency as compared to the respective “no additive” conditions.

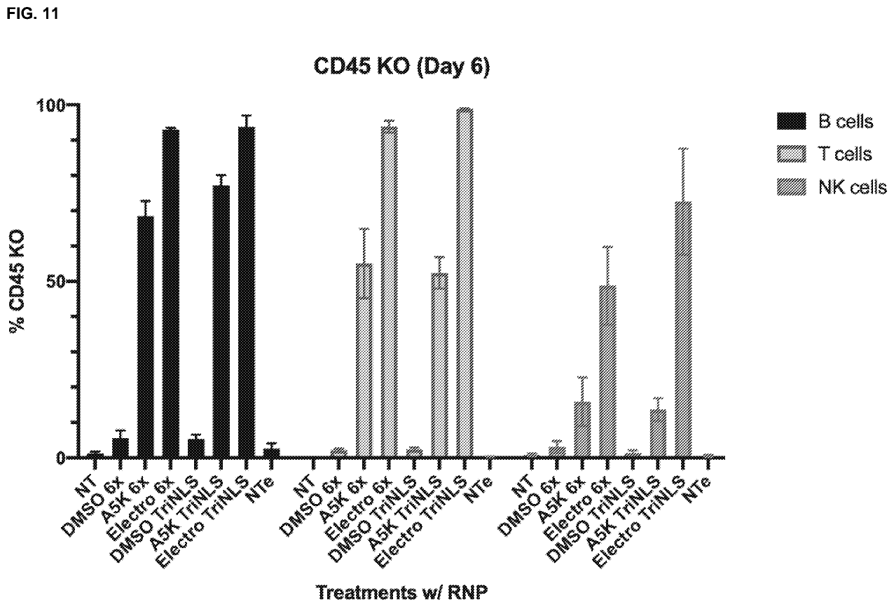

FIG. 11. Editing at the CD45 locus in B cells, T cells, and NK cells via either peptide coincubation or electroporation, as measured by flow cytometry of CD45 KO. Cas9-6×NLS (“6×NLS”) or Cas9-3×NLS (“triNLS”) were combined with the A5K peptide at 10 μM final concentration for peptide-based delivery, or electroporated according to normal protocols. T cells and B cells were split every 2 days until readout on Day 6 post delivery for CD45KO on an Attune NxT flow cytometer. NT—nontreated. NTe—cells electroporated but without Cas9.

As shown in FIG. 11, peptide mediated delivery is effective in allowing genome editing of primary human B cells, T cells, and NK cells. The A5K peptide (10 μM final concentration) applied with Cas9-6×NLS or Cas9-3×NLS and resulted in very high efficiency editing in primary human B cells (77% CD45 KO with Cas9-3×NLS). Slightly lower efficiency was observed in T cells (55% CD45 KO with Cas9 6×NLS). NK cells were more resistant to peptide-based editing under these conditions, as 16% CD45 KO with Cas9-6×NLS was observed.

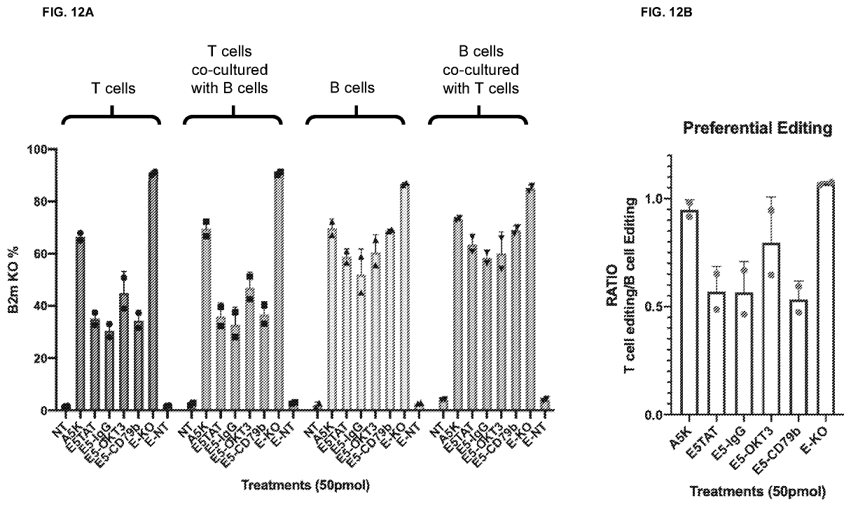

FIG. 12A and FIG. 12B. (A) Levels of β2M (β2M) KO in T cells and B cells, either cultured separately or as a co-culture of T cells and B cells, as measured by flow cytometry. Cells were treated with Cas9 RNPs and either the ASK peptide (peptide #22) or E5-TAT peptide (peptide #1). Treatments with targeting antibodies involved complexing prA-Cas9 RNPs with either a non-targeting control IgG, OKT3, or anti-CD79b and mixed with the E5-TAT peptide. Coincubation conditions were compared to electroporated RNPs (“E-KO”) or cells that were electroporated without treatment, as a negative control. (B) The ratio of editing in T cells over B cells in several treatment conditions.

As shown in FIG. 12A and FIG. 12B, the peptides E5-TAT (peptide #1) or A5K (peptide #22) promoted Cas9-mediated genome editing of human primary T cells and human primary B cells when those two cell types were present together in the same solution.

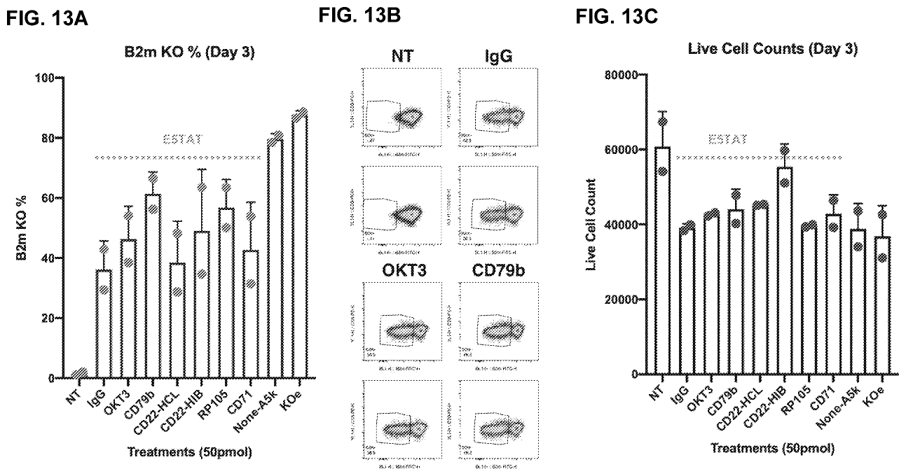

FIG. 13A-13C. (A) Percentage of primary human B cells displaying KO of β2M under different treatment conditions. prA-Cas9-3×NLS RNPs were complexed with an antibody (either control non-targeting IgG, OKT3, anti-CD79b, anti-CD22-HCL, antiCD22-HIB, RP1-5, antiCD71) and mixed with ESTAT (peptide #1, dashed line) at 10 μM final concentration or with no antibody (“none”) and mixed with A5K (peptide #22) at 10 μM final concentration and applied to cells. β2M levels were measured by flow cytometry 3 days after the treatment. “KOe” refers to knock-out mediated by Cas9 RNP being delivered via electroporation. (B) Representative flow scatter plots indicating gating strategy for analyzing β2M KO under four conditions. (C) Live cell counts of cells 3 days after the treatments as measured by flow cytometry. NT=nontreated cells. Replicates are from two distinct human donors.

As shown in FIG. 13A-13C, peptides mediated efficient editing of human primary B cells when the peptide was co-incubated with Cas9 RNP targeting the B2m gene for knock-out. Peptide-mediated delivery of Cas9 RNP resulted in editing rates comparable to that attained by electroporation-mediated delivery of Cas9 RNP, and the overall cell yield (the number of edited cells obtained) was not markedly different between the two techniques.

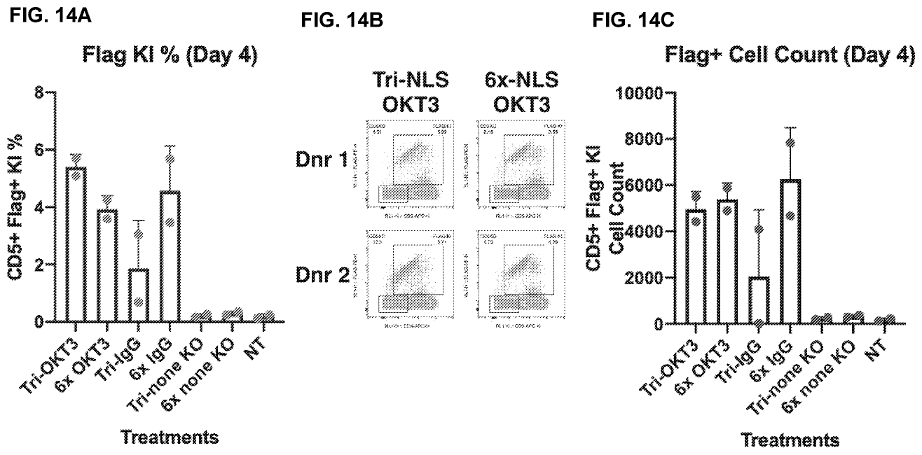

FIG. 14A-14C. Knock-in via HDR of a Flag tag into the CD5 locus in primary human CD4+ T cells. Biological replicates from two human donors. “Tri-OKT3”=“prA-Cas9-3×NLS” with the OKT3 antibody. “6×OKT3”=Cas9-6×NLS with the OKT3 antibody. “Tri-IgG”=“prA-Cas9-3×NLS” with non-targeting control IgG. “6× IgG”=Cas9-6×NLS with the non-targeting control IgG. “Tri-none-KO”=prA-Cas9-3×NLS with no antibody and no DNA present for KI. “6× none KO”=Cas9-6×NLS with no antibody and no DNA present for KI. “NT”=nontreated control cells. FIG. 14A: Percentage of T cells which are positive for Flag tag under different conditions. FIG. 14B: Representative flow plots from two conditions indicating gating strategy for quantifying KI of the Flag Tag in T cells. FIG. 14C: Cell count of CD5+ and Flag+ cells.

As shown in FIG. 14A-14C, peptides mediated delivery of Cas9 RNP nuclease as well as the “donor” template DNA required for mediating knock-in editing in primary human T cells. The Cas9 RNP was delivered along with a template DNA molecule encoding a Flag tag. Using peptide-mediated delivery (and no viral vector or electroporation), knock-in editing rates of ˜5% were attained.

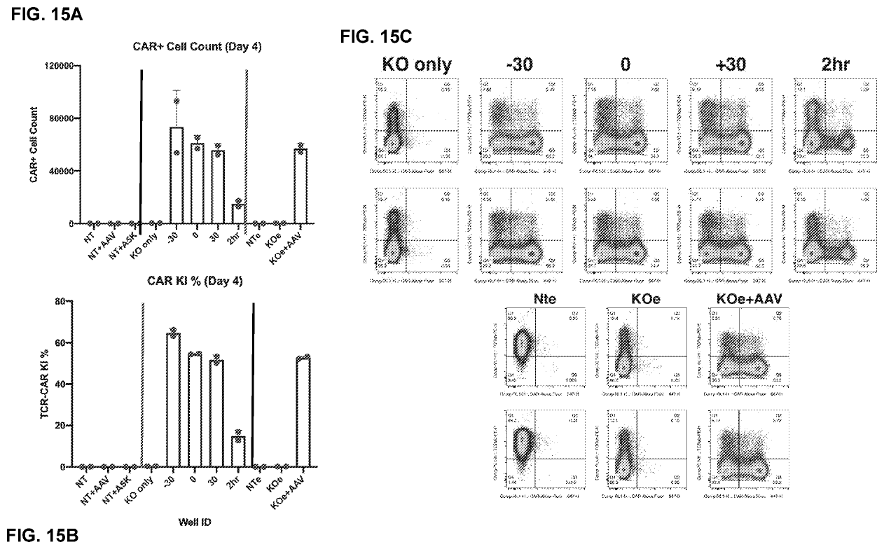

FIG. 15A-15C. Knock in of 19287-CAR at the TCR locus in CD4+ T cells where TRAC RNPs were delivered through coincubation with A5K peptide to perform TRAC KO and the CAR locus was delivered with AAV6, at 4 different timings (30 minutes before RNP treatment, at the same time, 30 minutes after, or 2 hours after). Coincubation method was applied to electroporation method (“KOe”) and showed comparable CAR knock in percentages. Cells were analyzed by flow cytometry for presence of CAR and plotted as (A) CAR+ cell count or (B) Percent of cells that were negative for TCR indicating KO and positive for CAR (C) Representative scatter plots from flow cytometry. Data points indicate biological replicates from two distinct human donors.

As shown in FIG. 15A-15C, peptide-mediated delivery of Cas9 RNP can be used in conjunction with an AAV viral vector carrying a “donor” template DNA. In this case, the Cas9 RNP nuclease was targeted to create a double-stranded break at the TRAC locus; the AAV was carrying a template DNA sequence for a chimeric antigen receptor (CAR). When both were delivered to the same cell, it was possible to perform knock-in of a CAR into the native TRAC locus, resulting in a precisely engineered CAR-T cell with endogenous regulation of the CAR. CAR-T cells were efficiently generated (without the need for electroporation) when the AAV was applied to the cells at the same time as the RNP-peptide formulation, or 30 minutes before, or 30 minutes after.

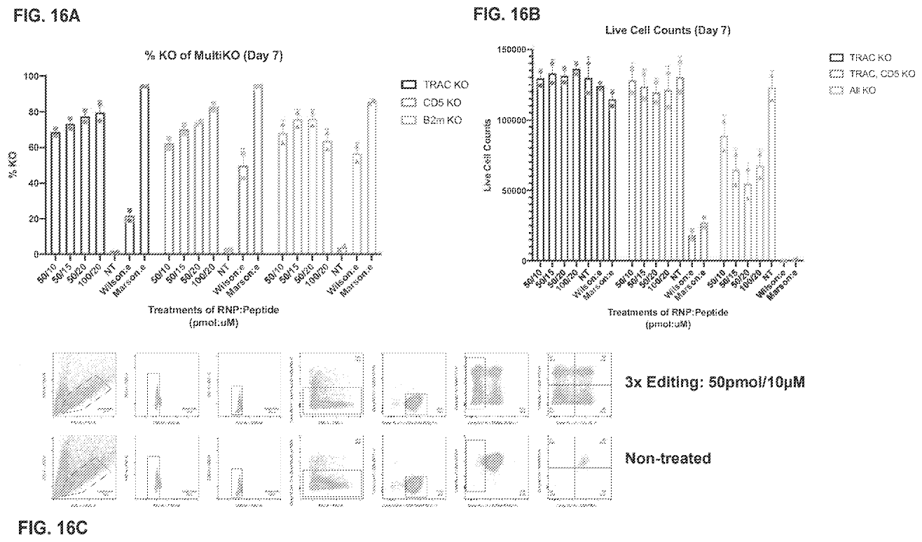

FIG. 16A-16C. Sequential editing of CD4+ primary T cells at three genomic loci (TRAC, CD5, B2M) through coincubation of Cas9 RNPs (either 50 or 100 pmol; the first number in the ##/##shorthand) with A5K peptide at either 10 μM. 15 μM, or 20 μM (the second number in the ##/##shorthand). (A) Percentage of T cells that were edited to cause a gene knock-out (KO) at the TRAC locus or the CD5 locus or the β2M locus. Two distinct electroporation conditions were used as comparators: Wilson: e is an electroporation condition approximating the co-incubation conditions, while Marsone is an electroporation condition that has been totally optimized in the Marson lab. (B) Percentage of T cells that were edited to cause a gene knock-out (KO) at the TRAC locus, the TRAC and CD5 locus, and the TRAC, CD5 and β2M locus. (C) Flow cytometry scatter plots of 3× edited cells at the TRAC, CD5, and β2M loci (top row) as compared to non-treated cells.

As shown in FIG. 16A-16C, peptide-mediated delivery of Cas9 RNP for genome editing of T cells can be used to perform sequential multiplex genome editing. In this example, a formulation of Cas9 RNP and A5K peptide was applied to T cells every 2 days, with each Cas9 RNP targeting a different gene for KO: first TRAC, then CD5, then β2M. Because each treatment of peptide-Cas9 left more cells alive (than does electroporation), a robust yield of cells—including triply edited cells—was obtained by the end of the procedure. In contrast, sequential electroporation resulted in very few edited cells after two rounds of editing, and essentially none after three rounds of editing.

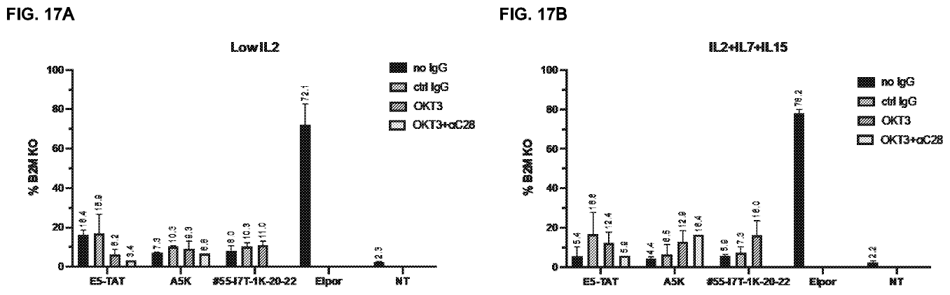

FIG. 17A-17B. Peptide mediated editing in T cells under different stimulation conditions. Primary human T cells which had been previously treated with cytokines (but not activated) were evaluated for peptide mediated genome editing via flow cytometry of β2M knockout levels. Three peptides were examined: E5-TAT. A5K. and Peptide #55, all at 10 μM final concentration in media. prA-Cas9-3×NLS RNPs were complexed with either no antibody, a control non-targeting IgG, anti-CD3 antibody, or anti-CD3 antibody and anti-CD28. Replicates are from two human donors. T cells were kept in media with low concentrations of IL-2 (50 U/mL) for 48 hours until delivery complexes were added (FIG. 17A; or T cells were treated with a mixture of three cytokines: IL-2 (200 U/mL), IL-7 (5 ng/ml), and IL-15 (5 ng/mL) at normal concentrations for 48 hours until delivery complexes were added (FIG. 17B).

Peptide-based delivery of prA-Cas9-3×NLS RNPs into T cells that had not been activated via beads was performed. The impact of two different low-level stimulation conditions was tested, where cells had been treated with low concentrations of IL-2 (FIG. 17A) or a mixture of 3 cytokines (FIG. 17B). As shown in FIG. 17A-17B, editing of the non-activated T cells was observed under all conditions, albeit at lower levels than when bead-based activation of the cells was performed.

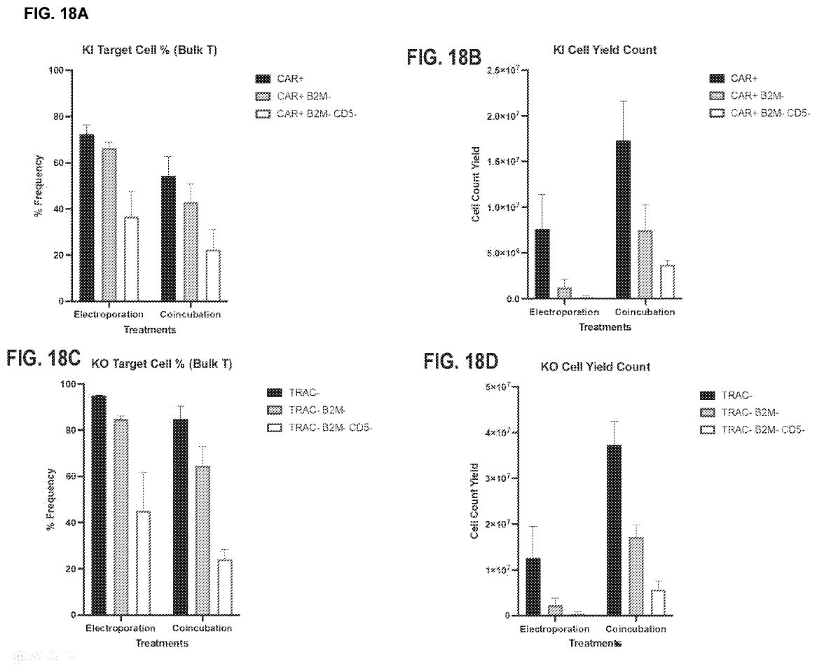

FIG. 18A-18D. Knock in of 19287-CAR at the TCR locus with subsequent sequential KO in CD3+ Bulk T cells. TRAC-targeting Cas9 RNPs were delivered either through electroporation or coincubation with A5K peptide (peptide #22) to perform TRAC KO and 1 hour later an AAV6 vector was applied to deliver the DNA HDR template encoding the CAR cassette. Two days later, a portion of those edited cells was subsequently edited to induce a knock out at the β2M locus (CAR+B2M−) either by electroporation or coincubation. Two days later, another portion of those edited cells was subsequently edited to induce a knock out at the CD5 locus (CAR+B2M-CD5−). The frequency of successfully edited cells (FIG. 18A) and total cell count yield (FIG. 18B) is reported as a percentage for both electroporation and coincubation conditions. The percentage value reported reflects the cells that contained all edits that were attempted for a given sub-set of cells. Each experimental condition (electroporation or coincubation) utilized 4 million cells. (FIG. 18C and FIG. 18D) Sequential knockout at the TRAC, β2M and CD5 loci, performed as in FIGS. 18A and 18B, but without the inclusion of AAV6 viral vector (i.e. CAR knock-in was not attempted). As in FIGS. 18A and 18B, the frequency of successfully edited cells (FIG. 18A) and total cell count yield (FIG. 18B) is reported as a percentage for both electroporation and coincubation conditions. Replicates are from 3 human donors.

As shown in FIG. 18A-18D, sequential editing via peptide-mediated coincubation of Cas9 RNPs in primary human CD3+ T cells results in similar percent frequencies of edited cells, but much higher yield (absolute number of edited cells) compared to sequential editing via electroporation.

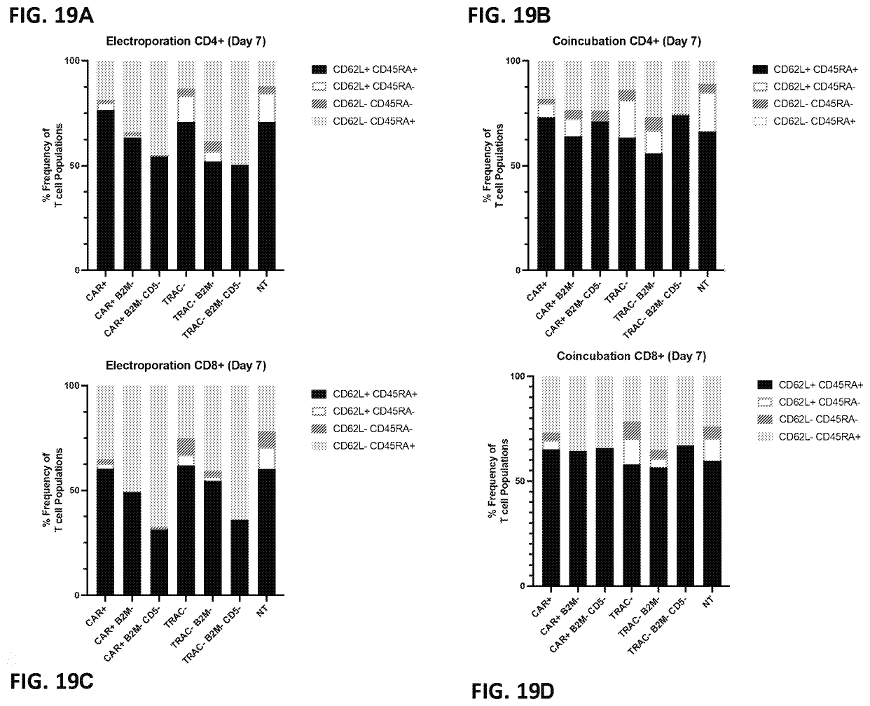

FIG. 19A-19D. Knock in of 19287-CAR at the TCR locus with subsequent sequential KO in CD3+ Bulk T cells, or cells treated only for KO (without AAV KI). Comparison of T cell phenotypes between sequential editing via electroporation (FIG. 19A and FIG. 19C) and sequential editing via peptide coincubation (FIG. 19B and FIG. 19D) in CD4+ T cells or CD8+ T cells. Total cell populations were assessed for CD62L and CD45RA phenotypes, regardless of edited phenotype.

As shown in FIG. 19A-19D, sequential editing via electroporation decreases the proportion of naïve T cells (CD62L+CD45RA+) present in both CD4+ and CD8+ populations. Sequential editing via RNP/peptide coincubation does not have a marked impact on the proportion of naïve T cells (CD62L+CD45RA+) present in CD4+ or CD8+ populations.

### Example 3: Base Editing

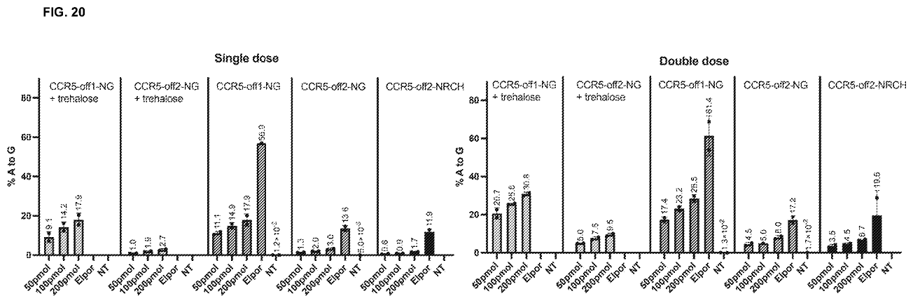

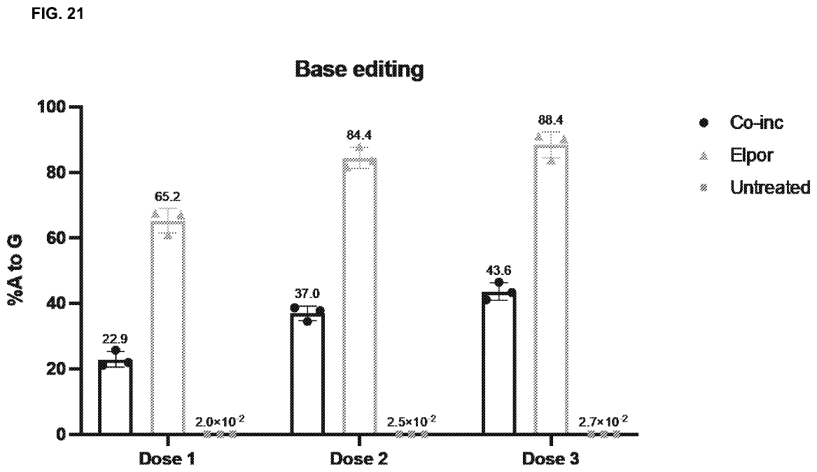

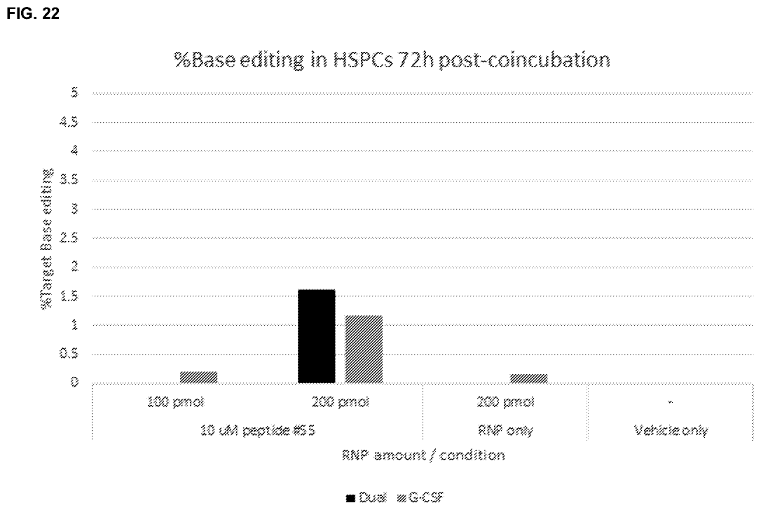

Peptides were used to deliver RNP complexes comprising a Cas effector-base editor fusion protein. Amino acid sequences of the Cas effector-base editor fusion proteins are presented in FIG. 26A FIG. 27A and FIG. 26C FIG. 26C. The results are shown in FIG. 20, FIG. 21, and FIG. 22.

FIG. 20. Adenine to Guanine base editing at the CCR5 locus in primary human T cells delivered via peptide-coincubation with the ASK peptide. Dose optimization was performed where either 50, 100 or 200 pmol of SpCas9-NG or SpCas9-NRCH PAM variants were applied to cells with 20 μM (final concentration) A5K peptide. The cells were either treated with a single dose or followed up with a second dose 2 days after the first. The addition of trehalose, a stabilizing additive to the buffer formulation, was also tested. The highest editing rates were achieved with a double dose of SpCas9-NG and the “CCR5-off1” guide at 200 pmol dose, giving 28.5% editing. Conditions were compared against an electroporated positive control of the formulation (without the peptide) and non treated (NT) as a negative control. Two biological replicates in cells from two human donors.

As shown in FIG. 20, peptides mediated delivery of a base editor construct RNP, promoting base editing in T cells to remove the start codon of CCR5, causing initiation of translation at a frame-shifted, downstream alternative start codon. Base editing efficiency can be increased via sequential, repeat application of peptide and base editor (delivered in this example either via peptides or electroporation). PAM variant enzymes were used in this example, either the “NG” variant or the “NRCH” variant.

FIG. 21. Adenine to Guanine base editing at the CCR5 locus in primary human T cells delivered via peptide-coincubation with the ASK peptide. ABE8e-SpCas9-NG PAM variants were applied to cells with 20 μM (final concentration) A5K peptide. The cells were either treated with a single dose or followed up with a second dose 2 days after the first or followed up again with a third dose 2 days after the second dose. The highest editing rates were achieved with a triple dose of SpCas9-NG and the “CCR5-off1” guide at 200 pmol dose, giving 43.6% editing. Conditions were compared against an electroporated positive control of the formulation (without the peptide) and untreated as a negative control. Three biological replicates in cells from three human donors.

As shown in FIG. 21, peptides mediated delivery of a base editor construct RNP (ABE8e-SpCas9-NG), promoting base editing in T cells to remove the start codon of CCR5, causing initiation of translation at a frame-shifted, downstream alternative start codon. Base editing efficiency can be increased via sequential, repeated application of peptide and base editor (delivered in this example either via peptides or electroporation). The “NG” PAM variant enzymes were used in this example. After 3 sequential doses of the base editor RNP with 20 μM A5K peptide (doses applied every 2 days) we observe 43.6% base editing at the CCR5 locus.

FIG. 22. Base editing in primary human HSPCs at the erythrocyte-specific BCL11a enhancer locus, applying ABE-8e-NG RNPs at 100 pmol or 200 pmol dose with 10 μM peptide #55 and analyzing genomic DNA for base editing via deep sequencing 72 hours after treatment.

As shown in FIG. 22, peptide #55 mediated delivery of a base editor construct RNP, promoting base editing in HSPCs to convert an “A” nucleotide to “G” in the erythroid-specific enhancer of Bcl11a.

### Example 4: DNA Vaccine

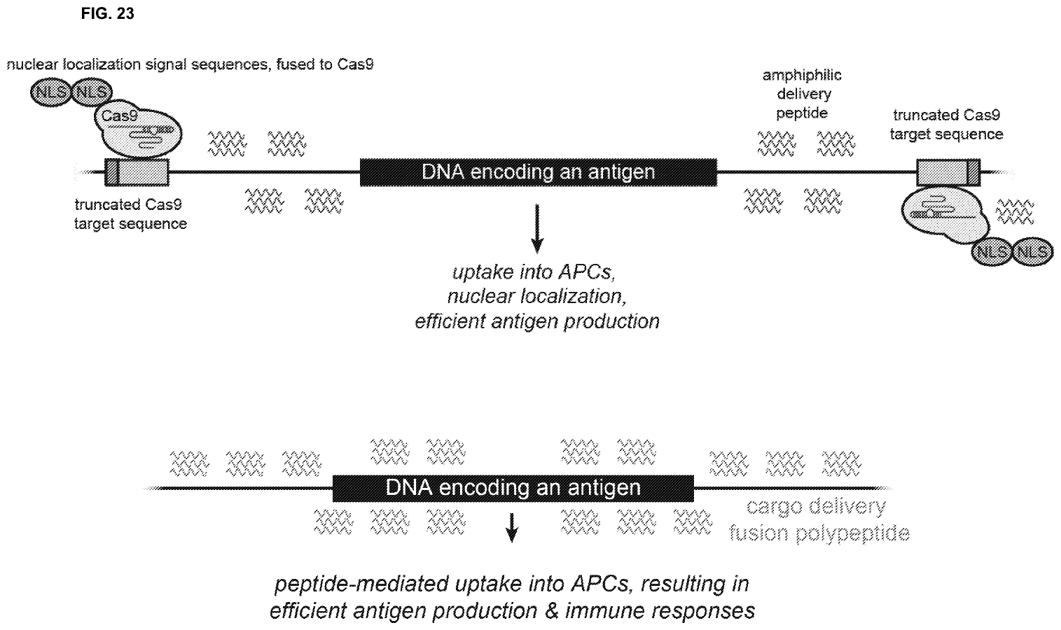

Use of an amphiphilic peptide to deliver a DNA vaccine (a DNA molecule encoding an antigenic polypeptide) was explored. FIG. 23 presents a schematic indicating how DNA is delivered via peptides in the context of DNA vaccines, where target cells express the antigen encoded in the delivered DNA allowing a robust immune response.

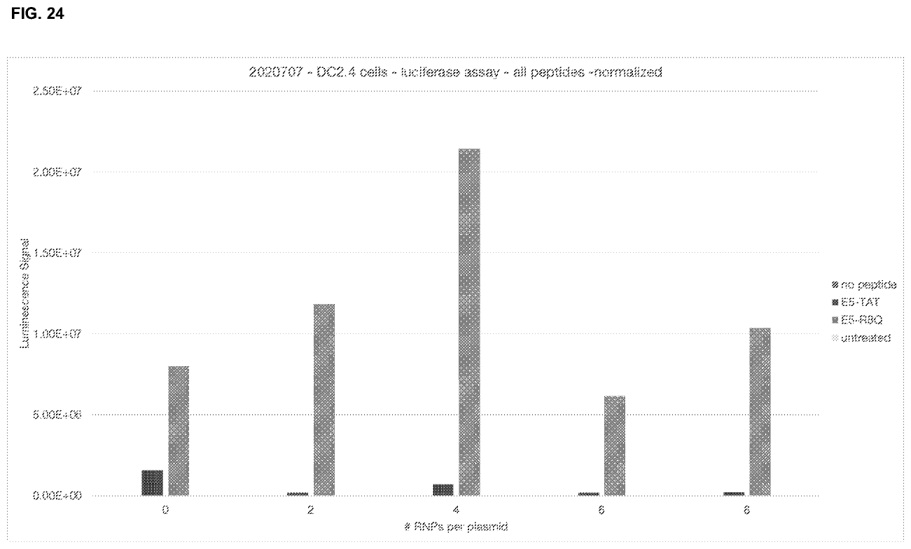

To test delivery into cells in vitro, DNA encoding luciferase was introduced into DC2.4 cells using E5-TAT or E5-R8Q. The results are shown in FIG. 24.

FIG. 24. Luciferase activity indicating successful peptide-mediated DNA delivery and protein expression in DC2.4 cells. Plasmid encoding luciferase was combined with either 0, 2, 4, 6, or 8 molar equivalents of dCas9 RNPs intended to bind the plasmid and aid protein expression through shuttling the DNA into the cell nuclei. Peptides E5-TAT or E5-R8Q (Peptide #37) were applied at 10 μM. Peptide E5-R8Q with 4 molar equivalents of RNPs provides the most robust luciferase expression. No luciferase expression was observed in the absence of peptide, or when the cells were untreated; the only apparent signal results when DNA was combined with E5-TAT or E5-R8Q.

As shown in FIG. 24, peptides E5-TAT (peptide #1) and E5-R8Q (peptide #37) mediated plasmid delivery into DC2.4, a cell line that is dendritic-like and thus models antigen-presenting cells (APCs). In this example, the plasmid being delivered bears a luciferase reporter gene as well as a number of truncated target sequence sites, which allow the Cas9 RNP to bind, but not to cut. In this example, the most effective delivery was observed with 4 molar equivalents of RNP being added per plasmid, and with E5-R8Q being used to promote delivery of the macromolecular cargo (an RNP-plasmid complex

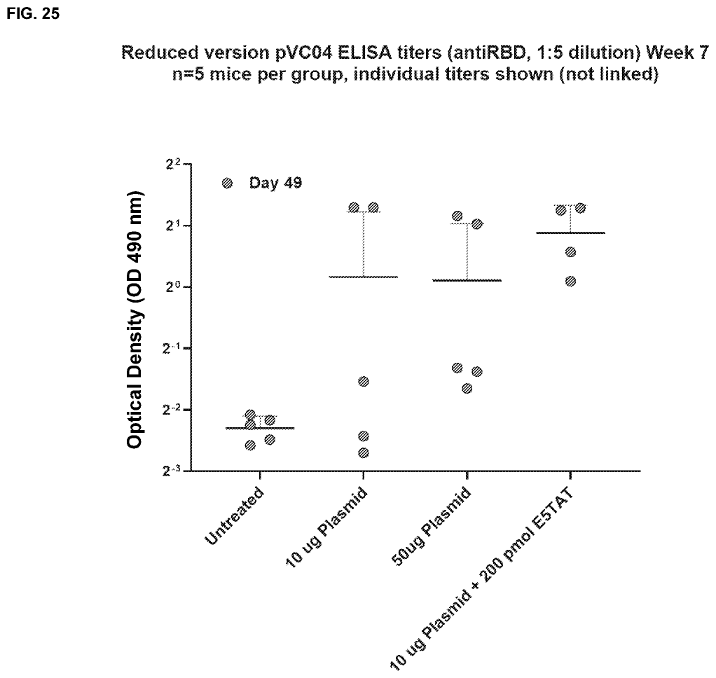

To test in vivo delivery, mice were injected with plasmid DNA encoding the receptor-binding domain (RBD) of the spike protein of SARS-COV-2, with or without an E5-TAT peptide. The results are shown in FIG. 25.

FIG. 25. Antibody titers as measured by ELISA in mice injected with plasmid DNA encoding the RBD of the Spike protein from SARS COV-2 with or without the addition of 200 pmol E5-TAT peptide. Anti-RBD titers were at their most robust when the 10 μg plasmid was co-delivered with 200 pmol E5-TAT.

As shown in FIG. 25, peptides promoted intracellular delivery of plasmid DNA encoding an antigen, resulting in vaccination of mice against the SARS-COV-2 spike protein RBD. In this example the ELISA titers were undetectable in the untreated animals. When antigen-encoding plasmid was administered, there was substantial animal-to-animal variability in the extent of detectable vaccination (e.g. anti-RBD titers). When the plasmid was delivered as part of a formulation that contains peptides, the mice exhibited consistently high titers of anti-RBD antibodies.

### Example 5: Peptide Screening

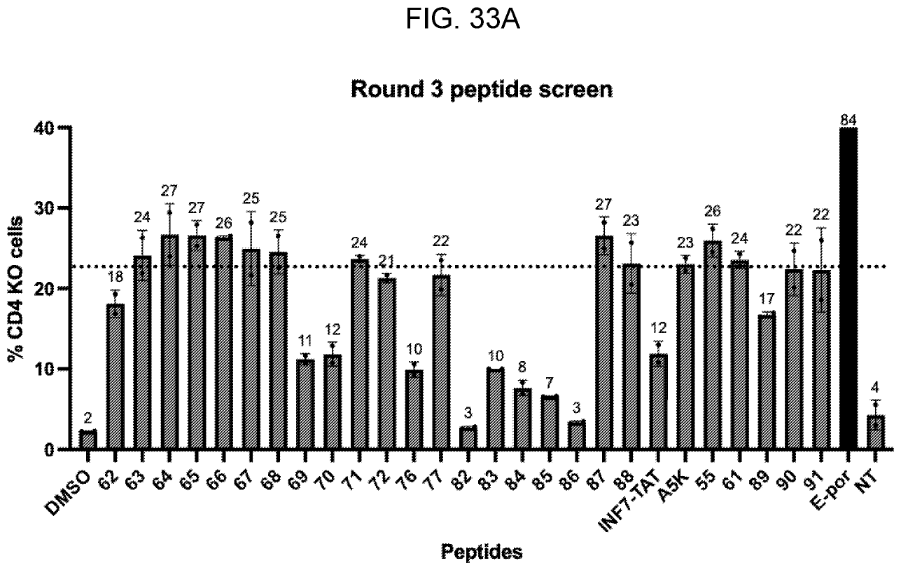

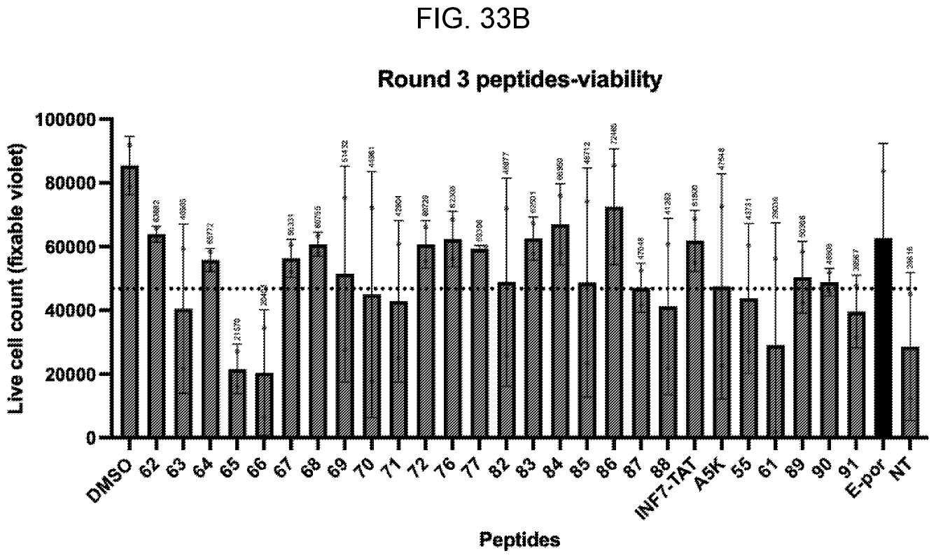

As shown in FIG. 33A-33B, screening of additional peptides revealed that some peptides featuring sequence elements drawn from “parent peptides” #1-37 promoted genome editing in T cells with improved editing efficiency and/or cell viability as compared to peptide #22, one of the best-performing parent peptides. A) Screening of peptides #62-91 (as well as select comparator peptides from the screen of “parent peptides” #1-61; INF7-TAT refers to peptide #11. A5K refers to peptide #22) for Cas9-mediated KO at the CD4 locus in human primary CD4+ T cells, as measured by flow cytometry. 50 pmol RNP was applied to cells with peptides at 10 μM concentration. (B) Live cell counts from each treatment condition as determined by live/dead fixable violet staining (Thermofisher) and flow cytometry. Replicates are from two distinct human cell donors. In both (A) and (B), “DMSO”=Cas9 without peptide; “NT”=cells that were not treated with Cas9 or peptide. Editing and viability was assessed three days after RNP delivery.

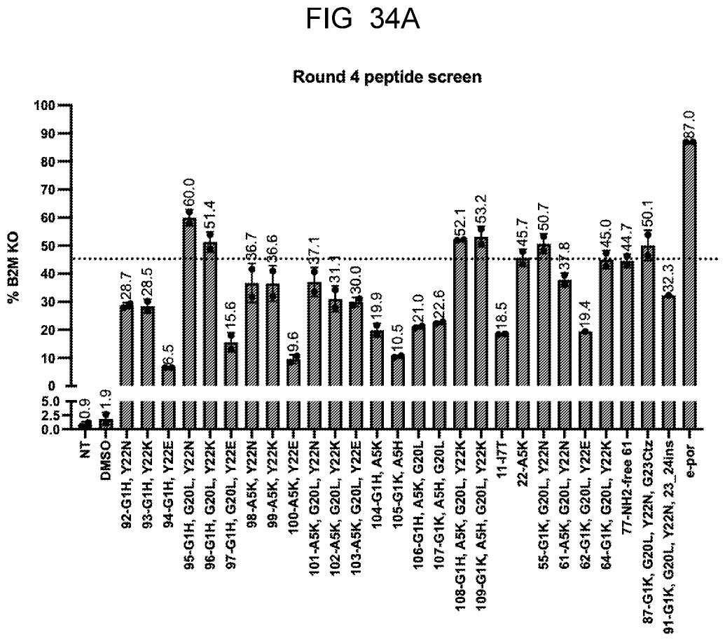

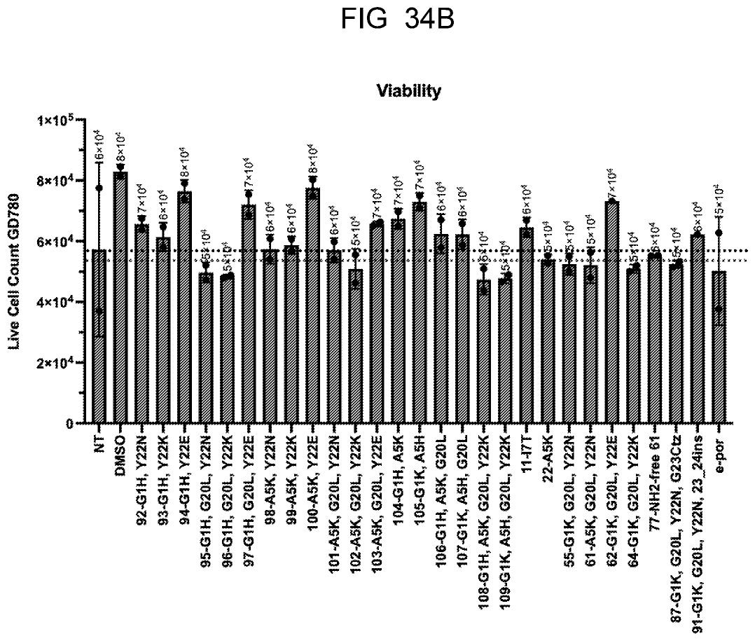

As shown in FIG. 34A-34B, screening of additional peptides revealed that some peptides featuring sequence elements drawn from “parent peptides” #1-91 promoted genome editing in T cells with improved editing efficiency and/or cell viability as compared to peptide #22, one of the best-performing parent peptides. A) Screening of peptides #92-109 (as well as select comparator peptides from the screen of previously screened peptides” #1-91) for Cas9-mediated KO at the β2M locus in human primary CD4+ T cells, as measured by flow cytometry. 50 pmol RNP was applied to cells with peptides at 10 μM concentration. (B) Live cell counts from each treatment condition as determined by live/dead fixable violet staining (Thermofisher) and flow cytometry. Replicates are from two distinct human cell donors. In both (A) and (B), “DMSO”=Cas9 without peptide; “NT”=cells that were not treated with Cas9 or peptide. Editing and viability was assessed three days after RNP delivery.

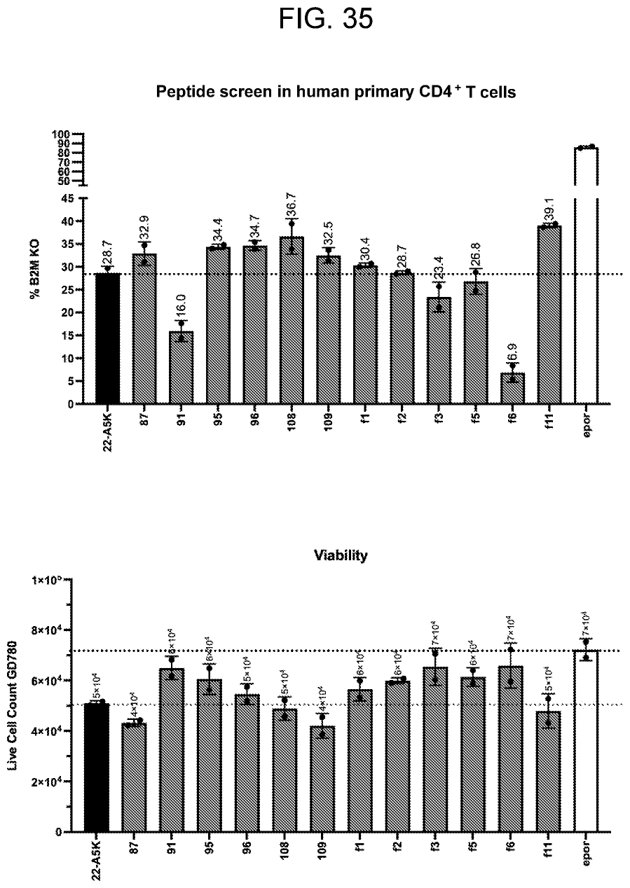

As shown in FIG. 35, screening of additional peptides bearing functional groups (hence “f”, for peptides #f1-15) reveals peptides that retain potency for RNP delivery (and genome editing) in primary human T cells, while some support improved editing efficiency and/or cell viability as compared to peptide #22, one of the best-performing parent peptides. Left: screening of peptides f1/f2/f3/f5/f6/f11 (as well as select comparator peptides from the previous screens) for Cas9-mediated β2M KO in human primary CD4+ T cells, as assayed by flow cytometry. 50 pmol RNP was applied to cells with peptides at 10 μM concentration. Right: ive cell counts from each treatment condition as determined by live/dead fixable violet staining (Thermofisher) and flow cytometry. Replicates are from two distinct human cell donors. Editing and viability was assessed three days after RNP delivery.

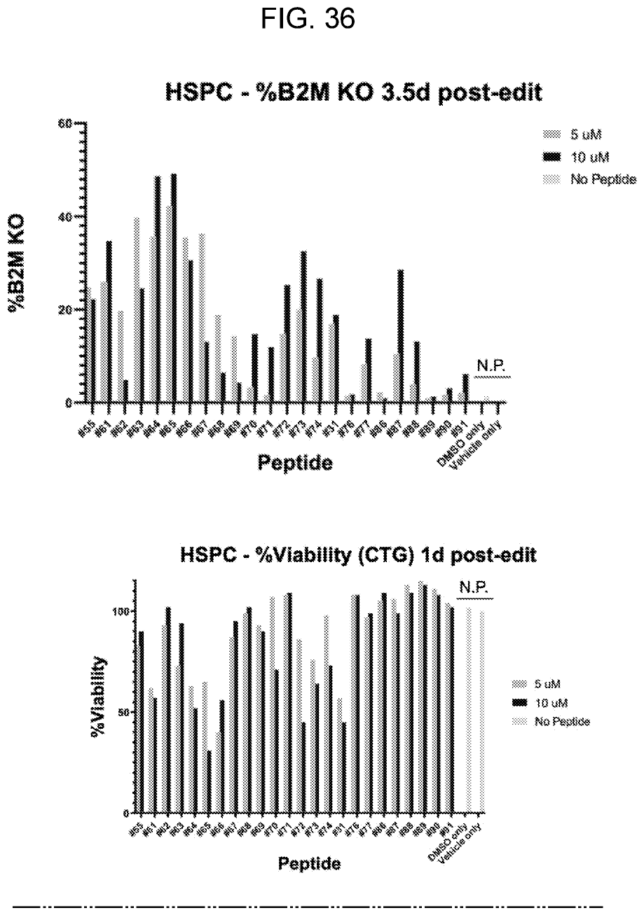

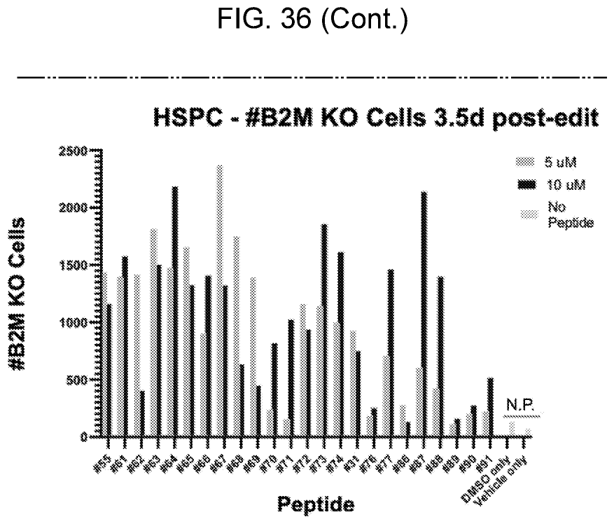

As shown in FIG. 36, screening of additional peptides revealed that some—but not all—peptides featuring sequence elements drawn from “parent peptides” #1-37 promoted genome editing in HSPCs with improved editing efficiency and/or cell viability as compared to peptide #55, one of the peptides that performed best in these regards in HSPCs in prior rounds of screening. In this screen, Cas9 RNP targeted β2M for KO and editing was detected via flow cytometry. Top left: screening of peptides #61-91 (alongside comparator peptides #31 and #55 from previous screens) for Cas9-mediated KO at the β2M locus in human primary CD34+ hematopoietic stem and progenitor cells (HSPCs), as measured by flow cytometry 3.5 days after treatment with 50 pmol RNP and peptides at 5 or 10 μM concentration (medium gray and black bars, respectively). Top right: number of β2M edited cells detected by flow cytometry at 3.5 days post-delivery, plotted for each peptide screened. Bottom left: viability assessed by Cell Titre Glo (CTG) assay 24 hours after treatment with RNP and peptide. “DMSO”=Cas9 without peptide; “Vehicle only”=cells that were not treated with Cas9 or peptide. The “no peptide” data points are marked with “N.P.” and are shown in light gray.

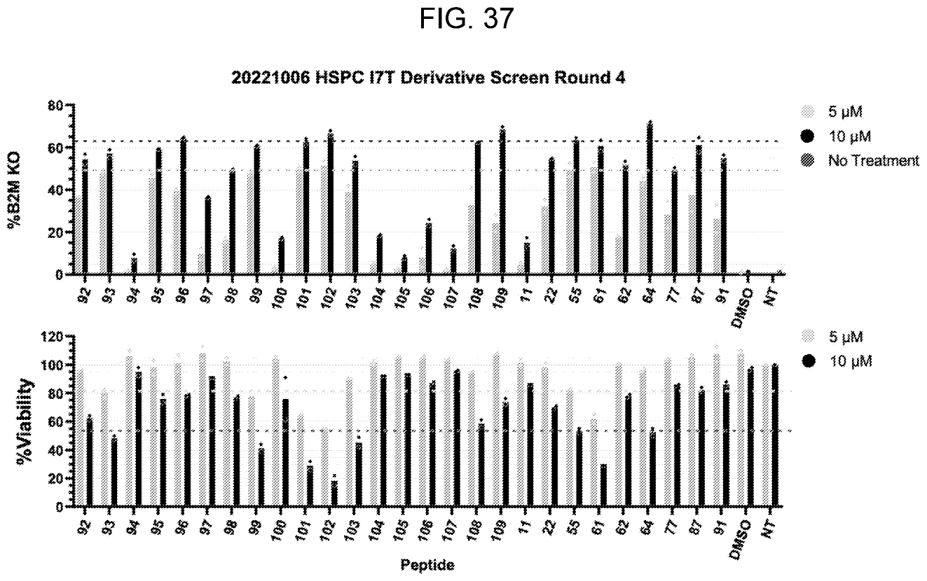

As shown in FIG. 37, screening of additional peptides revealed that some—but not all—peptides featuring sequence elements drawn from “parent peptides” #1-37 promoted genome editing in HSPCs with improved editing efficiency and/or cell viability as compared to peptide #55, one of the peptides that performed best in these regards in HSPCs in prior rounds of screening. In this screen, Cas9 RNP targeted β2M for KO and editing was detected via flow cytometry. Top: screening of peptides #92-109 (alongside some comparator peptides from previous screens) for Cas9-mediated KO at the β2M locus in human primary CD34+ hematopoietic stem and progenitor cells (HSPCs), as measured by flow cytometry 3.5 days after treatment with 50 pmol RNP and peptides at 5 or 10 μM concentration. Bottom: viability assessed by Cell Titre Glo (CTG) assay 24 hours after treatment with RNP and peptide. “DMSO”=Cas9 RNP without peptide; “NT”=cells that were not treated with Cas9 or peptide; colored data points and bars do not reflect peptide concentration in for “DMSO” and “NT” since no peptide was added to cells.

### Example 6: Gene Editing

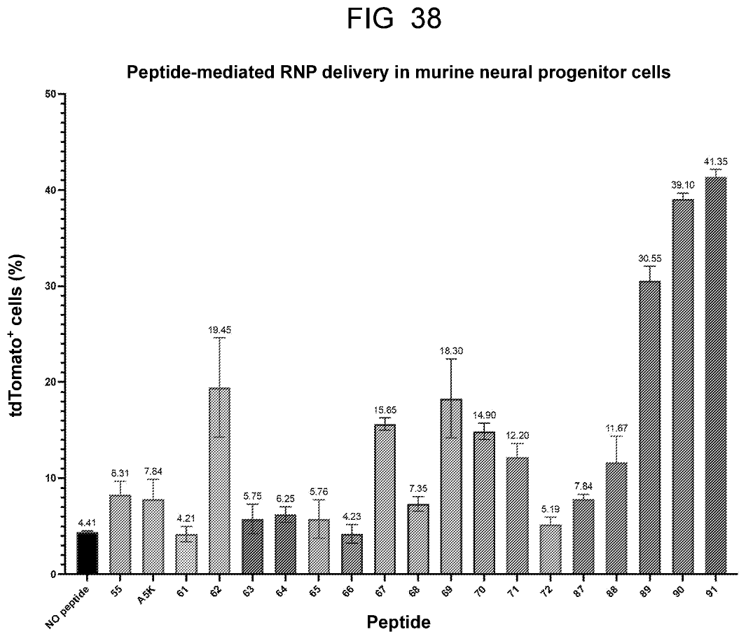

As shown in FIG. 38, screening of additional peptides described in Example 5 revealed that some—but not all—peptides featuring sequence elements drawn from “parent peptides” #1-37 promoted genome editing in Ai9-mouse derived neural progenitor cells (NPCs) with improved editing efficiency and/or cell viability as compared to peptides #22 and #55, two of the peptides that performed best in these regards in Ai9-derived NPCs in prior rounds of screening. Screening of peptides #61-91 was performed alongside comparator peptide #55 and peptide #22 (A5K) for Cas9-mediated cutting of the tdTomato repressor in Ai9 NPCs. Editing efficiency measured by expression of red fluorescent protein by flow cytometry 3 d after treatment with 50 pmol RNP and peptides at 10 μM concentration.

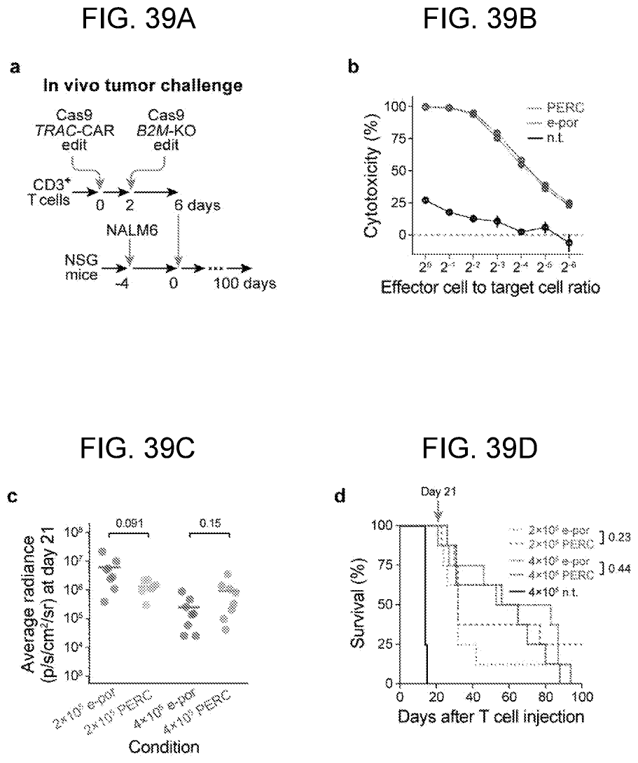

As shown in FIG. 39A-39D, peptide-mediated delivery of Cas9 RNP can facilitate the generation of CAR-T cells that can perform effective tumor killing in an in vivo xenograft mouse model, with efficacy comparable to that of cells generated using electroporation. a, Schematic of the in vivo tumor challenge experiment, using n=8 mice per group. b, NALM6 cytotoxicity assay. Error bars represent S.E.M from three technical replicates. c, Bioluminescence Imaging values on the last measurement day on which all CAR-T cell-injected mice were alive. P-values are from two-tailed Welch's unpaired t-tests. d, Kaplan-Meier survival analysis. P-values are from a log-rank test. P<0.001 for each comparison of an edited condition vs. n.t. cells.

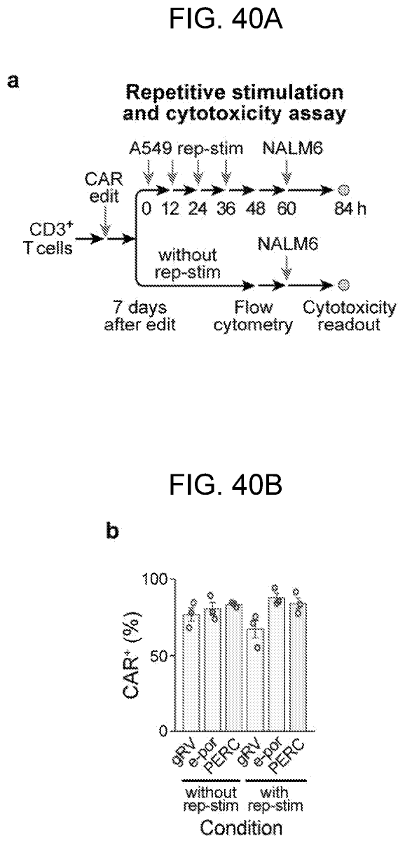

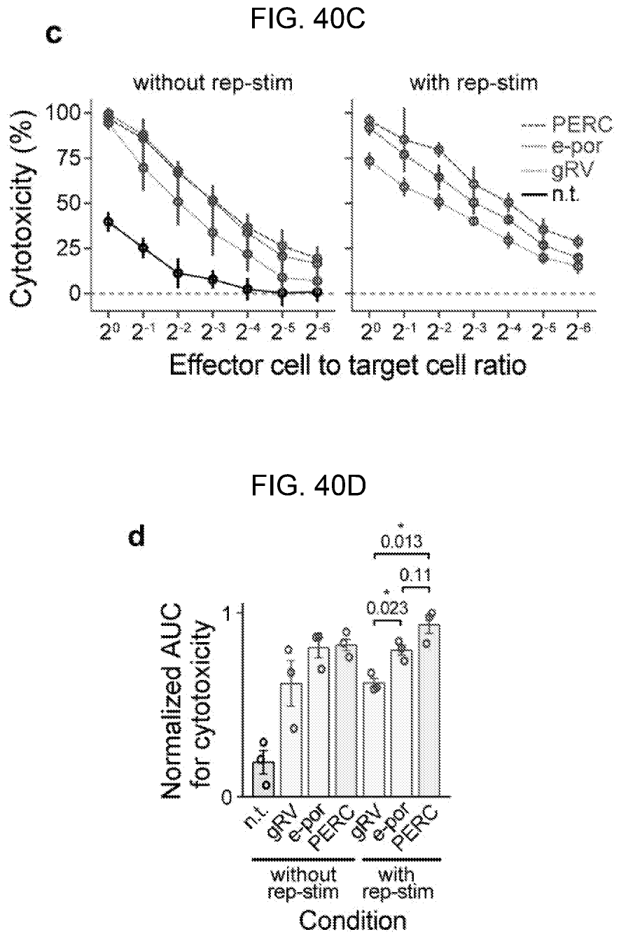

As shown in FIG. 40A-40D, peptide-mediated delivery of Cas9 RNP can facilitate the generation of CAR-T cells that can perform effective tumor killing in an ex vivo assay, with efficacy comparable to that of cells generated using electroporation and superior to that of CAR-T cells generated using gammaretrovirus for CAR delivery. a, Schematic of the repetitive stimulation and cytotoxicity assay. CD3+ T cells were edited to express a CAR using either gRV, Cas12a RNP electroporation and AAV, or Cas12a RNP PERC and AAV. b, Percentages of CAR+ cells in each condition with or without repetitive stimulation using CD19+A549 cells. c,d, NALM6 cytotoxicity assay for T cells in each condition, and area under the curve analysis. n=3 biological replicates from distinct human donors. Bars represent the mean. Error bars represent S.E.M. (3 biological replicates×3 technical replicates). P-values are from two-tailed Welch's unpaired t-tests.

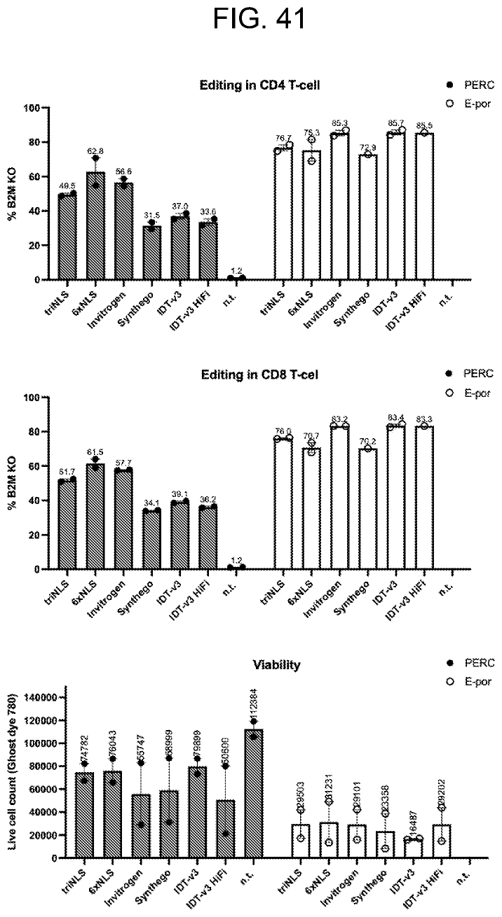

As shown in FIG. 41, peptide-mediated delivery of Cas9 RNPs is effective when various, distinct Cas9 protein constructs are used, from either academic or commercial origins. FIG. 41 shows a comparison of editing efficiency and viability following peptide-mediated delivery with peptide #22 (ASK; “PERC”) or electroporation (e-por) of various S. pyogenes Cas9 protein constructs. Two published NLS-rich constructs, triNLS (similar to “3×NLS” in Wu et al. doi: 10.1038/s41591-019-0401-y) and 6×NLS (analogous to “4×NLS” in Staahl et al. (2017) Nat. Biotechnol. 35:431), were purified in academic labs. The other four constructs are from commercial vendors. TrueCut-v2: Invitrogen TrueCut Cas9 Protein v2 (ThermoFisher). Synthego: SpCas9 2NLS (Synthego). IDT-v3: Alt-R S.p. Cas9 Nuclease V3 (IDT). IDT-v3 HiFi: Alt-R S.p. HiFi Cas9 Nuclease V3 (IDT).

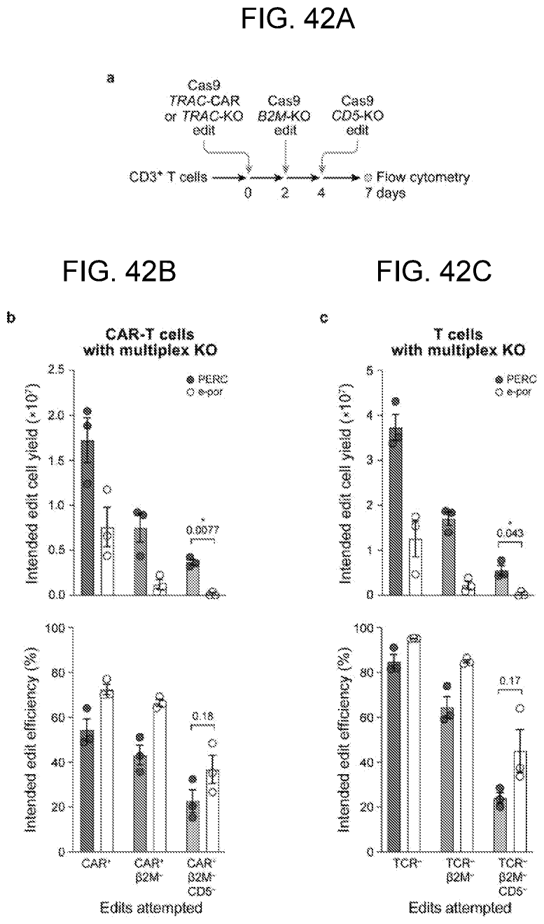

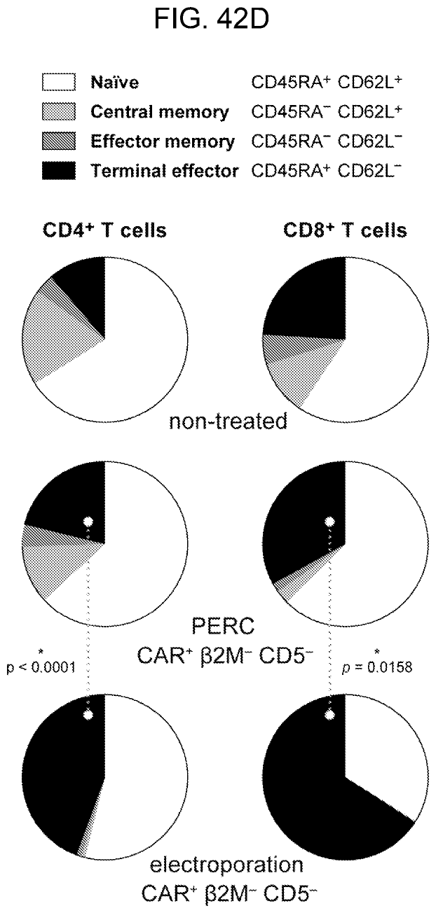

As shown in FIG. 42A-42D, peptide-mediated delivery (“PERC”) supports cell viability and maintenance of phenotype in sequential editing. a, Schematic of sequential editing of three loci in CD3⁺ T cells. b, Comparison of editing using PERC (peptide #22; A5K) vs. electroporation as measured by flow cytometry for TCR, CAR. β2M, and CD5 surface expression. Reported CAR+ cells are also TCR⁻. c, Editing without CAR AAV. Cell counts for each condition are scaled to an initial input of 4×10⁶ T cells. Bar graphs represent only the cells that have all attempted edits, and do not include cells that have only some of the attempted edits. n=3 biological replicates from distinct human donors. Bars represent the mean. Error bars represent S.E.M. P-values are from two-tailed Welch's unpaired t-tests. D. Comparison of CD4⁺ and CD8⁺ cell phenotypes between delivery methods (independent of editing outcome), as measured by flow cytometry for CD62L and CD45RA surface expression. Pie charts represent proportions of various cell phenotypes as observed following three serial rounds of either PERC (center pair) or electroporation (bottom pair) with comparison to non-treated cells (top pair). Pic segments represent the mean proportion of each phenotype. The dotted lines denote comparisons, with p-values from a two-way ANOVA and Holm-Šidák multiple comparisons test.

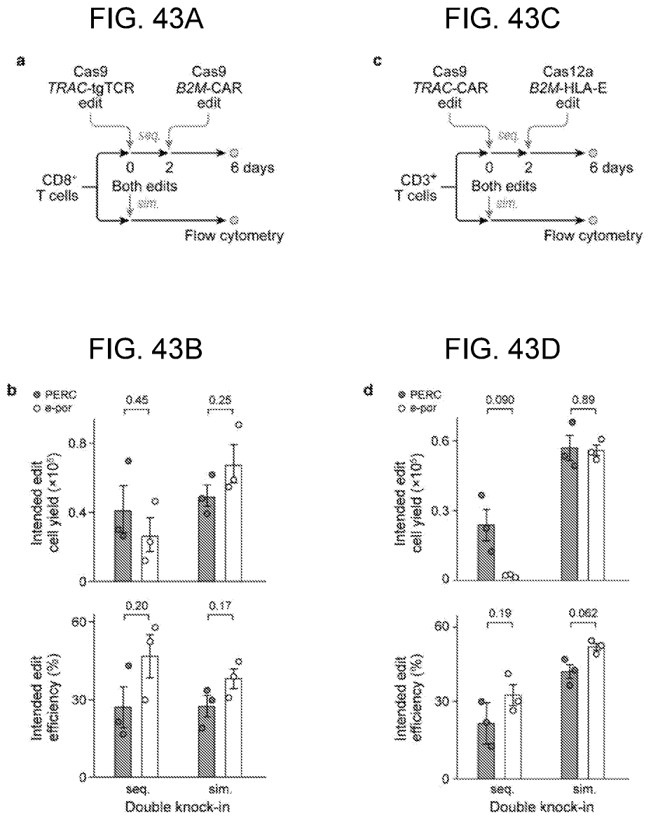

As shown in FIG. 43A-43D, peptide-mediated delivery (“PERC”) supports cell viability in the production of engineered T cells bearing multiple knock-in edits at distinct genomic loci. This can be performed using only Cas9 RNP, or a mixture of Cas9 RNP and Cas12a RNP (one for each genomic locus). a,b, Sequential and simultaneous double knock-in editing by PERC (peptide 22/A5K) or e-por (electroporation) in CD8⁺ T cells using Cas9 and PERC (peptide #22/A5K). c,d, Sequential and simultaneous double knock-in editing by PERC (peptide #22/A5K) or e-por (electroporation) in CD3⁺ T cells using Cas12a and Cas9. Bar graphs represent cells that bear both attempted knock-in edits. Flow cytometry was conducted six days after the first edit. n=3 biological replicates from distinct human donors. Bars represent the mean. Error bars represent S.E.M. P-values are from two-tailed Welch's unpaired t-tests.

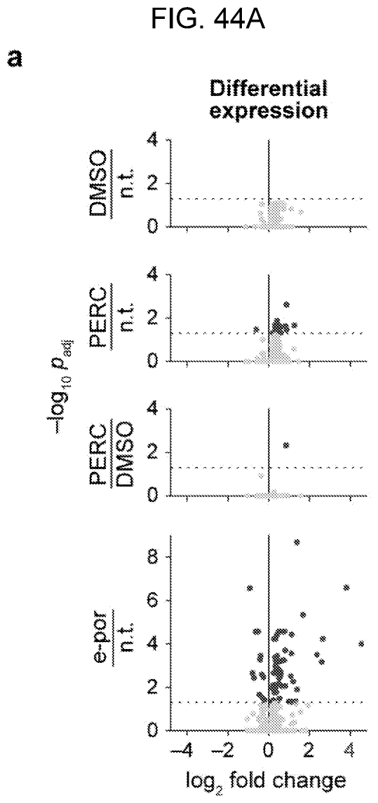

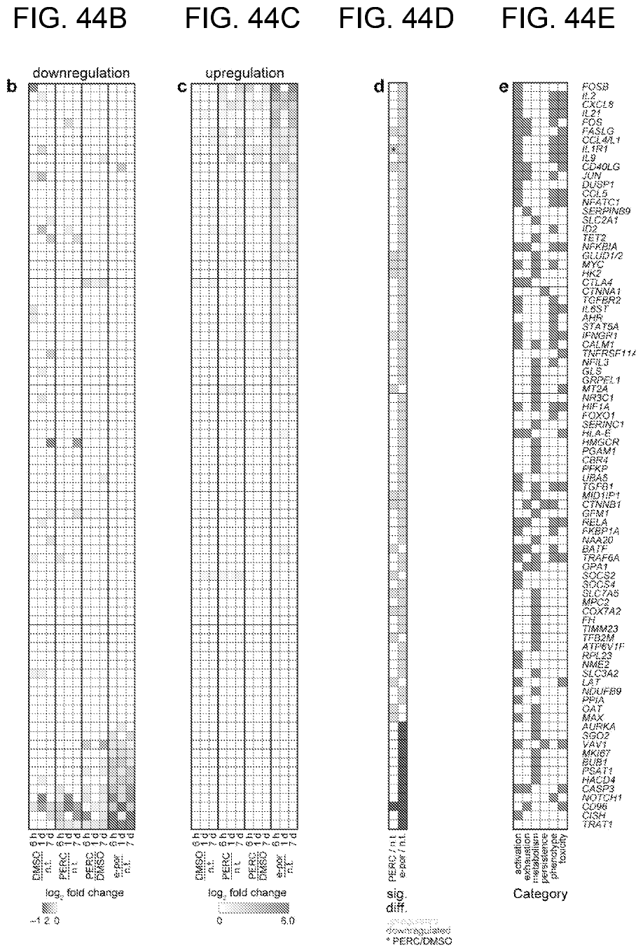

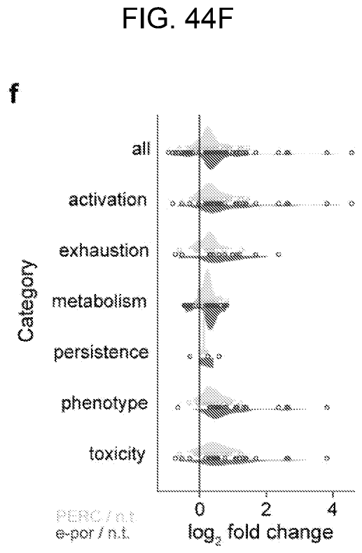

As shown in FIG. 44A-44F, peptide-mediated delivery (“PERC”) of CRISPR RNP supports T cell engineering while inducing minimal perturbation of T cell phenotype, especially as compared to electroporation of RNP. a, Volcano plots depict gene expression fold changes and adjusted p-values incorporating data from 6 h, 1 day, and 7 days after editing via RNP delivery using either peptide #22 (PERC) or electroporation (e-por). The “DMSO” condition exposes cells to RNP and 0.1% DMSO (the same amount contributed by delivery of the peptide, which is dissolved in DMSO) but with no peptide. This plot combines data from all three time points. b-e, Set of 84 genes that were significantly differentially downregulated (b) or upregulated (c) in one or more conditions in a. d, outcomes in each condition; the asterisk indicates the one gene that was significantly affected in PERC/DMSO. e, NanoString gene category annotations and gene labels for b/c/d. f, Fold changes across gene categories using the genes and annotations in b/c/d/e. The dots represent individual genes, and the shaded curves represent distributions of fold changes.

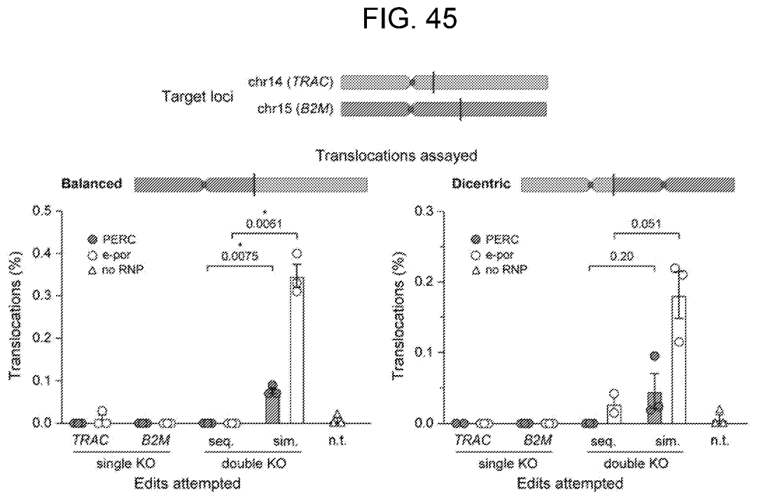

As shown in FIG. 45, peptide-mediated delivery (“PERC”) of CRISPR RNP supports multiplex genome editing with minimal induction of chromosomal translocations, especially as compared to simultaneous electroporation of RNP nucleases targeting multiple genomic loci. This figure shows analysis of translocation frequencies by ddPCR for treatment with one or two Cas9 nuclease RNPs (targeting either TRAC or β2M), sequentially or simultaneously. Editing was performed using PERC (peptide #22/A5K) or e-por (electroporation) in CD3⁺ T cells. In each experiment, n=2-3 biological replicates from distinct human donors. Bars represent the mean. Error bars represent S.E.M. P-values are from two-tailed Welch's unpaired t-tests.

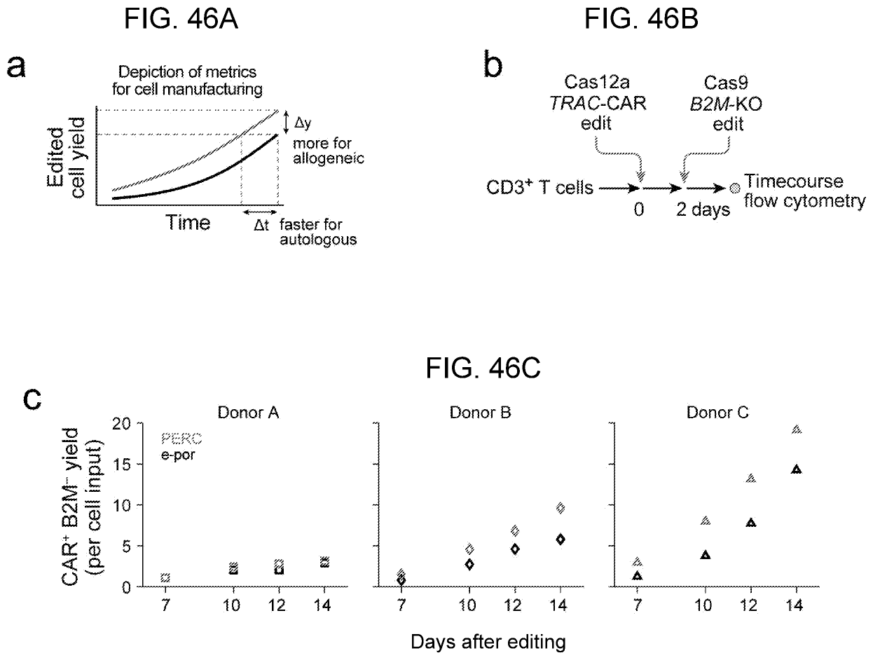

As shown in FIG. 46A-46C, peptide-mediated delivery (“PERC”) of CRISPR RNP supports improved cell yields and robust cell expansion over time following T cell engineering, especially as compared to electroporation of RNP. a, Depiction of metrics for evaluating improvement in cell manufacturing. b, Schematic of sequential editing. c, Expansion of edited cells over time, relative to the number of cells used for editing; n=3 biological replicates from distinct human donors, plotted separately. Editing was performed via PERC (peptide #22/A5K) or e-por (electroporation) in CD3⁺ T cells.

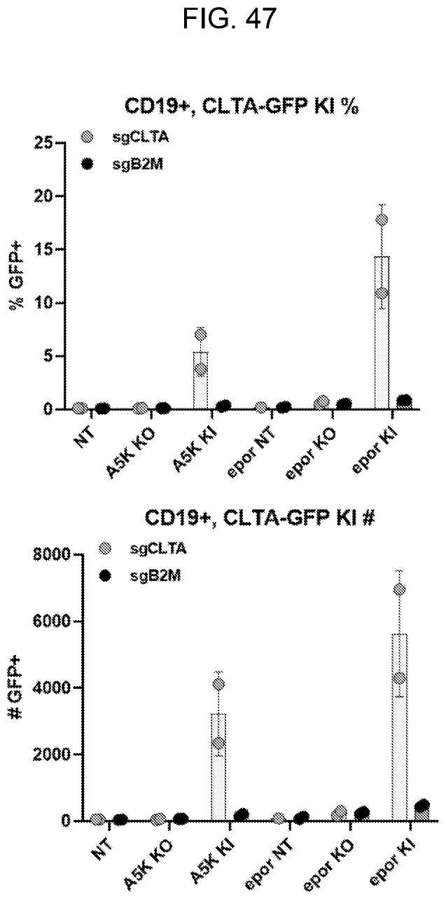

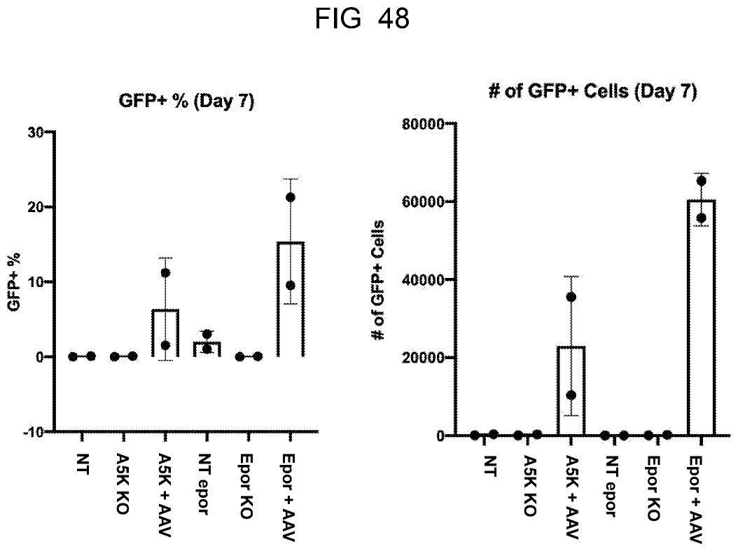

As shown in FIG. 47, peptide-mediated delivery (“PERC”) of CRISPR RNP supports precise knock-in of a gene into primary human B cells when AAV6 is used to provide the DNA donor template necessary for enabling homology-directed repair (HDR). This figure depicts primary human B cell knock-in creating a sfGFP fusion to the N-terminal side of clathrin by targeting the CLTA exon 1 (green) or as a control for off-target at the β2M locus (blue), using Cas9 RNP delivered via electroporation (epor) or via A5K peptide (A5K; peptide #22) and HDR template delivered via AAV6 (KI); with AAV6-free conditions (KO) and untreated cells (NT) shown for comparison. GFP expression was measured at day 7 by flow cytometry displayed as percent live cells expressing GFP (left) and count of live cells expressing GFP (right).

As shown in FIG. 48, peptide-mediated delivery (“PERC”) of CRISPR RNP supports precise knock-in of a gene into primary human NK cells when AAV6 is used to provide the DNA donor template necessary for enabling HDR. This figure depicts primary human NK cell knock-in, creating a sfGFP fusion to the N-terminal side of clathrin by targeting the CLTA exon 1 using Cas9 RNP delivered via peptide #22/A5K (A5K KO) or via electroporation (Epor) plus an HDR template delivered by AAV6 (+AAV); AAV-free (KO) and untreated cells (NT) conditions shown for comparison. GFP expression was measured at day 7 by flow cytometry, displayed as percent live cells expressing GFP (left) and count of live cells expressing GFP (right).

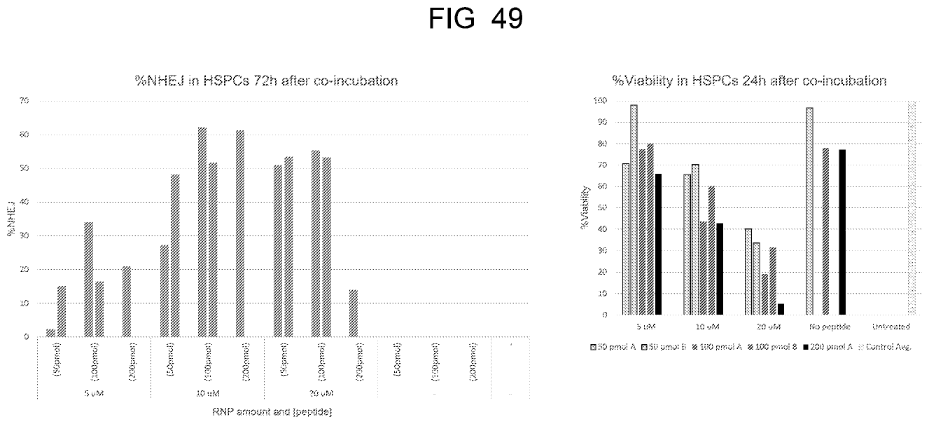

As shown in FIG. 49, peptide-mediated delivery of CRISPR Cas9 RNP supports high efficiency genome editing of primary human CD34⁺ HSPCs at the BCL11a locus, using a gRNA functionally identical to one that has been shown to produce clinical benefit for hemoglobinopaties after use in ex vivo editing and transplantation of CRISPR-edited HSPCs (Frangoul et al. (2021) N Engl J Med 384:252). This figure depicts outcomes following treatment of cultured primary human CD34⁺ HSPCs with various amounts of peptide #55 and various amounts of RNP containing Cas9 protein and a gRNA with the spacer sequence “1617” reported by Wu et al. Nat. Med. (2019) 25:5 and employed in Frangoul et al. (2021) supra. The left plot reports rates of editing detected by amplicon-based NGS of genomic DNA harvested 72 h after peptide-mediated delivery of Cas9 RNP containing “1617” gRNA targeting BCL11a. The right plot reports viability at 24 h following the same delivery events depicted in the left plot. Either 50, 100, or 200 pmol of RNP was delivered, in association with 0, 5, or 10 μM of peptide (measured based on the final concentration in the cell media). Two technical replicates were performed for the 50 and 100 pmol conditions; one technical replicate was performed for the 200 pmol conditions. This experiment was performed with 20,000 cells in 100 μL media.

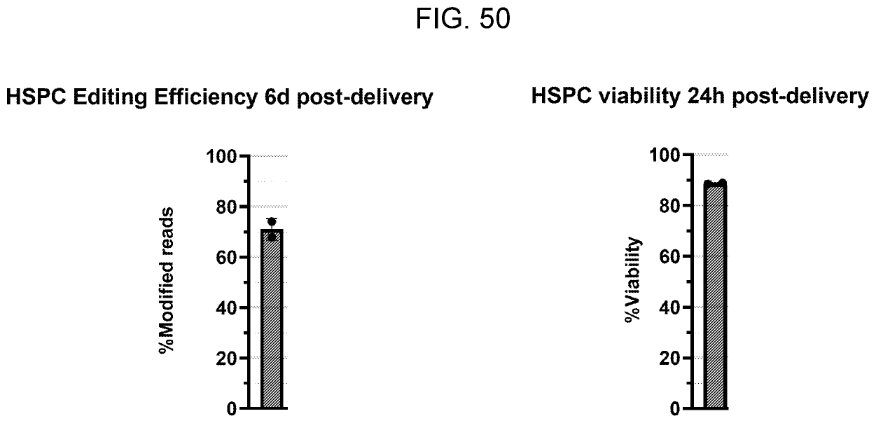

As shown in FIG. 50, peptide-mediated delivery of CRISPR Cas9 RNP supports high efficiency genome editing of primary human CD34⁺ HSPCs at the β2M locus. This figure depicts outcomes following treatment of cultured primary human CD34⁺ HSPCs with various amounts of peptide #55 and various amounts of Cas9 RNP. The left plot reports rates of editing detected by amplicon-based NGS of genomic DNA harvested 6 days after peptide-mediated delivery of Cas9 RNP containing gRNA targeting B2M. The right plot reports viability at 24 h following the same delivery events depicted in the left plot. 100 pmol of RNP was delivered in association with 10 μM peptide (measured based on the final concentration in the cell media). Two technical replicates were performed. This experiment was performed with 100,000 cells in 100 μL media.

### Example 7: Base Editing

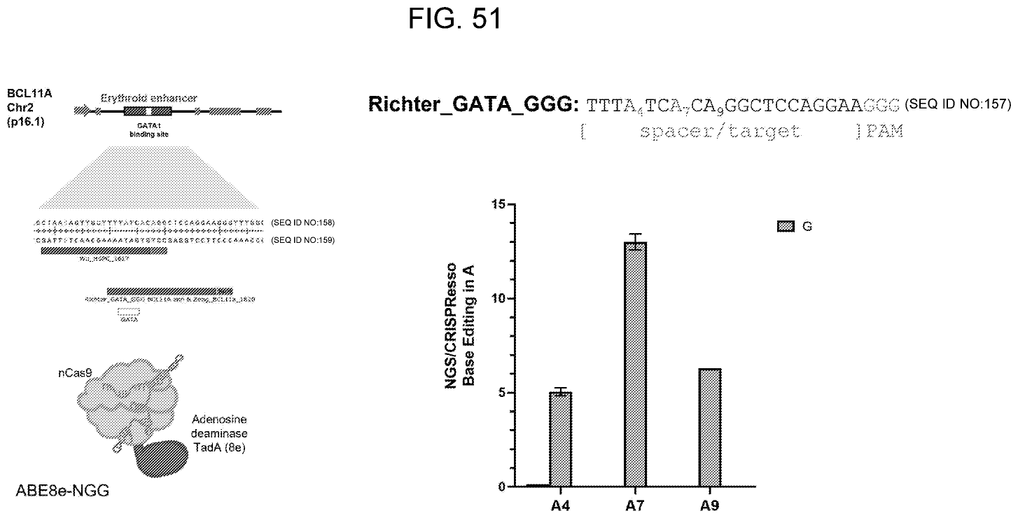

As shown in FIG. 51, base editing in primary human HSPCs at the erythrocyte-specific BCL11a enhancer locus, applying ABE-8e-NGG RNPs at 50 pmol dose with 10 μM peptide #55 and analyzing genomic DNA for base editing via deep sequencing 72 hours after treatment. This experiment uses a gRNA sequence reported in Richter et. al Nat. Biotech. (2020) 38:7, which can direct an adenine base editor to disrupt the erythroid-specific enhancer of BCL11a, the protein that represses expression of fetal hemoglobin.

### Example 8: In Vivo Gene Editing

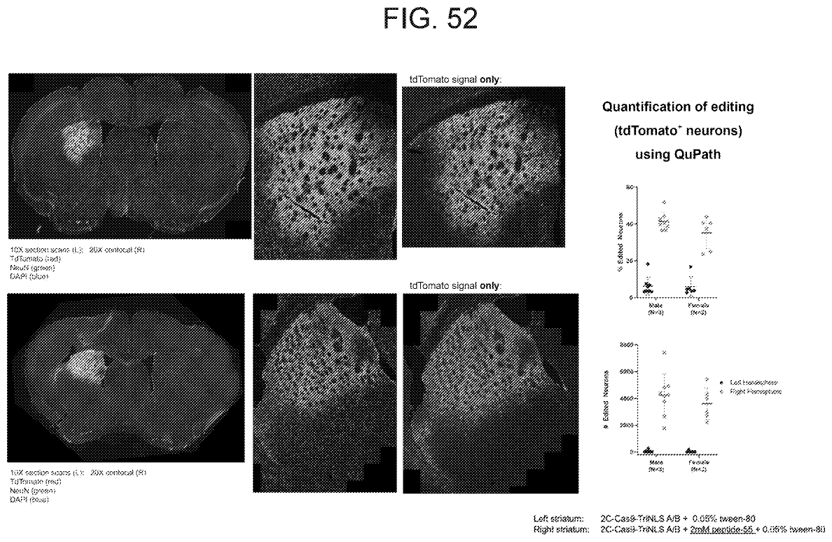

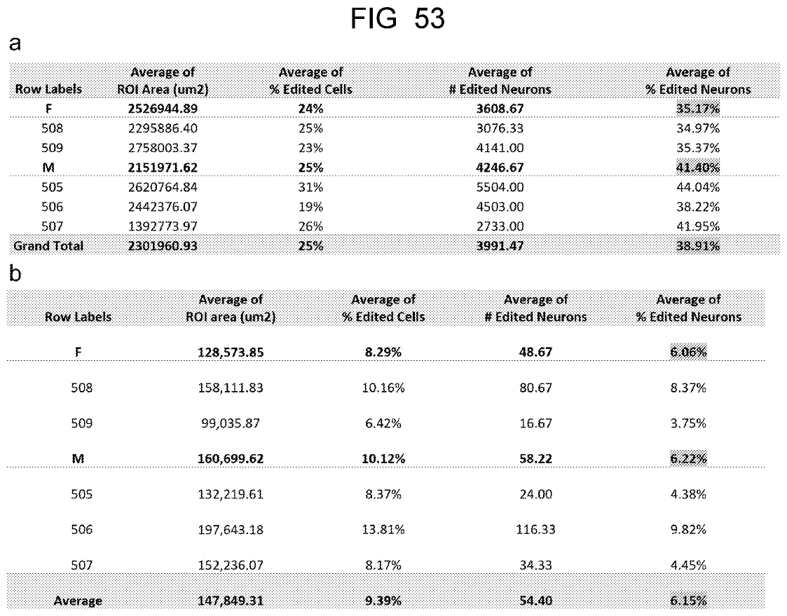

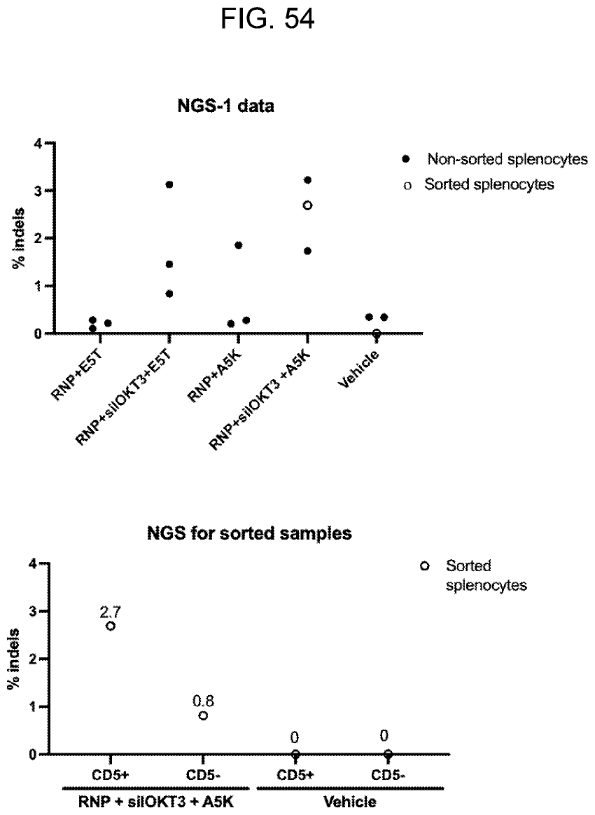

Use of peptides to promote genome editing in vivo was explored. Results are shown in FIG. 52, FIG. 53A-53B, and FIG. 54.

As shown in FIG. 52, formulations containing Cas9 RNP (loaded with two gRNAs—in equal amounts—that can induce excision of a repressor of the tdTomato RFP) and peptide #55 can promote genome editing in striatal neurons within the murine brain. 7 μL of RNP formulations was injected into the striatum of Ai9 mouse brain using convection-enhanced delivery (CED). Left striatum was injected with an RNP-tween formulation; the right striatum was injected with an RNP-peptide-tween formulation (including peptide 55). Reported amounts of peptide (2 mM) and tween-80 (0.05%) refer to the concentration in the 7 μL formulation that was injected. In the 7 μL formulation injected, the RNP concertation was 54.4 μM in the sample with peptide 55, and 52.6 uM in the sample without peptide 55. Histological sections were stained for NeuN (neurons), DAPI (nuclei), and the tdTomato (Invitrogen polyclonal rabbit anti-RFP). Images have been adjusted to most prominently display the red signal from tdTomato. n=2-3 mice.

FIG. 53A-53B report quantification of neuronal editing in Ai9 mice: (a) represents editing rates detected in RNP/tween-containing formulations that also contained peptide #55, (b) represents editing rates detected in RNP/tween-containing formulations that did not contain peptide.

As shown in FIG. 54, intravenously administered formulations containing Cas9 RNP and peptides can promote genome editing of human primary T cells in vivo, in the context of a humanized mouse model (NSG mice infused with human peripheral blood mononuclear cells). Editing efficiency was assessed by NGS in splenocytes derived from humanized mice administered with Cas9 RNP targeting the β2M locus and either peptide #1 (E5-TAT), or with peptide #22 (A5K) when paired with a CD3-targeting antibody. Splenocytes were further FACS-sorted for T-cells and non-T cell populations and indels were detected by NGS in that population. Editing rates were increased when the Cas9 RNP was non-covalently tethered (via a protein A fusion construct of Cas9) to the anti-CD3 antibody OKT3 (targeting the human T cell receptor), which was used in an “Fc-silenced” form that prevents binding by Fc receptor proteins.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A cargo delivery fusion polypeptide comprising:
a) an endosomolytic polypeptide; and
b) a cell penetrating polypeptide,
wherein the fusion polypeptide comprises an amino acid sequence of any one of Formulas I-VIII,
wherein the fusion polypeptide has a length of from about 32 amino acids to about 35 amino acids, and
wherein any two adjacent amino acids are independently linked by an amide bond or a non-amide bond.

2. The fusion polypeptide of claim 1, wherein the fusion polypeptide comprises an amino acid sequence of Formula I: KLFEX₁IEGFIENGWEX₂MIDX₃WX₄GX₅GRKKRRQRR (SEQ ID NO: 165), wherein
X₁ is A, R, or K;
X₂ is A or G;
X₃ is L or G;
X₄ is N or Y; and
X₅, if present, is Y.

3. The fusion polypeptide of claim 2, wherein the fusion polypeptide comprises an amino acid sequence selected from the group consisting of:
(peptide 19; SEQ ID NO: 19)
KLFEAIEGFIENGWEGMIDGWYGYGRKKRRQRR;
(peptide 40; SEQ ID NO: 40)
KLFEAIEGFIENGWEGMIDGWYGGRKKRRQRR;
(peptide 44; SEQ ID NO: 44)
KLFERIEGFIENGWEGMIDGWYGYGRKKRRQRR;
(peptide 45; SEQ ID NO: 45)
KLFEKIEGFIENGWEGMIDGWYGYGRKKRRQRR;
(peptide 46; SEQ ID NO: 46)
KLFEAIEGFIENGWEAMIDGWYGYGRKKRRQRR;
(peptide 47; SEQ ID NO: 47)
KLFEAIEGFIENGWEGMIDLWYGYGRKKRRQRR;
(peptide 48; SEQ ID NO: 48)
KLFEAIEGFIENGWEGMIDGWNGYGRKKRRQRR;
(peptide 53; SEQ ID NO: 53)
KLFEAIEGFIENGWEAMIDLWYGYGRKKRRQRR;
(peptide 54; SEQ ID NO: 54)
KLFEAIEGFIENGWEAMIDGWNGYGRKKRRQRR;
(peptide 55; SEQ ID NO: 55)
KLFEAIEGFIENGWEGMIDLWNGYGRKKRRQRR;
(peptide 56; SEQ ID NO: 56)
KLFEAIEGFIENGWEAMIDLWNGYGRKKRRQRR;
and
(peptide 57; SEQ ID NO: 57)
KLFEKIEGFIENGWEAMIDLWNGYGRKKRRQRR.

4. The fusion polypeptide of claim 1, wherein the fusion polypeptide comprises an amino acid sequence of Formula II: X₁LFEX₂IEGFIENGWEGMIDGWYGYGRKKRRQRR (SEQ ID NO: 166), wherein X₁ is R or G; and X₂ is R or K.

5. The fusion polypeptide of claim 4, wherein the fusion polypeptide comprises an amino acid sequence selected from the group consisting of:
(peptide 42; SEQ ID NO: 42)
RLFERIEGFIENGWEGMIDGWYGYGRKKRRQRR;
(peptide 43; SEQ ID NO: 43)
RLFEKIEGFIENGWEGMIDGWYGYGRKKRRQRR;
and
(peptide 41; SEQ ID NO: 41)
GLFERIEGFIENGWEGMIDGWYGYGRKKRRQRR.

6. The fusion polypeptide of claim 1, wherein the fusion polypeptide comprises an amino acid sequence of Formula III: GLFEAIEGFIENGWEX₁MIDX₂WNGYGRKKRRQRR (SEQ ID NO: 167), wherein X₁ is A or G; and X₂ is G or L.

7. The fusion polypeptide of claim 6, wherein the fusion polypeptide comprises an amino acid sequence selected from the group consisting of:
(peptide 50; SEQ ID NO: 50)
GLFEAIEGFIENGWEAMIDGWNGYGRKKRRQRR;
(peptide 51; SEQ ID NO: 51)
GLFEAIEGFIENGWEGMIDLWNGYGRKKRRQRR;
and
(peptide 51; SEQ ID NO: 51)
GLFEAIEGFIENGWEAMIDLWNGYGRKKRRQRR.

8. The fusion polypeptide of claim 1, wherein the fusion polypeptide comprises an amino acid sequence of Formula IV: GLFEAIEGFIENGWEX₁X₂IX₃LWYGYGRKKRRQRR (SEQ ID NO: 168), wherein:
X₁ is A or G;
X₂ is L or M; and
X₃ is D or E.

9. The fusion polypeptide of claim 8, wherein the fusion polypeptide comprises the amino acid sequence:
(peptide 49; SEQ ID NO: 49)
GLFEAIEGFIENGWEAMIDLWYGYGRKKRRQRR;
or
(peptide 58; SEQ ID NO: 58)
GLFEAIEGFIENGWEGLIELWYGYGRKKRRQRR.

10. The fusion polypeptide of claim 1, wherein the fusion polypeptide comprises an amino acid sequence of Formula V: GLFX₁AIAX₂FIX₃NGWX₄GLIX₅GWYGGRKKRRQRRR (SEQ ID NO: 208), wherein each of X₁, X₂, X₃, X₄, and X₅ is independently a non-coded amino acid.

11. The fusion polypeptide of claim 10, wherein the fusion polypeptide comprises the amino acid sequence:
(peptide 59; SEQ ID NO: 59)
GLFaAIAaFIaNGWaGLIaGWYGGRKKRRQRRR;
or
(peptide 60; SEQ ID NO: 60)
GLFaAIAaFIENGWEGLIDGWYGGRKKRRQRRR.

12. The fusion polypeptide of claim 10 or claim 11, wherein each of X₁, X₂, X₃, X₄, and X₅ is α-aminoadipic acid.

13. The fusion polypeptide of claim 1, wherein the fusion polypeptide comprises an amino acid sequence of Formula VI: KLFEX₁IX₂X₃FIENGWEGMIX₄X₅WX₆GYGRKKRRQRX₇ (SEQ ID NO: 170), wherein: X₁ is A or H; X₂ is E or A; X₃ is G or E; X₄ is D or E; X₅ is G or L; X₆ is E, H, K, R, or N; and X₇, if present, is R.

14. The fusion polypeptide of claim 13, wherein the fusion polypeptide comprises an amino acid sequence selected from:
(peptide 62; SEQ ID NO: 62)
KLFEAIEGFIENGWEGMIDLWEGYGRKKRRQRR;
(peptide 63; SEQ ID NO: 63)
KLFEAIEGFIENGWEGMIDLWHGYGRKKRRQRR;
(peptide 64; SEQ ID NO: 64)
KLFEAIEGFIENGWEGMIDLWKGYGRKKRRQRR;
(peptide 65; SEQ ID NO: 65)
KLFEAIEGFIENGWEGMIDLWRGYGRKKRRQRR;
(peptide 69; SEQ ID NO: 69)
KLFEAIEGFIENGWEGMIDLWNGYGRKKRRQR;
(peptide 71; SEQ ID NO: 71)
KLFEAIEGFIENGWEGMIELWNGYGRKKRRQRR;
(peptide 72; SEQ ID NO: 72)
KLFEAIAEFIENGWEGMIDLWNGYGRKKRRQRR;
(peptide 105; SEQ ID NO: 105)
KLFEHIEGFIENGWEGMIDGWYGYGRKKRRQRR;
(peptide 107; SEQ ID NO: 107)
KLFEHIEGFIENGWEGMIDLWYGYGRKKRRQRR;
and
(peptide 109; SEQ ID NO: 109)
KLFEHIEGFIENGWEGMIDLWKGYGRKKRRQRR.

15. The fusion polypeptide of claim 1, wherein the fusion polypeptide comprises an amino acid sequence of Formula VII: GLFEX₁IX₂X₃FIENGWEGMIDX₄WX₅GYGRKKRRQRR (SEQ ID NO: 171), wherein: X₁ is R, H, A, or K; X₂ is E or A; X₃ is G or E; X₄ is L or G; and X₅ is N, Y, K, or E.

16. The fusion polypeptide of claim 15, wherein the fusion polypeptide comprises an amino acid sequence selected from:
(peptide 66; SEQ ID NO: 66)
GLFERIEGFIENGWEGMIDLWNGYGRKKRRQRR;
(peptide 68; SEQ ID NO: 68)
GLFEHIEGFIENGWEGMIDLWNGYGRKKRRQRR;
(peptide 70; SEQ ID NO: 70)
GLFEAIAEFIENGWEGMIDGWYGYGRKKRRQRR;
(peptide 73; SEQ ID NO: 73)
GLFEKIEGFIENGWEAMIDGWYGYGRKKRRQRR;
(peptide 74; SEQ ID NO: 74)
GLFEKIEGFIENGWEGMIDLWYGYGRKKRRQRR;
(peptide 75; SEQ ID NO: 31)
GLFEAIEGFIENGWEGMIDGWNGYGRKKRRQRR;
(peptide 76; SEQ ID NO: 24)
GLFEAIEEFIENGWEGMIDGWYGYGRKKRRQRR;
(peptide 98; SEQ ID NO: 98)
GLFEKIEGFIENGWEGMIDGWNGYGRKKRRQRR;
(peptide 99; SEQ ID NO: 99)
GLFEKIEGFIENGWEGMIDGWKGYGRKKRRQRR;
(peptide 100; SEQ ID NO: 100)
GLFEKIEGFIENGWEGMIDGWEGYGRKKRRQRR;
(peptide 101; SEQ ID NO: 101)
GLFEKIEGFIENGWEGMIDLWNGYGRKKRRQRR;
(peptide 102; SEQ ID NO: 102)
GLFEKIEGFIENGWEGMIDLWKGYGRKKRRQRR;
and
(peptide 103; SEQ ID NO: 103)
GLFEKIEGFIENGWEGMIDLWEGYGRKKRRQRR.

17. The fusion polypeptide of claim 1, wherein the fusion polypeptide comprises an amino acid sequence of Formula VIII: HLFEX₁IEGFIENGWEGMIDX₂WX₃GYGRKKRRQRR (SEQ ID NO: 172), wherein: X₁ is A or K; X₂ is G or L; and X₃ is N, K, E, or Y.

18. The fusion polypeptide of claim 17, wherein the fusion polypeptide comprises an amino acid sequence selected from:
(peptide 92; SEQ ID NO: 92)
HLFEAIEGFIENGWEGMIDGWNGYGRKKRRQRR;
(peptide 93; SEQ ID NO: 93)
HLFEAIEGFIENGWEGMIDGWKGYGRKKRRQRR;
(peptide 94; SEQ ID NO: 94)
HLFEAIEGFIENGWEGMIDGWEGYGRKKRRQRR;
(peptide 95; SEQ ID NO: 67)
HLFEAIEGFIENGWEGMIDLWNGYGRKKRRQRR;
(peptide 96; SEQ ID NO: 96)
HLFEAIEGFIENGWEGMIDLWKGYGRKKRRQRR;
(peptide 97; SEQ ID NO: 97)
HLFEAIEGFIENGWEGMIDLWEGYGRKKRRQRR;
(peptide 104; SEQ ID NO: 104)
HLFEKIEGFIENGWEGMIDGWYGYGRKKRRQRR;
(peptide 106; SEQ ID NO: 106)
HLFEKIEGFIENGWEGMIDLWYGYGRKKRRQRR;
and
(peptide 108; SEQ ID NO: 108)
HLFEKIEGFIENGWEGMIDLWKGYGRKKRRQRR.

19. The fusion polypeptide of any one of claims 1-18, wherein all of the amino acids in the fusion polypeptide are linked by an amide bond.

20. The fusion polypeptide of any one of claims 1-18, wherein at least two adjacent amino acids are linked by a non-amide bond.

21. The fusion polypeptide of any one of claims 1-20, wherein one or more of the amino acids in the polypeptide comprises a modification.

22. The fusion polypeptide of claim 21, wherein the modification comprises a maleimide group, a methyltetrazine group, a 3-nitro-pyridine-2-carboxylic acid group, a 1,4-bis(bromomethyl)-benzene group, a poly(ethylene glycol) group, a 5-carboxyfluorescein group, a nitropyridine group, a pyridyl disulfide, and a pyridine.

23. The fusion polypeptide of claim 21 or 22, wherein the fusion polypeptide comprises an amino acid sequence of Formula IX: KLFEAIEGFIENGWEGMIDLWNX₁X₂YGRKKRRQRR (SEQ ID NO: 173), wherein: X₁, if present, is Gly; and X₂ is Cys(methyltetrazine) or Cys(3-nitro-2-pyridinesulfenyl).

24. The fusion polypeptide of claim 23, wherein the fusion polypeptide comprises an amino acid sequence selected from:
KLFEAIEGFIENGWEGMIDLWNC*YGRKKRRQRR (peptide 87; SEQ ID NO:87), wherein “C*” is Cys(methyltetrazine);
KLFEAIEGFIENGWEGMIDLWNGC*YGRKKRRQRR (peptide 88; SEQ ID NO:88), wherein “C*” is Cys(methyltetrazine);
KLFEAIEGFIENGWEGMIDLWNC*YGRKKRRQRR (peptide 89; SEQ ID NO:89), wherein “C*” is Cys(3-nitro-2-pyridinesulfenyl); and
KLFEAIEGFIENGWEGMIDLWNGC*YGRKKRRQRR (peptide 90; SEQ ID NO:90), wherein “C*” is Cys(3-nitro-2-pyridinesulfenyl).

25. The fusion polypeptide of claim 21 or 22, wherein the fusion polypeptide comprising an amino acid sequence of Formula X: KLFEAIEGFIENGWEGMIDLWNGX₁YGRKKRRQRRX₂ (SEQ ID NO: 174), wherein: X₁ is Cys(methyltetrazine-PEG4-maleimide), Cys(maleimide), Lys(PEG₂₃)₂, Lys(3-nitro-pyridine-2-carboxylic acid), Lys(PEG₂₃)₂, Lys(PEG₂₃)₂-(3-nitro-pyridine-2-carboxylic acid), or Cys (1,4-bis(bromomethyl)-benzene); and X₂ is Cys(3-nitro-2-pyridine-sulfenyl) or Lys(methyltetrazine-PEG4).

26. The fusion polypeptide of claim 25, wherein the fusion polypeptide comprises an amino acid sequence selected from the amino acid sequence of peptide f1, peptide f2, peptide f3, peptide f4, peptide f4, peptide f6, peptide f7, peptide f11, peptide f13, and peptide f14 depicted in FIG. 30.

27. The fusion polypeptide of any one of claims 1-26, wherein at least two adjacent amino acids are linked by a linker comprising one or more ethylene glycol monomers.

28. The fusion polypeptide of claim 27, wherein the linker is a polymer comprising 2, 4, 6, or 8 ethylene glycol monomers.

29. The fusion polypeptide of claim 1, wherein the fusion polypeptide comprises the amino acid sequence of any one of the peptides 19 and 40-60 depicted in FIG. 1.

30. The fusion polypeptide of claim 1, wherein the fusion polypeptide comprises the amino acid sequence of any one of the peptides depicted in FIG. 30.

31. The fusion polypeptide of claim 1, wherein the fusion polypeptide does not comprise the amino acid sequence of any one of the peptides designated 1-18 or 21-27 and depicted in FIG. 1.

32. A composition comprising the cargo delivery fusion polypeptide of any one of claims 1-31.

33. The composition of claim 32, further comprising a cargo, wherein the cargo comprises one or more of a nucleic acid, a polypeptide, and a ribonucleoprotein complex.

34. The composition of claim 33, wherein the cargo comprises a targeting moiety.

35. The composition of claim 32, comprising a nucleic acid comprising a nucleotide sequence encoding a gene product of interest.

36. The composition of claim 35, wherein the gene product of interest is an antigen.

37. The composition of claim 35 or claim 36, wherein the nucleic acid is a recombinant expression vector.

38. The composition of claim 37, wherein the recombinant expression vector is a recombinant viral vector.

39. The composition of claim 32, comprising:
a) a CRISPR-Cas effector polypeptide; or
b) a fusion polypeptide comprising:
i) a CRISPR-Cas effector polypeptide; and
ii) one or more heterologous polypeptides.

40. The composition of claim 39, comprising a CRISPR-Cas guide nucleic acid.

41. The composition of claim 39 or claim 40, comprising a donor DNA template.

42. The composition of any one of claims 39-41, wherein the CRISPR-Cas effector polypeptide is a type II CRISPR-Cas effector polypeptide, a type V CRISPR-Cas effector polypeptide, or a type VI CRISPR-Cas effector polypeptide.

43. The composition of claim 40, wherein the CRISPR-Cas guide nucleic acid is RNA.

44. The composition of claim 43, wherein the CRISPR-Cas guide nucleic acid is a single-molecule guide RNA or a dual-molecule guide RNA.

45. The composition of any one of claims 39-44, wherein the composition comprises a CRISPR-Cas effector fusion polypeptide comprising: i) a CRISPR-Cas effector fusion polypeptide; and ii) one or more nuclear localization signals.

46. The composition of any one of claims 39-44, wherein the composition comprises a CRISPR-Cas effector fusion polypeptide comprising: i) a CRISPR-Cas effector polypeptide; and ii) one or more heterologous effector polypeptides.

47. The composition of claim 46, wherein at least one of the one or more heterologous effector polypeptides is a single stranded nuclease, a double strand nuclease, a helicase, a methylase, a demethylase, an acetylase, a deacetylase, a deaminase, an integrase, a recombinase, a base editor, or a prime editor.

48. The composition of any one of claims 39-47, wherein the CRISPR-Cas effector polypeptide, or the CRISPR-Cas effector fusion polypeptide, comprises a covalently linked targeting moiety.

49. The composition of claim 48, wherein the targeting moiety is protein A, protein G, an aptamer, a darpin, or an antibody.

50. The composition of claim 49, comprising an antibody non-covalently bound to the affinity moiety.

51. The composition of claim 49 or claim 50, where the antibody specifically binds an epitope on the surface of a eukaryotic cell, thereby targeting the composition to the cell.

52. The composition of any one of claims 39-51, wherein the CRISPR-Cas effector polypeptide, or the CRISPR-Cas effector fusion polypeptide, comprises a non-polypeptide polymer.

53. The composition of claim 52, wherein the non-polypeptide polymer is poly(ethylene glycol).

54. The composition of any one of claims 40-53, wherein the CRISPR-Cas effector polypeptide and the guide nucleic acid are in a ribonucleoprotein (RNP) complex.

55. The composition of claim 54, wherein the molar ratio of cargo delivery fusion polypeptide to RNP is at least 3:1.

56. The composition of claim 55, wherein the molar ratio of cargo delivery fusion polypeptide to RNP is from 10:1 to 50:1.

57. The composition of any one of claims 32-56, wherein the cargo delivery fusion polypeptide is present in the composition in a concentration of from about 2 μM to about 50 μM.

58. The composition of any one of claims 32-57, comprising one or more of a solubilizing agent, a surfactant, a buffer, a salt, and a protease inhibitor.

59. The composition of any one of claims 32-58, comprising poly(ethylene glycol), a non-ionic surfactant, or both.

60. A method of delivering a cargo into a target population of eukaryotic cells, the method comprising contacting the cell with the composition of any one of claims 32-59, thereby generating a modified target population of eukaryotic cells comprising the cargo.

61. The method of claim 60, wherein the target population of eukaryotic cells comprises a T cell, a stem cell, a natural killer cell, a renal cell or a neural cell.

62. The method of claim 60, wherein the target eukaryotic cell is a hematopoietic stem cell or a hematopoietic progenitor cell.

63. The method of any one of claims 60-4362 wherein the cell is in vitro.

64. The method of claim 63, wherein at least 50% of the target population of eukaryotic cells retain viability after said contacting.

65. The method of claim 63 or claim 64, wherein the method comprises introducing into the modified target population of eukaryotic cells a second composition comprising a second cargo.

66. The method of claim 65, wherein said introducing is via electroporation or transfection.

67. The method of claim 66, wherein said transfection comprises contacting the modified target population of eukaryotic cells with a recombinant viral vector.

68. The method of any one of claims 60-62, wherein the cell is in vivo.

69. A method of delivering a DNA molecule into a eukaryotic cell, the method comprising contacting the cell with a composition comprising:
a) an amphiphilic cargo delivery fusion polypeptide comprising:
i) an endosomolytic polypeptide; and
ii) a cell penetrating polypeptide; and
b) a DNA molecule comprising a nucleotide sequence encoding an immunogenic polypeptide.

70. The method of claim 69, wherein the immunogenic polypeptide is a viral polypeptide.

71. The method of claim 69 or claim 70, wherein the endosomolytic polypeptide:
a) comprises the amino acid sequence GLFEAIAEFIENGWEGLIEGWYG (SEQ ID NO:163);
b) comprises from 1 to 5 amino acid substitutions relative to GLFEAIAEFIENGWEGLIEGWYG (SEQ ID NO:163);
c) comprises the amino acid sequence GLFEAIEGFIENGWEGMIDGWYG (SEQ ID NO:164); or
d) comprises from 1 to 5 amino acid substitutions relative to GLFEAIEGFIENGWEGMIDGWYG (SEQ ID NO:164),
wherein the endosomolytic polypeptide has a length of from about 20 amino acids to about 30 amino acids.

72. The method of any one of claims 69-71, wherein the cell penetrating polypeptide comprises the amino acid sequence YGRKKRRQRRR (SEQ ID NO:207), YGRKKRRQRR (SEQ ID NO: 160), or GRKKRRQRRR (SEQ ID NO:161), and has a length of from 10 amino acids to 15 amino acids.

73. A method of delivering a ribonucleoprotein (RNP) into a eukaryotic cell, the method comprising contacting the cell with a composition comprising:
a) an amphiphilic cargo delivery fusion polypeptide comprising:
i) an endosomolytic polypeptide; and
ii) a cell penetrating polypeptide; and
b) an RNP comprising:
i) a CRISPR-Cas effector polypeptide; and
ii) a guide nucleic acid.

74. The method of claim 73, wherein the endosomolytic polypeptide:
a) comprises the amino acid sequence GLFEAIAEFIENGWEGLIEGWYG (SEQ ID NO:163);
b) comprises from 1 to 5 amino acid substitutions relative to GLFEAIAEFIENGWEGLIEGWYG (SEQ ID NO:163);
c) comprises the amino acid sequence GLFEAIEGFIENGWEGMIDGWYG (SEQ ID NO:164); or
d) comprises from 1 to 5 amino acid substitutions relative to GLFEAIEGFIENGWEGMIDGWYG (SEQ ID NO:164),
wherein the endosomolytic polypeptide has a length of from about 20 amino acids to about 30 amino acids.

75. The method of claim 73 or claim 74, wherein the cell penetrating polypeptide comprises the amino acid sequence YGRKKRRQRRR (SEQ ID NO:207), YGRKKRRQRR (SEQ ID NO:160), or GRKKRRQRRR (SEQ ID NO:161), and has a length of from 10 amino acids to 15 amino acids.

76. The method of any one of claims 73-75, comprising introducing into the cell a DNA donor template.

77. The method of claim 76, wherein the donor template is present in a recombinant viral vector.

78. The method of claim 77, wherein the recombinant viral vector is a recombinant adenoassociated viral vector.

79. The method of any one of claims 73-78, wherein the donor template comprises a nucleotide sequence encoding a polypeptide.

80. The method of claim 79, wherein the polypeptide is a chimeric antigen receptor comprising a single-chain Fv or a nanobody specific for a cancer-associated antigen.

81. The method of any one of claims 73-80, wherein the eukaryotic cell is an immune cell.

82. The method of claim 81, wherein the immune cell is a T cell, a B cell, or an NK cell.

83. The method of any one of claims 73-82, wherein the eukaryotic cell is in vivo.

84. The method of any one of claims 73-82, wherein the eukaryotic cell is in vitro.


SEQUENCE LISTING (208 sequences)

SEQ ID NO: 1 - protein, 33 aa, synthetic construct
GLFEAIAEFIENGWEGLIEGWYGGRKKRRQRRR

SEQ ID NO: 2 - protein, 34 aa, synthetic construct
GLFEAIAEFIENGWEGLIEGWYGGRKKRRQRRRC

SEQ ID NO: 3 - protein, 34 aa, synthetic construct
CGLFEAIAEFIENGWEGLIEGWYGGRKKRRQRRR

SEQ ID NO: 4 - protein, 34 aa, synthetic construct
GLFEAIAEFIENGWEGLIEGWYGYGRKKRRQRRR

SEQ ID NO: 5 - protein, 35 aa, synthetic construct
GLFEAIAEFIENGWEGLIEGWYGYGRKKRRQRRRR

SEQ ID NO: 6 - protein, 34 aa, synthetic construct
GLFEAIAEFIENGWEGLIEGWYGAGRKKRRQRRR

SEQ ID NO: 7 - protein, 34 aa, synthetic construct
GLFEAIAEFIENGWEGLIEGWYGGGRKKRRQRRR

SEQ ID NO: 8 - protein, 32 aa, synthetic construct
GLFEAIAEFIENGWEGLIEGWYGRKKRRQRRR

SEQ ID NO: 9 - protein, 34 aa, synthetic construct
GLFEAIAEFIENGWEGLIEGWYGYGRRRRRQRRR

SEQ ID NO: 10 - protein, 33 aa, synthetic construct
GLFEAIAEFIENGWEGLIDGWYGGRKKRRQRRR

SEQ ID NO: 11 - protein, 33 aa, synthetic construct
GLFEAIEGFIENGWEGMIDGWYGYGRKKRRQRR

SEQ ID NO: 12 - protein, 34 aa, synthetic construct
GLFEAIEGFIENGWEGMIDGWYGYGRKKRRQRRC

SEQ ID NO: 13 - protein, 34 aa, synthetic construct
CGLFEAIEGFIENGWEGMIDGWYGYGRKKRRQRR

SEQ ID NO: 14 - protein, 32 aa, synthetic construct
GLFEAIEGFIENGWEGMIDGWYGGRKKRRQRR

SEQ ID NO: 15 - protein, 33 aa, synthetic construct
GLFHAIAHFIHGGWHGLIHGWYGYGRKKRRQRR

SEQ ID NO: 16 - protein, 33 aa, synthetic construct
GLFKAIAKFIKGGWKGLIKGWYGYGRKKRRQRR

SEQ ID NO: 17 - protein, 33 aa, synthetic construct
GLFEAIAEFIEGGWEGLIEGWYGYGRKKRRQRR

SEQ ID NO: 18 - protein, 33 aa, synthetic construct
RLFEAIEGFIENGWEGMIDGWYGYGRKKRRQRR

SEQ ID NO: 19 - protein, 33 aa, synthetic construct
KLFEAIEGFIENGWEGMIDGWYGYGRKKRRQRR

SEQ ID NO: 20 - protein, 33 aa, synthetic construct
GFFEAIEGFIENGWEGMIDGWYGYGRKKRRQRR

SEQ ID NO: 21 - protein, 33 aa, synthetic construct
GLFGAIEGFIENGWEGMIDGWYGYGRKKRRQRR

SEQ ID NO: 22 - protein, 33 aa, synthetic construct
GLFEKIEGFIENGWEGMIDGWYGYGRKKRRQRR

SEQ ID NO: 23 - protein, 33 aa, synthetic construct
GLFEAIWGFIENGWEGMIDGWYGYGRKKRRQRR

SEQ ID NO: 24 - protein, 33 aa, synthetic construct
GLFEAIEEFIENGWEGMIDGWYGYGRKKRRQRR

SEQ ID NO: 25 - protein, 33 aa, synthetic construct
GLFEAIEGLIENGWEGMIDGWYGYGRKKRRQRR

SEQ ID NO: 26 - protein, 33 aa, synthetic construct
GLFEAIEGFIWNGWEGMIDGWYGYGRKKRRQRR

SEQ ID NO: 27 - protein, 33 aa, synthetic construct
GLFEAIEGFIENGEEGMIDGWYGYGRKKRRQRR

SEQ ID NO: 28 - protein, 33 aa, synthetic construct
GLFEAIEGFIENGWRGMIDGWYGYGRKKRRQRR

SEQ ID NO: 29 - protein, 33 aa, synthetic construct
GLFEAIEGFIENGWEAMIDGWYGYGRKKRRQRR

SEQ ID NO: 30 - protein, 33 aa, synthetic construct
GLFEAIEGFIENGWEGMIDLWYGYGRKKRRQRR

SEQ ID NO: 31 - protein, 33 aa, synthetic construct
GLFEAIEGFIENGWEGMIDGWNGYGRKKRRQRR

SEQ ID NO: 32 - protein, 32 aa, synthetic construct
GLFEAIEGFIENGWEGMIDGWYGYGRKKRRQR

SEQ ID NO: 33 - protein, 33 aa, synthetic construct
GLFEAIEGFIENGWEGMIDGWYGYGHKKHHQHR

SEQ ID NO: 34 - protein, 33 aa, synthetic construct
RRQRRKKRGYGYWGDIMGEWGNEIFGEIAEFLG

SEQ ID NO: 35 - protein, 33 aa, synthetic construct
GLFGAIEGFIENGWEGLIDGWYGYGRKKRRQRR

SEQ ID NO: 36 - protein, 36 aa, synthetic construct
RLFEAIWGFIENGWEGMIDGWYGGGGYGRKKRRQRR

SEQ ID NO: 37 - protein, 33 aa, synthetic construct
GLFEAIAEFIENGWEGLIEGWYGGRRRRRQRRR

SEQ ID NO: 38-39 - skipped (2 entries)

SEQ ID NO: 40 - protein, 32 aa, synthetic construct
KLFEAIEGFIENGWEGMIDGWYGGRKKRRQRR

SEQ ID NO: 41 - protein, 33 aa, synthetic construct
GLFERIEGFIENGWEGMIDGWYGYGRKKRRQRR

SEQ ID NO: 42 - protein, 33 aa, synthetic construct
RLFERIEGFIENGWEGMIDGWYGYGRKKRRQRR

SEQ ID NO: 43 - protein, 33 aa, synthetic construct
RLFEKIEGFIENGWEGMIDGWYGYGRKKRRQRR

SEQ ID NO: 44 - protein, 33 aa, synthetic construct
KLFERIEGFIENGWEGMIDGWYGYGRKKRRQRR

SEQ ID NO: 45 - protein, 33 aa, synthetic construct
KLFEKIEGFIENGWEGMIDGWYGYGRKKRRQRR

SEQ ID NO: 46 - protein, 33 aa, synthetic construct
KLFEAIEGFIENGWEAMIDGWYGYGRKKRRQRR

SEQ ID NO: 47 - protein, 33 aa, synthetic construct
KLFEAIEGFIENGWEGMIDLWYGYGRKKRRQRR

SEQ ID NO: 48 - protein, 33 aa, synthetic construct
KLFEAIEGFIENGWEGMIDGWNGYGRKKRRQRR

SEQ ID NO: 49 - protein, 33 aa, synthetic construct
GLFEAIEGFIENGWEAMIDLWYGYGRKKRRQRR

SEQ ID NO: 50 - protein, 33 aa, synthetic construct
GLFEAIEGFIENGWEAMIDGWNGYGRKKRRQRR

SEQ ID NO: 51 - protein, 33 aa, synthetic construct
GLFEAIEGFIENGWEGMIDLWNGYGRKKRRQRR

SEQ ID NO: 52 - protein, 33 aa, synthetic construct
GLFEAIEGFIENGWEAMIDLWNGYGRKKRRQRR

SEQ ID NO: 53 - protein, 33 aa, synthetic construct
KLFEAIEGFIENGWEAMIDLWYGYGRKKRRQRR

SEQ ID NO: 54 - protein, 33 aa, synthetic construct
KLFEAIEGFIENGWEAMIDGWNGYGRKKRRQRR

SEQ ID NO: 55 - protein, 33 aa, synthetic construct
KLFEAIEGFIENGWEGMIDLWNGYGRKKRRQRR

SEQ ID NO: 56 - protein, 33 aa, synthetic construct
KLFEAIEGFIENGWEAMIDLWNGYGRKKRRQRR

SEQ ID NO: 57 - protein, 33 aa, synthetic construct
KLFEKIEGFIENGWEAMIDLWNGYGRKKRRQRR

SEQ ID NO: 58 - protein, 33 aa, synthetic construct
GLFEAIEGFIENGWEGLIELWYGYGRKKRRQRR

SEQ ID NO: 59 - protein, 33 aa, synthetic construct
  SITE 4: note = Wherein X is alpha-aminoadipic acid
  SITE 8: note = Wherein X is alpha-aminoadipic acid
  SITE 11: note = Wherein X is alpha-aminoadipic acid
  SITE 15: note = Wherein X is alpha-aminoadipic acid
  SITE 19: note = Wherein X is alpha-aminoadipic acid
GLFXAIAXFIXNGWXGLIXGWYGGRKKRRQRRR

SEQ ID NO: 60 - protein, 33 aa, synthetic construct
  SITE 4: note = Wherein X is alpha-aminoadipic acid
  SITE 8: note = Wherein X is alpha-aminoadipic acid
GLFXAIAXFIENGWEGLIDGWYGGRKKRRQRRR

SEQ ID NO: 61 - skipped

SEQ ID NO: 62 - protein, 33 aa, synthetic construct
KLFEAIEGFIENGWEGMIDLWEGYGRKKRRQRR

SEQ ID NO: 63 - protein, 33 aa, synthetic construct
KLFEAIEGFIENGWEGMIDLWHGYGRKKRRQRR

SEQ ID NO: 64 - protein, 33 aa, synthetic construct
KLFEAIEGFIENGWEGMIDLWKGYGRKKRRQRR

SEQ ID NO: 65 - protein, 33 aa, synthetic construct
KLFEAIEGFIENGWEGMIDLWRGYGRKKRRQRR

SEQ ID NO: 66 - protein, 33 aa, synthetic construct
GLFERIEGFIENGWEGMIDLWNGYGRKKRRQRR

SEQ ID NO: 67 - protein, 33 aa, synthetic construct
HLFEAIEGFIENGWEGMIDLWNGYGRKKRRQRR

SEQ ID NO: 68 - protein, 33 aa, synthetic construct
GLFEHIEGFIENGWEGMIDLWNGYGRKKRRQRR

SEQ ID NO: 69 - protein, 32 aa, synthetic construct
KLFEAIEGFIENGWEGMIDLWNGYGRKKRRQR

SEQ ID NO: 70 - protein, 33 aa, synthetic construct
GLFEAIAEFIENGWEGMIDGWYGYGRKKRRQRR

SEQ ID NO: 71 - protein, 33 aa, synthetic construct
KLFEAIEGFIENGWEGMIELWNGYGRKKRRQRR

SEQ ID NO: 72 - protein, 33 aa, synthetic construct
KLFEAIAEFIENGWEGMIDLWNGYGRKKRRQRR

SEQ ID NO: 73 - protein, 33 aa, synthetic construct
GLFEKIEGFIENGWEAMIDGWYGYGRKKRRQRR

SEQ ID NO: 74 - protein, 33 aa, synthetic construct
GLFEKIEGFIENGWEGMIDLWYGYGRKKRRQRR

SEQ ID NO: 75-76 - skipped (2 entries)

SEQ ID NO: 77 - protein, 33 aa, synthetic construct
GLFERIEGFIENGWEGMIDLWNGYGRRRRRQRR

SEQ ID NO: 78 - protein, 33 aa, synthetic construct
  SITE 1: note = Wherein the A at position 1 is beta alanine
ALFEAIEGFIENGWEGMIDGWYGYGRKKRRQRR

SEQ ID NO: 79 - protein, 33 aa, synthetic construct
GLFEEIEGFIENGWEGMIDGWYGYGRKKRRQRR

SEQ ID NO: 80 - protein, 33 aa, synthetic construct
GLFEAIEGFIENEWEGMIDGWYGYGRKKRRQRR

SEQ ID NO: 81 - protein, 33 aa, synthetic construct
GLFEAIEGFIENGWEGMIEGWYGYGRKKRRQRR

SEQ ID NO: 82 - protein, 33 aa, synthetic construct
GLFEAIEGFIENGWEGMIDGWYGYGHKKHHQHH

SEQ ID NO: 83 - skipped

SEQ ID NO: 84 - protein, 31 aa, synthetic construct
GLFEAIEGFIENGWEGMIDGWYGYGRKKRRQ

SEQ ID NO: 85 - protein, 30 aa, synthetic construct
GLFEAIEGFIENGWEGMIDGWYGYGRKKRR

SEQ ID NO: 86 - skipped

SEQ ID NO: 87 - protein, 33 aa, synthetic construct
  SITE 23: note = Wherein the C at position 23 has been chemically modified using methyltetrazine-PEG4-maleimide
KLFEAIEGFIENGWEGMIDLWNCYGRKKRRQRR

SEQ ID NO: 88 - protein, 34 aa, synthetic construct
  SITE 24: note = Wherein the C at position 24 has been chemically modified using methyltetrazine-PEG4-maleimide
KLFEAIEGFIENGWEGMIDLWNGCYGRKKRRQRR

SEQ ID NO: 89 - protein, 33 aa, synthetic construct; identical to SEQ ID NO: 87
  SITE 23: note = Wherein the C at position 23 refers to Cys(3-nitro-2-pyridinesulfenyl) incorporated into the peptide backbone

SEQ ID NO: 90 - protein, 34 aa, synthetic construct; identical to SEQ ID NO: 88
  SITE 24: note = Wherein the C at position 24 refers to Cys(3-nitro-2-pyridinesulfenyl) incorporated into the peptide backbone

SEQ ID NO: 91 - protein, 34 aa, synthetic construct; identical to SEQ ID NO: 88
  SITE 24: note = Wherein the C at position 24 refers to Cys(3-nitro-2-pyridinesulfenyl) incorporated into the peptide backbone
  SITE 24: note = 4 copies (monomeric units) of polyethylene glycol are incoporated into the peptide backbone, 2 before the residue at position 24 and 2 after the residue at position 24

SEQ ID NO: 92 - protein, 33 aa, synthetic construct
HLFEAIEGFIENGWEGMIDGWNGYGRKKRRQRR

SEQ ID NO: 93 - protein, 33 aa, synthetic construct
HLFEAIEGFIENGWEGMIDGWKGYGRKKRRQRR

SEQ ID NO: 94 - protein, 33 aa, synthetic construct
HLFEAIEGFIENGWEGMIDGWEGYGRKKRRQRR

SEQ ID NO: 95 - skipped

SEQ ID NO: 96 - protein, 33 aa, synthetic construct
HLFEAIEGFIENGWEGMIDLWKGYGRKKRRQRR

SEQ ID NO: 97 - protein, 33 aa, synthetic construct
HLFEAIEGFIENGWEGMIDLWEGYGRKKRRQRR

SEQ ID NO: 98 - protein, 33 aa, synthetic construct
GLFEKIEGFIENGWEGMIDGWNGYGRKKRRQRR

SEQ ID NO: 99 - protein, 33 aa, synthetic construct
GLFEKIEGFIENGWEGMIDGWKGYGRKKRRQRR

SEQ ID NO: 100 - protein, 33 aa, synthetic construct
GLFEKIEGFIENGWEGMIDGWEGYGRKKRRQRR

SEQ ID NO: 101 - protein, 33 aa, synthetic construct
GLFEKIEGFIENGWEGMIDLWNGYGRKKRRQRR

SEQ ID NO: 102 - protein, 33 aa, synthetic construct
GLFEKIEGFIENGWEGMIDLWKGYGRKKRRQRR

SEQ ID NO: 103 - protein, 33 aa, synthetic construct
GLFEKIEGFIENGWEGMIDLWEGYGRKKRRQRR

SEQ ID NO: 104 - protein, 33 aa, synthetic construct
HLFEKIEGFIENGWEGMIDGWYGYGRKKRRQRR

SEQ ID NO: 105 - protein, 33 aa, synthetic construct
KLFEHIEGFIENGWEGMIDGWYGYGRKKRRQRR

SEQ ID NO: 106 - protein, 33 aa, synthetic construct
HLFEKIEGFIENGWEGMIDLWYGYGRKKRRQRR

SEQ ID NO: 107 - protein, 33 aa, synthetic construct
KLFEHIEGFIENGWEGMIDLWYGYGRKKRRQRR

SEQ ID NO: 108 - protein, 33 aa, synthetic construct
HLFEKIEGFIENGWEGMIDLWKGYGRKKRRQRR

SEQ ID NO: 109 - protein, 33 aa, synthetic construct
KLFEHIEGFIENGWEGMIDLWKGYGRKKRRQRR

SEQ ID NO: 110 - protein, 33 aa, synthetic construct; identical to SEQ ID NO: 55
  SITE 23: note = Wherein two copies (monomeric units) of polyethylene glycol are incorporated into the peptide backbone after the amino acid at position 23 and before the residue at position 24

SEQ ID NO: 111 - protein, 33 aa, synthetic construct; identical to SEQ ID NO: 55
  SITE 23: note = Wherein four copies (monomeric units) of polyethylene glycol are incorporated into the peptide backbone after the amino acid at position 23 and before the residue at position 24

SEQ ID NO: 112 - protein, 33 aa, synthetic construct; identical to SEQ ID NO: 55
  SITE 23: note = Wherein eight copies (monomeric units) of polyethylene glycol are incorporated into the peptide backbone after the amino acid at position 23 and before the residue at position 24

SEQ ID NO: 113 - protein, 34 aa, synthetic construct; identical to SEQ ID NO: 88
  SITE 23: note = Wherein four copies (monomeric units) of polyethylene glycol are incorporated into the peptide backbone, two before the amino acid at position 23 and two after the residue at position 23
  SITE 23: note = Wherein the C at position 23 has been chemically modified using methyltetrazine-PEG4-maleimide

SEQ ID NO: 114 - protein, 34 aa, synthetic construct
  SITE 23: note = Wherein four copies (monomeric units) of polyethylene glycol are incorporated into the peptide backbone, two before the amino acid at position 23 and two after the residue at position 23
  SITE 23: note = Wherein the L at position 23 refers to Lys(3-Nitro-pyridine-2-carboxylic acid)
KLFEAIEGFIENGWEGMIDLWNGKYGRKKRRQRR

SEQ ID NO: 115 - protein, 34 aa, synthetic construct; identical to SEQ ID NO: 114
  SITE 23: note = Wherein four copies (monomeric units) of polyethylene glycol are incorporated into the peptide backbone, two before the amino acid at position 23 and two after the residue at position 23
  SITE 23: note = Wherein the L at position 23 refers to Lys(PEG23)2, a branched PEG moiety extending off of the peptide backbone

SEQ ID NO: 116 - protein, 38 aa, synthetic construct
  SITE 23: note = Wherein four copies (monomeric units) of polyethylene glycol are incorporated into the peptide backbone, two before the amino acid at position 23 and two after the residue at position 23
  SITE 23: note = Wherein the K at position 23 refers to Lys(PEG23)2)-(3-Nitro-pyridine-2-carboxylic acid) extending off of the peptide backbone
KLFEAIEGFIENGWEGMIDLWNGXKXXXYGRKKRRQRR

SEQ ID NO: 117 - protein, 34 aa, synthetic construct
  SITE 1: note = Wherein there is an acetyl group before the residue at position 1
  SITE 23: note = Wherein the the C at position 24 has been chemically modified using methyltetrazine-PEG4-maleimide
  SITE 23: note = Wherein four copies (monomeric units) of polyethylene glycol are incorporated into the peptide backbone, two before the amino acid at position 23 and two after the residue at position 23
RLFEAIEGFIENGWEGMIDLWNGCYGRRRRRQRR

SEQ ID NO: 118 - protein, 33 aa, synthetic construct
  SITE 1: note = Wherein there is an acetyl group before the residue at position 1
  SITE 23: note = Wherein the C at position 23 has been chemically modified using methyltetrazine-PEG4-maleimide
GLFERIEGFIENGWEGMIDLWNCYGRRRRRQRR

SEQ ID NO: 119 - protein, 34 aa, synthetic construct
  SITE 21: note = Wherein the K at position 21 has been chemically modified via conjugation to a succinimidyl ester form of 5-FAM (5-carboxyfluorescein)
  SITE 24: note = Wherein eight copies (monomeric units) of polyethylene glycol are incorporated into the peptide backbone, four before the amino acid at position 24 and four after the residue at position 24
  SITE 24: note = Wherein the C at position 24 is Cys(3-nitro-2-pyridinesulfenyl)
KLFEAIEGFIENGWEGMIDLKNGCYGRKKRRQRR

SEQ ID NO: 120 - protein, 34 aa, synthetic construct; identical to SEQ ID NO: 88
  SITE 24: note = Wherein four copies (monomeric units) of polyethylene glycol are incorporated into the peptide backbone, two before the amino acid at position 24 and two after the residue at position 24
  SITE 24: note = Wherein the C at position 24 has been chemically modified post-synthesis with 1,4-bis(bromomethyl)-benzene, creating a peptide dimer

SEQ ID NO: 121 - protein, 34 aa, synthetic construct
  SITE 34: note = Wherein two copies (monomeric units) of polyethylene glycol are incorporated into the peptide backbone before the amino acid at position 34
  SITE 34: note = Wherein the K at position 24 has been chemically modified via conjugation to methyltetrazine-PEG4-NHS ester
GLFEAIAEFIENGWEGLIEGWYGGRKKRRQRRRK

SEQ ID NO: 122 - protein, 34 aa, synthetic construct
  SITE 34: note = Wherein two copies (monomeric units) of polyethylene glycol are incorporated into the peptide backbone before the amino acid at position 34
  SITE 34: note = Wherein the K at position 24 has been chemically modified via conjugation to methyltetrazine-PEG4-NHS ester
KLFEAIEGFIENGWEGMIDLWNGYGRKKRRQRRK

SEQ ID NO: 123 - protein, 34 aa, synthetic construct
  SITE 34: note = Wherein the C at position 24 is Cys(3-nitro-2-pyridinesulfenyl)
KLFEAIEGFIENGWEGMIDLWNGYGRKKRRQRRC

SEQ ID NO: 124 - protein, 34 aa, synthetic construct
  SITE 34: note = Wherein the C at position 24 is Cys(3-nitro-2-pyridinesulfenyl)
GLFEKIEGFIENGWEGMIDGWYGYGRKKRRQRRC

SEQ ID NO: 125 - RNA, 23 nt, synthetic construct
  source 1..23: mol_type = other RNA
ttgcttctggtgctgcaactngg

SEQ ID NO: 126 - RNA, 23 nt, synthetic construct
  source 1..23: mol_type = other RNA
gagtagcgcgagcacagctangg

SEQ ID NO: 127 - RNA, 23 nt, synthetic construct
  source 1..23: mol_type = other RNA
cctgcctccgctctactcacngg

SEQ ID NO: 128 - RNA, 23 nt, synthetic construct
  source 1..23: mol_type = other RNA
cagtcgcttcctgcctcggangg

SEQ ID NO: 129 - RNA, 22 nt, synthetic construct
  source 1..22: mol_type = other RNA
cagggttctggatatctgtngg

SEQ ID NO: 130 - RNA, 23 nt, synthetic construct
  source 1..23: mol_type = other RNA
aaagaattgatttgataccgngg

SEQ ID NO: 131 - RNA, 23 nt, synthetic construct
  source 1..23: mol_type = other RNA
gtatgctatacgaagttattngg

SEQ ID NO: 132 - RNA, 23 nt, synthetic construct
  source 1..23: mol_type = other RNA
aagtaaaacctctacaaatgngg

SEQ ID NO: 133 - RNA, 23 nt, synthetic construct
  source 1..23: mol_type = other RNA
taatccatcttgttccaccctgt

SEQ ID NO: 134 - RNA, 24 nt, synthetic construct
  source 1..24: mol_type = other RNA
atccatcttgttccaccctgtgca

SEQ ID NO: 135 - RNA, 23 nt, synthetic construct
  source 1..23: mol_type = other RNA
ctaacagttgcttttatcacngg

SEQ ID NO: 136 - RNA, 20 nt, synthetic construct
  source 1..20: mol_type = other RNA
agtacatgaattatgagata

SEQ ID NO: 137 - protein, 1827 aa, synthetic construct
SNATMKRTADGSEFESPKKKRKVSEVEFSHEYWMRHALTLAKRAWDEREVPVGAVLVHNNRVIGEGWNRPIGRHDPTAHAAIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGDSGGSKRTADGSEFEPKKKRKVGSGSKRPAATKKAGQAKKKK

SEQ ID NO: 138 - protein, 18 aa, synthetic construct
KRTADGSEFESPKKKRKV

SEQ ID NO: 139 - protein, 37 aa, synthetic construct
KRTADGSEFEPKKKRKVGSGSKRPAATKKAGQAKKKK

SEQ ID NO: 140 - protein, 387 aa, synthetic construct
SEVEFSHEYWMRHALTLAKRAWDEREVPVGAVLVHNNRVIGEGWNRPIGRHDPTAHAAIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPE

SEQ ID NO: 141 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 142 - protein, 1827 aa, synthetic construct
SNATMKRTADGSEFESPKKKRKVSEVEFSHEYWMRHALTLAKRAWDEREVPVGAVLVHNNRVIGEGWNRPIGRHDPTAHAAIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTVAYSVLVVAKVEKGKSKKLKSVKELLGITIMERSSFEKNPIDFLEAKGYKEVKKDLIIKLPKYSLFELENGRKRMLASAGVLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTINRKQYNTTKEVLDATLIRQSITGLYETRIDLSQLGGDSGGSKRTADGSEFEPKKKRKVGSGSKRPAATKKAGQAKKKK

SEQ ID NO: 143 - protein, 1368 aa, Streptococcus pyogenes [sequence omitted]

SEQ ID NO: 144 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 145 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 146 - protein, 1306 aa, Acidaminococcus sp.
TQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 147 - protein, 1397 aa, synthetic construct
SNATSDKKYSIGLDIGTNSVGWAVITDEYKVPSKKFKVLGNTDRHSIKKNLIGALLFDSGETAEATRLKRTARRRYTRRKNRISYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVED

SEQ ID NO: 148 - protein, 1430 aa, synthetic construct
SRAAPAAKKKKLDNATSDKKYSIGLDIGTNSVGWAVITDEYKVPSKKFKVLGNTDRHSIKKNLIGALLFDSGETAEATRLKRTARRRYTRRKNRISYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIESFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVDGSGSKRPAATKKAGQAKKKKLE

SEQ ID NO: 149 - protein, 1436 aa, synthetic construct
SNATPKKKRKVGGSPKKKRKVGGSPKKKRKVGGSPKKKRKVGIHGVPAATMDKKYSIGLDIGTNSVGWAVITDEYKVPSKKFKVLGNTDRHSIKKNLIGALLFDSGETAEATRLKRTARRRYTRRKNRISYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIESFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVEDPKKKRKV

SEQ ID NO: 150 - protein, 1715 aa, synthetic construct
SNATVDNKFNKEQQNAFYEILHLPNLNEEQRNAFIQSLKDDPSQSANLLAEAKKLNDAQAPKGSAGASDKKYSIGLDIGTNSVGWAVITDEYKVPSKKFKVLGNTDRHSIKKNLIGALLFDSGETAEATRLKRTARRRYTRRKNRISYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVDIGSGSNGSSGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGSPKKKRKVE

SEQ ID NO: 151 - protein, 1493 aa, synthetic construct
SRAAPAAKKKKLDNATVDNKFNKEQQNAFYEILHLPNLNEEQRNAFIQSLKDDPSQSANLLAEAKKLNDAQAPKGSAGASDKKYSIGLDIGTNSVGWAVITDEYKVPSKKFKVLGNTDRHSIKKNLIGALLFDSGETAEATRLKRTARRRYTRRKNRISYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVDGSGSKRPAATKKAGQAKKKKLE

SEQ ID NO: 152 - protein, 1827 aa, synthetic construct
SNATMKRTADGSEFESPKKKRKVSEVEFSHEYWMRHALTLAKRAWDEREVPVGAVLVHNNRVIGEGWNRPIGRHDPTAHAAIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRISYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKVGSGSKRPAATKKAGQAKKKK

SEQ ID NO: 153 - RNA, 24 nt, synthetic construct
  source 1..24: mol_type = other RNA
tttcagtgggggtgaattcagtgt

SEQ ID NO: 154 - RNA, 23 nt, synthetic construct
  source 1..23: mol_type = other RNA
ggggccactagggacaggattgg

SEQ ID NO: 155 - RNA, 23 nt, synthetic construct
  source 1..23: mol_type = other RNA
acaccgctcccataaagccatgg

SEQ ID NO: 156 - RNA, 23 nt, synthetic construct
  source 1..23: mol_type = other RNA
tttatcacaggctccaggaaggg

SEQ ID NO: 157 - DNA, 23 bp, synthetic construct
  source 1..23: mol_type = other DNA
tttatcacaggctccaggaaggg

SEQ ID NO: 158 - DNA, 41 bp, synthetic construct
  source 1..41: mol_type = other DNA
gctaacagttgcttttatcacaggctccaggaagggtttgg

SEQ ID NO: 159 - DNA, 41 bp, synthetic construct
  source 1..41: mol_type = other DNA
cgattgtcaacgaaaatagtgtccgaggtccttcccaaacc

SEQ ID NO: 160 - protein, 10 aa, synthetic construct
YGRKKRRQRR

SEQ ID NO: 161 - protein, 10 aa, synthetic construct
GRKKRRQRRR

SEQ ID NO: 162 - protein, 9 aa, synthetic construct
GRKKRRQRR

SEQ ID NO: 163 - protein, 23 aa, synthetic construct
GLFEAIAEFIENGWEGLIEGWYG

SEQ ID NO: 164 - protein, 23 aa, synthetic construct
GLFEAIEGFIENGWEGMIDGWYG

SEQ ID NO: 165 - protein, 33 aa, synthetic construct
  VARIANT 5: note = Wherein X is A, R, or K
  VARIANT 16: note = Wherein X is A or G
  VARIANT 20: note = Wherein X is L or G
  VARIANT 22: note = Wherein X is N or Y
  VARIANT 24: note = Wherein the amino acid may or may not be present, and if present is Y
KLFEXIEGFIENGWEXMIDXWXGXGRKKRRQRR

SEQ ID NO: 166 - protein, 33 aa, synthetic construct
  VARIANT 1: note = Wherein X is R or G
  VARIANT 5: note = Wherein X is R or K
XLFEXIEGFIENGWEGMIDGWYGYGRKKRRQRR

SEQ ID NO: 167 - protein, 36 aa, synthetic construct
  VARIANT 16: note = Wherein X is A or G
  VARIANT 20: note = Wherein X is G or L
GLFEAIEGFIENGWEXMIDXWNGYGRKKRRQRRREF

SEQ ID NO: 168 - protein, 33 aa, synthetic construct
  VARIANT 16: note = Wherein X is A or G
  VARIANT 17: note = Wherein X is L or M
  VARIANT 19: note = Wherein X is D or E
GLFEAIEGFIENGWEXXIXLWYGYGRKKRRQRR

SEQ ID NO: 169 - protein, 37 aa, synthetic construct
  VARIANT 4: note = Wherein X is a non-coded amino acid
  VARIANT 8: note = Wherein X is a non-coded amino acid
  VARIANT 11: note = Wherein X is a non-coded amino acid
  VARIANT 15: note = Wherein X is a non-coded amino acid
  VARIANT 19: note = Wherein X is a non-coded amino acid
GLFXAIAXFIXNGWXGLIXGWYGGRKKRRQRRRNICK

SEQ ID NO: 170 - protein, 33 aa, synthetic construct
  VARIANT 5: note = Wherein X is A or H
  VARIANT 7: note = Wherein X is E or A
  VARIANT 8: note = Wherein X is G or E
  VARIANT 19: note = Wherein X is D or E
  VARIANT 20: note = Wherein X is G or L
  VARIANT 22: note = Wherein X is E, H, K, R, or N
  VARIANT 33: note = Wherein the amino acid at position 33 may or may not be present, and if present is R
KLFEXIXXFIENGWEGMIXXWXGYGRKKRRQRX

SEQ ID NO: 171 - protein, 33 aa, synthetic construct
  VARIANT 5: note = Wherein X is R, H, A, or K
  VARIANT 7: note = Wherein X is E or A
  VARIANT 8: note = Wherein X is G or E
  VARIANT 20: note = Wherein X is L or G
  VARIANT 22: note = Wherein X is N, Y, K, or E
GLFEXIXXFIENGWEGMIDXWXGYGRKKRRQRR

SEQ ID NO: 172 - protein, 33 aa, synthetic construct
  VARIANT 5: note = Wherein X is A or K
  VARIANT 20: note = Wherein X is G or L
  VARIANT 22: note = Wherein X is N, K, E, or Y
HLFEXIEGFIENGWEGMIDXWXGYGRKKRRQRR

SEQ ID NO: 173 - protein, 34 aa, synthetic construct
  VARIANT 23: note = Wherein the amino acid at position 23 may be present or absent, and if present is Gly
  VARIANT 24: note = Wherein X is Cys(methyltetrazine) or Cys(3-nitro-2-pyridinesulfenyl)
KLFEAIEGFIENGWEGMIDLWNXXYGRKKRRQRR

SEQ ID NO: 174 - protein, 35 aa, synthetic construct
  VARIANT 24: note = Wherein X is Cys(methyltetrazine-PEG4-maleimide), Cys(maleimide), Lys(PEG23)2, Lys(3-nitro-pyridine-2-carboxylic acid), Lys(PEG23)2, Lys(PEG23)2-(3-nitro-pyridine-2-carboxylic acid), or Cys(1,4-bis(bromomethyl)-benzene)
  VARIANT 35: note = Wherein X is Cys(3-nitro-2-pyridine-sulfenyl) or Lys(methyltetrazine-PEG4)
KLFEAIEGFIENGWEGMIDLWNGXYGRKKRRQRRX

SEQ ID NO: 175 - protein, 32 aa, synthetic construct
LFEAIEGFIENGWEGMIDGWYGYGRKKRRQRR

SEQ ID NO: 176 - DNA, 125 bp, synthetic construct
  source 1..125: mol_type = other DNA
atgaagatcatcctgtggctgtgtgtgttcggcctgttcctggccaccatgttccccatcagctggcagatgcccgtggagtcggcctgtcctccgaggactccgccagctccgagagcttcgcc

SEQ ID NO: 177 - protein, 167 aa, Enterobacter sp.
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 178 - protein, 178 aa, Enterobacter sp.
MRRAFITGVFFLSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 179 - protein, 160 aa, Staphylococcus aureus
MGSHMTNDIYFMTLAIEEAKKAAQLGEVPIGAIITKDDEVIARAHNLRETLQQPTAHAEHIAIERAAKVLGSWRLEGCTLYVTLEPCVMCAGTIVMSRIPRVVYGADDPKGGCSGSLMNLLQQSNFNHRAIVDKGVLKEACSTLLTTFFKNLRANKKSTN

SEQ ID NO: 180 - protein, 161 aa, Bacillus subtilis
MTQDELYMKEAIKEAKKAEEKGEVPIGAVLVINGEIIARAHNLRETEQRSIAHAEMLVIDEACKALGTWRLEGATLYVTLEPCPMCAGAVVLSRVEKVVFGAFDPKGGCSGTLMNLLQEERFNHQAEVVSGVLEEECGGMLSAFFRELRKKKKAARKNLSE

SEQ ID NO: 181 - protein, 183 aa, Salmonella enterica
MPPAFITGVTSLSDVELDHEYWMRHALTLAKRAWDEREVPVGAVLVHNHRVIGEGWNRPIGRHDPTAHAEIMALRQGGLVLQNYRLLDTTLYVTLEPCVMCAGAMVHSRIGRVVFGARDAKTGAAGSLIDVLHHPGMNHRVEIIEGVLRDECATLLSDFFRMRRQEIKALKKADRAEGAGPAV

SEQ ID NO: 182 - protein, 164 aa, Shewanella putrefaciens
MDEYWMQVAMQMAEKAEAAGEVPVGAVLVKDGQQIATGYNLSISQHDPTAHAEILCLRSAGKKLENYRLLDATLYITLEPCAMCAGAMVHSRIARVVYGARDEKTGAAGTVVNLLQHPAFNHQVEVTSGVLAEACSAQLSRFFKRRRDEKKALKLAQRAQQGIE

SEQ ID NO: 183 - protein, 161 aa, Caulobacter crescentus
MRTDESEDQDHRMMRLALDAARAAAEAGETPVGAVILDPSTGEVIATAGNGPIAAHDPTAHAEIAAMRAAAAKLGNYRLTDLTLVVTLEPCAMCAGAISHARIGRVVFGADDPKGGAVVHGPKFFAQPTCHWRPEVTGGVLADESADLLRGFFRARRKAKI

SEQ ID NO: 184 - protein, 179 aa, Geobacter sulfurreducens
MSSLKKTPIRDDAYWMGKAIREAAKAAARDEVPIGAVIVRDGAVIGRGHNLREGSNDPSAHAEMIAIRQAARRSANWRLTGATLYVTLEPCLMCMGAIILARLERVVFGCYDPKGGAAGSLYDLSADPRLNHQVRLSPGVCQEECGTMLSDFFRDLRRRKKAKATPALFIDERKVPPEP

SEQ ID NO: 185 - protein, 198 aa, Homo sapiens [sequence omitted]

SEQ ID NO: 186 - protein, 188 aa, Homo sapiens
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKENHERTFKAWEGLHENSVRLSRQLRRILLPLYEVDDLRDAFRTLGL

SEQ ID NO: 187 - protein, 173 aa, Haemophilus influenzae
MDAAKVRSEFDEKMMRYALELADKAEALGEIPVGAVLVDDARNIIGEGWNLSIVQSDPTAHAEIIALRNGAKNIQNYRLLNSTLYVTLEPCTMCAGAILHSRIKRLVFGASDYKTGAIGSRFHFFDDYKMNHTLEITSGVLAEECSQKLSTFFQKRREEKKIEKALLKSLSDK

SEQ ID NO: 188 - protein, 7 aa, Simian virus 40
PKKKRKV

SEQ ID NO: 189 - protein, 16 aa, synthetic construct
KRPAATKKAGQAKKKK

SEQ ID NO: 190 - protein, 9 aa, synthetic construct
PAAKRVKLD

SEQ ID NO: 191 - protein, 11 aa, synthetic construct
RQRRNELKRSP

SEQ ID NO: 192 - protein, 38 aa, synthetic construct
NQSSNFGPMKGGNFGGRSSGPYGGGGQYFAKPRNQGGY

SEQ ID NO: 193 - protein, 42 aa, synthetic construct
RMRIZFKNKGKDTAELRRRRVEVSVELRKAKKDEQILKRRNV

SEQ ID NO: 194 - protein, 8 aa, synthetic construct
VSRKRPRP

SEQ ID NO: 195 - protein, 8 aa, synthetic construct
PPKKARED

SEQ ID NO: 196 - protein, 8 aa, Homo sapiens
PQPKKKPL

SEQ ID NO: 197 - protein, 12 aa, Mus musculus
SALIKKKKKMAP

SEQ ID NO: 198 - protein, 5 aa, Influenza virus
DRLRR

SEQ ID NO: 199 - protein, 7 aa, Influenza virus
PKQKKRK

SEQ ID NO: 200 - protein, 10 aa, Hepatitis B virus
RKLKKKIKKL

SEQ ID NO: 201 - protein, 10 aa, Mus musculus
REKKKFLKRR

SEQ ID NO: 202 - protein, 20 aa, Homo sapiens
KRKGDEVDGVDEVAKKKSKK

SEQ ID NO: 203 - protein, 17 aa, Homo sapiens
RKCLQAGMNLEARKTKK

SEQ ID NO: 204 - protein, 17 aa, synthetic construct
RPAATKKAGQAKKKKLD

SEQ ID NO: 205 - protein, 9 aa, synthetic construct
PKKKRKVED

SEQ ID NO: 206 - protein, 9 aa, synthetic construct
PKKKRKVDT

SEQ ID NO: 207 - protein, 11 aa, synthetic construct
YGRKKRRQRRR

SEQ ID NO: 208 - protein, 33 aa, synthetic construct; identical to SEQ ID NO: 59
  VARIANT 4: note = Wherein X is a non-coded amino acid
  VARIANT 8: note = Wherein X is a non-coded amino acid
  VARIANT 11: note = Wherein X is a non-coded amino acid
  VARIANT 18: note = Wherein X is a non-coded amino acid
  VARIANT 19: note = Wherein X is a non-coded amino acid